(19) United States
(12) Patent Application Publication
(10) Pub. No.: US 2016/0264934 A1
(43) Pub. Date: Sep. 15, 2016

(54) METHODS FOR MODULATING AND ASSAYING m6A IN STEM CELL POPULATIONS

(71) Applicants: THE GENERAL HOSPITAL CORPORATION, Boston, MA (US); The Regents of the University of California, Oakland, CA (US)

(72) Inventors: Cosmas GIALLOURAKIS, Boston, MA (US); Alan C. MULLEN, Brookline, MA (US); Yi XING, Torrance, CA (US)

(73) Assignees: THE GENERAL HOSPITAL CORPORATION, Boston, MA (US); The Regents of the University of California, Oakland, CA (US)

(21) Appl. No.: 15/067,780

(22) Filed: Mar. 11, 2016

Related U.S. Application Data
(60) Provisional application No. 62/131,490, filed on Mar. 11, 2015.

(51) Int. Cl. C12N 5/0735; A01N 1/02; C12Q 1/68; G01N 33/573; C12N 5/077; C12N 5/0793

(52) U.S. Cl. CPC C12N 5/0606; C12N 5/0657; C12N 5/0619; C12Q 1/6888; G01N 33/573; A01N 1/0226; C12N 2501/72; C12N 2506/02; C12Q 2600/158; C12Y 201/01062; C12Y 201/01

(57) ABSTRACT

The present invention generally relates to methods, assays and kits to maintain a human stem cell population in an undifferentiated state by inhibiting the expression or function of METTL3 and/or METTL4, and m⁶A fingerprint methods, assays, arrays and kits to assess the cell state of a human stem cell population by assessing m⁶A levels (e.g. m⁶A peak intensities) of a set of target genes disclosed herein to determine if the stem cell is in an undifferentiated or differentiated state.

### CROSS REFERENCE TO RELATED APPLICATIONS

This application claims benefit under 35 U.S.C. §119(e) of U.S. Provisional Application No. 62/131,490 filed on Mar. 11, 2015, the contents of each of which are incorporated herein by reference in their entireties.

### GOVERNMENT SUPPORT

This invention was made, in part, with government support under NIH Grant Number DK090122 awarded by National Institutes of Health. The Government of the U.S. has certain rights in the invention.

### FIELD OF THE INVENTION

The present invention relates to arrays and methods for characterizing stem cell populations assessing transcription wide distribution of m⁶A methylation to characterize and permit selection of stem cell lines for further use, and to modulation of METTL3, e.g., inhibition to maintain stem cells in an undifferentiated state or activation of METTL3 to promote differentiation along endoderm lineages.

### BACKGROUND OF THE INVENTION

Reversible chemical modifications on messenger RNAs have emerged as prevalent phenomena that may open a new field of “RNA epigenetics”, akin to the diverse roles that DNA modifications play in epigenetics (reviewed by Fu and He, 2012; Sibbritt et al., 2013). N6-methyl-adenosine (m⁶A) is the most prevalent modification of mRNAs in somatic cells, and dysregulation of this modification has already been linked to obesity, cancer, and other human diseases (Sibbritt et al., 2013). m⁶A has been observed in a wide range of organisms, and the known methylation complex is conserved across eukaryotes (Bokar et al., 1997, Bujnicki, 2002 #375). In budding yeast, the m⁶A methylation program is activated by starvation and required for sporulation (Agarwala et al., 2012; Clancy et al., 2002; Schwartz et al., 2013; Shah and Clancy, 1992). In Arabidopsis, the methylase responsible for m⁶A modification, MTA, is essential for embryonic development, plant growth and patterning (Bodi et al., 2012; Zhong et al., 2008), and the Drosophila homolog IME4 is expressed in ovaries and testes and is essential for viability (Hongay and Orr-Weaver, 2011).

While m⁶A has been suggested to affect almost all aspects of RNA metabolism, the molecular function of this modification remains incompletely understood (Niu et al., 2013). Importantly, m⁶A modification(s) are reversible in mammalian cells. The fat-mass and obesity associated protein, FTO, has m⁶A demethylase activity (Jia et al., 2011) and, ALKBH5, also a member of the alphaketoglutarate-dependent dioxygenases protein family, has also been shown to act as m⁶A demethylase, with particular importance in spermatic development (Zheng et al., 2013) Manipulating global m⁶A levels has implicated m⁶A modifications in a variety of cellular processes including nuclear RNA export, control of protein translation and splicing (Dominissini et al., 2012; Gulati et al., 2013; Hess et al., 2013; Zheng et al., 2013). Recently, it has been suggested that m⁶A modification may also play a role in controlling transcript stability based on the functional characterization of the YTH domain family of “reader” proteins which specifically bind m⁶A sites and recruit the linked transcripts to RNA decay bodies (Kang et al., 2014; Wang et al., 2014a).

Whereas the DNA methylome undergoes dramatic reprogramming during early embryonic life, the developmental origins and functions of m⁶A in mammals are incompletely understood. Furthermore, the degree of evolutionary conservation of m⁶A sites is not known in ESCs. Therefore, there is a need in the art for effective and efficient methods for assessing m⁶A mRNA methylome in stem cells and human stem cells, for example, to characterize and validate cells, including human pluripotent stem cells, and for determining the quality and cell state of a human stem cell populations, e.g., prior to its use, e.g., in therapeutic administration, disease modeling, drug development and screening and toxicity assays etc.

### SUMMARY OF THE INVENTION

The present invention is directed to, in part, methods, compositions and kits to maintain a stem cell population, such as a human stem cell population, in an undifferentiated state, comprising contacting the stem cell population with an inhibitor of METTL3 or METTL4. In some embodiments, the methods, compositions and kits as disclosed herein relate to methods to prevent a stem cell population differentiating along an endoderm lineage. Other aspects of the technology described herein relates to methods, compositions and kits to promote a stem cell population to differentiate along an endoderm lineage. Moreover, another aspect of the technology described herein relates to methods, assays, arrays and kits for performing m⁶A analysis of RNA from stem cell populations to characterize the cell state of the cell population, which can be used, for example, as a quality control for the stem cell population. In some embodiments, the stem cell population is a human stem cell population, e.g., a hESC cell population or other human stem cell line.

N6-methyl-adenosine (m⁶A) is the most abundant covalent modification on messenger RNAs in somatic cells and is linked to human diseases, but its functions in mammalian development are poorly understood. Furthermore, while the m⁶A RNA modification pathway is linked to developmental decisions in lower eukaryotes, little is known concerning the dynamic extent, conservation and potential function(s) of the m⁶A modification in human development. Herein, the inventors demonstrate a genome-wide analysis of m⁶A modifications in human embryonic stem cells (hESCs) differentiated towards endoderm. m⁶A sites are observed on thousands of transcripts including those encoding master regulators of hESC identity and differentiation. A comparative genomic analysis of m⁶A maps in mouse and human ESCs reveals a conserved set of methylated genes and sites of modification. Moreover, human endoderm differentiation is distinguished by the dynamic regulation of rn6A peak intensities. Importantly, we demonstrate that hESCs are reliant on the m⁶A methyltransferase component METTL3 for normal endoderm differentiation. Thus, the inventors reveal a novel layer of hESC regulation at the epitranscriptomic level.

Further, it is to be understood that m6A modification also is involved in differentiation to other cell types, such as, but not limited thereto, iPSCs, adult stem cells, Sertoli cells and neural stem cells, for example.

Moreover, the inventors have performed global sequence analysis of mRNAs immuneprecipitated with a m⁶A RNA-specific antibody to define the mRNA methylome in human embryonic stem cells. In particular, the inventors have discovered a function of m⁶A by mapping the m⁶A methylome in both mouse and human embryonic stem cells (ESCs). The inventors discovered that thousands of messenger and long noncoding RNAs have conserved m⁶A modification, including transcripts encoding multiple core pluripotency transcription factors, including but not limited to Nanog and Sox2. m⁶A was discovered to be enriched over 3′ untranslated regions at defined sequence motifs, and importantly marks unstable transcripts, including transcripts that need to be turned over upon differentiation. Importantly, the inventors have discovered that the m⁶A-modified mRNAs include multiple core pluripotency factors and transcripts involved in development and the cell cycle, and were frequently located near stop codons, at the beginning of 3′ untranslated regions (3′UTR) and in the long internal exons, indicating that m6A site is tied to functional roles in regulating the RNA life cycle and marks the RNA for turn-over. In particular, the inventors discovered that while unmodified transcripts and m6A-modified transcripts had similar rates of transcription, the m⁶A mRNAs had shorter half-lives and reduced translation efficiencies, demonstrating a role for m⁶A-modification in influencing human stem cell RNA turn-over and the fate of the transcript.

To date, the functions of m⁶A in mammalian cells have only been examined by RNAi knockdown. Depletion of METTL3 and METTL14 in human cancer cell lines led decreased cell viability and apoptosis, leading to the interpretation that m⁶A is important for cell viability (Dominissini et al., 2012; Liu et al., 2014).

Here, the inventors assessed the conservation of the m⁶A methylome at the level of gene targets and function in human ESCs. Using genetic inactivation or depletion of mouse and human Mettl3 (one of the known m⁶A methylases), the inventors discovered a decrease in m⁶A levels (i.e. m⁶A erasure) on select target genes, a prolonged Nanog expression upon differentiation, and impaired ESC's exit from self-renewal towards differentiation into several lineages in vitro and in vivo. Importantly, the inventors demonstrate that inhibition or knock-down of Mettl3 in human ESC increased self-renewal and proliferation, but reduced their ability to different ate along specific lineages, in particular endoderm lineages. This is in contrast to the report by Wang and colleagues (Wang et al., 2014, Nat. Cell Biol., 16, 191-198) which report Mettl3 and Mettl4 knockdown in mouse ESCs lead to decreased self-renewal and regeneration, and ectopic differentiation (see., review articles Jalkanen et al., Cell Stem Cell, 2014, 15(669-670), “Stem cell RNA epigenetics: M⁶Arking your territory” and Zhao et al., Genome Biology, 2015, 16; 45, “Fate by RNA methylation: m6A steers stem cell pluripotency”.). Furthermore, Geula et al., (Science, 2015; 347(6225); 1002-1006) show that in native pluripotent mouse ESCs, knockdown of Mettl3 blocked differentiation, whereas knockdown of Mettl3 in differentiation-primed mouse ESCs (mESCs) reduced stem cell self-renewal. This is in contrast with the present invention which demonstrate that knock-down of METTL3 in human ESCs led to the unexpected finding of increased self-renewal and proliferation, and that m⁶A and Mettl3 in particular are not required for ESC growth but rather, are required for stem cells to adopt new cell fates.

Thus, the inventors have discovered that, in human stem cell populations in particular, m⁶A on RNA demonstrates the transcriptome flexibility and is required for human stem cells to differentiate to specific lineages. In particular, the inventors have discovered that m⁶A-modifications in the RNA (in mRNA transcripts, non-coding regions and in non-coding RNAs) of human stem cell populations serve as stem cells internal “quality control” as the m⁶A marks the mRNA as having passed a quality control test in the cell, as stem cells cannot differentiate without m⁶A-modifications on key transcripts.

Thus, a key concept of the technology described herein relates to the discovery that inhibition of the METTL3 enzyme prevents human stem cells from differentiating. Stated a different way, the inventors have discovered a process which “locks” hESCs into their pluripotent state (see FIG. 5). Depleting METTL3 or METTL4 levels (e.g., using RNAi) and/or inhibiting METTL3 or METTL4 enzyme function, (e.g., using METTL3 or METTL4 small molecule inhibitors) allows human stem cell populations to remain in a pluripotent, undifferentiated state, and prevents them from spontaneously differentiating along specific lineages. This is useful for maintaining human stem cell populations for long periods of time, e.g., in culture and after multiple passages without the risk of the human stem cell line differentiating and/or changing phenotype. Furthermore, if a specific hESC or iPSC cell subclone is identified that has particular beneficial properties, inhibition of METTL3 and/or METTL4 is useful to propogate the stem cell line and prevent them from differentiating, therefore enabling consistency amoung aliquots of a stem cell population. Importantly, while much of the field of stem cell research focuses on methods to differentiate stem cells into specific lineages, there limited options on methods to keep a stem cell population in an undifferentiated state. This is useful as stem cells are typically cultured in a defined media to prevent differentiation, however, and some cells spontaneously differentiate regardless of the culture media used.

Another aspect of the technology disclosed herein relates to the use of the intensity of m6A sites of methylation (i.e., m6A peak intensity) as a quantitative metric or measure to distinguish cell states. Stated another way, the intensity of m6A sites of methylation (i.e., m6A peak intensity) of a set of specific target gene, e.g., at least 10 or more selected from Table 1 or Table 2, can be used to “fingerprint” a cell state, e.g., determine the cell state of the stem cell population, i.e., to determine if the stem cell population is pluripotent (i.e., in an undifferentiated pluripotent state) or if the human stem cell population has differentiated along a cell lineage pathway. Importantly, using the intensity of m6A sites of methylation (i.e., m6A peak intensity) of specific target genes is independent of gene expression levels, which is the current standard of analysis of stem cell populations.

Accordingly, another aspect of the technology described herein relates to methods, compositions, assays, arrays and kits to characterize a stem cell population, such as a human stem cell population, comprising performing m⁶A analysis on the RNA obtained from the population of stem cells, and assessing the intensity of the m⁶A levels of the mRNA of at least 10 genes selected from any of those in Table 1, or Table 2 as disclosed herein.

Another aspect of the technology described herein relates to methods, compositions, assays, arrays and kits for assessing m⁶A levels in the RNA obtained from a population of stem cells, e.g., human stem cells. In some embodiments, the method comprises (i) measuring the m⁶A levels of least 10 mRNA transcripts selected from any of those listed in Table 1 or Table 2, for example by contacting an array with RNA isolated from a cell population, where the array comprises at least 10 or more oligonucleotides that hybridize to at least 10 mRNA transcripts, or to at least 10 3′UTR or other untranslated regions of at least 10 genes selected from any of those listed in Table 1 or Table 2, and (ii) contacting the array with at least one reagent which binds to m6A in the RNA, such as an anti-m⁶A antibody, or fragment thereof, such as an anti-m⁶A antibody which is fluorescently labeled or otherwise has a detectable label, therefore allowing the measurements of the levels of m6A in the at least selected 10 mRNA transcripts, or to at least 10 3′UTR or other untranslated regions of at least 10 genes selected from any of those listed in Table 1 or Table 2.

A further aspect of the technology described herein relates to methods, compositions, assays, arrays and kits for use in a method for determining the cell state of a stem cell population comprising performing the assay of claim 10, and comparing the levels of m6A (i.e., peak intensities) of at least 10 genes selected from any of Table 1 in the RNA from the stem cell population with the levels of m6A (i.e., peak intensities) in a reference stem cell population, and based on this comparison, determining the cell state of the stem cell population.

Another aspect of the present invention relates to a kit comprising: (i) an array composition for characterizing the cell state of a population of stem cells, comprising at least 10 oligonucleotides that hybridize to the RNA (i.e., mRNA transcripts, 3′UTR or other untranslated RNAs) of at least 10 genes selected from any of those in Table 1 or Table 2 as disclosed herein; and (ii) at least one regent to detect the m6A in RNA, such as, for example, an anti-m⁶A antibody, or fragment thereof, for example an anti-m6A antibody or fragment thereof which is detectably labeled (e.g., with a florescent label, colorimetric marker etc.).

In some embodiments, the kit comprises a computer readable medium comprising instructions on a computer to compare the measured levels of m6A (i.e., peak intensities) from the test stem cell population with reference levels of the same RNA transcripts assessed. In some embodiments, the kit comprises instructions to access to a software program available online (e.g., on a cloud) to compare the measured levels of the m6A (i.e., peak intensities) from the test stem cell population, e.g., human stem cell population, with reference levels of m6A for the same RNAs assessed from a reference stem cell population, e.g., human stem cell population.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIGS. 1A-H show topology and characterization of m⁶A target genes. FIG. 1A shows UCSC Genome browser plots of m⁶A-seq reads along indicated mRNAs. Grey reads are from non-immunoprecipitated control input libraries and red reads anti-m⁶A immunoprecipitation libraries. The y-axis represents normalized number of reads. Blue thick boxes represent the open reading frame while the blue line represents the untranslated regions. FIG. 1B is a model of genes involved in maintenance of stem cell state (adapted from Young et al., 2011). Red hexagons represent modified mRNAs. FIG. 1C is a heatmap with log 10 (p-vlaue) of gene set enrichment analysis for m⁶A modified genes. FIG. 1D shows a sequence motif identified after analysis of m⁶A enrichment regions. FIG. 1E shows the normalized distribution of m⁶A peaks across 5′ UTR, CDS and 3′UTR of mRNAs for peaks common to all samples. FIG. 1F shows the graphical representation of frequency of m⁶A peaks and methylation motifs in genes, divided into 5 distinct regions. FIG. 1G shows multi-exon coding and non-coding RNAs exhibit enrichment of m⁶A sites near the last exon-exon splice junction. The distribution of m⁶A peaks across the length of the mRNAs (n=5070) and non-coding RNAs (n=51) is shown. FIG. 1H is a scatter plot representation of m⁶A enrichment score (on the X axis) and gene expression level (on the Y axis) for each m⁶A peak. FIG. 1I shows a Box plot representing the half-life for transcripts with at least one modification site and transcripts with no modification site identified.

FIGS. 2A-2F show characterization of Mettl3 knock out cells. FIG. 2A is a western blot for Mettl3 and PARP in wild type and two cell lines with CRISPR induced loss of protein. DD, DNA damaging agent. Actin is used as loading control. FIG. 2B shows m⁶A ratio determined by 2D-TLC in wild type and Mettl3 KO. FIG. 2C shows alkaline phosphatase staining of wild type and Mettl3 knock out cells. FIG. 2D is a box plot representation of colony radius for wild type and Mettl3 mutant cells. Experiments were performed in triplicate, with at least 50 colonies measured for each replicate. FIG. 2E shows nanog staining of colonies of wild type and two cell lines with CRISPR induced loss of protein. FIG. 2F is a cell proliferation assay showing wild type and two cell lines with CRISPR induced loss of Mettl3 protein.

FIGS. 3A-3F show mettl3 loss of function impairs ESC ability to differentiate. FIG. 3A shows the percentage of embryoid bodies with beating activity in Mettl3 KO and wild type control cells (right panel). Representative images of bodies stained for MHC and DAPI (center panel) and mRNA levels of Nanog and Myh6 in Mettl3 KO cells in relation to wild type control cells. * represents p-value<0.05. FIG. 3B shows the percentage of colonies with Tuj1 projections in Mettl3 KO and wild type control cells (right panel). Representative images of bodies stained for Tuj1 and DAPI (center panel) and mRNA levels of Nanog and Tuj1 in Mettl3 KO cells in relation to wild type control cells. * represents p-value<0.05. FIG. 3C shows the weight differences between teratomas generated from wild type and Mettl3 knock out cells. Tumors are paired by animal (n=5). FIG. 3D shows the representative sections of teratomas stained with hematoxylin and eosin at low magnification. The bar represents 1000 FIG. 3E shows immunohistochemistry images with antibody against Ki67. FIG. 3F shows immunohistochemistry images with antibody against Nanog. The bar represents 100 μm.

FIGS. 4A-4F shows the impact of loss of Mettl3 on the mESC methylome. FIG. 4A shows the cumulative distribution function of log 2 peak intensity of m6A modified sites. FIG. 4B shows the sequencing read density for input (grey) and after m⁶A IP (red) for Nanog. Blue thick boxes represent the open reading frame while the blue line represents the untranslated regions. FIG. 4C is a heatmap representing IP enrichment values for peaks with statistically significant difference between wild type and Mettl3 mutant. FIG. 4D is a model of genes involved in maintenance of stem cell state (adapted from Young et al., 2011), representing transcripts with loss of m6A modification in Mettl3−/− cells. FIG. 4E shows the percentage of input recovered after m⁶A IP measured by nanostring. FIG. 4F shows the mRNA levels of Nanog and Oct4 after PolII inhibition relative to untreated sample in wild type and Mettl3 KO cells.

FIGS. 5A-5J show m⁶A-seq profiling of hESC during endoderm differentiation. FIG. 5A shows m⁶A-seq was performed in resting (i.e. undifferentiated) human H1-ESCs (T0) and after 48 hrs of Activin A induction towards endoderm (mesoendoderm) (T48). FIG. 5B is a Venn diagram of the overlap between high-confidence T0 and T48 m⁶A peaks and methylated genes (parenthesis). FIG. 5C shows a sequence motif identified after analysis of m⁶A enrichment regions. FIG. 5D shows UCSC Genome browser plots of m⁶A-seq reads along indicated RNAs. Grey reads are from non-immunoprecipitated control input libraries and red (T0) or blue (T48) reads are from anti-m⁶A immunoprecipitation libraries. The y-axis represents normalized number of reads. Blue thick boxes represent the open reading frame while the blue line represents the untranslated regions. Key regulators of stem cell maintenance (left) and master regulators of endoderm differentiation (right) are represented. FIG. 5E shows a Scatterplot of m⁶A peak intensities between two different time points (T0 versus T48) of the same biological replicate with only “high-confidence” T0 or T48 specific peaks supported by both biological replicates highlighted. FIG. 5F shows UCSC Genome browser plots of m⁶A-seq reads along indicated mRNAs in undifferentiated (T0) versus differentiated cells (T48). The grey reads are from non-immunoprecipitated control input libraries. The red and blue reads are from the anti-m⁶A RIP of T=0 and T=48 samples respectively. The y-axis represents normalized number of reads. Blue thick boxes represent the open reading frame while the blue line represents the untranslated regions. FIG. 5G shows that differential intensities of m⁶A peaks (DMPIs) identify hESC cell states T0 vs T48 hrs. Z score scaled Log 2 peak intensities of DMPIs are color-coded according to the legend. The peaks and samples are both clustered by average linkage hierarchical clustering using 1-Pearson correlation coefficient of log 2 peak intensity as the distance metric. FIG. 5H show the number of peaks per exon normalized by the number of motifs (on sense strand) in the exon. The error bars represent standard deviations from 1000 times of bootstrapping. FIG. 5I show the normalized distribution of m⁶A peaks across the 5′UTR, CDS, and 3′UTR of mRNAs for T0 and T48 m⁶A peaks. FIG. 5J is a box plot representing the half-life for transcripts, with transcripts separated according to enrichment score. Genes with higher levels of m⁶A enrichment in hESCs tend to exhibit lower mRNA stability in human induced pluripotent cells (iPSCs).

FIGS. 6A-6F show the evolutionary conservation and divergence of the m⁶A epi-transcriptomes of human and mouse ESCs. FIG. 6A is a Venn diagram showing a 62% overlap between methylated genes in M. musculus (purple) and H. sapiens (red) embryonic stem cells (p value=3.5×10⁻⁹²; Fisher exact test). FIG. 6B shows the m⁶A peaks that could be mapped to orthologous genomic windows between mouse and human were identified. The intensities of m⁶A-seq signals in human and mouse ESCs were shown for m⁶A peaks found to be unique in mouse (blue), unique in human (red), and conserved between human and mouse (black). FIG. 6C is a boxplot of peak intensities of m⁶A sites conserved (“common”) or not conserved (“specific”) in mouse and human ESCs. (p values=1.3×10⁻¹⁵ and 8.7×10⁻²³ respectively). FIG. 6D, FIG. 6E and FIG. 6F show UCSC Genome browser plots of m⁶A-seq reads along indicated mRNAs. The grey reads are from non-immunoprecipitated control input libraries and the purple and red reads are from the anti-m⁶A RIP of mESCs and hESCs (T0) respectively. FIG. 6D shows representative examples of species-specific m⁶A modifications in mouse ESCs. FIG. 6E shows species-specific m⁶A modifications in human ESCs. FIG. 6F shows representative examples of conserved m⁶A modifications at the gene and site level are represented. Genes such CHD6 have a conserved m⁶A peak location at its 3′UTR as well as mouse and human specific m⁶A peaks at conserved but distinct exons.

FIGS. 7A-7F shows METTL3 is required for normal human ESC endoderm differentiation.

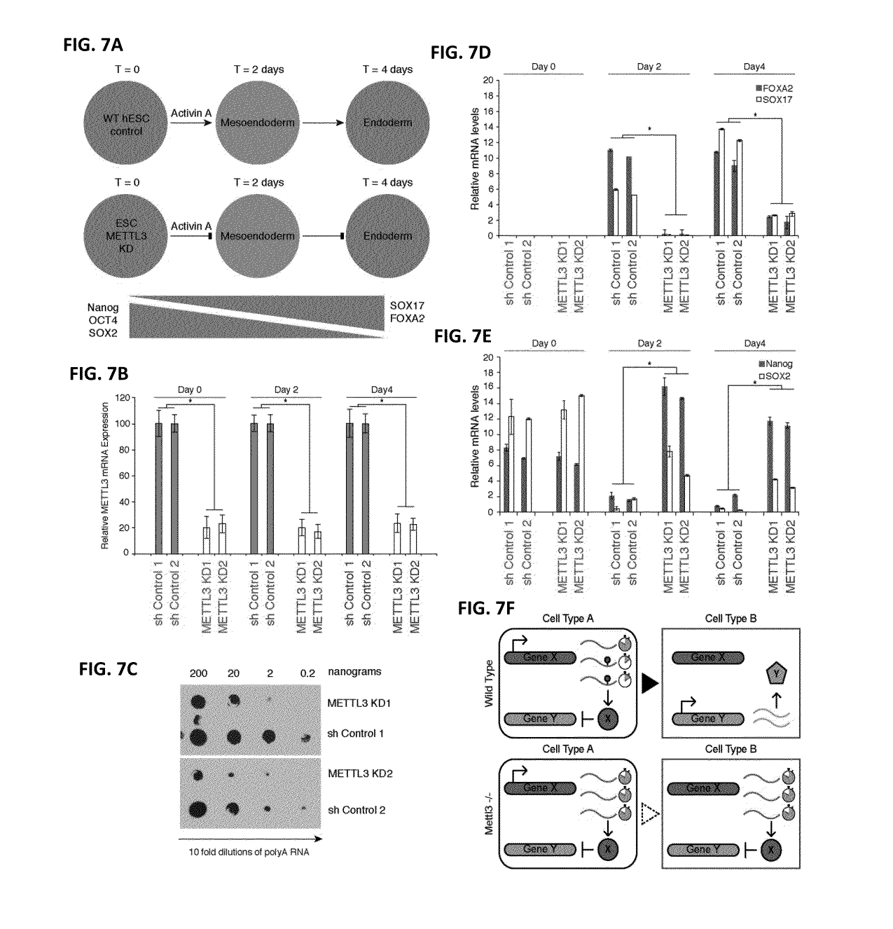

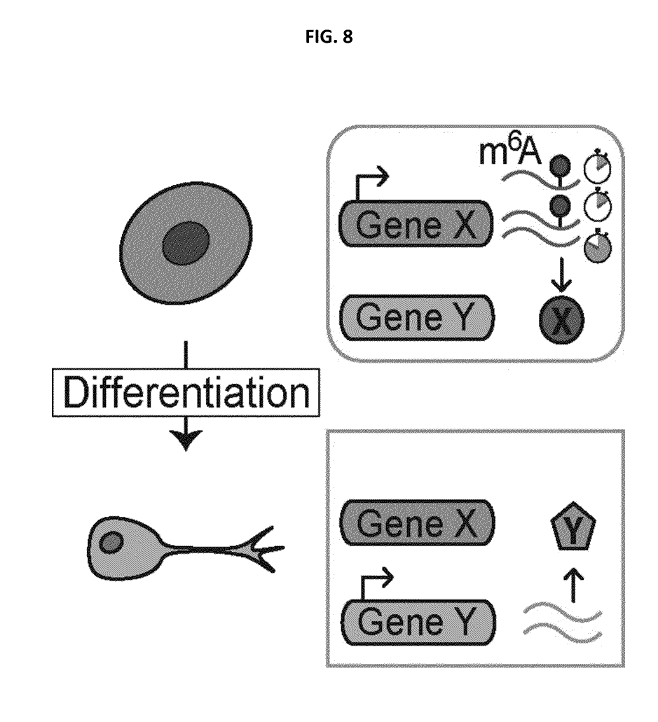

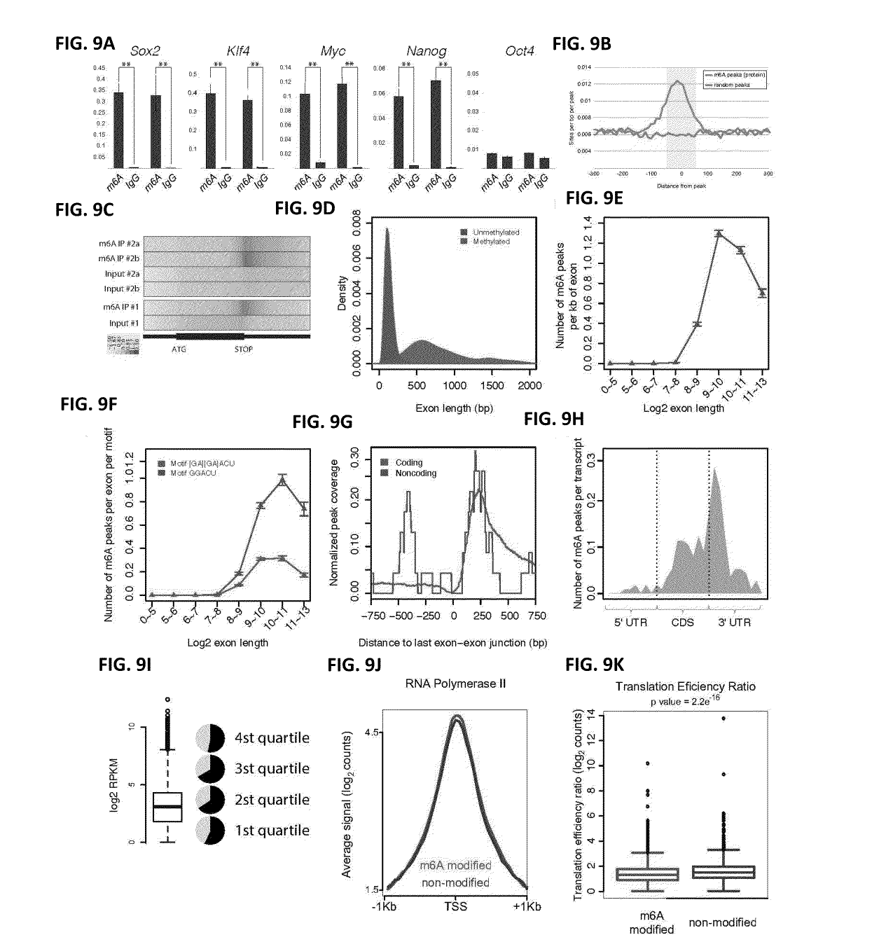

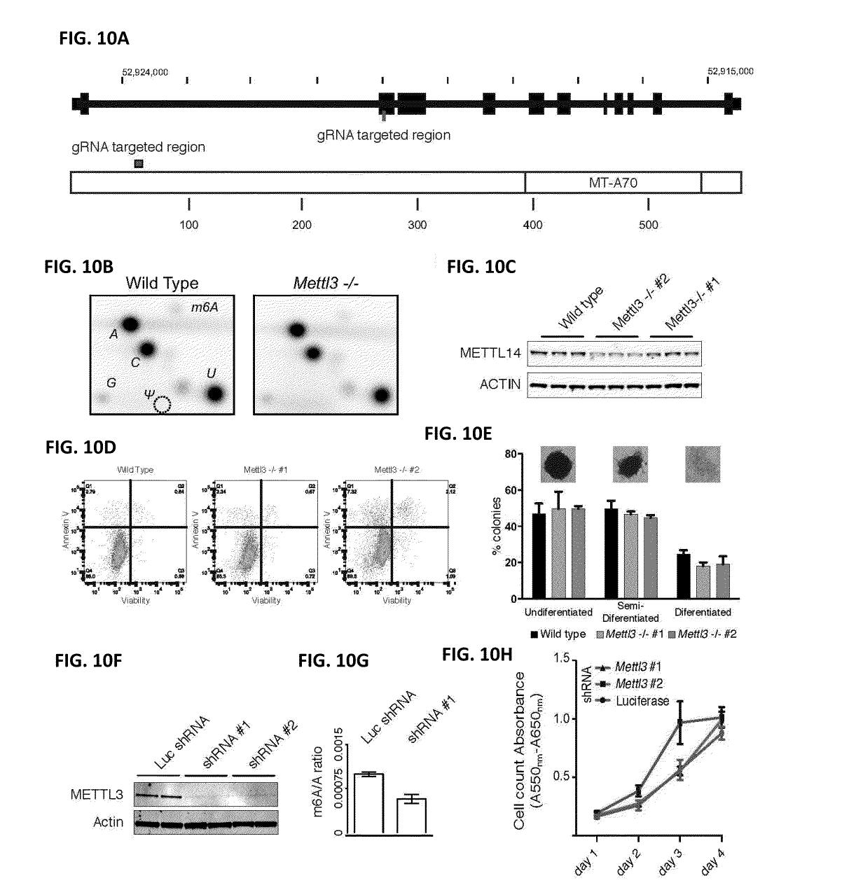

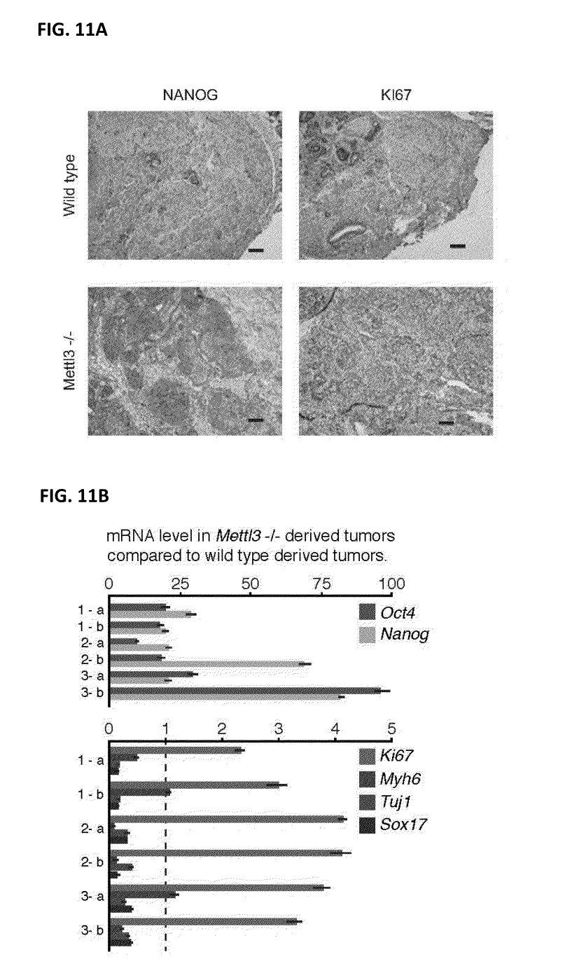

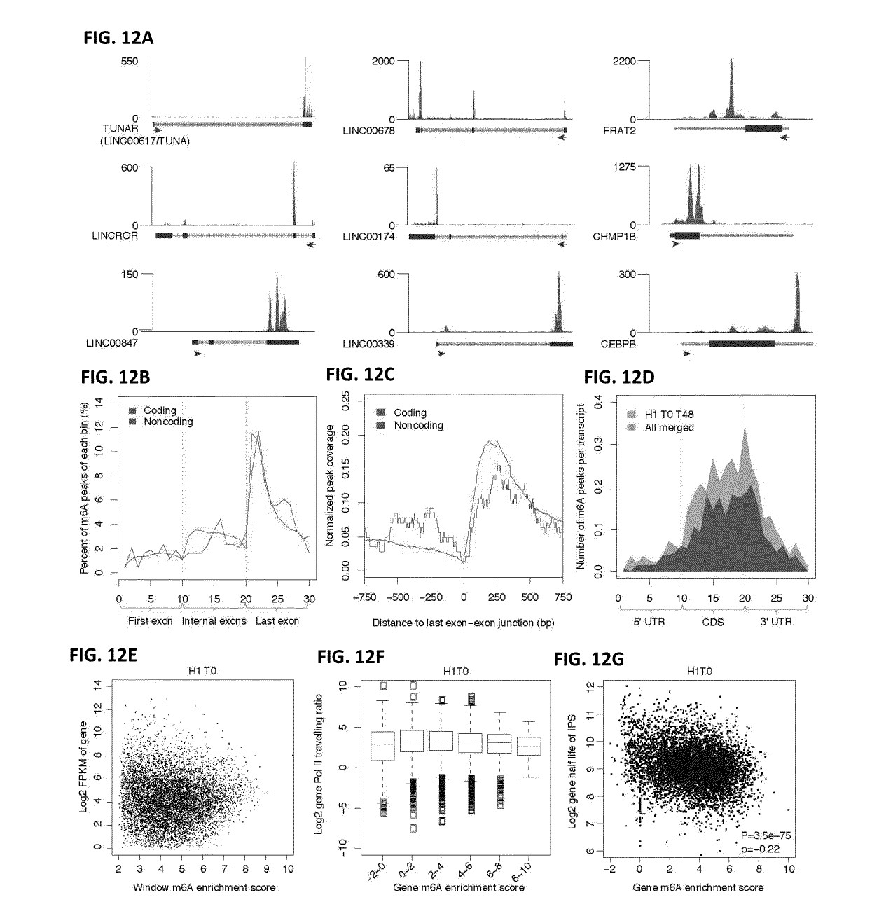

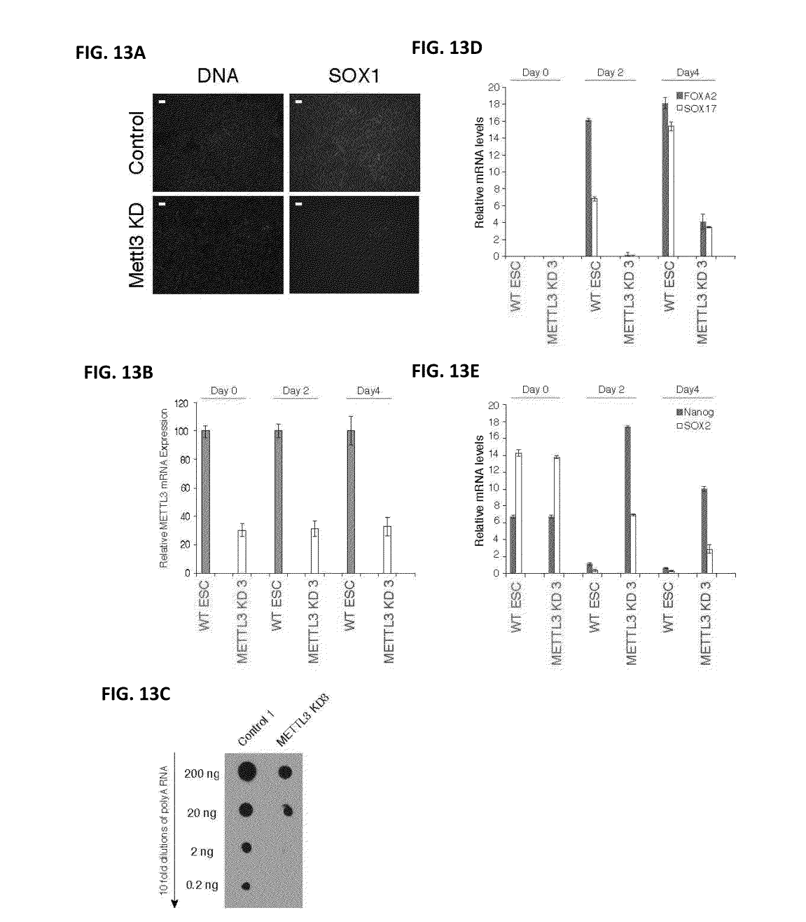

Model of METTL3 function(s). FIG. 7A shows hESC cells transfected with anti-METTL3 shRNA (KD) as well control shRNA and stable hESC colonies were obtained after drug selection. Two independent clones were subjected to endodermal differentiation with Activin A and examined at various indicated time points. A schematic of the trends of gene expression for indicated markers of stem maintenance and endoderm differentiation is also shown. FIG. 7B shows Knockdown of METTL3 leads to a reduction in METTL3 mRNA levels. qRT-PCR for METTL3 mRNA was performed from RNA extracted from hESC cells with control shRNA versus anti-METTL3 shRNA (KD) across the three indicated time points during endodermal differentiation (n=2 independent generally ES cell knockdown and control clones shown; error bars represent standard deviation of qPCRx3 per time point). FIG. 7C shows knockdown of METTL3 leads to a reduction in m⁶A levels. An anti-m⁶A dot blot was performed on 10× fold dilutions of polyA selected RNA from hESC cells derived from control shRNA versus anti-METTL3 shRNA clones. FIG. 7D shows knockdown of METTL3 prevents the normal reduction of stem maintenance/marker genes. qRT-PCR was performed for indicated genes and time points. (n=2 independent generally ES cell knockdown and control clones shown; error bars represent standard deviation of qPCRx3 per time point). FIG. 7E shows knockdown of METTL3 leads to a delayed and reduced induction of endodermal marker genes. qRT-PCR was performed on indicated genes and time points (n=2 independent generately ES cell knockdown and control clones shown; error bars represent standard deviation of qPCR×3 per time point). FIG. 7F shows that m6A marks transcripts for faster turn-over. Upon transition to new cell fate, m6A marked transcripts are readily removed to allow the expression of new gene expression networks. In the absence of m6A, the unwanted presence of transcripts will disturb the proper balanced required for cell fate transitions.

FIG. 8 is a schematic representation showing that selected mRNA transcripts (i.e., core pluripotent factor transcripts) are m⁶A and translated for a time period, allowing self-renewal and proliferation of the pluripotent human stem cell, whereas after differentiation, the non m⁶A mRNA transcripts are predominantly translated.

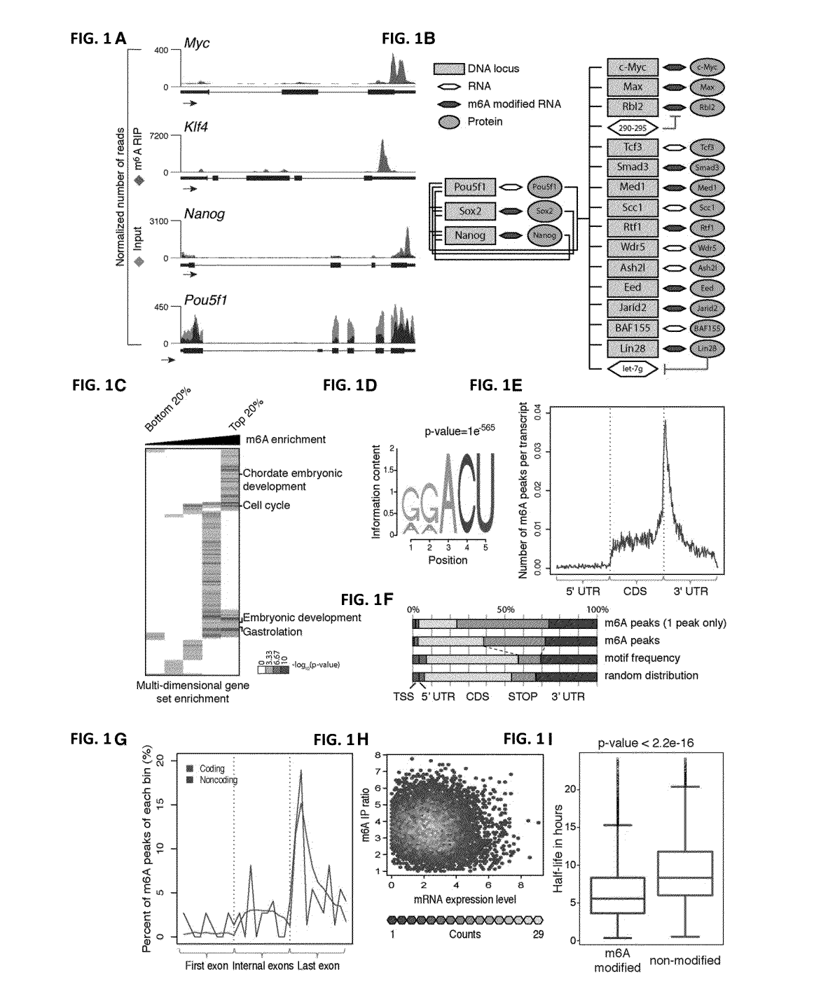

FIGS. 9A-9K shows topology and characterization if n6A target genes and is related to FIG. 1. FIG. 9A shows m6A enrichment determined by qRT-PCR. Vertical axis represents percentage of recovery. Error bars represent standard deviation of the ΔΔCT value. ** represents p-value<0.01. FIG. 9B is a histogram representing motif density in m6A peaks (Blue) and a random control group of windows (Red). FIG. 9C shows metagene representation of read density obtained in input and after m6A enrichment for genes with at least one modification. Black thick box represents the open reading frame while the black line represents the untranslated regions. The CDS and 3′ UTR are divided in 100 bins, while the 5′ UTR is divided in 50 bins. FIG. 9D shows the exon length distribution of methylated vs unmethylated internal exons of coding genes is shown. FIG. 9E shows the number of peaks per exon normalized by exon length is shown for different bins of exon length. The error bars represent standard deviations from 1000 times of bootstrapping. FIG. 9F shows the number of peaks per exon normalized by the number of motifs (on sense strand) in the exon is shown. The error bars represent standard deviations from 1000 times of bootstrapping. FIG. 9G shows the density of m6A-seq read coverage increases sharply downstream of the last exon-exon splice junction in both coding and non-coding RNAs. FIG. 9H shows the percentage of m6A peaks that fall into normalized bins across the 5′UTR, CDS, and 3′UTR of single-exon genes is shown.

FIG. 91 shows pie charts representing the fraction of genes with m6A modification for each quartile of expression. Black area represents modified genes. FIG. 9J shows the average coverage of Pol2 signal at the transcriptional start site of modified and unmodified genes. FIG. 9K is a box plot representing translation efficiency as measured by ribosome profile.

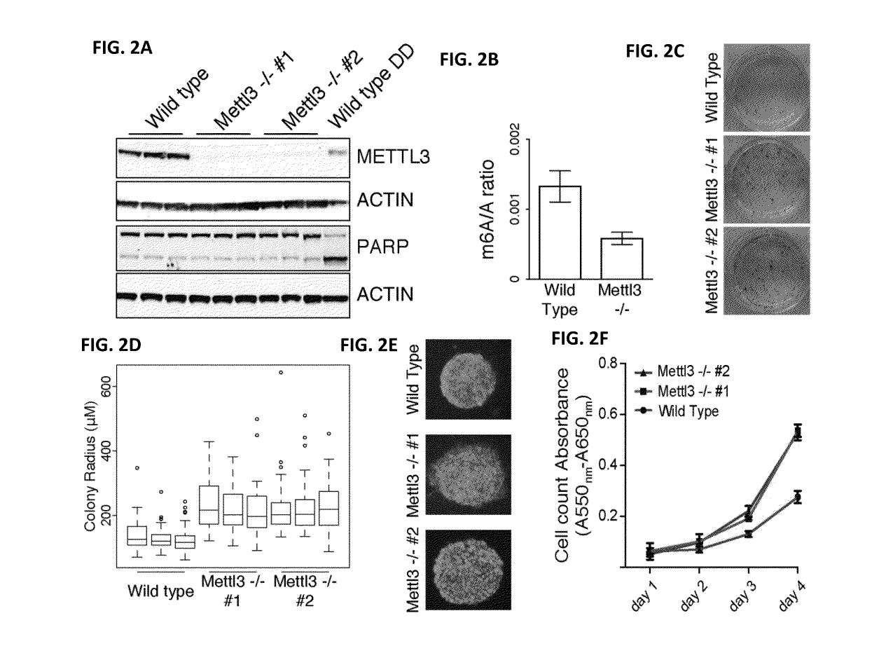

FIGS. 10A-10H show the characterization of Mettl3 knockout cells (FIG. 10 is related to FIG. 2F). FIG. 10A is representative example of DNA sequencing of mutations induced by CRISPR genome engineering. The grey areas indicate codons in the open reading frame. Representation of the Mettl3 locus, and Mettl3 protein, with the CRISPR targeted region marked in red. FIG. 10B shows representative examples of 2D-TLC plates for mESC wild type and Mettl3−/− mutant. Nucleotide positions are indicated in the leftmost panel. FIG. 10C is a Western blot for Mettl14 in wild type and two cell lines with Mettl3 KO cell lines. Actin is used as loading control. FIG. 10D shows FACS plots of Annexin V and Aqua Live/Dead fixable Viability dye for Wild type and two Mettl3 KO cell lines. FIG. 10E shows quantification of colony morphologies for Wild type and two Mettl3 KO cell lines. Experiment performed in triplicate, with at least 50 colonies counted per replicate. Error bars represent standard deviation. FIG. 10F is a Western blot for Mettl3 in wild type and two independent Mettl3 shRNAs. Actin is used as loading control. FIG. 10G shows the m6A ratio, determined by 2D-TLC, in wild type and Mettl3 shRNA line. FIG. 10H shows a cell proliferation assay of wild type and two independent Mettl3 shRNA lines.

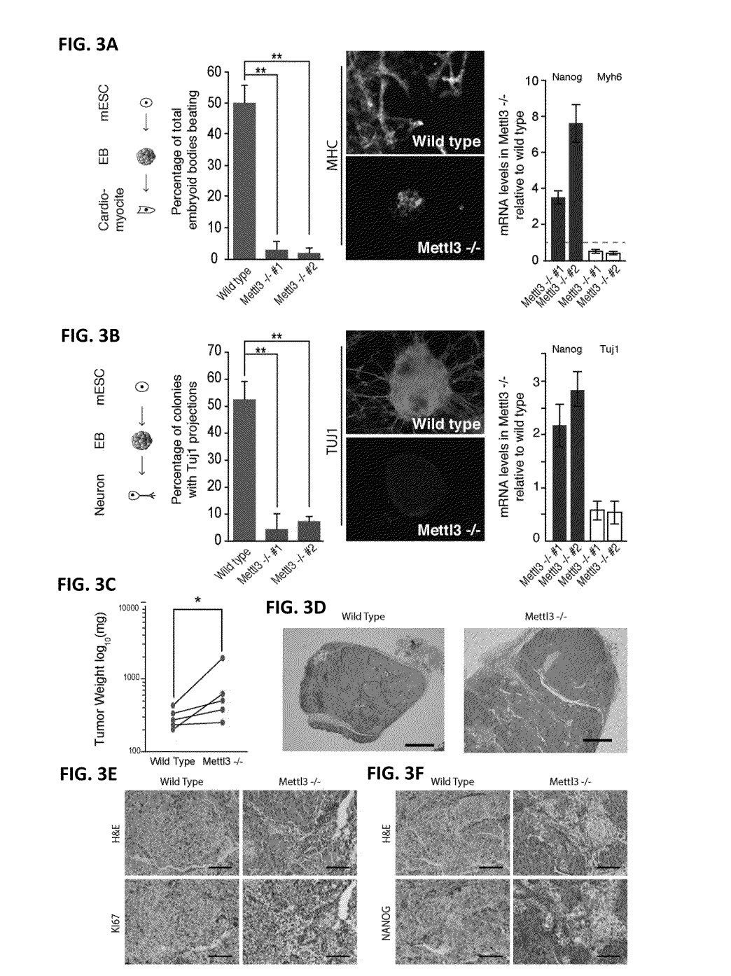

FIGS. 11A-11B shows Mettl3 loss of function impairs ESC ability to differentiate (and is related to FIGS. 2E and 2F). FIG. 11A shows representative sections of teratomas stained with hematoxylin and eosin (left), and immunohistochemistry with antibody against Nanog (center) and Ki67 (right). The bar represents 100 μm. (related to FIG. 3D). FIG. 11B shows relative mRNA levels between mettl3−/− derived tumors and wild-type derived tumors for Oct4, Nanog, Ki67, Myh6, Tuj1 and Sox17. Error bars represent standard deviation of the ΔΔCT value.

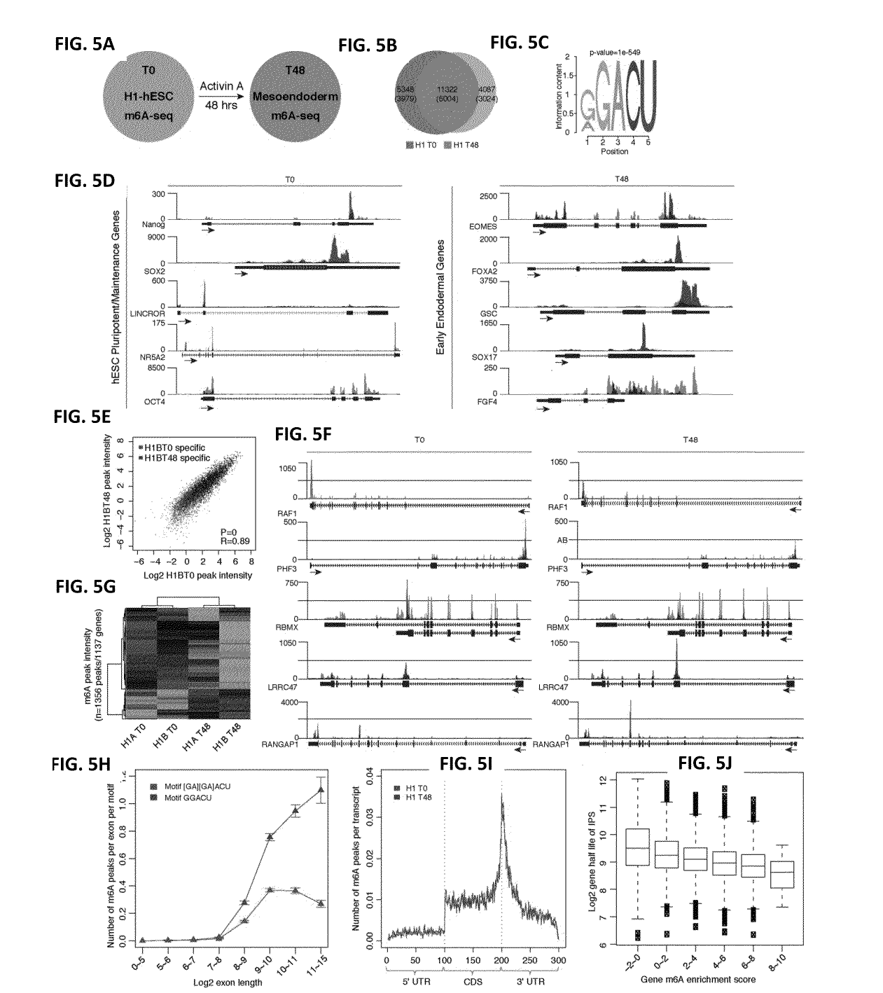

FIGS. 12A-12G show m⁶A-seq profiling of hESC during endoderm differentiation (and is related to FIG. 5.) FIG. 12A shows representative examples of m6A location in multi-exon non-coding RNAs and single-exon mRNAs. UCSC Genome browser plots of m6A-seq reads (red) along indicated RNAs in undifferentiated hESCs (i.e. T0). The grey reads are from non-immunoprecipitated control input libraries. The read density is calculated from the average of the two replicate T0 samples. Arrow indicates the direction of transcription. Related to FIG. 5D. FIG. 12B shows multi-exon coding and non-coding RNAs exhibit enrichment of m6A sites near the last exon-exon splice junction. The distribution of m6A peaks across the length of the mRNAs (n=9489) and noncoding RNAs (n=207) is shown. The 5′ most (first) exon, all internal exons, and the 3′ most (last) exon are divided into 10 bins and the percentage of m6A peaks that fall within each bin are shown (FIG. 12B is related to FIG. 5I). FIG. 12C shows the density of m6A-seq read coverage increases sharply downstream of the last exon-exon splice junction in both coding (n=5231) and non-coding RNAs (n=68) (FIG. 12C is related to FIG. 5I). FIG. 12D shows single-exon genes tend to have more m6A sites at their 3′ end. The percentage of m6A peaks that fall into normalized bins across the 5′UTR, CDS, and 3′UTR of single-exon genes is shown for hESC cells (T0 and T48 combined, n=137) as well as in merged data (“All merged”; n=200) from hESCs, 293T (Meyer et al., 2012) and HepG2 (Dominissini et al., 2012). (FIG. 12 D is related to FIG. 5I). FIG. 12E is a scatter plot representation of m6A enrichment score (on the X axis) and gene expression level in FPKM (on the Y axis) for each m6A peak (FIG. 12E is related to FIG. 5J). FIG. 12F shows m6A peak intensity is not correlated with nascent RNA transcription based on pausing index. The m6A enrichment scores vs GRO-seq determined the Pol II traveling ratio is plotted. The pausing index equal GRO-seq density at promoter defined as −300 and +300 of TSS divided by GRO-seq density in the gene body defines as +300 to end of the gene. (FIG. 12F is related to FIG. 5J). FIG. 12G shows mRNA half-life is anti-correlated with m6A enrichment in genes. (FIG. 12G is related to FIG. 5J).

FIGS. 13A-13E show METTL3 is required for normal human ESC endoderm differentiation (and is related to FIG. 7). FIG. 13A shows staining for SOX1 and DNA of neural stem cells in METTL3 knock down (KD) and control cells. FIG. 13B shows knockdown of METTL3 leads to a reduction in METTL3 mRNA levels. qRT-PCR for METTL3 mRNA was performed from RNA extracted from control WT hESC cells versus hESCs with anti-METTL3 shRNA (KD) clone #3 across the three indicated time points during endoderm differentiation. Error bars represent standard deviation across 3 replicates per time point. (FIG. 13B is related to FIG. 7B). FIG. 13C shows knockdown of METTL3 leads to a functional reduction in m6A levels. An anti-m6A dot blot was performed on 10× fold dilution of polyA selected RNA from wildtype (WT) hESC cells versus anti-METTL3 knockdown (KD) clone #3. (FIG. 13C is related to FIG. 7C). FIG. 13D shows knockdown of METTL3 leads to a delayed and reduced induction of endodermal marker genes. qRT-PCR was performed on indicated genes and time points. Error bars represent standard deviation across 3 replicates per time point. (FIG. 13D is related to FIGS. 7D and 7E). FIG. 13E shows knockdown of METTL3 leads prevents the normal reduction of stem maintenance/marker genes. qRT-PCR was performed for indicated genes and time points. Error bars represent standard deviation across 3 replicates per time point. (FIG. 13E is related to FIGS. 7D and 7E).

### DETAILED DESCRIPTION OF THE INVENTION

The present invention is directed to, in part, methods, compositions and kits to maintain a stem cell population, such as a human stem cell population, in an undifferentiated state, comprising contacting the stem cell population with an inhibitor of METTL3 or METTL4. In some embodiments, the methods, compositions and kits as disclosed herein relate to methods to prevent a stem cell population differentiating along an endoderm lineage. Other aspects of the technology described herein relates to methods, compositions and kits to promote a stem cell population to differentiate along an endoderm lineage. Moreover, another aspect of the technology described herein relates to methods, assays, arrays and kits for performing m6A analysis of RNA from stem cell populations to characterize the cell state of the cell population, which can be used, for example, as a quality control for the stem cell population. In some embodiments, the stem cell population is a human stem cell population, e.g., a hESC cell population or other human stem cell line.

The present invention is also directed to an array comprising nucleic acid sequences that hybridize to a set of RNA sequences (RNA transcripts, including mRNA transcripts and 3′UTR regions, and untranslated RNA sequences), or subsets thereof, which can be used to assess the m6A levels for use in characterizing the cell state of a stem cell population, e.g., human stem cell population. Aspects of the present invention relate to arrays, assays, systems, kits and methods to rapidly and inexpensively assess m6A levels (i.e., m6A peak intensities) in a set of RNA sequences (e.g., RNA transcripts, including mRNA transcripts and 3′UTR regions, and untranslated RNA sequences) to assess stem cell populations, including human stem cell populations, for their general quality (e.g., pluripotent capacity and cell state) and differentiation capacity.

As disclosed herein in the Examples, the inventors have discovered the function of m⁶A in human embryonic stem cells (ESCs), and surprisingly discovered that m⁶A is present on transcripts encoding multiple core pluripotency transcription factors, including but not limited to Nanog and Sox2, and was also enriched in 3′ untranslated regions at defined sequence motifs, and importantly marks unstable transcripts, including transcripts that need to be turned over upon differentiation. Using genetic inactivation or depletion of human Mettl3 in hESCs, the inventors discovered a decrease in m⁶A levels on select target genes, a prolonged Nanog expression upon differentiation, and impaired ESC's exit from self-renewal towards differentiation into several lineages in vitro and in vivo. In contrast to prior reports of Mettl3 knockdown in mESCs, knockdown of Mettl3 in hESC lead to the unexpected result of increased self-renewal and proliferation of hESC, and reduced ability to differentiate along specific lineages, in particular endoderm lineages.

Thus, the inventors have discovered that, in human stem cell populations in particular, m⁶A on RNA demonstrates the transcriptome flexibility and is required for human stem cells to differentiate to specific lineages. In particular, the inventors have discovered that m⁶A-modifications in the RNA (in mRNA transcripts, non-coding regions and in non-coding RNAs) of human stem cell populations serve as stem cells internal “quality control” as the m⁶A marks the mRNA as having passed a quality control test in the cell, as stem cells cannot differentiate without m⁶A-modifications on key transcripts.

As disclosed herein in the Examples, the inventors have surprisingly discovered that inhibition of METTL3 and/or METTL4 in human stem cell populations can be used to maintain the cells in a pluripotent state, and promote self-renewal and proliferation. Also disclosed herein in the Examples, the inventors have surprisingly discovered that the levels of m⁶A (i.e., m⁶A peak intensity) of a subset of RNA transcripts can accurately predict the cell state of a human stem cell population.

Another aspect of the present invention relates to a method for assessing m6A levels in set of RNA transcripts in a population of stem cells, which is useful to predict the functionality and suitability of a stem cell line, e.g., a pluripotent stem cell line for a desired use.

In some embodiments, the level of m⁶A (i.e., m⁶A peak intensity) of a subset of RNA transcripts measured in the methods, arrays, assays, kits and systems as disclosed herein includes at least 10, or at least 20 genes selected from any combination of the genes listed in Table 1 or Table 2.

In some embodiments, the differentiation assays, methods, systems and kits as disclosed herein can be used to characterize and determine the differentiation potential of a variety of stem cell lines, e.g., a pluripotent stem cell lines, such as, but not limited to embryonic stem cells, adult stem cells, autologous adult stem cells, iPS cells, and other pluripotent stem cell lines, such as reprogrammed cells, direct reprogrammed cells or partially reprogrammed cells. In some embodiments, a stem cell line is a human stem cell line. In some embodiments, a stem cell line, e.g., a pluripotent stem cell line is a genetically modified stem cell line. In some embodiments, where the stem cell line, e.g., a pluripotent stem cell line is for therapeutic use or for transplantation into a subject, a stem cell line is an autologous stem cell line, e.g., derived from a subject to which a population of stem cells will be transplanted back into, and in alternative embodiments, a stem cell line, e.g., a pluripotent stem cell line is an allogeneic pluripotent stem cell line.

### DEFINITIONS

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

The term “nucleic acid” or “nucleic acid sequence” as used herein is defined as a molecule comprised of two or more deoxyribonucleotides or ribonucleotides. The exact length of the sequence will depend on many factors, which in turn depends on the ultimate function or use of the sequence. The sequence can be generated in any manner, including chemical synthesis, DNA replication, reverse transcription, or a combination thereof. Due to the amplifying nature of the present invention, the number of deoxyribonucleotide or ribonucleotide bases within a nucleic acid sequence can be virtually unlimited. The term “oligonucleotide,” as used herein, is interchangeably synonymous with the term “nucleic acid sequence”.

As used herein, oligonucleotide sequences that are complementary to one or more of the genes described herein, refers to oligonucleotides that are capable of hybridizing under stringent conditions to at least part of the nucleotide sequence of said genes. Such hybridizable oligonucleotides will typically exhibit at least about 75% sequence identity at the nucleotide level to said genes, preferably about 80% or 85% sequence identity or more preferably about 90% or 95% or more sequence identity to said genes.

The term “primer” as used herein refers to a sequence of nucleic acid which is complementary or substantially complementary to a portion of the target gene of interest. Typically 2 primers (e.g., a 3′ primer and a 5′ primer) are complementary to different portions of the target gene of interest and can be used to amplify a portion of the mRNA of the target gene by RT-PCR.

The phrase “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The phrase “hybridizing specifically to” refers to the binding, duplexing or hybridizing of a molecule substantially to or only to a particular nucleotide sequence or sequences under stringent conditions when that sequence is present in a complex mixture (e.g., total cellular) DNA or RNA.

The term “biomarker” means any gene, protein, or an EST derived from that gene, the expression or level of which changes between certain conditions. Where the expression of the gene correlates with a certain condition, the gene is a biomarker for that condition.

As used herein, the term “gene” has its meaning as understood in the art. However, it will be appreciated by those of ordinary skill in the art that the term “gene” can include gene regulatory sequences (e.g., promoters, enhancers, etc.) and/or intron sequences. It will further be appreciated that definitions of gene include references to nucleic acids that do not encode proteins but rather encode functional RNA molecules such as tRNAs. For clarity, the term gene generally refers to a portion of a nucleic acid that encodes a protein; the term can optionally encompass regulatory sequences. This definition is not intended to exclude application of the term “gene” to non-protein coding expression units but rather to clarify that, in most cases, the term as used in this document refers to a protein coding nucleic acid. In some cases, the gene includes regulatory sequences involved in transcription, or message production or composition. In other embodiments, the gene comprises transcribed sequences that encode for a protein, polypeptide or peptide. In keeping with the terminology described herein, an “isolated gene” can comprise transcribed nucleic acid(s), regulatory sequences, coding sequences, or the like, isolated substantially away from other such sequences, such as other naturally occurring genes, regulatory sequences, polypeptide or peptide encoding sequences, etc. In this respect, the term “gene” is used for simplicity to refer to a nucleic acid comprising a nucleotide sequence that is transcribed, and the complement thereof.

The term “signature” as used herein refers to the m6A levels present on a set of target genes (or RNA species or mRNA transcipts).

The term a “similarity value” is a number that represents the degree of similarity between two things being compared. For example, a similarity value can be a number that indicates the overall similarity between a cell sample expression profile using specific phenotype-related biomarkers and a control specific to that template. The similarity value can be expressed as a similarity metric, such as a correlation coefficient, or a classification probability or can simply be expressed as the expression level difference, or the aggregate of the expression level differences, between a cell sample expression profile and a baseline template.

The term “expression” refers to the cellular processes involved in producing RNA and proteins and as appropriate, secreting proteins, including where applicable, but not limited to, for example, transcription, translation, folding, modification and processing. “Expression products” include RNA transcribed from a gene and polypeptides obtained by translation of mRNA transcribed from a gene.

As used herein, the terms “measuring m6A levels,” “obtaining m6A level,” and “detecting m6A levels” and the like, includes methods that quantify m6A levels on RNA species, for example, a transcript of a gene, or non-coding RNA. In some embodiments, the assay provides an indicator of the cell state of a stem cell population (e.g., if it is an undifferentiated state or differentiated state). In some embodiments, the indicator is a numerical value (e.g., the value from a t-test from the comparison of the average ΔCt for each target gene measured as compared to reference ΔCt of the same gene for a reference m6A level or peak intensity, as disclosed herein in the Examples). In some embodiments, the assay can provide a “yes” or “no” result without necessarily providing quantification, indicating that the stem cell population analysed is in an undifferentiated (i.e., pluripotent) state or not, respectively. Alternatively, a measured m6A levels or m6A peak intensity can be expressed as any quantitative value, for example, a fold-change in m6A peak intensity, up or down, relative to a control level of m6A peak intensity of the same gene in another sample, or a log ratio of expression, or any visual representation thereof, such as, for example, a “heatmap” where a color intensity is representative of the m6A peak intensity for a given RNA species.

The terms “m6A” and “m⁶A” are used interchangeably herein and refers to N(6)-methyladenosine residues in RNA species in a cell, including m⁶A modifications in any region of a mRNA molecule (including coding regions and non-coding regions such as untranslated 3′UTR and STOP codons), and untranslated RNA molecules, such as linc RNA and miRNA molecules or other multi-exon non-coding RNAs and single-exon mRNAs.

The term “m6A intensity profile” or “m6A signature profile” as used herein is intended to refer to the m6A levels of a gene, or a set of genes, in a stem cell population. In one embodiments the term “gene profile” refers to the m6A peak intensity levels or of a set of 10 or more genes listed in Table 1 or Table 2, or any selection of the genes of between 10-20, or 20-30, or 30-50, or 50-100, or 100-200, or 200-300, or 300-400, or 400-600 listed in Table 1 or Table 2, which are described herein.

The term “differential expression” in the context of the present invention means the gene is up-regulated or down-regulated in comparison to its normal variation of expression in a pluripotent stem cell. Statistical methods for calculating differential expression of genes are discussed elsewhere herein.

The term “genes of Table 1 or Table 2” is used interchangeably herein with “gene listed in Table 1 or Table 2” and refers to the RNA species or gene products of genes listed in Table 1 and/or Table 2, respectively. By “gene product” is meant any product of transcription or translation of the genes, whether produced by natural or artificial means. In some embodiments, the genes referred to herein are those listed in Table 1. The same applies to “genes of Table 2”, but refers to the gene products of genes listed in Table 2.

The term “hybridization” or “hybridizes” as used herein involves the annealing of a complementary sequence to the target nucleic acid (the sequence to be detected). The ability of two polymers of nucleic acid containing complementary sequences to find each other and anneal through base pairing interaction is a well-recognized phenomenon. The initial observations of the “hybridization” process by Marmur and Lane, Proc. Natl. Acad. Sci. USA, 46:453 (1960) and Doty et al., Proc. Natl. Acad. Sci. USA, 46:461 (1960) have been followed by the refinement of this process into an essential tool of modern biology.

The terms “complementary” or “substantially complementary” as used herein refer to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementarity over a stretch of at least 14 to 25 nucleotides, preferably at least about 75%, more preferably at least about 90% complementarity. See M. Kanehisa, Nucleic Acids Res., 12:203 (1984), incorporated herein by reference. The term “at least a portion of as used herein, refers to the complimentarity between a circular DNA template and an oligonucleotide primer of at least one base pair.

Partially complementary sequences will hybridize under low stringency conditions. This is not to say that conditions of low stringency are such that non-specific binding is permitted; low stringency conditions require that the binding of two sequences to one another be a specific (i.e., selective) interaction. The absence of non-specific binding can be tested by the use of a second target which lacks even a partial degree of complementarity (e.g., less than about 30% identity); in the absence of non-specific binding the probe will not hybridize to the second non-complementary target.

The term “stringency” refers to the degree of specificity imposed on a hybridization reaction by the specific conditions used for a reaction. When used in reference to nucleic acid hybridization, stringency typically occurs in a range from about Tₘ−5° C. (5° C. below the Tₘ of the probe) to about 20° C., 25° C. below Tₘ. As will be understood by those of skill in the art, a stringent hybridization can be used to identify or detect identical polynucleotide sequences or to identify or detect similar or related polynucleotide sequences. Under “stringent conditions” a nucleic acid sequence of interest will hybridize to its exact complement and closely related sequences. Suitably stringent hybridization conditions for nucleic acid hybridization of a primer or short probe include, e.g., 3×SSC, 0.1% SDS, at 50° C.

When used in reference to nucleic acid hybridization the art knows well that numerous equivalent conditions can be employed to comprise either low or high stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution can be varied to generate conditions of either low or high stringency hybridization different from, but equivalent to, the above listed conditions.

The term “solid surface” as used herein refers to a material having a rigid or semi-rigid surface. Such materials will preferably take the form of chips, plates (e.g., microtiter plates), slides, small beads, pellets, disks or other convenient forms, although other forms can be used. In some embodiments, at least one surface of the solid surface will be substantially flat. In other embodiments, a roughly spherical shape is preferred.

The term “reprogramming” as used herein refers to a process that alters or reverses the differentiation state of a differentiated cell (e.g. a somatic cell). Stated another way, reprogramming refers to a process of driving the differentiation of a cell backwards to a more undifferentiated or more primitive type of cell. Complete reprogramming involves complete reversal of at least some of the heritable patterns of nucleic acid modification (e.g., methylation), chromatin condensation, epigenetic changes, genomic imprinting, etc., that occur during cellular differentiation as a zygote develops into an adult. Reprogramming is distinct from simply maintaining the existing undifferentiated state of a cell that is already pluripotent or maintaining the existing less than fully differentiated state of a cell that is already a multipotent cell (e.g., a hematopoietic stem cell). Reprogramming is also distinct from promoting the self-renewal or proliferation of cells that are already pluripotent or multipotent.

The term “induced pluripotent stem cell” or “iPSC” or “iPS cell” refers to a cell derived from a complete reversion or reprogramming of the differentiation state of a differentiated cell (e.g. a somatic cell). As used herein, an iPSC is fully reprogrammed and is a cell which has undergone complete epigenetic reprogramming. As used herein, an iPSC is a cell which cannot be further reprogrammed to a more immature state (e.g., an iPSC cell is terminally reprogrammed).

The term “pluripotent” as used herein refers to a cell with the capacity, under different conditions, to differentiate to cell types characteristic of all three germ cell layers (endoderm, mesoderm and ectoderm). A pluripotent stem cell typically has the potential to divide in vitro for a long period of time, e.g., greater than one year or more than 30 passages.

The term “differentiated cell” refers to any primary cell that is not, in its native form, pluripotent as that term is defined herein. The term a “differentiated cell” also encompasses cells that are partially differentiated, such as multipotent cells, or cells that are stable non-pluripotent partially reprogrammed cells. It should be noted that placing many primary cells in culture can lead to some loss of fully differentiated characteristics. However, such cells are included in the term differentiated cells and the loss of fully differentiated characteristics does not render these cells non-differentiated cells (e.g. undifferentiated cells) or pluripotent cells. The transition of a differentiated cell to pluripotency requires a reprogramming stimulus beyond the stimuli that lead to partial loss of differentiated character in culture. Reprogrammed cells also have the characteristic of the capacity of extended passaging without loss of growth potential, relative to primary cell parents, which generally have capacity for only a limited number of divisions in culture. In some embodiments, the term “differentiated cell” also refers to a cell of a more specialized cell type derived from a cell of a less specialized cell type (e.g., from an undifferentiated cell or a reprogrammed cell) where the cell has undergone a cellular differentiation process.

As used herein, the term “adult cell” refers to a cell found throughout the body after embryonic development.

In the context of cell ontogeny, the term “differentiate”, or “differentiating” is a relative term meaning a “differentiated cell” is a cell that has progressed further down the developmental pathway than its precursor cell. Thus in some embodiments, a reprogrammed cell as this term is defined herein, can differentiate to lineage-restricted precursor cells (such as a mesodermal stem cell), which in turn can differentiate into other types of precursor cells further down the pathway (such as an tissue specific precursor, for example, a cardiomyocyte precursor), and then to an end-stage differentiated cell, which plays a characteristic role in a certain tissue type, and can or cannot retain the capacity to proliferate further.

The term “embryonic stem cell” is used to refer to the pluripotent stem cells of the inner cell mass of the embryonic blastocyst (see U.S. Pat. Nos. 5,843,780, 6,200,806, which are incorporated herein by reference). Such cells can similarly be obtained from the inner cell mass of blastocysts derived from somatic cell nuclear transfer (see, for example, U.S. Pat. Nos. 5,945,577, 5,994,619, 6,235,970, which are incorporated herein by reference). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “phenotype” refers to one or a number of total biological characteristics that define the cell or organism under a particular set of environmental conditions and factors, regardless of the actual genotype.

The term “cell culture medium” (also referred to herein as a “culture medium” or “medium”) as referred to herein is a medium for culturing cells containing nutrients that maintain cell viability and support proliferation. The cell culture medium can contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The term “self-renewing media” or “self-renewing culture conditions” refers to a medium for culturing stem cells which contains nutrients that allow a stem cell line to propagate in an undifferentiated state. Self-renewing culture media is well known to those of ordinary skill in the art and is ordinarily used for maintenance of stem cells as embroid bodies (EBs), where the stem cells divide and replicate in an undifferentiated state.

The term “cell line” refers to a population of largely or substantially identical cells that has typically been derived from a single ancestor cell or from a defined and/or substantially identical population of ancestor cells. The cell line can have been or can be capable of being maintained in culture for an extended period (e.g., months, years, for an unlimited period of time). Cell lines include all those cell lines recognized in the art as such. It will be appreciated that cells acquire mutations and possibly epigenetic changes over time such that at least some properties of individual cells of a cell line can differ with respect to each other.

The term “lineages” as used herein describes a cell with a common ancestry or cells with a common developmental fate. By way of an example only, stating that a cell that is of endoderm origin or is of “endodermal lineage” means the cell was derived from an endodermal cell and can differentiate along the endodermal lineage restricted pathways, such as one or more developmental lineage pathways which give rise to definitive endoderm cells, which in turn can differentiate into liver cells, thymus, pancreas, lung and intestine.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2 SD) or greater difference in a value of the marker. The term refers to statistical evidence that there is a difference. It is defined as the probability of making a decision to reject the null hypothesis when the null hypothesis is actually true. Statistical significance can be determined by t-test or using a p-value.

As used herein, the term “DNA” is defined as deoxyribonucleic acid.

The term “differentiation” as used herein refers to the cellular development of a cell from a primitive stage towards a more mature (i.e. less primitive) cell.

The term “directed differentiation” as used herein refers to forcing differentiation of a cell from an undifferentiated (e.g. more primitive cell) to a more mature cell type (i.e. less primitive cell) via genetic and/or environmental manipulation. In some embodiments, a reprogrammed cell as disclosed herein is subject to directed differentiation into specific cell types, such as neuronal cell types, muscle cell types and the like.

The term “disease modeling” as used herein refers to the use of laboratory cell culture or animal research to obtain new information about human disease or illness. In some embodiments, a reprogrammed cell produced by the methods as disclosed herein can be used in disease modeling experiments.

The term “drug screening” as used herein refers to the use of cells and tissues in the laboratory to identify drugs with a specific function.

The term “marker” as used interchangeably with “biomarker” and describes the characteristics and/or phenotype of a cell. Markers can be used for selection of cells comprising characteristics of interest. Markers will vary with specific cells. Markers are characteristics, whether morphological, functional or biochemical (enzymatic) characteristics of the cell of a particular cell type, or molecules expressed by the cell type. Preferably, such markers are gene transcripts or their translation products (e.g., proteins). However, a marker can consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers can be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

As used herein an “antibody” refers to IgG, IgM, IgA, IgD or IgE molecules or antigen-specific antibody fragments thereof (including, but not limited to, a Fab, F(ab′)₂, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria.

As described herein, an “antigen” is a molecule that is bound by a binding site comprising the complementarity determining regions (CDRs) of an antibody agent. Typically, antigens are bound by antibody ligands and are capable of raising an antibody response in vivo. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule.

As used herein, the term “antibody reagent” refers to a polypeptide that includes at least one immunoglobulin variable domain or immunoglobulin variable domain sequence and which specifically binds to a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

The terms “antigen-binding fragment” or “antigen-binding domain”, which are used interchangeably herein are used to refer to one or more fragments of a full length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized. In certain embodiments, specific binding is indicated by a dissociation constant on the order of ≦10⁻⁸ M, ≦10⁻⁹ M, ≦10¹⁰ M or below.

As used herein, “expression level” refers to the number of mRNA molecules and/or polypeptide molecules encoded by a given gene that are present in a cell or sample. Expression levels can be increased or decreased relative to a reference level.

As used herein, the term “iRNA agent” or “RNAi agent” refers to an agent that contains RNA as that term is defined herein, and which mediates the targeted cleavage of an RNA transcript via an RNA-induced silencing complex (RISC) pathway. In one embodiment, an iRNA as described herein inhibits the expression METTL3/Lnk a stem cell or progenitor cell, e.g., HSC or a mammal.

As used herein, “target sequence” refers to a contiguous portion of the nucleotide sequence of a messenger RNA (mRNA) molecule formed during the transcription of a gene, including mRNA that is a product of RNA processing of a primary transcription product. The target portion of the sequence will be at least long enough to serve as a specific binding site for an iRNA agent and/or as a substrate for iRNA-directed cleavage at or near that portion. For example, the target sequence will generally be from 9-36 nucleotides in length, e.g., 15-30 nucleotides in length, including all sub-ranges therebetween. As non-limiting examples, the target sequence can be from 15-30 nucleotides, 15-26 nucleotides, 15-23 nucleotides, 15-22 nucleotides, 15-21 nucleotides, 15-20 nucleotides, 15-19 nucleotides, 15-18 nucleotides, 15-17 nucleotides, 18-30 nucleotides, 18-26 nucleotides, 18-23 nucleotides, 18-22 nucleotides, 18-21 nucleotides, 18-20 nucleotides, 19-30 nucleotides, 19-26 nucleotides, 19-23 nucleotides, 19-22 nucleotides, 19-21 nucleotides, 19-20 nucleotides, 20-30 nucleotides, 20-26 nucleotides, 20-25 nucleotides, 20-24 nucleotides, 20-23 nucleotides, 20-22 nucleotides, 20-21 nucleotides, 21-30 nucleotides, 21-26 nucleotides, 21-25 nucleotides, 21-24 nucleotides, 21-23 nucleotides, or 21-22 nucleotides.

As used herein, the term “strand comprising a sequence” refers to an oligonucleotide comprising a chain of nucleotides that is described by the sequence referred to using the standard nucleotide nomenclature.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing. Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an iRNA, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of gene expression via a RISC pathway. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but are not limited to, G:U Wobble or Hoogstein base pairing.

The terms “complementary,” “fully complementary” and “substantially complementary” herein can be used with respect to the base matching between the sense strand and the antisense strand of a dsRNA, or between the antisense strand of an iRNA agent and a target sequence, as will be understood from the context of their use.

As used herein, a polynucleotide that is “substantially complementary to at least part of a messenger RNA (mRNA) refers to a polynucleotide that is substantially complementary to a contiguous portion of the mRNA of interest (e.g., an mRNA encoding METTL3). For example, a polynucleotide is complementary to at least a part of a mRNA if the sequence is substantially complementary to a non-interrupted portion of the mRNA.

The term” double-stranded RNA” or “dsRNA,” as used herein, refers to an iRNA that includes an RNA molecule or complex of molecules having a hybridized duplex region that comprises two anti-parallel and substantially complementary nucleic acid strands, which will be referred to as having “sense” and “antisense” orientations with respect to a target RNA. The duplex region can be of any length that permits specific degradation of a desired target RNA through a RISC pathway, but will typically range from 9 to 36 base pairs in length, e.g., 15-30 base pairs in length. Considering a duplex between 9 and 36 base pairs, the duplex can be any length in this range, for example, 9, 10, 11, 12, 13, 14, 15, 16, 17, 18, 19, 20, 21, 22, 23, 24, 25, 26, 27, 28, 29, 30, 31, 32, 33, 34, 35, or 36 and any sub-range therein between, including, but not limited to 15-30 base pairs, 15-26 base pairs, 15-23 base pairs, 15-22 base pairs, 15-21 base pairs, 15-20 base pairs, 15-19 base pairs, 15-18 base pairs, 15-17 base pairs, 18-30 base pairs, 18-26 base pairs, 18-23 base pairs, 18-22 base pairs, 18-21 base pairs, 18-20 base pairs, 19-30 base pairs, 19-26 base pairs, 19-23 base pairs, 19-22 base pairs, 19-21 base pairs, 19-20 base pairs, 20-30 base pairs, 20-26 base pairs, 20-25 base pairs, 20-24 base pairs, 20-23 base pairs, 20-22 base pairs, 20-21 base pairs, 21-30 base pairs, 21-26 base pairs, 21-25 base pairs, 21-24 base pairs, 21-23 base pairs, or 21-22 base pairs. dsRNAs generated in the cell by processing with Dicer and similar enzymes are generally in the range of 19-22 base pairs in length. One strand of the duplex region of a dsDNA comprises a sequence that is substantially complementary to a region of a target RNA. The two strands forming the duplex structure can be from a single RNA molecule having at least one self-complementary region, or can be formed from two or more separate RNA molecules. Where the duplex region is formed from two strands of a single molecule, the molecule can have a duplex region separated by a single stranded chain of nucleotides (herein referred to as a “hairpin loop”) between the 3′-end of one strand and the 5′-end of the respective other strand forming the duplex structure. The hairpin loop can comprise at least one unpaired nucleotide; in some embodiments the hairpin loop can comprise at least 3, at least 4, at least 5, at least 6, at least 7, at least 8, at least 9, at least 10, at least 20, at least 23 or more unpaired nucleotides. Where the two substantially complementary strands of a dsRNA are comprised by separate RNA molecules, those molecules need not, but can be covalently connected. Where the two strands are connected covalently by means other than a hairpin loop, the connecting structure is referred to as a “linker.” The term “siRNA” is also used herein to refer to a dsRNA as described above.

The skilled artisan will recognize that the term “RNA molecule” or “ribonucleic acid molecule” encompasses not only RNA molecules as expressed or found in nature, but also analogs and derivatives of RNA comprising one or more ribonucleotide/ribonucleoside analogs or derivatives as described herein or as known in the art. Strictly speaking, a “ribonucleoside” includes a nucleoside base and a ribose sugar, and a “ribonucleotide” is a ribonucleoside with one, two or three phosphate moieties. However, the terms “ribonucleoside” and “ribonucleotide” can be considered to be equivalent as used herein. The RNA can be modified in the nucleobase structure or in the ribose-phosphate backbone structure, e.g., as described herein below. However, the molecules comprising ribonucleoside analogs or derivatives must retain the ability to form a duplex. As non-limiting examples, an RNA molecule can also include at least one modified ribonucleoside including but not limited to a 2′-O-methyl modified nucleoside, a nucleoside comprising a 5′ phosphorothioate group, a terminal nucleoside linked to a cholesteryl derivative or dodecanoic acid bisdecylamide group, a locked nucleoside, an abasic nucleoside, a 2′-deoxy-2′-fluoro modified nucleoside, a 2′-amino-modified nucleoside, 2′-alkyl-modified nucleoside, morpholino nucleoside, a phosphoramidate or a non-natural base comprising nucleoside, or any combination thereof. Alternatively, an RNA molecule can comprise at least two modified ribonucleosides, at least 3, at least 4, at least 5, at least 6, at least 7, at least 8, at least 9, at least 10, at least 15, at least 20 or more, up to the entire length of the dsRNA molecule. The modifications need not be the same for each of such a plurality of modified ribonucleosides in an RNA molecule. In one embodiment, modified RNAs contemplated for use in methods and compositions described herein are peptide nucleic acids (PNAs) that have the ability to form the required duplex structure and that permit or mediate the specific degradation of a target RNA via a RISC pathway.

In one aspect, a modified ribonucleoside includes a deoxyribonucleoside. In such an instance, an iRNA agent can comprise one or more deoxynucleosides, including, for example, a deoxynucleoside overhang(s), or one or more deoxynucleosides within the double stranded portion of a dsRNA. However, it is self evident that under no circumstances is a double stranded DNA molecule encompassed by the term “iRNA.”

In one aspect, an RNA interference agent includes a single stranded RNA that interacts with a target RNA sequence to direct the cleavage of the target RNA. Without wishing to be bound by theory, long double stranded RNA introduced into plants and invertebrate cells is broken down into siRNA by a Type III endonuclease known as Dicer (Sharp et al., Genes Dev. 2001, 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleaves the target to induce silencing (Elbashir, et al., (2001) Genes Dev. 15:188). Thus, in one aspect the technology described herein relates to a single stranded RNA that promotes the formation of a RISC complex to effect silencing of the target gene.

As used herein, the term “nucleotide overhang” refers to at least one unpaired nucleotide that protrudes from the duplex structure of an iRNA, e.g., a dsRNA. For example, when a 3′-end of one strand of a dsRNA extends beyond the 5′-end of the other strand, or vice versa, there is a nucleotide overhang. A dsRNA can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′ end, 3′ end or both ends of either an antisense or sense strand of a dsRNA.

In one embodiment, the antisense strand of a dsRNA has a 1-10 nucleotide overhang at the 3′ end and/or the 5′ end. In one embodiment, the sense strand of a dsRNA has a 1-10 nucleotide overhang at the 3′ end and/or the 5′ end. In another embodiment, one or more of the nucleotides in the overhang is replaced with a nucleoside thiophosphate.

The terms “blunt” or “blunt ended” as used herein in reference to a dsRNA or dsDNA mean that there are no unpaired nucleotides or nucleotide analogs at a given terminal end of a dsRNA or dsDNA molecule, i.e., no nucleotide overhang. One or both ends of a dsRNA or dsDNA can be blunt. Where both ends of a dsRNA or dsDNA are blunt, the dsRNA or dsDNA is said to be blunt ended. To be clear, a “blunt ended” dsRNA or dsDNA is a dsRNA or dsDNA that is blunt at both ends, i.e., no nucleotide overhang at either end of the molecule. Most often such a molecule will be double-stranded over its entire length. In contrast “sticky ends” refers to dsDNA or dsRNA molecule that has at least 1 or more (typically 2-5 or more) nucleotide overhang.

The term “antisense strand” or “guide strand” refers to the strand of an iRNA, e.g., a dsRNA, which includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′ and/or 3′ terminus.

The term “sense strand,” or “passenger strand” as used herein, refers to the strand of an iRNA that includes a region that is substantially complementary to a region of the antisense strand as that term is defined herein.

The terms “microRNA” or “miRNA” or “mir” or “miR” are used interchangeably herein, are endogenous RNAs, some of which are known to regulate the expression of protein-coding genes at the posttranscriptional level. As used herein, the term “microRNA” refers to any type of micro-interfering RNA, including but not limited to, endogenous microRNA and artificial microRNA. “MicroRNA” also means a non-coding RNA between 18 and 25 nucleobases in length, which is the product of cleavage of a pre-miRNA by the enzyme Dicer. Examples of mature miRNAs are found in the miRNA database known as miRBase (http://microma.sanger.ac.uk/). In certain embodiments, microRNA is abbreviated as “miRNA” or “miR.” Typically, endogenous microRNA are small RNAs encoded in the genome which are capable of modulating the productive utilization of mRNA. A mature miRNA is a single-stranded RNA molecule of about 21-23 nucleotides in length which is complementary to a target sequence, and hybridizes to the target RNA sequence to inhibit expression of a gene which encodes a miRNA target sequence. miRNAs themselves are encoded by genes that are transcribed from DNA but not translated into protein (non-coding RNA); instead they are processed from primary transcripts known as pri-miRNA to short stem-loop structures called pre-miRNA and finally to functional miRNA. Mature miRNA molecules are partially complementary to one or more messenger RNA (mRNA) molecules, and their main function is to downregulate gene expression. MicroRNA sequences have been described in publications such as, Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into the precursor molecule.

A “mature microRNA” (mature miRNA) typically refers to a single-stranded RNA molecules of about 21-23 nucleotides in length, which regulates gene expression. miRNAs are encoded by genes from whose DNA they are transcribed, but miRNAs are not translated into protein; instead each primary transcript (pri-miRNA) is processed into a short stem-loop structure (precursor microRNA) before undergoing further processing into a functional mature miRNA. Mature miRNA molecules are partially complementary to one or more messenger RNA (mRNA) molecules, and their main function is to down-regulate gene expression. As used throughout, the term “microRNA” or “miRNA” includes both mature microRNA and precursor microRNA.

A mature miRNA is produced as a result of a series of miRNA maturation steps; first a gene encoding the miRNA is transcribed. The gene encoding the miRNA is typically much longer than the processed mature miRNA molecule; miRNAs are first transcribed as primary transcripts or “pri-miRNA” with a cap and poly-A tail, which is subsequently processed to short, about 70-nucleotide “stem-loop structures” known as “pre-miRNA” in the cell nucleus. This processing is performed in animals by a protein complex known as the Microprocessor complex, consisting of the nuclease Drosha and the double-stranded RNA binding protein Pasha. These pre-miRNAs are then processed to mature miRNAs in the cytoplasm by interaction with the endonuclease Dicer, which also initiates the formation of the RNA-induced silencing complex (RISC). This complex is responsible for the gene silencing observed due to miRNA expression and RNA interference. The pathway is different for miRNAs derived from intronic stem-loops; these are processed by Drosha but not by Dicer. In some instances, a given region of DNA and its complementary strand can both function as templates to give rise to at least two miRNAs. Mature miRNAs can direct the cleavage of mRNA or they can interfere with translation of the mRNA, either of which results in reduced protein accumulation, rendering miRNAs capable of modulating gene expression and related cellular activities.

“Pri-miRNA” or “pri-miR” means a non-coding RNA having a hairpin structure that is a substrate for the double-stranded RNA-specific ribonuclease Drosha. A “pri-miRNA” is a precursor to a mature miRNA molecule which comprises; (i) a microRNA sequence and (ii) stem-loop component which are both flanked (i.e. surrounded on each side) by “microRNA flanking sequences”, where each flanking sequence typically ends in either a cap or poly-A tail. Pri-microRNA, (also referred to as large RNA precursors), are composed of any type of nucleic acid based molecule capable of accommodating the microRNA flanking sequences and the microRNA sequence. Examples of pri-miRNAs and the individual components of such precursors (flanking sequences and microRNA sequence) are provided herein. The nucleotide sequence of the pri-miRNA precursor and its stem-loop components can vary widely. In one aspect a pre-miRNA molecule can be an isolated nucleic acid; including microRNA flanking sequences and comprising a stem-loop structure and a microRNA sequence incorporated therein. A pri-miRNA molecule can be processed in vivo or in vitro to an intermediate species caller “pre-miRNA”, which is further processed to produce a mature miRNA.

A “pre-miRNA” or “pre-miR” means a non-coding RNA having a hairpin structure, which is the product of cleavage of a pri-miR by the double-stranded RNA-specific ribonuclease known as DroshaA. The term “pre-miRNA” refers to the intermediate miRNA species in the processing of a pri-miRNA to mature miRNA, where pri-miRNA is processed to pre-miRNA in the nucleus, whereupon pre-miRNA translocates to the cytoplasm where it undergoes additional processing in the cytoplasm to form mature miRNA. Pre-miRNAs are generally about 70 nucleotides long, but can be less than 70 nucleotides or more than 70 nucleotides.

The term “miRNA precursor” means a transcript that originates from a genomic DNA and that comprises a non-coding, structured RNA comprising one or more miRNA sequences. For example, in certain embodiments a miRNA precursor is a pre-miRNA. In certain embodiments, a miRNA precursor is a pri-miRNA

As used herein, the phrase “inhibit the expression of,” refers to at an least partial reduction of gene expression of a gene encoding METTL3 in a cell treated with METTL3 inhibitor (e.g., an iRNA composition as described herein) compared to the expression of METTL3 in an untreated cell.

The terms “silence,” “inhibit the expression of,” “down-regulate the expression of,” “suppress the expression of,” and the like, in so far as they refer to METTL3, herein refer to the at least partial suppression of the expression of a gene encoding METTL3, as manifested by a reduction of the amount of mRNA encoding METTL3 which can be isolated from or detected in a first cell or group of cells in which that gene is transcribed and which has or have been treated such that the expression of METTL3 is inhibited, as compared to a second cell or group of cells substantially identical to the first cell or group of cells but which has or have not been so treated (control cells). The degree of inhibition is usually expressed in terms of

$$( \frac{[ mRNA in control cells ] - [ mRNA in treated cells ]}{[ mRNA in control cells ]} ) \times 100 \%$$

Alternatively, the degree of inhibition can be given in terms of a reduction of a parameter that is functionally linked to gene expression, e.g., the amount of protein encoded by a gene, or the number of cells displaying a certain phenotype. In principle, gene silencing can be determined in any cell expressing, either constitutively or by genomic engineering, and by any appropriate assay. However, when a reference is needed in order to determine whether a given iRNA (or gene editing procedure) inhibits the expression of the gene encoding METTL3 by a certain degree and therefore is encompassed by the technology described herein, the assays provided in the Examples below shall serve as such reference.

For example, in certain instances, expression of METTL3 is suppressed by at least about 10%, 15%, 20%, 25%, 30%, 35%, 40%, 45%, or 50% by administration of an iRNA featured herein. In some embodiments, a gene encoding METTL3 in a cell is suppressed by at least about 60%, 70%, or 80% or more than 80% by administration of an iRNA or gene editing procedures (i.e., CRISPR/Cas9 or CRISPR/Cpf1) as featured herein. In some embodiments, a gene encoding METTL3 is suppressed by at least about 85%, 90%, 95%, 98%, 99% or more by administration of an iRNA (or gene editing procedures) as described herein.

“Introducing into a cell,” when referring to an iRNA, means facilitating or effecting uptake or absorption into the cell, as is understood by those skilled in the art. Absorption or uptake of an iRNA can occur through unaided diffusive or active cellular processes, or by auxiliary agents or devices. The meaning of this term is not limited to cells in vitro; an iRNA can also be “introduced into a cell,” wherein the cell is part of a living organism. In such an instance, introduction into the cell will include the delivery to the organism. For example, for in vivo delivery, iRNA can be injected into a tissue site or administered systemically. In vivo delivery can also be by a beta-glucan delivery system, such as those described in U.S. Pat. Nos. 5,032,401 and 5,607,677, and U.S. Publication No. 2005/0281781 which are hereby incorporated by reference in their entirety. In vitro introduction into a cell includes methods known in the art such as electroporation and lipofection. Further approaches are described herein below or are known in the art.

The term “computer” can refer to any non-human apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a micro-computer; a server; an interactive television; a hybrid combination of a computer and an interactive television; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

The term “computer-readable medium” can refer to any storage device used for storing data accessible by a computer, as well as any other means for providing access to data by a computer. Examples of a storage-device-type computer-readable medium include, but is not limited to: a magnetic hard disk; a floppy disk; an optical disk, such as a CD-ROM and a DVD; DATs, a USB drive, a magnetic tape; a memory chip. A computer-readable medium is a tangible media not a signal, and does not include carrier waves or other wave forms for data transmission.

The term “software” is used interchangeably herein with “program” and refers to prescribed rules to operate a computer. Examples of software include: software; code segments; instructions; computer programs; and programmed logic.

The term a “computer system” can refer to a system having a computer, where the computer comprises a computer-readable medium embodying software to operate the computer.

The phrase “displaying or outputting” or providing an “indication” of the result of the m6A levels or peak intensities, or a prediction result, means that the results of a gene expression are communicated to a user using any medium, such as for example, orally, writing, visual display, etc., computer readable medium or computer system. It will be clear to one skilled in the art that outputting the result is not limited to outputting to a user or a linked external component(s), such as a computer system or computer memory, but can alternatively or additionally be outputting to internal components, such as any computer readable medium. It will be clear to one skilled in the art that the various sample classification methods disclosed and claimed herein, can, but need not be, computer-implemented, and that, for example, the displaying or outputting step can be done by, for example, by communicating to a person orally or in writing (e.g., in handwriting).

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

Other than in the operating examples, or where otherwise indicated, all numbers expressing quantities of ingredients or reaction conditions used herein should be understood as modified in all instances by the term “about.” The term “about” when used in connection with percentages can mean±1%. The present invention is further explained in detail by the following, including the Examples, but the scope of the invention should not be limited thereto.

It is understood that the detailed description and the Examples that follow are illustrative only and are not to be taken as limitations upon the scope of the invention. Various changes and modifications to the disclosed embodiments, which will be apparent to those of skill in the art, can be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. All statements as to the date or representation as to the contents of these documents are based on the information available to the applicants and do not constitute any admission as to the correctness of the dates or contents of these documents.

I. Modification of METTL3 and/or METTL4

Herein, the inventors have surprisingly discovered that, in human ESCs, m6A is present on transcripts encoding multiple core pluripotency transcription factors, including but not limited to Nanog and Sox2, and is also enriched in 3′ untranslated regions at defined sequence motifs, and importantly marks unstable transcripts, including transcripts that need to be turned over upon differentiation. When human Mettl3 was knocked down in hESCs, the inventors discovered a decrease in m6A levels on select target genes, a prolonged Nanog expression upon differentiation, and impaired ESC's exit from self-renewal towards differentiation into several lineages in vitro and in vivo. Importantly, knockdown of Mettl3 in hESC lead to the unexpected result of increased self-renewal and proliferation of hESC, and reduced ability to differentiate along specific lineages, in particular endoderm lineages. Thus, modulation of Mettl3 and/or Mettl4 can be used to promote self-renewal and prevent differentiation (by inhibition of Mettl3 and/or Mettl4), or alternatively promote differentiation into specific cell lineages (e.g., by increasing m⁶A on specific RNA species in a stem cell population).

A. Inhibition of METTL3 and/or METTL4.

One aspect of the technology as disclosed herein relates to, in part, methods, compositions and kits to maintain a stem cell population, such as a human stem cell population, in an undifferentiated state, comprising contacting the stem cell population with an inhibitor of METTL3 or METTL4. In some embodiments, the methods, compositions and kits as disclosed herein relate to methods to prevent a stem cell population differentiating along an endoderm lineage.

Mettl3 inhibition in a stem cell population, e.g., a human stem cell population can be performed by one of ordinary skill in the art, for example, inhibition of METTL3 can result in a decrease in METTL3 protein level, a decrease in METTL3 mRNA level, a decrease in METTL3 protein activity, or combinations thereof. The inhibition of METTL3 can be done using a variety of methods known in the art including, but not limited to, genome editing, gene silencing, disruption of normal METTL3 protein activity, and combinations thereof.

In some embodiments, METTL3 can be inhibited in the stem cells and/or progenitor cells before the cells are expanded and/or enriched. In some embodiments, the stem cells and/or progenitor cells are expanded and/or enriched prior to METTL3 inhibition.

In some embodiments, METTL3 and/or METTL4 can control all stages of differentiation. Accordingly, the technology described herein of inhibiting METTL3 and/or METTL4 function or gene expression for a certain period of time can be used to prevent differentiation of any cell type, and/or keep a cell in a particular state of differentiation. For example, without being limited to theory, if we wanted to increase the number of hair stem cells on the scalp for a period of time (i.e. to expand the number of hair stem cells), then the a METTL3 and/or METTL4 inhibitor can be applied to the skin stem cell population, (e.g., on the scalp for a period of time), after which the expanded stem cell population can be allowed to differentiate and repopulate the scalp with hair. Put another way, manipulation of METTL3 and/or METTL4 may allow the expansion of a number of human stem cells, including adult human stem cells), which is useful for expanding small populations of stem cells, as well as isolated stem cell populations (e.g., isolated from a human subject, or rare stem cell populations). In other words, the technology described herein of temporarily inhibiting METTL3 and/or METTL4 in a stem cell population can be used for production of industrial scale stem cells populations from a limited, or small quantity of initial stem cell population.

### METTL3 Antagonists

In some embodiments, the inhibition of METTL3 comprises contacting the population of stem cells and/or progenitor cells with an antagonist of METTL3. As used herein, the term “antagonist of METTL3” refers to any agent that decreases the level and/or activity of METTL3. The term “antagonist of METTL3” refers to an agent which decreases the expression and/or activity METTL3 in a stem cell population by at least 10%, e.g. by at least 10%, at least 20%, at least 30%, at least 40%, at least 50%, at least 60%, at least 70%, at least 80%, or at least 90%. Examples of antagonists of METTL3 include, but are not limited to, an inorganic molecule, an organic molecule, a nucleic acid, a nucleic acid analog or derivative, a peptide, a peptidomimetic, a protein, an antibody or an antigen-binding fragment thereof, and combinations thereof.

In some embodiments, the antagonist of METTL3 is a nucleic acid or a nucleic acid analog or derivative thereof, also referred to as a nucleic acid agent herein. As will be appreciated by those skilled in the art, the depiction of a single strand also defines the sequence of the complementary strand. Thus, a nucleic acid also encompasses the complementary strand of a depicted single strand.

Without limitation, the nucleic acid agent can be single-stranded or double-stranded. A single-stranded nucleic acid agent can have double-stranded regions, e.g., where there is internal self-complementarity, and a double-stranded nucleic acid agent can have single-stranded regions. The nucleic acid can be of any desired length. In particular embodiments, nucleic acid can range from about 10 to 100 nucleotides in length. In various related embodiments, nucleic acid agents, single-stranded, double-stranded, and triple-stranded, can range in length from about 10 to about 50 nucleotides, from about 20 to about 50 nucleotides, from about 15 to about 30 nucleotides, from about 20 to about 30 nucleotides in length. In some embodiments, a nucleic acid agent is from about 9 to about 39 nucleotides in length. In some other embodiments, a nucleic acid agent is at least 30 nucleotides in length.

The nucleic acid agent can comprise modified nucleosides as known in the art. Modifications can alter, for example, the stability, solubility, or interaction of the nucleic acid agent with cellular or extracellular components that modify activity. In certain instances, it can be desirable to modify one or both strands of a double-stranded nucleic acid agent. In some cases, the two strands will include different modifications. In other instances, multiple different modifications can be included on each of the strands. The various modifications on a given strand can differ from each other, and can also differ from the various modifications on other strands. For example, one strand can have a modification, and a different strand can have a different modification. In other cases, one strand can have two or more different modifications, and the another strand can include a modification that differs from the at least two modifications on the first strand.

In some embodiments, the antagonist of METTL3 is a single-stranded and double-stranded nucleic acid agent that is effective in inducing RNA interference, referred to as siRNA, RNAi agent, or iRNA agent herein. iRNA agents suitable for inducing RNA interference in METTL3 are disclosed, for example, in WO2013/019857, the contents of which are incorporated herein by reference in their entirety.

### RNAi Inhibitors of METTL3

In one embodiment, the iRNA agent includes double-stranded ribonucleic acid (dsRNA) molecules for inhibiting the expression of a gene encoding METTL3 or METTL4 in a cell, e.g., a cell in a population of human stem cells and/or progenitor cells, where the dsRNA includes an antisense strand having a region of complementarity which is complementary to at least a part of an mRNA formed in the expression of a gene encoding METTL3 or METTL4, and where the region of complementarity is 30 nucleotides or less in length, generally 19-24 nucleotides in length, and where the dsRNA, upon contact with or introduction to a cell expressing the gene METTL3 or METTL4, inhibits the expression of the gene by at least 10% as assayed by, for example, a PCR or branched DNA (bDNA)-based method, or by a protein-based method, such as by immunoassay or Western blot. Expression of METTL3 or METTL4 in cell culture can be assayed by measuring METTL3 or METTL4 mRNA levels, respectively, such as by bDNA or TaqMan assay, or by measuring protein levels, such as by immunofluorescence analysis, using, for example, Western Blotting or flow cytometric techniques.

In some embodiments, the iRNA agent is an antisense oligonucleotide. One of skill in the art is well aware that single-stranded oligonucleotides can hybridize to a complementary target sequence and prevent access of the translation machinery to the target RNA transcript, thereby preventing protein synthesis. The single-stranded oligonucleotide can also hybridize to a complementary RNA and the RNA target can be subsequently cleaved by an enzyme such as RNase H and thus preventing translation of target RNA. Alternatively, or in addition, the single-stranded oligonucleotide can modulate the expression of a target sequence via RISC mediated cleavage of the target sequence, i.e., the single-stranded oligonucleotide acts as a single-stranded RNAi agent. A “single-stranded RNAi agent” as used herein, is an RNAi agent which is made up of a single molecule. A single-stranded RNAi agent can include a duplexed region, formed by intra-strand pairing, e.g., it can be, or include, a hairpin or pan-handle structure.

In some embodiments, the iRNA agent is a small hairpin RNA or short hairpin RNA (shRNA), a sequence of RNA that makes a tight hairpin turn that can be used to silence target gene expression via RNA interference (RNAi).

Without wishing to be bound by theory, METTL3 (also known by aliases methyltransferase like 3,M6A, “mRNA (2′-O-methyladenosine-N(6)-)-methyltransferase”, MT-A70, “N6-adenosine-methyltransferase 70 kDa subunit”, Spo8) is a member of methyltransferase like family. The amino acid sequence of human METTL3 has Accession number NP_062826.2 and the following sequence:

(SEQ ID NO: 2)
MSDTWSSIQAHKKQLDSLRERLQRRRKQDSGHLDLRNPEAALSPTFRSDS
PVPTAPTSGGPKPSTASAVPELATDPELEKKLLHHLSDLALTLPTDAVSI
CLAISTPDAPATQDGVESLLQKFAAQELIEVKRGLLQDDAHPTLVTYADH
SKLSAMMGAVAEKKGPGEVAGTVTGQKRRAEQDSTTVAAFASSLVSGLNS
SASEPAKEPAKKSRKHAASDVDLEIESLLNQQSTKEQQSKKVSQEILELL
NTTTAKEQSIVEKFRSRGRAQVQEFCDYGTKEECMKASDADRPCRKLHFR
RIINKHTDESLGDCSFLNTCFHMDTCKYVHYEIDACMDSEAPGSKDHTPS
QELALTQSVGGDSSADRLFPPQWICCDIRYLDVSILGKFAVVMADPPWDI
HMELPYGTLTDDEMRRLNIPVLQDDGFLFLWVTGRAMELGRECLNLWGYE
RVDEIIWVKTNQLQRIIRTGRTGHWLNHGKEHCLVGVKGNPQGFNQGLDC
DVIVAEVRSTSHKPDEIYGMIERLSPGTRKIELFGRPHNVQPNWITLGNQ
LDGIHLLDPDVVARFK QRYPDGIISKPKNL

Inhibition of the METTL3 gene can be by gene silencing RNAi molecules according to methods commonly known by a skilled artisan. For example, a gene silencing siRNA oligonucleotide duplexes targeted specifically to human METTL3 (GenBank No: NM_019852.4) can readily be used to knockdown METTL3 expression. METTL3 mRNA can be successfully targeted using siRNAs; and other siRNA molecules may be readily prepared by those of skill in the art based on the known sequence of the target mRNA. To avoid doubt, the sequence of a human METTL3 is provided at, for example, GenBank Accession Nos. NM_019852.4 (SEQ ID NO: 1). Accordingly, in avoidance of any doubt, one of ordinary skill in the art can design nucleic acid inhibitors, such as RNAi (RNA silencing) agents to mRNA nucleic acid sequence of human METTL3 of NM_019852.4 (SEQ ID NO: 1) which is as follows:

| (SEQ ID NO: 1) | |
|---|---|
| 1 | aaatgacttt tctgtcttgc tcagctccag gggtcatttt ccggttagcc ttcggggtgt |
| 61 | ccgcgtgaga attggctata tcctggagcg agtgctggga ggtgctagtc cgccgcgcct |
| 121 | tattcgagag gtgtcagggc tgggagacta ggatgtcgga cacgtggagc tctatccagg |
| 181 | cccacaagaa gcagctggac tctctgcggg agaggctgca gcggaggcgg aagcaggact |
| 241 | cggggcactt ggatctacgg aatccagagg cagcattgtc tccaaccttc cgtagtgaca |
| 301 | gcccagtgcc tactgcaccc acctctggtg gccctaagcc cagcacagct tcagcagttc |
| 361 | ctgaattagc tacagatcct gagttagaga agaagttgct acaccacctc tctgatctgg |
| 421 | ccttaacatt gcccactgat gctgtgtcca tctgtcttgc catctccacg ccagatgctc |
| 481 | ctgccactca agatggggta gaaagcctcc tgcagaagtt tgcagctcag gagttgattg |
| 541 | aggtaaagcg aggtctccta caagatgatg cacatcctac tcttgtaacc tatgctgacc |
| 601 | attccaagct ctctgccatg atgggtgctg tggcagaaaa gaagggccct ggggaggtag |
| 661 | cagggactgt cacagggcag aagcggcgtg cagaacagga ctcgactaca gtagctgcct |
| 721 | ttgccagttc gttagtctct ggtctgaact cttcagcatc ggaaccagca aaggagccag |
| 781 | ccaagaaatc aaggaaacat gctgcctcag atgttgatct ggagatagag agccttctga |
| 841 | accaacagtc cactaaggaa caacagagca agaaggtcag tcaggagatc ctagagctat |
| 901 | taaatactac aacagccaag gaacaatcca ttgttgaaaa atttcgctct cgaggtcggg |
| 961 | cccaagtgca agaattctgt gactatggaa ccaaggagga gtgcatgaaa gccagtgatg |
| 1021 | ctgatcgacc ctgtcgcaag ctgcacttca gacgaattat caataaacac actgatgagt |
| 1081 | ctttaggtga ctgctctttc cttaatacat gtttccacat ggatacctgc aagtatgttc |
| 1141 | actatgaaat tgatgcttgc atggattctg aggcccctgg cagcaaagac cacacgccaa |
| 1201 | gccaggagct tgctcttaca cagagtgtcg gaggtgattc cagtgcagac cgactcttcc |
| 1261 | cacctcagtg gatctgttgt gatatccgct acctggacgt cagtatcttg ggcaagtttg |
| 1321 | cagttgtgat ggctgaccca ccctgggata ttcacatgga actgccctat gggaccctga |
| 1381 | cagatgatga gatgcgcagg ctcaacatac ccgtactaca ggatgatggc tttctcttcc |
| 1441 | tctgggtcac aggcagggcc atggagttgg ggagagaatg tctaaacctc tgggggtatg |
| 1501 | aacgggtaga tgaaattatt tgggtgaaga caaatcaact gcaacgcatc attcggacag |
| 1561 | gccgtacagg tcactggttg aaccatggga aggaacactg cttggttggt gtcaaaggaa |
| 1621 | atccccaagg cttcaaccag ggtctggatt gtgatgtgat cgtagctgag gttcgttcca |
| 1681 | ccagtcataa accagatgaa atctatggca tgattgaaag actatctcct ggcactcgca |
| 1741 | agattgagtt atttggacga ccacacaatg tgcaacccaa ctggatcacc cttggaaacc |
| 1801 | aactggatgg gatccaccta ctagacccag atgtggttgc acggttcaag caaaggtacc |
| 1861 | cagatggtat catctctaaa cctaagaatt tatagaagca cttccttaca gagctaagaa |
| 1921 | tccatagcca tggctctgta agctaaacct gaagagtgat atttgtacaa tagctttctt |
| 1981 | ctttatttaa ataaacattt gtattgtagt tgggattctg aaaaaaaaaa aaaaaaaa |

Without wishing to be bound by theory, METTL4 (also known by aliases methyltransferase like 4, FLJ23017 and HsT661) is a member of methyltransferase like family. The amino acid sequence of human METTL4 has Accession number NP_073751.3 and the following sequence:

(SEQ ID NO: 7)
MSVVHQLSAGWLLDHLSFINKINYQLHQHHEPCCRKKEFTTSVHFESLQM
DSVSSSGVCAAFIASDSSTKPENDDGGNYEMFTRKFVFRPELFDVTKPYI
TPAVHKECQQSNEKEDLMNGVKKEISISIIGKKRKRCVVFNQGELDAMEY
HTKIRELILDGSLQLIQEGLKSGFLYPLFEKQDKGSKPITLPLDACSLSE
LCEMAKHLPSLNEMEHQTLQLVEEDTSVTEQDLFLRVVENNSSFTKVITL
MGQKYLLPPKSSFLLSDISCMQPLLNYRKTFDVIVIDPPWQNKSVKRSNR
YSYLSPLQIQQIPIPKLAAPNCLLVTWVTNRQKHLRFIKEELYPSWSVEV
VAEWHWVKITNSGEFVFPLDSPHKKPYEGLILGRVQEKTALPLRNADVNV
LPIPDHKLIVSVPCTLHSHKPPLAEVLKDYIKPDGEYLELFARNLQPGWT
SWGNEVLKFQHVDYFIAVESGS

Similarly, inhibition of the METTL4 gene can be by gene silencing RNAi molecules according to methods commonly known by a skilled artisan. For example, a gene silencing siRNA oligonucleotide duplexes targeted specifically to human METTL4 (GenBank No: NM_022840.4) can readily be used to knockdown METTL4 expression. METTL4 mRNA can be successfully targeted using siRNAs; and other siRNA molecules may be readily prepared by those of skill in the art based on the known sequence of the target mRNA. To avoid doubt, the sequence of a human METTL4 is provided at, for example, GenBank Accession Nos. NM_022840.4 (SEQ ID NO: 8). Accordingly, in avoidance of any doubt, one of ordinary skill in the art can design nucleic acid inhibitors, such as RNAi (RNA silencing) agents to mRNA nucleic acid sequence of human METTL4 of NM_022840.4 (SEQ ID NO: 8) which is as follows:

| (SEQ ID NO: 8) | |
|---|---|
| 1 | atgcgaccgc ctcgtcgctg gaaggctgcg tgctggtcgc gcccagctgc gtcaccccag |
| 61 | gaactggggt ctgtgggcca gtgtggccgt ctctacgaag actggcacga cccctaaagt |
| 121 | taggtcggaa gacctgtggg cagcttgagc gccgaggagt gccctgaacg ctcaactcgc |
| 181 | cctggaaacg tttttccgta cagcaacatg gcggcgccca tggactctta gaaaaggaga |
| 241 | aagctttttc tctgtggact ggaaggggca tttttcatga tcactattta gatgggtgct |
| 301 | gttttcatga ggagagtctg ggaaggcggc gtccgctttt ctgacaaggg aagaggctac |
| 361 | tttgtccttt taaggattca atgacttcct gacttggagg atgtggacct agtggctaga |
| 421 | cccaaggacc aaagcaagaa gtcgtggggg gcccaggaag acaggaggat cacattggga |
| 481 | ttccagacat aagatcaggt tttaaccccc tttggccaaa ttttggctga aaatgttgaa |
| 541 | ttatcaactc tgaaattaaa aagaaagttt atattaaaac attgcaattt tccttagaat |
| 601 | ttctgtatat attaacatca tgaatgataa attctcttca atgtgcatgt caggtttttg |
| 661 | tacttgtata tcaaatctat ctgtgtgtat gaagtgtatg tttattgaaa tacaagatat |
| 721 | ttaagaagct gatctggaaa gttggatttt cattctagtt cctaattccc agaggctttt |
| 781 | ttaaaggaag ggaatgtctg tggtacacca gttgtcagct gggtggttac tggatcatct |
| 841 | ttcttttatc aacaagataa actatcaact tcaccagcat catgaacctt gttgccgtaa |
| 901 | aaaggagttc actacttctg ttcactttga gtctcttcaa atggattctg tgtcctcctc |
| 961 | tggagtctgt gctgcattta ttgcttctga ctcttccact aagccagaga atgatgatgg |
| 1021 | aggaaattat gaaatgttca cacgaaaatt tgtttttcga cctgaactgt ttgatgtcac |
| 1081 | caaaccttat ataactccag ctgttcataa agaatgccag caaagtaatg aaaaggaaga |
| 1141 | tctgatgaat ggtgttaaaa aagaaatctc catttctatt attgggaaga agcgtaaaag |
| 1201 | atgtgttgtt ttcaatcaag gtgaattgga tgctatggaa taccatacaa agatcaggga |
| 1261 | gctgattttg gatggatctt tacagttgat ccaggaaggt ctcaaaagtg gttttcttta |
| 1321 | tccacttttt gaaaaacagg acaagggtag taagcccatt actttaccac ttgacgcctg |
| 1381 | cagtttgtca gaattatgtg aaatggcaaa gcatttgcct tctctgaatg aaatggaaca |
| 1441 | tcagacatta caattggtgg aagaggatac atctgttaca gaacaggatt tatttttgcg |
| 1501 | agttgttgaa aacaactcta gctttacaaa agtgattact ttaatgggac agaaatacct |
| 1561 | gctaccaccg aaaagcagtt ttcttttatc tgacatttct tgtatgcaac cacttctaaa |
| 1621 | ctataggaaa acatttgatg taattgtgat agatccacca tggcagaaca aatcagttaa |
| 1681 | aagaagtaat aggtacagtt atttgtcacc cctgcaaata cagcaaatac ctatccctaa |
| 1741 | attggctgct ccaaactgtc ttcttgttac ttgggtgacc aatagacaga agcacctacg |
| 1801 | ttttataaag gaagaacttt atccctcttg gtctgtggag gtagttgctg agtggcactg |
| 1861 | ggtaaaaata accaattcag gagaatttgt gttcccatta gattctccac acaaaaagcc |
| 1921 | ctacgaaggt cttatactgg ggagggttca agaaaaaact gctctaccat tgaggaatgc |
| 1981 | agatgtaaac gtgctcccca ttccagacca caaattaatt gtcagcgtgc cctgtactct |
| 2041 | tcactcacat aagccaccgc ttgctgaggt tttaaaagac tacatcaagc cagatgggga |
| 2101 | atatttggag ttgtttgctc gaaatttaca gccaggttgg actagttggg gcaatgaagt |
| 2161 | tctcaaattt cagcatgtgg attattttat tgctgtggag tctggaagct gactatgatc |
| 2221 | ttgattaaag tagtggtttc ttcattgttt cctcaccact tttcccttaa ttctaagtca |
| 2281 | tttttttatt ttgttaccaa cccatattct tagaatataa acaggacttg tttttttcag |
| 2341 | taagggacca gaagtgacta gccttcatgt aattttaaga tgaattttac ttgagttgca |
| 2401 | ctaacattct atgttattct agactataca aattaagtgg taagcagtta taaagacggc |
| 2461 | aagaccatgc tattgaaaaa gttcagaaaa catacaccgt ggaccagagg tcttaatcct |
| 2521 | atctatggat gtgttttgtg tgacccatac agtgttgtaa aaaacactta gaaccattat |
| 2581 | tctaaaaaat ggggctattt cacattaaag tccagatttc tgcttctttt taaacatcag |
| 2641 | aggctctggc tacacagagg cctttgttct ttcctggcat cagtctgcag gaccaagcgg |
| 2701 | tggtggctca cttgggaaga gccttgtgct ctccactttg ccacagtacc actgccacca |
| 2761 | tgctgctcac ttatgtcatc cacttggccc ttgtatgacc tgaatttgca acctctggta |
| 2821 | tactgttatg ttctggagaa aatattcaaa gatctgccaa atactgcatt agtatactga |
| 2881 | gtttatacag catttttgta gggttttaaa ttgcattcaa ggtcactttc caagcacttt |
| 2941 | ctggttttgc ttgtttttct agaagaaaat gaaaagctat tccttataat aaacatggca |
| 3001 | gcaagtaaac agtgtgattg tgaaaaaaat attatttata gattttctac aaataaatat |
| 3061 | ttgtctacca agtaaaatat tttgactgaa atgattcttt gaaatgcata ttgatttatt |
| 3121 | atgtattgac tttttaaaaa ttgaggtata attttcacaa aattctccaa ttttcagtgt |
| 3181 | caaattcagt gaattttgaa aacatatata cagttgtctg tctgccacag tgatcatgat |
| 3241 | acagaacact ttctttaccc tgaaaacttc tcatttttcc ttttgcagtc aatcccctgc |
| 3301 | tcctatcctt ggcccctggc aaacactggt ttgctttcta tcattagttc tgctgtttga |
| 3361 | gaatttcata taaatggaat catgcaatgt gtaatctatt gtgcctggct tctttcacgt |
| 3421 | agcattttga gaaaagcatt tatactattt acagattgtt gacaaatatt tatccactaa |
| 3481 | gtaaaatgtt agactgaaat gattctttga caagcttgcc aatttactga ttttgtcaaa |
| 3541 | gaaaaatatg ttatttttga agtttgttca tcctttgagt gtgtgagtat agtatcagag |
| 3601 | gcttaatttt gtatttatgg agctattcta acttgttatt taaaaggaaa aaggtattaa |
| 3661 | acttgaagca aacttctcat gatctcaaaa aaaaaaaaaa aaaa |

In some embodiments, the shRNA for targeting METTL3 has a nucleotide sequence of that is substantially complementary to at least part of the target sequence GCTGCACTTCAGACGAATTAT (SEQ ID NO: 3) or a fragment of at least 10, at least 15, at least 20, or at least 25 contiguous nucleotides thereof. In some embodiments, the siRNA to METTL3 is GCUACCGUAUGGGACAUUA (SEQ ID NO: 4) or a fragment of at least 10, at least 15, at least 20, or at least 25 contiguous nucleotides thereof.

In some embodiments, an antagonist of METTL3 is an antigomir to a miRNA (also referred to as “miR”). miRs that have been shown to target METTL3 include, but are not limited to; miR-423-3p and miR-1226-3p, miR-330-5p, miR-668-3p, miR-1224-5p, and miR-1981, as disclosed in Chen et al., (Cell Stem Cell, 2015; 16(3), 289-301; “m6A RNA Methylation Is Regulated by MicroRNAs and Promotes Reprogramming to Pluripotency”). In some embodiments, an inhibitor of METTL3 is an antigomir to miR-423-3p and/or to miR-1226-3p, i.e., an anti-miR-423-3p and/or anti-miR-1226-3p, which decreases the METTL3 interaction or binding on the mRNA. In some embodiments, an anti-miR-423-3p comprises ACUGAGGGGCCUCAGACCGAGCU (SEQ ID NO: 5) or a fragment of at least 10, at least 15, at least 20, or at least 24 contiguous nucleotides thereof. In some embodiments, an anti-miR-1226-3p comprises CUAGGGAACACAGGGCUGGUGA (SEQ ID NO: 6) or a fragment of at least 10, at least 15, at least 20, or at least 24 contiguous nucleotides thereof.

In general, any method of delivering a nucleic acid molecule can be adapted for use with the nucleic acid agents described herein. Methods of delivering RNA interference agents, e.g., an siRNA, or vectors containing an RNA interference agent, to the target cells, e.g., stem cells and/or progenitor cells, for uptake include injection of a composition containing the RNA interference agent, e.g., an siRNA, or directly contacting the cell with a composition comprising an RNA interference agent, e.g., an siRNA. In another embodiment, RNA interference agent, e.g., an siRNA may be injected directly into any blood vessel, such as vein, artery, venule or arteriole, via, e.g., hydrodynamic injection or catheterization. Administration may be by a single injection or by two or more injections. The RNA interference agent is delivered in a pharmaceutically acceptable carrier. One or more RNA interference agents may be used simultaneously. In one embodiment, specific cells are targeted with RNA interference, limiting potential side effects. The method can use, for example, a complex or a fusion molecule comprising a cell targeting moiety and an RNA interference binding moiety that is used to deliver RNA interference effectively into cells. For example, an antibody-protamine fusion protein when mixed with siRNA, binds siRNA and selectively delivers the siRNA into cells expressing an antigen recognized by the antibody, resulting in silencing of gene expression only in those cells that express the antigen. The siRNA or RNA interference-inducing molecule binding moiety is a protein or a nucleic acid binding domain or fragment of a protein, and the binding moiety is fused to a portion of the targeting moiety. The location of the targeting moiety can be either in the carboxyl-terminal or amino-terminal end of the construct or in the middle of the fusion protein. A viral-mediated delivery mechanism can also be employed to deliver siRNAs to cells in vitro and in vivo as described in Xia, H. et al. (2002) Nat Biotechnol 20(10):1006). Plasmid- or viral-mediated delivery mechanisms of shRNA may also be employed to deliver shRNAs to cells in vitro and in vivo as described in Rubinson, D. A., et al. ((2003) Nat. Genet. 33:401-406) and Stewart, S. A., et al. ((2003) RNA 9:493-501). The RNA interference agents, e.g., the siRNAs or shRNAs, can be introduced along with components that perform one or more of the following activities: enhance uptake of the RNA interfering agents, e.g., siRNA, by the cell, inhibit annealing of single strands, stabilize single strands, or otherwise facilitate delivery to the target cell and increase inhibition of the target gene, e.g., METTL3. The dose of the particular RNA interfering agent will be in an amount necessary to effect RNA interference, e.g., post translational gene silencing (PTGS), of the particular target gene, thereby leading to inhibition of target gene expression or inhibition of activity or level of the protein encoded by the target gene.

### Oligonucleotide Modifications

In some embodiments, RNAi agents that inhibit METTL3 for use in the aspects of the invention as disclosed herein can include oligonucleotide modifications. Unmodified oligonucleotides can be less than optimal in some applications, e.g., unmodified oligonucleotides can be prone to degradation by e.g., cellular nucleases. However, chemical modifications to one or more of the subunits of oligonucleotide can confer improved properties, e.g., can render oligonucleotides more stable to nucleases. Typical oligonucleotide modifications can include one or more of: (i) alteration, e.g., replacement, of one or both of the non-linking phosphate oxygens and/or of one or more of the linking phosphate oxygens in the phosphodiester intersugar linkage; (ii) alteration, e.g., replacement, of a constituent of the ribose sugar, e.g., of the 2′ hydroxyl on the ribose sugar; (iii) wholesale replacement of the phosphate moiety with “dephospho” linkers; (iv) modification or replacement of a naturally occurring base with a non-natural base; (v) replacement or modification of the ribose-phosphate backbone, e.g. peptide nucleic acid (PNA); (vi) modification of the 3′ end or 5′ end of the oligonucleotide, e.g., removal, modification or replacement of a terminal phosphate group or conjugation of a moiety, e.g., conjugation of a ligand, to either the 3′ or 5′ end of oligonucleotide; and (vii) modification of the sugar, e.g., six membered rings.

The terms replacement, modification, alteration, and the like, as used in this context, do not imply any process limitation, e.g., modification does not mean that one must start with a reference or naturally occurring ribonucleic acid and modify it to produce a modified ribonucleic acid bur rather modified simply indicates a difference from a naturally occurring molecule. As described below, modifications, e.g., those described herein, can be provided as asymmetrical modifications.

A modification described herein can be the sole modification, or the sole type of modification included on multiple nucleotides, or a modification can be combined with one or more other modifications described herein. The modifications described herein can also be combined onto an oligonucleotide, e.g. different nucleotides of an oligonucleotide have different modifications described herein.

Described herein are iRNA agents that inhibit the expression of METTL3. In one embodiment, the iRNA agent includes double-stranded ribonucleic acid (dsRNA) molecules for inhibiting the expression of METTL3 in a cell ex vivo, e.g., in HSPCs ex vivo obtained from blood or UCB, where the dsRNA includes an antisense strand having a region of complementarity which is complementary to at least a part of an mRNA formed in the expression of METTL3, and where the region of complementarity is 30 nucleotides or less in length, generally 19-24 nucleotides in length, and where the dsRNA, upon contact with or introduction to a cell expressing the gene encoding METTL3, inhibits the expression of the gene by at least 10% as assayed by, for example, a PCR or branched DNA (bDNA)-based method, or by a protein-based method, such as by immunoassay or Western blot. Expression of METTL3 in cell culture, such as a stem cell population, can be assayed by measuring mRNA levels of METTL3, such as by bDNA or TaqMan assay, or by measuring protein levels, such as by immunofluorescence analysis, using, for example, Western Blotting or flow cytometric techniques.

A dsRNA includes two RNA strands that are complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of METTL3 mRNA, e.g, SEQ ID NO: 1 as disclosed herein. The other strand (the sense strand) includes a region that is complementary to the antisense strand, such that the two strands hybridize and form a duplex structure when combined under suitable conditions. Generally, the duplex structure is between 15 and 30 inclusive, more generally between 18 and 25 inclusive, yet more generally between 19 and 24 inclusive, and most generally between 19 and 21 base pairs in length, inclusive. Similarly, the region of complementarity to the target sequence is between 15 and 30 inclusive, more generally between 18 and 25 inclusive, yet more generally between 19 and 24 inclusive, and most generally between 19 and 21 nucleotides in length, inclusive. In some embodiments, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

One of skill in the art will also recognize that the duplex region is a primary functional portion of a dsRNA, e.g., a duplex region of 9 to 36, e.g., 15-30 base pairs. Thus, in one embodiment, to the extent that it becomes processed to a functional duplex of e.g., 15-30 base pairs that targets a desired RNA for cleavage, an RNA molecule or complex of RNA molecules having a duplex region greater than 30 base pairs is a dsRNA. Thus, an ordinarily skilled artisan will recognize that in one embodiment, then, an miRNA is a dsRNA. In another embodiment, a dsRNA is not a naturally occurring miRNA. In another embodiment, an iRNA agent useful to target expression of METTL3 is not generated in the target cell by cleavage of a larger dsRNA.

A dsRNA as described herein can further include one or more single-stranded nucleotide overhangs. The dsRNA can be synthesized by standard methods known in the art as further discussed below, e.g., by use of an automated DNA synthesizer, such as are commercially available from, for example, Biosearch, Applied Biosystems, Inc. In one embodiment, a gene encoding METTL3 is a human gene. In another embodiment the gene encoding METTL3 is a mouse or rat gene.

In one aspect, a dsRNA will include at least two nucleotide sequences, a sense and an anti-sense sequence, wherein the sense strand is SEQ ID NO: 1. In this aspect, one of the two sequences is complementary to the other of the two sequences, with one of the sequences being substantially complementary to a sequence of the METTL3 mRNA. As described elsewhere herein and as known in the art, the complementary sequences of a dsRNA can also be contained as self-complementary regions of a single nucleic acid molecule, as opposed to being on separate oligonucleotides.

The skilled person is well aware that dsRNAs having a duplex structure of between 20 and 23, but specifically 21, base pairs have been hailed as particularly effective in inducing RNA interference (Elbashir et al., EMBO 2001, 20:6877-6888). However, others have found that shorter or longer RNA duplex structures can be effective as well. In the embodiments, a dsRNAs described herein can include at least one strand of a length of minimally 21 nt. It can be reasonably expected that shorter duplexes having one of the sequences of Tables 2-7 minus only a few nucleotides on one or both ends can be similarly effective as compared to the dsRNAs described above. Hence, dsRNAs having a partial sequence of at least 15, 16, 17, 18, 19, 20, or more contiguous nucleotides from one of the sequences of SEQ ID NO: 3 or 4, and differing in their ability to inhibit the expression of a gene encoding METTL3 by not more than 5, 10, 15, 20, 25, or 30% inhibition from a dsRNA comprising the full sequence, are contemplated according to the technology described herein.

While a target sequence is generally 15-30 nucleotides in length, there is wide variation in the suitability of particular sequences in this range for directing cleavage of any given target RNA. Various software packages and the guidelines set out herein provide guidance for the identification of optimal target sequences for any given gene target, but an empirical approach can also be taken in which a “window” or “mask” of a given size (as a non-limiting example, 21 nucleotides) is literally or figuratively (including, e.g., in silico) placed on the target RNA sequence to identify sequences in the size range that can serve as target sequences. By moving the sequence “window” progressively one nucleotide upstream or downstream of an initial target sequence location, the next potential target sequence can be identified, until the complete set of possible sequences is identified for any given target size selected. This process, coupled with systematic synthesis and testing of the identified sequences (using assays as described herein or as known in the art) to identify those sequences that perform optimally can identify those RNA sequences that, when targeted with an iRNA agent, mediate the best inhibition of target gene expression. Thus, it is contemplated that further optimization of inhibition efficiency can be achieved by progressively “walking the window” one nucleotide upstream or downstream of the given sequences to identify sequences with equal or better inhibition characteristics.

Further, it is contemplated that for any sequence identified by a sequence identifier NO: 3 or 4, can be further optimization could be achieved by systematically either adding or removing nucleotides to generate longer or shorter sequences and testing those and sequences generated by walking a window of the longer or shorter size up or down the target RNA from that point. Again, coupling this approach to generating new candidate targets with testing for effectiveness of iRNAs based on those target sequences in an inhibition assay as known in the art or as described herein can lead to further improvements in the efficiency of inhibition. Further still, such optimized sequences can be adjusted by, e.g., the introduction of modified nucleotides as described herein or as known in the art, addition or changes in overhang, or other modifications as known in the art and/or discussed herein to further optimize the molecule (e.g., increasing serum stability or circulating half-life, increasing thermal stability, enhancing transmembrane delivery, targeting to a particular location or cell type, increasing interaction with silencing pathway enzymes, increasing release from endosomes, etc.) as an expression inhibitor.

An iRNA as described herein can contain one or more mismatches to the target sequence. In one embodiment, an iRNA as described herein contains no more than 3 mismatches. If the antisense strand of the iRNA contains mismatches to a target sequence, it is preferable that the area of mismatch not be located in the center of the region of complementarity. If the antisense strand of the iRNA contains mismatches to the target sequence, it is preferable that the mismatch be restricted to be within the last 5 nucleotides from either the 5′ or 3′ end of the region of complementarity. For example, for a 23 nucleotide iRNA agent RNA strand which is complementary to a region of a gene encoding METTL3, the RNA strand generally does not contain any mismatch within the central 13 nucleotides. The methods described herein or methods known in the art can be used to determine whether an iRNA containing a mismatch to a target sequence is effective in inhibiting the expression of METTL3. Consideration of the efficacy of iRNAs with mismatches in inhibiting expression of METTL3 is important, especially if the particular region of complementarity to the METTL3 gene is known to have polymorphic sequence variation within the population.

In one embodiment, at least one end of a dsRNA has a single-stranded nucleotide overhang of 1 to 4, generally 1 or 2 nucleotides. dsRNAs having at least one nucleotide overhang have unexpectedly superior inhibitory properties relative to their blunt-ended counterparts. In yet another embodiment, the RNA of an iRNA, e.g., a dsRNA, is chemically modified to enhance stability or other beneficial characteristics. The nucleic acids featured in the technology described herein can be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In particular embodiments, the modified RNA will have a phosphorus atom in its internucleoside backbone.

Modified RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, each of which is herein incorporated by reference

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH.sub.2 component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference.

In other embodiments, suitable RNA mimetics suitable are contemplated for use in iRNAs, in which both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found, for example, in Nielsen et al., Science, 1991, 254, 1497-1500.

Antisense molecules or antisense oligonucleotides (ASOs) are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. See for example, Vermeulen et al., RNA 13: 723-730 (2007) and in WO2007/095387 and WO 2008/036825; Yue, et al., Curr. Genomics, 10(7):478-92 (2009) and Lennox Gene Ther. 18(12):1111-20 (2011), which are incorporated by reference herein in their entireties.

Thus, antisense molecules that inhibit METTL3 and/or METTL4 can be designed and made using standard nucleic acid synthesis techniques or obtained from a commercial entity, e.g., Regulus Therapeutics (San Diego, Calif.). Optionally, the antisense molecule is single-stranded and comprises RNA and/or DNA. Optionally, the backbone of the molecule is modified by various chemical modifications to improve the in vitro and in vivo stability and to improve the in vivo delivery of antisense molecules. Modifications of antisense molecules include, but are not limited to, 2′-O-methyl modifications, 2′-O-methyl modified ribose sugars with terminal phosphorothioates and a cholesterol group at the 3′ end, 2′-O-methoxyethyl (2′-MOE) modifications, 2′-fluoro modifications, and 2′,4′ methylene modifications (referred to as “locked nucleic acids” or LNAs). Thus, inhibitory nucleic acids include, for example, modified oligonucleotides (2′-O-methylated or 2′-O-methoxyethyl), locked nucleic acids (LNA; see, e.g, Valóczi et al., Nucleic Acids Res. 32(22):e175 (2004)), morpholino oligonucleotides (see, e.g, Kloosterman et al., PLoS Biol 5(8):e203 (2007)), peptide nucleic acids (PNAs), PNA-peptide conjugates, and LNA/2′-O-methylated oligonucleotide mixmers (see, e.g., Fabiani and Gait, RNA 14:336-46 (2008)). Optionally, the antisense molecule is an antagomir. Antagomirs are oligonucleotides comprising 2′-O-methyl modified ribose sugars with terminal phosphorothioates and a cholesterol group at the 3′ end.

miRs comprising LNA (typically identified in capitals, DNA in lower case, complete phosphorothioate backbone, where a capital C denotes LNA methylcytosine, are described in Lanford et al., Science 327(5962:198-201 (2010), which is incorporated by reference herein in its entirety. See also Elmen et al., Nature 452:896-9 (2008); and Elmen et al., Nucleic Acids Res. 36:1153-1162 (2008), which are incorporated by reference herein in their entireties. Optionally, the nucleic acid comprises a targeting sequence of miR-103, miR-105, miR-107 and miR-155. Such miRNA-binding nucleic acids are referred to as miRNA decoys or miRNA sponges. For example, mRNAs with multiple copies of the miRNA target can be engineered into the 3′ UTR of the mRNA creating an miRNA “sponge.” The miRNA inhibitors function by sequestering the cellular miRNAs away from the mRNAs that normally would be targeted by them. Such nucleic acid decoys can be delivered, e.g., by viral vectors, and expressed to inhibit the activity of any of miR-103, miR-105, miR-107 and miR-155.

Ribozymes are nucleic acid molecules that are capable of catalyzing a chemical reaction, either intramolecularly or intermolecularly. Typically, ribozymes cleave RNA or DNA substrates. There are a number of different types of ribozymes that catalyze chemical reactions which are based on ribozymes found in natural systems, such as hammerhead ribozymes, and hairpin ribozymes. There are also a number of ribozymes that are not found in natural systems, but which have been engineered to catalyze specific reactions. See, for example, U.S. Pat. Nos. 5,807,718, and 5,910,408. Representative examples of how to make and use ribozymes to catalyze a variety of different reactions can be found in, for example, U.S. Pat. Nos. 5,837,855, 5,877,022, 5,972,704, 5,989,906, and 6,017,756.

Small Molecule Inhibitors of METTL3

In some embodiments, the antagonist of METTL3 is a small molecule. As used herein, the term “small molecule” refers to a natural or synthetic molecule having a molecular mass of less than about 5 kD, organic or inorganic compounds having a molecular mass of less than about 5 kD, less than about 2 kD, or less than about 1 kD.

In some embodiments, the antagonist of METTL3 can have an IC50 of less than 50 μM, e.g., the antagonist of METTL3 can have an IC50 of from about 50 μM to about 5 nM, or less than 5 nM. For example, in some embodiments, an antagonist of METTL3 has an IC50 of from about 50 μM to about 25 μM, from about 25 μM to about 10 μM, from about 10 μM to about 5 μM, from about 5 μM to about 1 μM, from about 1 μM to about 500 nM, from about 500 nM to about 400 nM, from about 400 nM to about 300 nM, from about 300 nM to about 250 nM, from about 250 nM to about 200 nM, from about 200 nM to about 150 nM, from about 150 nM to about 100 nM, from about 100 nM to about 50 nM, from about 50 nM to about 30 nM, from about 30 nM to about 25 nM, from about 25 nM to about 20 nM, from about 20 nM to about 15 nM, from about 15 nM to about 10 nM, from about 10 nM to about 5 nM, or less than about 5 nM.

In some embodiments, the antagonist of METTL3 can be an anti-METTL3 antibody molecule or an antigen-binding fragment thereof. Suitable antibodies include, but are not limited to, polyclonal, monoclonal, chimeric, humanized, recombinant, single chain, F_{ab}, F_{ab′}, F_{sc}, Rᵥ, and F_{(ab′)2} fragments. In some embodiments, neutralizing antibodies can be used as anti-METTL3 antibodies. Antibodies are readily raised in animals such as rabbits or mice by immunization with the antigen. Immunized mice are particularly useful for providing sources of B cells for the manufacture of hybridomas, which in turn are cultured to produce large quantities of monoclonal antibodies. In general, an antibody molecule obtained from humans can be classified in one of the immunoglobulin classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

Antibodies provide high binding avidity and unique specificity to a wide range of target antigens and haptens. Monoclonal antibodies useful in the practice of the methods disclosed herein include whole antibody and fragments thereof and are generated in accordance with conventional techniques, such as hybridoma synthesis, recombinant DNA techniques and protein synthesis.

The METTL3 polypeptide, or a portion or fragment thereof, can serve as an antigen, and additionally can be used as an immunogen to generate antibodies that immunospecifically bind the antigen, using standard techniques for polyclonal and monoclonal antibody preparation. Preferably, the antigenic peptide comprises at least 10 amino acid residues, or at least 15 amino acid residues, or at least 20 amino acid residues, or at least 30 amino acid residues.

Useful monoclonal antibodies and fragments can be derived from any species (including humans) or can be formed as chimeric proteins which employ sequences from more than one species. Human monoclonal antibodies or “humanized” murine antibody can also be used in accordance with the present invention. For example, murine monoclonal antibody can be “humanized” by genetically recombining the nucleotide sequence encoding the murine Fv region (i.e., containing the antigen binding sites) or the complementarily determining regions thereof with the nucleotide sequence encoding a human constant domain region and an Fc region. Humanized targeting moieties are recognized to decrease the immunoreactivity of the antibody or polypeptide in the host recipient, permitting an increase in the half-life and a reduction in the possibility of adverse immune reactions in a manner similar to that disclosed in European Patent Application No. 0,411,893 A2. The murine monoclonal antibodies should preferably be employed in humanized form. Antigen binding activity is determined by the sequences and conformation of the amino acids of the six complementarily determining regions (CDRs) that are located (three each) on the light and heavy chains of the variable portion (Fv) of the antibody. The 25-kDa single-chain Fv (scFv) molecule, composed of a variable region (VL) of the light chain and a variable region (VH) of the heavy chain joined via a short peptide spacer sequence, is one option for minimizing the size of an antibody agent. ScFvs provide additional options for preparing and screening a large number of different antibody fragments to identify those that specifically bind. Techniques have been developed to display scFv molecules on the surface of filamentous phage that contain the gene for the scFv. scFv molecules with a broad range or antigenic-specificities can be present in a single large pool of scFv-phage library.

Chimeric antibodies are immunoglobin molecules characterized by two or more segments or portions derived from different animal species. Generally, the variable region of the chimeric antibody is derived from a non-human mammalian antibody, such as murine monoclonal antibody, and the immunoglobin constant region is derived from a human immunoglobin molecule. Preferably, both regions and the combination have low immunogenicity as routinely determined.

Anti-METTL3 antibodies are commercially available through vendors such as Thermo Scientific, Sigma Aldrich, Atlas Antibodies, and R&D Systems.

Gene Editing

While it is preferred that METTL3 and/or METTL4 inhibition in a stem cell population is reversible or transient, thereby allowing the cell to differentiate along a lineage at a later timepoint, in some embodiments, the inhibition of METTL3 comprises contacting the population of stem cells and/or progenitor cells with a genome-editing agent for targeted excision of the METTL3 and/or METTL4 gene from at least one stem cell. As used herein, the term “genome-editing agent” refers to a compound or a composition that can modify a nucleotide sequence in the genome of an organism. In some embodiments, the genome-editing agent can excise a specific nucleotide sequence from the target genome. In some embodiments, the genome-editing agent can disrupt the function of a specific nucleotide sequence, for example, by breaking one or more bonds in the sequence. Genome editing can be achieved through processes such as nuclease-mediated mutagenesis, chemical mutagenesis, radiation mutagenesis, or meganuclease-mediated mutagenesis.

In some embodiment, the genome-editing agent comprises a DNA-binding member and a nuclease, wherein the DNA-binding member localizes the nuclease to a target site which is then cut by the nuclease.

In some embodiments, the genome-editing agent is a CRISPR/Cas system. In some embodiments, the CRISPR/Cas system is CRISPR/Cas9, which is disclosed in U.S. Pat. No. 8,697,359 and US Application 2015/0291966, which is corporated herein in its entirety by reference. In alternative embodiments, the CRISPR/Cas system is CRISPR/Cpf1, as disclosed in Zetsche et al., 2015; Cell 163(3); 759-777 “Cpf1 Is a Single RNA-Guided Endonuclease of a Class 2 CRISPR-Cas System”, which is incorporated herein in its entirety by reference. The CRISPR/Cas is an engineered nuclease system based on a bacterial system that can be used for genome engineering. It is based on part of the adaptive immune response of many bacteria and archea. When a virus or plasmid invades a bacterium, segments of the invader's DNA are converted into CRISPR RNAs (crRNA) by the ‘immune’ response. This crRNA then associates, through a region of partial complementarity, with another type of RNA called tracrRNA to guide the Cas9 or Cpf1 nuclease to a region homologous to the crRNA in the target DNA called a “protospacer”. Cas9 cleaves the DNA to generate blunt ends at the double-strand break (DSB) at sites specified by a 20-nucleotide guide sequence contained within the crRNA transcript. Cas9 requires both the crRNA and the tracrRNA for site specific DNA recognition and cleavage. This system has now been engineered such that the crRNA and tracrRNA can be combined into one molecule (the “single guide RNA”), and the crRNA equivalent portion of the single guide RNA can be engineered to guide the Cas9 nuclease to target any desired sequence (see Jinek et al (2012) Science 337, p. 816-821, Jinek et al, (2013), eLife 2:e00471, and David Segal, (2013) eLife 2:e00563). In alternative embodiments, the CRISPR/Cpf1 system is used, where Cpf1 requires only one RNA template in the gene-editing complex and cleaves the DNA resulting in a 5 nt staggered cut distal to the 5′ T-rich PAM, resulting in sticky ends (rather than blunt ends as when Cas9 is used). In some embodiments, a replacement gene can be used in the place of a METTL3 gene, e.g., a marker gene or in some embodiments, an cell death gene which is operatively linked to an inducible promoter, thereby allowing specific inducable cell death of the modified (i.e., METTL3 gene deleted) cells with a drug to turn on expression from the inducible promoter, should it be necessary to eliminate such modified cells after they are transplanted into a subject. Accordingly, the CRISPR/Cas (cas9 or cpf1) system can be engineered to create a double strand break (i.e., blunt ends (i.e., using cas9)) or sticky ends (i.e., using cpf1)) at a desired target in a genome, and repair of the double strand break can be influenced by the use of repair inhibitors to cause an increase in error prone repair.

There are at least three types of CRISPR/Cas systems which all incorporate RNAs and Cas proteins. Types I and III both have Cas endonucleases that process the pre-crRNAs, that, when fully processed into crRNAs, assemble a multi-Cas protein complex that is capable of cleaving nucleic acids that are complementary to the crRNA. The Type II CRISPR (exemplified by Cas9) is one of the most well characterized systems. The Cas9 protein has at least two nuclease domains: one nuclease domain is similar to a HNH endonuclease, while the other resembles a Ruv endonuclease domain. The HNH-type domain appears to be responsible for cleaving the DNA strand that is complementary to the crRNA while the Ruv domain cleaves the non-complementary strand.

In some embodiments, Cas protein can be a “functional derivative” of a naturally occurring Cas protein. As used herein, a “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof.

As used herein, “Cas polypeptide” encompasses a full-length Cas polypeptide, an enzymatically active fragment of a Cas polypeptide, and enzymatically active derivatives of a Cas polypeptide or fragment thereof. Suitable derivatives of a Cas polypeptide or a fragment thereof include, but are not limited to, mutants, fusions, covalent modifications of Cas protein or a fragment thereof.

Cas proteins and Cas polypeptides can be obtained from a cell or synthesized chemically or by a combination of these two procedures. The cell can be a cell that naturally produces Cas protein, or a cell that naturally produces Cas protein and is genetically engineered to produce the endogenous Cas protein at a higher expression level or to produce a Cas protein from an exogenously introduced nucleic acid, which encodes a Cas that is same or different from the endogenous Cas. The cell can be a cell that does not naturally produce Cas protein and is genetically engineered to produce a Cas protein.

The CRISPR/Cas system can also be used to inhibit gene expression. Lei et al. (2013) Cell 152(5):1173-1183) have shown that a catalytically dead Cas9 lacking endonuclease activity, when coexpressed with a guide RNA, generates a DNA recognition complex that can specifically interfere with transcriptional elongation, RNA polymerase binding, or transcription factor binding. This system, called CRISPR interference (CRISPRi), can efficiently repress expression of targeted genes.

Additionally, Cas proteins have been developed which comprise mutations in their cleavage domains to render them incapable of inducing a DSB, and instead introduce a nick into the target DNA. In particular, the Cas nuclease comprises two nuclease domains, the HNH and RuvC-like, for cleaving the sense and the antisense strands of the target DNA, respectively. The Cas nuclease can thus be engineered such that only one of the nuclease domains is functional, thus creating a Cas nickase.

The Cas9 related CRISPR/Cas system comprises two RNA non-coding components: tracrRNA and a pre-crRNA array containing nuclease guide sequences (spacers) interspaced by identical direct repeats (DRs). To use a CRISPR/Cas system to accomplish genome editing, both functions of these RNAs must be present (see Cong et al, (2013) Sciencexpress 1/10.1126/science 1231143). In some embodiments, the tracrRNA and pre-crRNAs are supplied via separate expression constructs or as separate RNAs. In other embodiments, a chimeric RNA is constructed where an engineered mature crRNA (conferring target specificity) is fused to a tracrRNA (supplying interaction with the Cas9) to create a chimeric cr-RNA-tracrRNA hybrid (also termed a single guide RNA).

The Cpf1 system, is related to the CRISPR/Cas9 system, although the Cpf1 protein is very different from Cas9, but is present in some bacteria with CRISPR. Cpf1 and Cas9 work differently, in that Cas9 requires two RNA molecules to cut DNA; Cpf1 needs only one. The proteins also cut DNA at different places, offering researchers more options when selecting a site to edit. Cpf1 also cuts DNA in a different way. Cas9 cuts both strands in a DNA molecule at the same position, leaving behind ‘blunt’ ends. In contrast, Cpf1 leaves one strand longer than the other, creating a ‘sticky’ end, reducing chances of abnormal/random DNA being inserted at the cleavage site, and also allowing better control of DNA to be inserted at the Cpf1 cleavage site. Cuts left by Cas9 tend to be repaired by sticking the two ends back together, that can leave errors. In contrast, Cpf1 sticky end cleavage allows more accurate and frequent insertions.

In some embodiments, the genome-editing agent is a ZFN. A ZFN generally comprises a zinc finger DNA binding protein and a DNA-cleavage domain. As used herein, a “zinc finger DNA binding protein” or “zinc finger DNA binding domain” is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein (ZFP). Zinc finger binding domains can be “engineered” to bind to a predetermined nucleotide sequence. Non-limiting examples of methods for engineering zinc finger proteins are design and selection. A designed zinc finger protein is a protein not occurring in nature whose design/composition results principally from rational criteria. Rational criteria for design include application of substitution rules and computerized algorithms for processing information in a database storing information of existing ZFP designs and binding data.

In some embodiments, the genome-editing agent is a TALEN. As used herein, the term “transcription activator-like effector nuclease” or “TAL effector nuclease” or “TALEN” refers to a class of artificial restriction endonucleases that are generated by fusing a TAL effector DNA binding domain to a DNA cleavage domain. In some embodiments, the TALEN is a monomeric TALEN that can cleave double stranded DNA without assistance from another TALEN. The term “TALEN” is also used to refer to one or both members of a pair of TALENs that are engineered to work together to cleave DNA at the same site. TALENs that work together can be referred to as a left-TALEN and a right-TALEN, which references the handedness of DNA.

In some embodiments, a combination of genome-editing agents can be used.

In some embodiments, a CRISPR/Cas, TALEN, or ZFN molecule (e.g. a peptide and/or peptide/nucleic acid complex) can be introduced into a cell, e.g. a cultured stem cell or progenitor cell, such that the presence of the CRISPR/Cas, TALEN, or ZFN molecule is transient and will not be detectable in the progeny that cell. In some embodiments, a nucleic acid encoding a CRISPR/Cas, TALEN, or ZFN molecule (e.g. a peptide and/or multiple nucleic acids encoding the parts of a peptide/nucleic acid complex) can be introduced into a cell, e.g. a cultured stem cell or progenitor cell, such that the nucleic acid is present in the cell transiently and the nucleic acid encoding the CRISPR/Cas, TALEN, or ZFN molecule as well as the CRISPR/Cas, TALEN, or ZFN molecule itself will not be detectable in the progeny of that cell. In some embodiments, a nucleic acid encoding a CRISPR/Cas, TALEN, or ZFN molecule (e.g. a peptide and/or multiple nucleic acids encoding the parts of a peptide/nucleic acid complex) can be introduced into a cell, e.g. a cultured stem cell or progenitor cell, such that the nucleic acid is maintained in the cell (e.g. incorporated into the genome) and the nucleic acid encoding the CRISPR/Cas, TALEN, or ZFN molecule and/or the CRISPR/Cas, TALEN, or ZFN molecule will be detectable in the progeny of that cell.

The genome-editing agents can be delivered to a target cell by any suitable means. In some embodiments, the genome-editing agent (e.g., CRISPR/Cas, TALEN, or ZFN) is a protein and can be delivered by any suitable means for delivering a protein into a cell such as electroporation, sonoporation, microinjection, liposomal delivery, and nanomaterial-based delivery.

The genome-editing agent can also be encoded by a nucleotide sequence. In some embodiments, the genome-editing agent can be delivered using a vector known to those of ordinary skill in the art. Viral vector systems which can be utilized in the present invention include, but are not limited to, (a) adenovirus vectors; (b) retrovirus vectors; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; (j) a helper-dependent or gutless adenovirus; (k) a lentiviral vector; (l) adenovirus vectors; and (m) herpesvirus vectors. See, also, U.S. Pat. Nos. 6,534,261; 6,607,882; 6,824,978; 6,933,113; 6,979,539; 7,013,219; and 7,163,824, each of which are incorporated by reference herein in their entireties. Replication-defective viruses can also be advantageous.

In some embodiments, a plasmid expression vector can be used. Plasmid expression vectors include, but are not limited to, pcDNA3.1, pET vectors (Novagen®), pGEX vectors (GE Life Sciences), and pMAL vectors (New England labs. Inc.) for protein expression in E. coli host cell such as BL21, BL21(DE3) and AD494(DE3)pLysS, Rosetta (DE3), and Origami(DE3) ((Novagen®); the strong CMV promoter-based pcDNA3.1 (Invitrogen™ Inc.) and pClneo vectors (Promega) for expression in mammalian cell lines such as CHO, COS, HEK-293, Jurkat, and MCF-7; replication incompetent adenoviral vector vectors pAdeno X, pAd5F35, pLP-Adeno-X-CMV (Clontech®), pAd/CMV/V5-DEST, pAd-DEST vector (Invitrogen™ Inc.) for adenovirus-mediated gene transfer and expression in mammalian cells; pLNCX2, pLXSN, and pLAPSN retrovirus vectors for use with the Retro-X™ system from Clontech for retroviral-mediated gene transfer and expression in mammalian cells; pLenti4/V5-DEST™, pLenti6/V5-DEST™, and pLenti6.2/V5-GW/lacZ (INVITROGEN™ Inc.) for lentivirus-mediated gene transfer and expression in mammalian cells; adenovirus-associated virus expression vectors such as pAAV-MCS and pAAV-IRES-hrGFP for adeno-associated virus-mediated gene transfer and expression in mammalian cells.

The vector may or may not be incorporated into the cell genome. The constructs may include viral sequences for transfection, if desired. Alternatively, the construct may be incorporated into vectors capable of episomal replication, e.g., EPV and EBV vectors.

When one or more ZFPs, TALENs, CRISPR/Cas molecules are introduced into the cell, the ZFPs, TALENs, CRISPR/Cas molecules can be carried on the same vector or on different vectors. When multiple vectors are used, each vector can comprise a sequence encoding one or multiple ZFPs, TALENs, CRISPR/Cas molecules.

Non-viral based delivery methods can also be used to introduce nucleic acids encoding engineered ZFPs, CRISPR/Cas molecules, and/or TALENs into cells (e.g., stem cells and/or progenitor cells). Methods of non-viral delivery of nucleic acids include electroporation, sonoporation, lipofection, microinjection, biolistics, virosomes, liposomes, immunoliposomes, polycation or lipid-nucleic acid conjugates, naked DNA, mRNA, artificial virions, and agent-enhanced uptake of DNA.

Additional exemplary nucleic acid delivery systems include those provided by Amaxa® Biosystems (Cologne, Germany), Maxcyte, Inc. (Rockville, Md.), BTX Molecular Delivery Systems (Holliston, Mass.) and Copernicus Therapeutics Inc, (see for example U.S. Pat. No. 6,008,336). Lipofection is described in e.g., U.S. Pat. No. 5,049,386, U.S. Pat. No. 4,946,787; and U.S. Pat. No. 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024.

More details about genome-editing techniques can be found, for example, in “Targeted Genome Editing Using Site-Specific Nucleases: ZFNs, TALENs, and the CRISPR/Cas9 System” by Takashi Yamamoto (Springer, 2015), the contents of which are incorporated herein by reference for the teaching on genome editing.

B. Activation of METTL3 and/or METTL4

Other aspects of the technology described herein relates to methods, compositions and kits to promote a stem cell population to differentiate along an endoderm lineage, for example, by activation of m⁶A methyltransferases, such as METTL3 and/or METTL4 or by increasing m⁶A RNA levels in the stem cell population. Methods to increase activity of METTL3 and/METTL4 are well known in the art, and include, for example, increasing or overexpressing METTL3 and/or METTL4 in a population of stem cells, e.g., human stem cells. In some embodiments, the human stem cells are pluripotent stem cells. In alternative embodiments, methods to increase m6A levels of target genes in stem cell populations include, but are not limited to inhibitors of fat-mass and obesity associated protein (FTO) and ALKBH5 (which are both m⁶A demethylases). Inhibition of FTO and/or ALKBH5 by inhibition of gene expression or function would increase m⁶A levels in the target genes and thus increase differentiation of the stem cell population).

Methods to inhibition FTO and/or ALKBH5 are known by persons of ordinary skill in the art and encompassed for use in the methods to promote differentiation of a stem cell population as disclosed herein. In some embodiments, an inhibitor of FTO is rhein, which inhibits FTO with an IC50 value of 30 μM using m6A-containing 15-mer ss-RNA as substrate and a high-performance liquid chromatography (HPLC)-based assay (as disclosed in Scott L. et al. A Genome-Wide Association Study of Type 2 Diabetes in Finns Detects Multiple Susceptibility Variants. Science 2007, 316, 1341-1345). Additionally, in some embodiments, an inhibitor of FTO is meclofenamic acid (MA), which is a highly selective inhibitor of FTO (IC50: 8 μM) over ALKBH5 (no inhibition) using HPLC-based assays (Huang Y., et al. Meclofenamic Acid Selectively Inhibits FTO Demethylation of m6A Over ALKBH5. Nucleic Acids Res, 2015; 43(1):373-84).

In some embodiments, the method relates to increasing the levels of the human METTL3 protein corresponding to SEQ ID NO:2, or a portion or functional fragment thereof which is capable of increasing m⁶A on RNA species in human stem cell populations to a similar level, (e.g., at least 80%) of the level of m6A that occurs with the wild-type human METTL3 protein of SEQ ID NO: 2. In some embodiments, human METTL3 mRNA of SEQ ID NO: 1 is introduced into a human stem cell population.

In some embodiments, the method relates to increasing the levels of the human METTL4 protein corresponding to SEQ ID NO:7, or a portion or functional fragment thereof which is capable of increasing m⁶A on RNA species in human stem cell populations to a similar level, (e.g., at least 80%) of the level of m6A that occurs with the wild-type human METTL4 protein of SEQ ID NO: 7. In some embodiments, human METTL4 mRNA of SEQ ID NO: 8 is introduced into a human stem cell population.

In some embodiments, methods to increase m6A in cell populations comprises contacting the cell population with a miR, such as, miR-423-3p and miR-1226-3p, which increases METTL3 interaction with mRNA transcripts.

Delivery of Nucleic Acid Inhibitors of METTL3/METTL4 or mRNAs Expressing METTL3/METTL4 to a Stem Cell Population.

In some embodiments, a nucleic inhibitor to METTL3 and/or METTL4, or a nucleic acid encoding METTL3 and/or METTL4 protein or a functional fragment thereof is delivered into a specific target cell, e.g., a stem cell population using a vector and gene expression systems which are known by persons of ordinary skill in the art.

The term “vectors” refers to a nucleic acid molecule capable of transporting another nucleic acid to which it has been linked; a plasmid is a species of the genus encompassed by “vector”. The term “vector” typically refers to a nucleic acid sequence containing an origin of replication and other entities necessary for replication and/or maintenance in a host cell. Vectors capable of directing the expression of genes and/or nucleic acid sequence to which they are operatively linked are referred to herein as “expression vectors”. In general, expression vectors of utility are often in the form of “plasmids” which refer to circular double stranded DNA loops which, in their vector form are not bound to the chromosome, and typically comprise entities for stable or transient expression or the encoded DNA. Other expression vectors can be used in the methods as disclosed herein for example, but are not limited to, plasmids, episomes, bacterial artificial chromosomes, yeast artificial chromosomes, bacteriophages or viral vectors, and such vectors can integrate into the host's genome or replicate autonomously in the particular cell. A vector can be a DNA or RNA vector. Other forms of expression vectors known by those skilled in the art which serve the equivalent functions can also be used, for example self replicating extrachromosomal vectors or vectors which integrates into a host genome.

Vectors include, but are not limited to, plasmids, cosmids, phagemids, viruses, other vehicles derived from viral or bacterial sources that have been manipulated by the insertion or incorporation of the nucleic acid sequences for producing the microRNA, and free nucleic acid fragments which can be attached to these nucleic acid sequences. Viral and retroviral vectors are a preferred type of vector and include, but are not limited to, nucleic acid sequences from the following viruses: retroviruses, such as: Moloney murine leukemia virus; Murine stem cell virus, Harvey murine sarcoma virus; marine mammary tumor virus; Rous sarcoma virus; adenovirus; adeno-associated virus; SV40-type viruses; polyoma viruses; Epstein-Barr viruses; papilloma viruses; herpes viruses; vaccinia viruses; polio viruses; and RNA viruses such as any retrovirus. One of skill in the art can readily employ other vectors known in the art.

Viral vectors are generally based on non-cytopathic eukaryotic viruses in which non-essential genes have been replaced with the nucleic acid sequence of interest. Non-cytopathic viruses include retroviruses, the life cycle of which involves reverse transcription of genomic viral RNA into DNA with subsequent proviral integration into host cellular DNA.

Retroviruses have been approved for human gene therapy trials. Genetically altered retroviral expression vectors have general utility for the high efficiency transduction of nucleic acids in viva. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W.H. Freeman Co., New York (1990) and Murry, E. J. Ed. “Methods in Molecular L Biology,” vol. 7, Humana Press, Inc., Cliffton, N.J. (1991).

In some embodiments the “in vivo expression elements” are any regulatory nucleotide sequence, such as a promoter sequence or promoter-enhancer combination, which facilitates the efficient expression of the nucleic acid to produce the microRNA. The in vivo expression element may, for example, be a mammalian or viral promoter, such as a constitutive or inducible promoter and/or a tissue specific promoter. Examples of which are well known to one of ordinary skill in the art. Constitutive mammalian promoters include, but are not limited to, polymerase promoters as well as the promoters for the following genes: hypoxanthine phosphoribosyl transferase (HPTR), adenine deaminase, pyruvate kinase, and beta.-actin. Exemplary viral promoters which function constitutively in eukaryotic cells include, but are not limited to, promoters from the simian virus, papilloma virus, adenovirus, human immunodeficiency virus (HIV), Rous sarcoma virus, cytomegalovirus, the long terminal repeats (LTR) of moloney leukemia virus and other retroviruses, and the thymidine kinase promoter of herpes simplex virus. Other constitutive promoters are known to those of ordinary skill in the art. Inducible promoters are expressed in the presence of an inducing agent and include, but are not limited to, metal-inducible promoters and steroid-regulated promoters. For example, the metallothionein promoter is induced to promote transcription in the presence of certain metal ions. Other inducible promoters are known to those of ordinary skill in the art.

Examples of tissue-specific promoters include, but are not limited to, the promoter for creatine kinase, which has been used to direct expression in muscle and cardiac tissue and immunoglobulin heavy or light chain promoters for expression in B cells. Other tissue specific promoters include the human smooth muscle alpha-actin promoter. Exemplary tissue-specific expression elements for the liver include but are not limited to HMG-COA reductase promoter, sterol regulatory element 1, phosphoenol pyruvate carboxy kinase (PEPCK) promoter, human C-reactive protein (CRP) promoter, human glucokinase promoter, cholesterol L 7-alpha hydroylase (CYP-7) promoter, beta-galactosidase alpha-2,6 sialylkansferase promoter, insulin-like growth factor binding protein (IGFBP-1) promoter, aldolase B promoter, human transferrin promoter, and collagen type I promoter. Exemplary tissue-specific expression elements for the prostate include but are not limited to the prostatic acid phosphatase (PAP) promoter, prostatic secretory protein of 94 (PSP 94) promoter, prostate specific antigen complex promoter, and human glandular kallikrein gene promoter (hgt-1). Exemplary tissue-specific expression elements for gastric tissue include but are not limited to the human H+/K+-ATPase alpha subunit promoter. Exemplary tissue-specific expression elements for the pancreas include but are not limited to pancreatitis associated protein promoter (PAP), elastase 1 transcriptional enhancer, pancreas specific amylase and elastase enhancer promoter, and pancreatic cholesterol esterase gene promoter. Exemplary tissue-specific expression elements for the endometrium include, but are not limited to, the uteroglobin promoter. Exemplary tissue-specific expression elements for adrenal cells include, but are not limited to, cholesterol side-chain cleavage (SCC) promoter. Exemplary tissue-specific expression elements for the general nervous system include, but are not limited to, gamma-gamma enolase (neuron-specific enolase, NSE) promoter. Exemplary tissue-specific expression elements for the brain include, but are not limited to, the neurofilament heavy chain (NF-H) promoter. Exemplary tissue-specific expression elements for lymphocytes include, but are not limited to, the human CGL-1/granzyme B promoter, the terminal deoxy transferase (TdT), lambda 5, VpreB, and lck (lymphocyte specific tyrosine protein kinase p561ck) promoter, the humans CD2 promoter and its 3′ transcriptional enhancer, and the human NK and T cell specific activation (NKG5) promoter. Exemplary tissue-specific expression elements for the colon include, but are not limited to, pp60c-src tyrosine kinase promoter, organ-specific neoantigens (OSNs) promoter, and colon specific antigen-P promoter.

Other elements aiding specificity of expression in a tissue of interest can include secretion leader sequences, enhancers, nuclear localization signals, endosmolytic peptides, etc. Preferably, these elements are derived from the tissue of interest to aid specificity. In general, the in vivo expression element shall include, as necessary, 5′ non-transcribing and 5′ non-translating sequences involved with the initiation of transcription. They optionally include enhancer sequences or upstream activator sequences.

Mammalian expression vectors can comprise an origin of replication, a suitable promoter, polyadenylation site, transcriptional termination sequences, and 5′ flanking non-transcribed sequences. DNA sequences derived from the SV40 viral genome, for example, SV40 origin, early promoter, enhancer, splice, and polyadenylation sites may be used to provide the required non-transcribed genetic elements.

Other described ways to deliver a nucleic inhibitor to METTL3 and/or METTL4, or a nucleic acid encoding METTL3 and/or METTL4 protein or a functional fragment thereof) as disclosed herein is via vectors, such as lentiviral constructs, and introducing molecules into cells using electroporation. In some embodiments, FIV lentivirus vectors which are based on the feline immunodeficiency virus (FIV) retrovirus and the HIV lentivirus vector system, which is based on the human immunodeficiency virus (HIV), are used. Alternatively, electroporation is also useful in the present invention, although it is generally only used to deliver siRNAs into cells in vitro.

In one embodiment, a vector encoding an nucleic inhibitor to METTL3 and/or METTL4, or a nucleic acid encoding METTL3 and/or METTL4 protein or a functional fragment thereof is delivered into a specific target cell, e.g., a stem cell population. Nucleic acid sequences necessary for expression in mammalian cells often utilize a combination of one or more promoters, enhancers, and termination and polyadenylation signals.

One can also use localization sequences to deliver an inhibitor to METTL3 and/or METTL4, or a nucleic acid encoding METTL3 and/or METTL4 protein or a functional fragment thereofintracellularly to a cell compartment of interest. Typically, the delivery system first binds to a specific receptor on the cell. Thereafter, the targeted cell internalizes the delivery system, which is bound to the cell. For example, membrane proteins on the cell surface, including receptors and antigens can be internalized by receptor mediated endocytosis after interaction with the ligand to the receptor or antibodies. (Dautry-Varsat, A., et al., Sci. Am. 250:52-58 (1984)). This endocytic process is exploited by the present delivery system. Because this process may damage inhibitor to METTL3 and/or METTL4, or a nucleic acid encoding METTL3 and/or METTL4 protein or a functional fragment thereof, for example a RNAi or siRNA agent, or anti-miR as it is being internalized, it may be desirable to use a segment containing multiple repeats of the RNA interference-inducing molecule of interest. One can also include sequences or moieties that disrupt endosomes and lysosomes. See, e.g., Cristiano, R. J., et al., Proc. Natl. Acad. Sci. USA 90:11548-11552 (1993); Wagner, E., et al., Proc. Natl. Acad. Sci. USA 89:6099-6103 (1992); Cotten, M., et al., Proc. Natl. Acad. Sci. USA 89:6094-6098 (1992).

In some embodiments, inhibitor to METTL3 and/or METTL4, or a nucleic acid encoding METTL3 and/or METTL4 protein or a functional fragment thereof can be complexed with desired targeting moieties by mixing a RNAi molecules with a targeting moiety in the presence of complexing agents. Examples of such complexing agents include, but are not limited to, poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. In some embodiments, the complexing agents include chitosan, N-trimethylchitosan, poly-L-lysine, polyhistidine, polyornithine, polyspermines, protamine, polyvinylpyridine, polythiodiethylaminomethylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DE AE-albumin and DEAE-dextran, polymethylacrylate, polyhexylacrylate, poly(D,L-lactic acid), poly(DL-lactic-co-glycolic acid (PLGA), alginate, and polyethyleneglycol (PEG), and polyethylenimine.

In alternative embodiments, inhibitor to METTL3 and/or METTL4, or a nucleic acid encoding METTL3 and/or METTL4 protein or a functional fragment thereof is complexed to a complexing agent, e.g., such as a protamine or an RNA-binding domain, such as an siRNA-binding fragment or nucleic acid binding fragment of protamine. Protamine is a polycationic peptide with molecular weight about 4000-4500 Da. Protamine is a small basic nucleic acid binding protein, which serves to condense the animal's genomic DNA for packaging into the restrictive volume of a sperm head (Warrant, R. W., et al., Nature 271:130-135 (1978); Krawetz, S. A., et al., Genomics 5:639-645 (1989)). The positive charges of the protamine can strongly interact with negative charges of the phosphate backbone of nucleic acid, such as RNA, resulting in a neutral and stable interference RNA-protamine complex.

In one embodiment, the protamine fragment is encoded by a nucleic acid sequence disclosed in International Patent Application: PCT/US05/029111, which is incorporated herein in its entirety by reference. The methods, reagents and references that describe a preparation of a nucleic acid-protamine complex in detail are disclosed in the U.S. Patent Application Publication Nos. US200210132990 and US200410023902, and are herein incorporated by reference in their entirety.

### II. Fingerprinting of m6A Levels and Analysis of Stem Cell Populations

Another aspect of the technology disclosed herein relates to the use of the intensity of m6A sites of methylation (i.e., m6A peak intensity) as a quantitative metric or measure to distinguish cell states. Stated another way, the intensity of m6A sites of methylation (i.e., m6A peak intensity) of a set of specific target gene, e.g., at least 10 or more selected from Table 1 or Table 2, can be used to “fingerprint” a cell state, e.g., determine the cell state of the stem cell population, i.e., to determine if the stem cell population is pluripotent (i.e., in an undifferentiated pluripotent state) or if the human stem cell population has differentiated along a cell lineage pathway. Importantly, using the intensity of m6A sites of methylation (i.e., m6A peak intensity) of specific target genes is idependent of gene expression levels, which is the current standard of analysis of stem cell populations.

Accordingly, another aspect of the technology described herein relates to methods, assays, arrays and kits for performing m6A analysis of RNA from stem cell populations to characterize the cell state of the cell population, which can be used, for example, as a quality control for the stem cell population. In some embodiments, the stem cell population is a human stem cell population, e.g., a hESC cell population or other human stem cell line.

Accordingly, another aspect of the technology described herein relates to methods, compositions, assays, arrays and kits to characterize a stem cell population, such as a human stem cell population, comprising performing m⁶A analysis on the RNA obtained from the population of stem cells, and assessing the intensity of the m⁶A levels of the mRNA of at least 10 genes selected from any of those in Table 1, or Table 2 as disclosed herein.

Another aspect of the technology described herein relates to methods, compositions, assays, arrays and kits for assessing m⁶A levels in the RNA obtained from a population of stem cells, e.g., human stem cells. In some embodiments, the method comprises (i) measuring the m⁶A levels of least 10 mRNA transcripts selected from any of those listed in Table 1 or Table 2, for example by contacting an array with RNA isolated from a cell population, where the array comprises at least 10 or more oligonucleotides that hybridize to at least 10 mRNA transcripts, or to at least 10 3′UTR or other untranslated regions of at least 10 genes selected from any of those listed in Table 1 or Table 2, and (ii) contacting the array with at least one reagent which binds to m6A in the RNA, such as an anti-m⁶A antibody, or fragment thereof, such as an anti-m⁶A antibody which is fluorescently labeled or otherwise has a detectable label, therefore allowing the measurements of the levels of m6A in the at least selected 10 mRNA transcripts, or to at least 10 3′UTR or other untranslated regions of at least 10 genes selected from any of those listed in Table 1 or Table 2.

A further aspect of the technology described herein relates to methods, compositions, assays, arrays and kits for use in a method for determining the cell state of a stem cell population comprising performing the assay of claim 10, and comparing the levels of m6A (i.e., peak intensities) of at least 10 genes selected from any of Table 1 in the RNA from the stem cell population with the levels of m6A (i.e., peak intensities) in a reference stem cell population, and based on this comparison, determining the cell state of the stem cell population.

Another aspect of the present invention relates to a kit comprising: (i) an array composition for characterizing the cell state of a population of stem cells, comprising at least 10 oligonucleotides that hybridize to the RNA (i.e., mRNA transcripts, 3′UTR or other untranslated RNAs) of at least 10 genes selected from any of those in Table 1 or Table 2 as disclosed herein; and (ii) at least one regent to detect the m6A in RNA, such as, for example, an anti-m⁶A antibody, or fragment thereof, for example an anti-m6A antibody or fragment thereof which is detectably labeled (e.g., with a florescent label, colorimetric marker etc.).

In some embodiments, the kit comprises a computer readable medium comprising instructions on a computer to compare the measured levels of m6A (i.e., peak intensities) from a test stem cell population with reference levels of the same RNA transcripts assessed. In some embodiments, the kit comprises instructions to access to a software program available online (e.g., on a cloud) to compare the measured levels of the m6A (i.e., peak intensities) from the test stem cell population, e.g., human stem cell population, with reference levels of m6A for the same RNAs assessed from a reference stem cell population, e.g., human stem cell population.

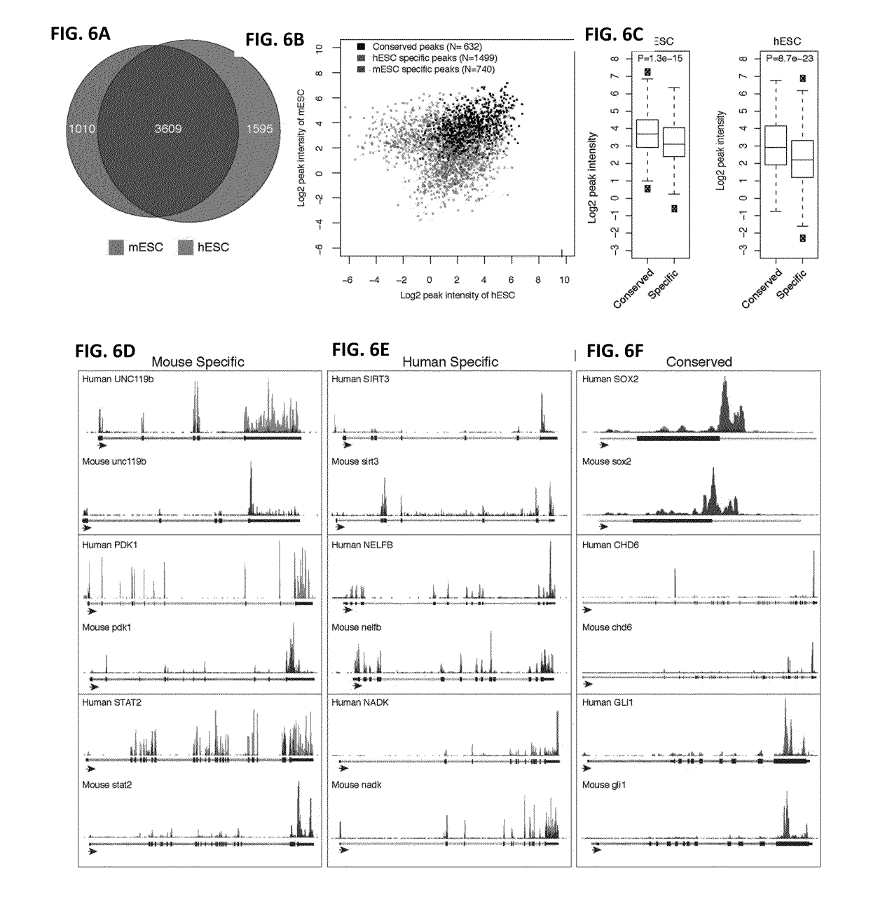

TABLE 1
hESC and mESC Common Peaks
Table 1: List of genes for measuring m⁶A levels in stem cell populations. Table 1 is related to
FIG. 6 and provides the Ensemble Gene ID of human and mouse and chromosome coordinates
of common m⁶A peaks.

| | | | SEQ ID | | | |
|---|---|---|---|---|---|---|
| | | | NO: | | | |
| | | | (for | | | |
| | | Human | human | | | |
| Human Ensembl | Mouse Ensembl Ggene | Gene | Gene | Human | Human | |
| Ggene ID | ID | Symbol | ID) | chromosome | start | Human end |
| ENSG00000064703 | ENSMUSG00000027905 | DDX20 | 9 | chr1 | 112308858 | 112308958 |
| ENSG00000086015 | ENSMUSG00000003810 | MAST2 | 10 | chr1 | 46500659 | 46500760 |
| ENSG00000168036 | ENSMUSG00000006932 | CTNNB1 | 11 | chr3 | 41240966 | 41241066 |
| ENSG00000168036 | ENSMUSG00000006932 | CTNNB1 | 12 | chr3 | 41280873 | 41280978 |
| ENSG00000168036 | ENSMUSG00000006932 | CTNNB1 | 13 | chr3 | 41281311 | 41281411 |
| ENSG00000185127 | ENSMUSG00000050088 | C6orf120 | 14 | chr6 | 170102894 | 170102994 |
| ENSG00000109118 | ENSMUSG00000037791 | PHF12 | 15 | chr17 | 27239936 | 27240045 |
| ENSG00000109113 | ENSMUSG00000002059 | RAB34 | 16 | chr17 | 27041474 | 27041574 |
| ENSG00000042088 | ENSMUSG00000021177 | TDP1 | 17 | chr14 | 90429848 | 90429948 |
| ENSG00000205765 | ENSMUSG00000041935 | C5orf51 | 18 | chr5 | 41917289 | 41917389 |
| ENSG00000182272 | ENSMUSG00000055629 | B4GALNT4 | 19 | chr11 | 377163 | 377270 |
| ENSG00000184708 | ENSMUSG00000020454 | EIF4ENIF1 | 20 | chr22 | 31835776 | 31835876 |
| ENSG00000141682 | ENSMUSG00000024521 | PMAIP1 | 21 | chr18 | 57570000 | 57570100 |
| ENSG00000145041 | ENSMUSG00000040325 | VPRBP | 22 | chr3 | 51457387 | 51457488 |
| ENSG00000145041 | ENSMUSG00000040325 | VPRBP | 23 | chr3 | 51475542 | 51475642 |
| ENSG00000157978 | ENSMUSG00000037295 | LDLRAP1 | 24 | chr1 | 25893495 | 25893595 |
| ENSG00000185728 | ENSMUSG00000047213 | YTHDF3 | 25 | chr8 | 64099129 | 64099229 |
| ENSG00000154370 | ENSMUSG00000020455 | TRIM11 | 26 | chr1 | 228582552 | 228582652 |
| ENSG00000205268 | ENSMUSG00000069094 | PDE7A | 27 | chr8 | 66631525 | 66631625 |
| ENSG00000213024 | ENSMUSG00000043858 | NUP62 | 28 | chr19 | 50411493 | 50411593 |
| ENSG00000134247 | ENSMUSG00000027864 | PTGFRN | 29 | chr1 | 117504014 | 117504114 |
| ENSG00000134247 | ENSMUSG00000027864 | PTGFRN | 30 | chr1 | 117529590 | 117529690 |
| ENSG00000143442 | ENSMUSG00000038902 | POGZ | 31 | chr1 | 151377307 | 151377407 |
| ENSG00000143442 | ENSMUSG00000038902 | POGZ | 32 | chr1 | 151377594 | 151377694 |
| ENSG00000161204 | ENSMUSG00000003234 | ABCF3 | 33 | chr3 | 183911477 | 183911577 |
| ENSG00000247596 | ENSMUSG00000023277 | TWF2 | 34 | chr3 | 52262944 | 52263044 |
| ENSG00000048649 | ENSMUSG00000035623 | RSF1 | 35 | chr11 | 77378075 | 77378175 |
| ENSG00000057757 | ENSMUSG00000028669 | PITHD1 | 36 | chr1 | 24113930 | 24114030 |
| ENSG00000135048 | ENSMUSG00000024754 | TMEM2 | 37 | chr9 | 74300031 | 74300134 |
| ENSG00000135048 | ENSMUSG00000024754 | TMEM2 | 38 | chr9 | 74360151 | 74360251 |
| ENSG00000142798 | ENSMUSG00000028763 | HSPG2 | 39 | chr1 | 22149583 | 22149683 |
| ENSG00000135912 | ENSMUSG00000033257 | TTLL4 | 40 | chr2 | 219603558 | 219603658 |
| ENSG00000092148 | ENSMUSG00000035247 | HECTD1 | 41 | chr14 | 31576238 | 31576338 |
| ENSG00000177732 | ENSMUSG00000051817 | SOX12 | 42 | chr20 | 307350 | 307450 |
| ENSG00000166484 | ENSMUSG00000001034 | MAPK7 | 43 | chr17 | 19284120 | 19284224 |
| ENSG00000105281 | ENSMUSG00000001918 | SLC1A5 | 44 | chr19 | 47278621 | 47278721 |
| ENSG00000172819 | ENSMUSG00000001288 | RARG | 45 | chr12 | 53605118 | 53605218 |
| ENSG00000090097 | ENSMUSG00000023495 | PCBP4 | 46 | chr3 | 51991534 | 51991634 |
| ENSG00000121210 | ENSMUSG00000033767 | KIAA0922 | 47 | chr4 | 154557652 | 154557760 |
| ENSG00000099954 | ENSMUSG00000071226 | CECR2 | 48 | chr22 | 18027962 | 18028062 |
| ENSG00000099954 | ENSMUSG00000071226 | CECR2 | 49 | chr22 | 18028899 | 18028999 |
| ENSG00000075413 | ENSMUSG00000007411 | MARK3 | 50 | chr14 | 103969405 | 103969505 |
| ENSG00000169375 | ENSMUSG00000042557 | SIN3A | 51 | chr15 | 75664067 | 75664167 |
| ENSG00000169375 | ENSMUSG00000042557 | SIN3A | 52 | chr15 | 75664369 | 75664475 |
| ENSG00000169375 | ENSMUSG00000042557 | SIN3A | 53 | chr15 | 75684619 | 75684719 |
| ENSG00000111802 | ENSMUSG00000035958 | TDP2 | 54 | chr6 | 24651003 | 24651104 |
| ENSG00000142655 | ENSMUSG00000028975 | PEX14 | 55 | chr1 | 10689962 | 10690063 |
| ENSG00000134186 | ENSMUSG00000027881 | PRPF38B | 56 | chr1 | 109242126 | 109242233 |
| ENSG00000135900 | ENSMUSG00000026248 | MRPL44 | 57 | chr2 | 224824419 | 224824519 |
| ENSG00000166326 | ENSMUSG00000027189 | TRIM44 | 58 | chr11 | 35685077 | 35685177 |
| ENSG00000089876 | ENSMUSG00000030986 | DHX32 | 59 | chr10 | 127569423 | 127569523 |
| ENSG00000123066 | ENSMUSG00000018076 | MED13L | 60 | chr12 | 116428930 | 116429030 |
| ENSG00000123066 | ENSMUSG00000018076 | MED13L | 61 | chr12 | 116429280 | 116429380 |
| ENSG00000123066 | ENSMUSG00000018076 | MED13L | 62 | chr12 | 116429524 | 116429624 |
| ENSG00000132680 | ENSMUSG00000028060 | KIAA0907 | 63 | chr1 | 155883746 | 155883846 |
| ENSG00000068001 | ENSMUSG00000010047 | HYAL2 | 64 | chr3 | 50357457 | 50357557 |
| ENSG00000115275 | ENSMUSG00000030036 | MOGS | 65 | chr2 | 74688345 | 74688445 |
| ENSG00000058600 | ENSMUSG00000030880 | POLR3E | 66 | chr16 | 22345114 | 22345224 |
| ENSG00000165671 | ENSMUSG00000021488 | NSD1 | 67 | chr5 | 176562562 | 176562672 |
| ENSG00000165671 | ENSMUSG00000021488 | NSD1 | 68 | chr5 | 176638151 | 176638251 |
| ENSG00000165671 | ENSMUSG00000021488 | NSD1 | 69 | chr5 | 176638780 | 176638880 |
| ENSG00000165671 | ENSMUSG00000021488 | NSD1 | 70 | chr5 | 176721213 | 176721313 |
| ENSG00000165671 | ENSMUSG00000021488 | NSD1 | 71 | chr5 | 176721632 | 176721732 |
| ENSG00000165671 | ENSMUSG00000021488 | NSD1 | 72 | chr5 | 176722272 | 176722382 |
| ENSG00000165671 | ENSMUSG00000021488 | NSD1 | 73 | chr5 | 176722658 | 176722758 |
| ENSG00000183258 | ENSMUSG00000021494 | DDX41 | 74 | chr5 | 176938714 | 176938814 |
| ENSG00000254726 | ENSMUSG00000074480 | MEX3A | 75 | chr1 | 156046763 | 156046863 |
| ENSG00000100393 | ENSMUSG00000055024 | EP300 | 76 | chr22 | 41513565 | 41513665 |
| ENSG00000100393 | ENSMUSG00000055024 | EP300 | 77 | chr22 | 41573454 | 41573554 |
| ENSG00000196950 | ENSMUSG00000025986 | SLC39A10 | 78 | chr2 | 196545586 | 196545686 |
| ENSG00000165322 | ENSMUSG00000041225 | ARHGAP12 | 79 | chr10 | 32197224 | 32197324 |
| ENSG00000165322 | ENSMUSG00000041225 | ARHGAP12 | 80 | chr10 | 32197586 | 32197686 |
| ENSG00000156966 | ENSMUSG00000079445 | B3GNT7 | 81 | chr2 | 232263472 | 232263572 |
| ENSG00000082258 | ENSMUSG00000026349 | CCNT2 | 82 | chr2 | 135712134 | 135712234 |
| ENSG00000097007 | ENSMUSG00000026842 | ABL1 | 83 | chr9 | 133760467 | 133760567 |
| ENSG00000257365 | ENSMUSG00000033373 | FNTB | 84 | chr14 | 65528222 | 65528322 |
| ENSG00000026652 | ENSMUSG00000023827 | AGPAT4 | 85 | chr6 | 161557469 | 161557569 |
| ENSG00000087510 | ENSMUSG00000028640 | TFAP2C | 86 | chr20 | 55212845 | 55212945 |
| ENSG00000187954 | ENSMUSG00000053929 | CYHR1 | 87 | chr8 | 145677121 | 145677221 |
| ENSG00000178921 | ENSMUSG00000020899 | PFAS | 88 | chr17 | 8172544 | 8172647 |
| ENSG00000105447 | ENSMUSG00000053801 | GRWD1 | 89 | chr19 | 48956110 | 48956214 |
| ENSG00000108671 | ENSMUSG00000017428 | PSMD11 | 90 | chr17 | 30808141 | 30808241 |
| ENSG00000115241 | ENSMUSG00000029147 | PPM1G | 91 | chr2 | 27604368 | 27604468 |
| ENSG00000127774 | ENSMUSG00000047260 | TMEM93 | 92 | chr17 | 3572778 | 3572878 |
| ENSG00000166685 | ENSMUSG00000018661 | COG1 | 93 | chr17 | 71197612 | 71197712 |
| ENSG00000115020 | ENSMUSG00000025949 | PIKFYVE | 94 | chr2 | 209220007 | 209220107 |
| ENSG00000258429 | ENSMUSG00000078931 | PDF | 95 | chr16 | 69362669 | 69362773 |
| ENSG00000124067 | ENSMUSG00000017765 | SLC12A4 | 96 | chr16 | 67978451 | 67978551 |
| ENSG00000197965 | ENSMUSG00000026566 | MPZL1 | 97 | chr1 | 167759643 | 167759743 |
| ENSG00000182831 | ENSMUSG00000022507 | C16orf72 | 98 | chr16 | 9196990 | 9197090 |
| ENSG00000117523 | ENSMUSG00000040225 | PRRC2C | 99 | chr1 | 171510359 | 171510468 |
| ENSG00000103126 | ENSMUSG00000024182 | AXIN1 | 100 | chr16 | 338078 | 338178 |
| ENSG00000198561 | ENSMUSG00000034101 | CTNND1 | 101 | chr11 | 57569486 | 57569586 |
| ENSG00000167470 | ENSMUSG00000035621 | MIDN | 102 | chr19 | 1250180 | 1250288 |
| ENSG00000111605 | ENSMUSG00000055531 | CPSF6 | 103 | chr12 | 69663341 | 69663441 |
| ENSG00000108604 | ENSMUSG00000078619 | SMARCD2 | 104 | chr17 | 61920149 | 61920249 |
| ENSG00000119777 | ENSMUSG00000038828 | TMEM214 | 105 | chr2 | 27263653 | 27263753 |
| ENSG00000137166 | ENSMUSG00000023991 | FOXP4 | 106 | chr6 | 41566767 | 41566867 |
| ENSG00000137161 | ENSMUSG00000023973 | CNPY3 | 107 | chr6 | 42906787 | 42906887 |
| ENSG00000165650 | ENSMUSG00000074746 | PDZD8 | 108 | chr10 | 119042750 | 119042850 |
| ENSG00000067840 | ENSMUSG00000002006 | PDZD4 | 109 | chrX | 153070066 | 153070166 |
| ENSG00000204138 | ENSMUSG00000066043 | PHACTR4 | 110 | chr1 | 28800345 | 28800445 |
| ENSG00000157933 | ENSMUSG00000029050 | SKI | 111 | chr1 | 2160997 | 2161097 |
| ENSG00000159140 | ENSMUSG00000022961 | SON | 112 | chr21 | 34929945 | 34930045 |
| ENSG00000196576 | ENSMUSG00000036606 | PLXNB2 | 113 | chr22 | 50728117 | 50728217 |
| ENSG00000113161 | ENSMUSG00000021670 | HMGCR | 114 | chr5 | 74656186 | 74656286 |
| ENSG00000142207 | ENSMUSG00000039929 | URB1 | 115 | chr21 | 33719790 | 33719890 |
| ENSG00000113615 | ENSMUSG00000036391 | SEC24A | 116 | chr5 | 133996908 | 133997010 |
| ENSG00000143624 | ENSMUSG00000027933 | INTS3 | 117 | chr1 | 153746075 | 153746175 |
| ENSG00000171456 | ENSMUSG00000042548 | ASXL1 | 118 | chr20 | 31023428 | 31023528 |
| ENSG00000171456 | ENSMUSG00000042548 | ASXL1 | 119 | chr20 | 31024564 | 31024664 |
| ENSG00000140534 | ENSMUSG00000046591 | C15orf42 | 120 | chr15 | 90168613 | 90168713 |
| ENSG00000100226 | ENSMUSG00000042535 | GTPBP1 | 121 | chr22 | 39112725 | 39112825 |
| ENSG00000163811 | ENSMUSG00000041057 | WDR43 | 122 | chr2 | 29169524 | 29169624 |
| ENSG00000046604 | ENSMUSG00000044393 | DSG2 | 123 | chr18 | 29125852 | 29125952 |
| ENSG00000065883 | ENSMUSG00000041297 | CDK13 | 124 | chr7 | 40027525 | 40027625 |
| ENSG00000065883 | ENSMUSG00000041297 | CDK13 | 125 | chr7 | 40133978 | 40134081 |
| ENSG00000065883 | ENSMUSG00000041297 | CDK13 | 126 | chr7 | 40134465 | 40134565 |
| ENSG00000019995 | ENSMUSG00000030967 | ZRANB1 | 127 | chr10 | 126631514 | 126631614 |
| ENSG00000019995 | ENSMUSG00000030967 | ZRANB1 | 128 | chr10 | 126631726 | 126631826 |
| ENSG00000176208 | ENSMUSG00000017550 | ATAD5 | 129 | chr17 | 29220647 | 29220747 |
| ENSG00000132254 | ENSMUSG00000030881 | ARFIP2 | 130 | chr11 | 6498232 | 6498332 |
| ENSG00000052749 | ENSMUSG00000035049 | RRP12 | 131 | chr10 | 99116643 | 99116743 |
| ENSG00000144567 | ENSMUSG00000049339 | FAM134A | 132 | chr2 | 220047134 | 220047234 |
| ENSG00000146676 | ENSMUSG00000094483 | PURB | 133 | chr7 | 44922586 | 44922686 |
| ENSG00000146676 | ENSMUSG00000094483 | PURB | 134 | chr7 | 44923094 | 44923197 |
| ENSG00000090905 | ENSMUSG00000052707 | TNRC6A | 135 | chr16 | 24800791 | 24800891 |
| ENSG00000170881 | ENSMUSG00000037075 | RNF139 | 136 | chr8 | 125499780 | 125499880 |
| ENSG00000148143 | ENSMUSG00000060206 | ZNF462 | 137 | chr9 | 109688675 | 109688775 |
| ENSG00000148143 | ENSMUSG00000060206 | ZNF462 | 138 | chr9 | 109773391 | 109773491 |
| ENSG00000104332 | ENSMUSG00000031548 | SFRP1 | 139 | chr8 | 41166322 | 41166422 |
| ENSG00000178252 | ENSMUSG00000066357 | WDR6 | 140 | chr3 | 49052670 | 49052770 |
| ENSG00000120709 | ENSMUSG00000034300 | FAM53C | 141 | chr5 | 137682590 | 137682690 |
| ENSG00000100376 | ENSMUSG00000022434 | FAM118A | 142 | chr22 | 45736465 | 45736569 |
| ENSG00000126883 | ENSMUSG00000001855 | NUP214 | 143 | chr9 | 134073152 | 134073259 |
| ENSG00000161638 | ENSMUSG00000000555 | ITGA5 | 144 | chr12 | 54789820 | 54789920 |
| ENSG00000078618 | ENSMUSG00000053510 | NRD1 | 145 | chr1 | 52344004 | 52344104 |
| ENSG00000101412 | ENSMUSG00000027490 | E2F1 | 146 | chr20 | 32264513 | 32264613 |
| ENSG00000171603 | ENSMUSG00000039953 | CLSTN1 | 147 | chr1 | 9790176 | 9790276 |
| ENSG00000171604 | ENSMUSG00000046668 | CXXC5 | 148 | chr5 | 139060543 | 139060648 |
| ENSG00000022567 | ENSMUSG00000079020 | SLC45A4 | 149 | chr8 | 142228759 | 142228865 |
| ENSG00000169635 | ENSMUSG00000050240 | HIC2 | 150 | chr22 | 21800152 | 21800252 |
| ENSG00000169635 | ENSMUSG00000050240 | HIC2 | 151 | chr22 | 21800585 | 21800686 |
| ENSG00000136940 | ENSMUSG00000009030 | PDCL | 152 | chr9 | 125582347 | 125582456 |
| ENSG00000136940 | ENSMUSG00000009030 | PDCL | 153 | chr9 | 125582639 | 125582739 |
| ENSG00000114019 | ENSMUSG00000032531 | AMOTL2 | 154 | chr3 | 134076023 | 134076123 |
| ENSG00000103507 | ENSMUSG00000030802 | BCKDK | 155 | chr16 | 31123671 | 31123771 |
| ENSG00000146067 | ENSMUSG00000021495 | FAM193B | 156 | chr5 | 176951261 | 176951361 |
| ENSG00000146067 | ENSMUSG00000021495 | FAM193B | 157 | chr5 | 176951694 | 176951794 |
| ENSG00000135763 | ENSMUSG00000031976 | URB2 | 158 | chr1 | 229770975 | 229771075 |
| ENSG00000135763 | ENSMUSG00000031976 | URB2 | 159 | chr1 | 229773778 | 229773878 |
| ENSG00000163481 | ENSMUSG00000026171 | RNF25 | 160 | chr2 | 219528782 | 219528886 |
| ENSG00000140262 | ENSMUSG00000032228 | TCF12 | 161 | chr15 | 57578404 | 57578514 |
| ENSG00000145604 | ENSMUSG00000054115 | SKP2 | 162 | chr5 | 36152995 | 36153095 |
| ENSG00000101407 | ENSMUSG00000027650 | TTI1 | 163 | chr20 | 36641249 | 36641349 |
| ENSG00000101407 | ENSMUSG00000027650 | TTI1 | 164 | chr20 | 36641767 | 36641867 |
| ENSG00000139182 | ENSMUSG00000008153 | CLSTN3 | 165 | chr12 | 7310712 | 7310812 |
| ENSG00000113360 | ENSMUSG00000022191 | DROSHA | 166 | chr5 | 31526727 | 31526827 |
| ENSG00000175931 | ENSMUSG00000020802 | UBE2O | 167 | chr17 | 74392433 | 74392533 |
| ENSG00000082213 | ENSMUSG00000022195 | C5orf22 | 168 | chr5 | 31538511 | 31538611 |
| ENSG00000112983 | ENSMUSG00000003778 | BRD8 | 169 | chr5 | 137500558 | 137500658 |
| ENSG00000086062 | ENSMUSG00000028413 | B4GALT1 | 170 | chr9 | 33113372 | 33113472 |
| ENSG00000176915 | ENSMUSG00000029501 | ANKLE2 | 171 | chr12 | 133306514 | 133306621 |
| ENSG00000176915 | ENSMUSG00000029501 | ANKLE2 | 172 | chr12 | 133331392 | 133331492 |
| ENSG00000168137 | ENSMUSG00000034269 | SETD5 | 173 | chr3 | 9512354 | 9512454 |
| ENSG00000168137 | ENSMUSG00000034269 | SETD5 | 174 | chr3 | 9517516 | 9517616 |
| ENSG00000168137 | ENSMUSG00000034269 | SETD5 | 175 | chr3 | 9517778 | 9517878 |
| ENSG00000163166 | ENSMUSG00000024384 | IWS1 | 176 | chr2 | 128262332 | 128262432 |
| ENSG00000160710 | ENSMUSG00000027951 | ADAR | 177 | chr1 | 154557261 | 154557369 |
| ENSG00000146247 | ENSMUSG00000032253 | PHIP | 178 | chr6 | 79650447 | 79650547 |
| ENSG00000156304 | ENSMUSG00000022983 | SCAF4 | 179 | chr21 | 33043670 | 33043770 |
| ENSG00000143970 | ENSMUSG00000037486 | ASXL2 | 180 | chr2 | 25964998 | 25965098 |
| ENSG00000188021 | ENSMUSG00000050148 | UBQLN2 | 181 | chrX | 56591658 | 56591758 |
| ENSG00000182372 | ENSMUSG00000026317 | CLN8 | 182 | chr8 | 1728659 | 1728759 |
| ENSG00000126461 | ENSMUSG00000038406 | SCAF1 | 183 | chr19 | 50156594 | 50156694 |
| ENSG00000145632 | ENSMUSG00000021701 | PLK2 | 184 | chr5 | 57750268 | 57750368 |
| ENSG00000168918 | ENSMUSG00000026288 | INPP5D | 185 | chr2 | 234115576 | 234115684 |
| ENSG00000164715 | ENSMUSG00000038970 | LMTK2 | 186 | chr7 | 97823595 | 97823695 |
| ENSG00000030582 | ENSMUSG00000034708 | GRN | 187 | chr17 | 42430159 | 42430259 |
| ENSG00000173786 | ENSMUSG00000006782 | CNP | 188 | chr17 | 40120545 | 40120645 |
| ENSG00000178188 | ENSMUSG00000030733 | SH2B1 | 189 | chr16 | 28878041 | 28878145 |
| ENSG00000121057 | ENSMUSG00000018428 | AKAP1 | 190 | chr17 | 55183430 | 55183530 |
| ENSG00000125484 | ENSMUSG00000035666 | GTF3C4 | 191 | chr9 | 135553562 | 135553663 |
| ENSG00000198700 | ENSMUSG00000041879 | IPO9 | 192 | chr1 | 201845316 | 201845422 |
| ENSG00000182963 | ENSMUSG00000034520 | GJC1 | 193 | chr17 | 42881776 | 42881876 |
| ENSG00000182963 | ENSMUSG00000034520 | GJC1 | 194 | chr17 | 42882363 | 42882463 |
| ENSG00000197256 | ENSMUSG00000032194 | KANK2 | 195 | chr19 | 11303870 | 11303970 |
| ENSG00000123552 | ENSMUSG00000040455 | USP45 | 196 | chr6 | 99893940 | 99894040 |
| ENSG00000171552 | ENSMUSG00000007659 | BCL2L1 | 197 | chr20 | 30309594 | 30309694 |
| ENSG00000100105 | ENSMUSG00000020453 | PATZ1 | 198 | chr22 | 31722789 | 31722895 |
| ENSG00000100105 | ENSMUSG00000020453 | PATZ1 | 199 | chr22 | 31740743 | 31740843 |
| ENSG00000100105 | ENSMUSG00000020453 | PATZ1 | 200 | chr22 | 31740937 | 31741037 |
| ENSG00000185033 | ENSMUSG00000030539 | SEMA4B | 201 | chr15 | 90772185 | 90772292 |
| ENSG00000143363 | ENSMUSG00000015711 | PRUNE | 202 | chr1 | 151006508 | 151006608 |
| ENSG00000102967 | ENSMUSG00000031730 | DHODH | 203 | chr16 | 72058172 | 72058272 |
| ENSG00000062650 | ENSMUSG00000041408 | WAPAL | 204 | chr10 | 88259886 | 88259986 |
| ENSG00000143013 | ENSMUSG00000028266 | LMO4 | 205 | chr1 | 87810689 | 87810789 |
| ENSG00000088367 | ENSMUSG00000027624 | EPB41L1 | 206 | chr20 | 34817404 | 34817504 |
| ENSG00000181555 | ENSMUSG00000044791 | SETD2 | 207 | chr3 | 47098795 | 47098895 |
| ENSG00000162402 | ENSMUSG00000028514 | USP24 | 208 | chr1 | 55532974 | 55533074 |
| ENSG00000162402 | ENSMUSG00000028514 | USP24 | 209 | chr1 | 55534353 | 55534453 |
| ENSG00000108578 | ENSMUSG00000020840 | BLMH | 210 | chr17 | 28575929 | 28576029 |
| ENSG00000158636 | ENSMUSG00000035401 | C11orf30 | 211 | chr11 | 76261122 | 76261222 |
| ENSG00000172795 | ENSMUSG00000024472 | DCP2 | 212 | chr5 | 112349067 | 112349167 |
| ENSG00000159322 | ENSMUSG00000025236 | ADPGK | 213 | chr15 | 73044722 | 73044822 |
| ENSG00000159322 | ENSMUSG00000025236 | ADPGK | 214 | chr15 | 73044897 | 73044997 |
| ENSG00000166068 | ENSMUSG00000027351 | SPRED1 | 215 | chr15 | 38643376 | 38643476 |
| ENSG00000103356 | ENSMUSG00000030871 | EARS2 | 216 | chr16 | 23546495 | 23546595 |
| ENSG00000107651 | ENSMUSG00000055319 | SEC23IP | 217 | chr10 | 121658063 | 121658163 |
| ENSG00000111530 | ENSMUSG00000020114 | CAND1 | 218 | chr12 | 67699642 | 67699742 |
| ENSG00000143379 | ENSMUSG00000015697 | SETDB1 | 219 | chr1 | 150923233 | 150923341 |
| ENSG00000143379 | ENSMUSG00000015697 | SETDB1 | 220 | chr1 | 150933327 | 150933427 |
| ENSG00000143379 | ENSMUSG00000015697 | SETDB1 | 221 | chr1 | 150936842 | 150936942 |
| ENSG00000171492 | ENSMUSG00000046079 | LRRC8D | 222 | chr1 | 90400967 | 90401067 |
| ENSG00000171940 | ENSMUSG00000052056 | ZNF217 | 223 | chr20 | 52192477 | 52192577 |
| ENSG00000083857 | ENSMUSG00000070047 | FAT1 | 224 | chr4 | 187517937 | 187518043 |
| ENSG00000083857 | ENSMUSG00000070047 | FAT1 | 225 | chr4 | 187521195 | 187521295 |
| ENSG00000068097 | ENSMUSG00000000976 | HEATR6 | 226 | chr17 | 58120927 | 58121027 |
| ENSG00000068097 | ENSMUSG00000000976 | HEATR6 | 227 | chr17 | 58121203 | 58121303 |
| ENSG00000099381 | ENSMUSG00000042308 | SETD1A | 228 | chr16 | 30977180 | 30977280 |
| ENSG00000099381 | ENSMUSG00000042308 | SETD1A | 229 | chr16 | 30990852 | 30990952 |
| ENSG00000099381 | ENSMUSG00000042308 | SETD1A | 230 | chr16 | 30991343 | 30991443 |
| ENSG00000009954 | ENSMUSG00000002748 | BAZ1B | 231 | chr7 | 72856467 | 72856567 |
| ENSG00000009954 | ENSMUSG00000002748 | BAZ1B | 232 | chr7 | 72891680 | 72891780 |
| ENSG00000009954 | ENSMUSG00000002748 | BAZ1B | 233 | chr7 | 72891974 | 72892074 |
| ENSG00000009954 | ENSMUSG00000002748 | BAZ1B | 234 | chr7 | 72892449 | 72892549 |
| ENSG00000144524 | ENSMUSG00000026240 | COPS7B | 235 | chr2 | 232673305 | 232673405 |
| ENSG00000132383 | ENSMUSG00000000751 | RPA1 | 236 | chr17 | 1800592 | 1800692 |
| ENSG00000129474 | ENSMUSG00000022178 | AJUBA | 237 | chr14 | 23450606 | 23450706 |
| ENSG00000070366 | ENSMUSG00000038290 | SMG6 | 238 | chr17 | 2202589 | 2202689 |
| ENSG00000152952 | ENSMUSG00000032374 | PLOD2 | 239 | chr3 | 145788480 | 145788580 |
| ENSG00000010322 | ENSMUSG00000021910 | NISCH | 240 | chr3 | 52521541 | 52521641 |
| ENSG00000010322 | ENSMUSG00000021910 | NISCH | 241 | chr3 | 52526156 | 52526256 |
| ENSG00000184863 | ENSMUSG00000048271 | RBM33 | 242 | chr7 | 155567886 | 155567996 |
| ENSG00000184867 | ENSMUSG00000033436 | ARMCX2 | 243 | chrX | 100912393 | 100912493 |
| ENSG00000108219 | ENSMUSG00000037824 | TSPAN14 | 244 | chr10 | 82277982 | 82278082 |
| ENSG00000182544 | ENSMUSG00000045665 | MFSD5 | 245 | chr12 | 53648059 | 53648159 |
| ENSG00000072274 | ENSMUSG00000022797 | TFRC | 246 | chr3 | 195778804 | 195778905 |
| ENSG00000146834 | ENSMUSG00000029726 | MEPCE | 247 | chr7 | 100029121 | 100029221 |
| ENSG00000164040 | ENSMUSG00000049940 | PGRMC2 | 248 | chr4 | 129192367 | 129192467 |
| ENSG00000239306 | ENSMUSG00000006456 | RBM14 | 249 | chr11 | 66391816 | 66391916 |
| ENSG00000198728 | ENSMUSG00000025223 | LDB1 | 250 | chr10 | 103867637 | 103867737 |
| ENSG00000181026 | ENSMUSG00000030609 | AEN | 251 | chr15 | 89169752 | 89169852 |
| ENSG00000142949 | ENSMUSG00000033295 | PTPRF | 252 | chr1 | 44056683 | 44056783 |
| ENSG00000142949 | ENSMUSG00000033295 | PTPRF | 253 | chr1 | 44087837 | 44087937 |
| ENSG00000041802 | ENSMUSG00000022538 | LSG1 | 254 | chr3 | 194362699 | 194362799 |
| ENSG00000151465 | ENSMUSG00000039128 | CDC123 | 255 | chr10 | 12238167 | 12238267 |
| ENSG00000151461 | ENSMUSG00000043241 | UPF2 | 256 | chr10 | 11962806 | 11962906 |
| ENSG00000003393 | ENSMUSG00000026024 | ALS2 | 257 | chr2 | 202565602 | 202565702 |
| ENSG00000143924 | ENSMUSG00000032624 | EML4 | 258 | chr2 | 42557252 | 42557352 |
| ENSG00000123358 | ENSMUSG00000023034 | NR4A1 | 259 | chr12 | 52448563 | 52448663 |
| ENSG00000163113 | ENSMUSG00000038495 | OTUD7B | 260 | chr1 | 149915755 | 149915855 |
| ENSG00000114948 | ENSMUSG00000025964 | ADAM23 | 261 | chr2 | 207482464 | 207482564 |
| ENSG00000109572 | ENSMUSG00000004319 | CLCN3 | 262 | chr4 | 170641332 | 170641432 |
| ENSG00000167862 | ENSMUSG00000018858 | ICT1 | 263 | chr17 | 73017077 | 73017177 |
| ENSG00000158615 | ENSMUSG00000046062 | PPP1R15B | 264 | chr1 | 204375200 | 204375300 |
| ENSG00000158615 | ENSMUSG00000046062 | PPP1R15B | 265 | chr1 | 204379076 | 204379176 |
| ENSG00000101337 | ENSMUSG00000068040 | TM9SF4 | 266 | chr20 | 30753247 | 30753350 |
| ENSG00000101337 | ENSMUSG00000068040 | TM9SF4 | 267 | chr20 | 30753978 | 30754078 |
| ENSG00000137815 | ENSMUSG00000027304 | RTF1 | 268 | chr15 | 41772942 | 41773042 |
| ENSG00000165494 | ENSMUSG00000041328 | PCF11 | 269 | chr11 | 82879692 | 82879792 |
| ENSG00000165494 | ENSMUSG00000041328 | PCF11 | 270 | chr11 | 82880587 | 82880687 |
| ENSG00000116191 | ENSMUSG00000026594 | RALGPS2 | 271 | chr1 | 178885673 | 178885773 |
| ENSG00000117139 | ENSMUSG00000042207 | KDM5B | 272 | chr1 | 202698936 | 202699036 |
| ENSG00000159873 | ENSMUSG00000020482 | CCDC117 | 273 | chr22 | 29182192 | 29182292 |
| ENSG00000170037 | ENSMUSG00000032782 | CNTROB | 274 | chr17 | 7836272 | 7836372 |
| ENSG00000104853 | ENSMUSG00000002981 | CLPTM1 | 275 | chr19 | 45496192 | 45496292 |
| ENSG00000117318 | ENSMUSG00000007872 | ID3 | 276 | chr1 | 23884686 | 23884786 |
| ENSG00000086758 | ENSMUSG00000025261 | HUWE1 | 277 | chrX | 53574997 | 53575097 |
| ENSG00000083093 | ENSMUSG00000044702 | PALB2 | 278 | chr16 | 23641466 | 23641566 |
| ENSG00000140598 | ENSMUSG00000038563 | EFTUD1 | 279 | chr15 | 82443896 | 82443996 |
| ENSG00000156471 | ENSMUSG00000021518 | PTDSS1 | 280 | chr8 | 97345895 | 97345995 |
| ENSG00000147257 | ENSMUSG00000055653 | GPC3 | 281 | chrX | 132887658 | 132887758 |
| ENSG00000136848 | ENSMUSG00000026883 | DAB2IP | 282 | chr9 | 124522263 | 124522363 |
| ENSG00000163125 | ENSMUSG00000028106 | RPRD2 | 283 | chr1 | 150444686 | 150444787 |
| ENSG00000163251 | ENSMUSG00000045005 | FZD5 | 284 | chr2 | 208631902 | 208632002 |
| ENSG00000163251 | ENSMUSG00000045005 | FZD5 | 285 | chr2 | 208632239 | 208632339 |
| ENSG00000215251 | ENSMUSG00000079043 | FASTKD5 | 286 | chr20 | 3127689 | 3127789 |
| ENSG00000135862 | ENSMUSG00000026478 | LAMC1 | 287 | chr1 | 183111784 | 183111884 |
| ENSG00000141568 | ENSMUSG00000039275 | FOXK2 | 288 | chr17 | 80559458 | 80559558 |
| ENSG00000141568 | ENSMUSG00000039275 | FOXK2 | 289 | chr17 | 80560100 | 80560200 |
| ENSG00000165006 | ENSMUSG00000028437 | UBAP1 | 290 | chr9 | 34241944 | 34242044 |
| ENSG00000164284 | ENSMUSG00000024580 | GRPEL2 | 291 | chr5 | 148730644 | 148730744 |
| ENSG00000205213 | ENSMUSG00000050199 | LGR4 | 292 | chr11 | 27389623 | 27389723 |
| ENSG00000124177 | ENSMUSG00000057133 | CHD6 | 293 | chr20 | 40033200 | 40033300 |
| ENSG00000124177 | ENSMUSG00000057133 | CHD6 | 294 | chr20 | 40033590 | 40033691 |
| ENSG00000072071 | ENSMUSG00000013033 | LPHN1 | 295 | chr19 | 14273705 | 14273805 |
| ENSG00000072071 | ENSMUSG00000013033 | LPHN1 | 296 | chr19 | 14273986 | 14274086 |
| ENSG00000160299 | ENSMUSG00000001151 | PCNT | 297 | chr21 | 47783554 | 47783654 |
| ENSG00000075702 | ENSMUSG00000037020 | WDR62 | 298 | chr19 | 36594530 | 36594630 |
| ENSG00000102921 | ENSMUSG00000031652 | N4BP1 | 299 | chr16 | 48595033 | 48595133 |
| ENSG00000102921 | ENSMUSG00000031652 | N4BP1 | 300 | chr16 | 48595778 | 48595878 |
| ENSG00000147130 | ENSMUSG00000031310 | ZMYM3 | 301 | chrX | 70472763 | 70472863 |
| ENSG00000107021 | ENSMUSG00000039678 | TBC1D13 | 302 | chr9 | 131570315 | 131570415 |
| ENSG00000132153 | ENSMUSG00000032480 | DHX30 | 303 | chr3 | 47888285 | 47888385 |
| ENSG00000138162 | ENSMUSG00000030852 | TACC2 | 304 | chr10 | 123970682 | 123970782 |
| ENSG00000112655 | ENSMUSG00000023972 | PTK7 | 305 | chr6 | 43128845 | 43128945 |
| ENSG00000137522 | ENSMUSG00000070426 | RNF121 | 306 | chr11 | 71707404 | 71707504 |
| ENSG00000145982 | ENSMUSG00000021420 | FARS2 | 307 | chr6 | 5369244 | 5369344 |
| ENSG00000197081 | ENSMUSG00000023830 | IGF2R | 308 | chr6 | 160526145 | 160526255 |
| ENSG00000121083 | ENSMUSG00000020483 | DYNLL2 | 309 | chr17 | 56166648 | 56166752 |
| ENSG00000014919 | ENSMUSG00000040018 | COX15 | 310 | chr10 | 101474207 | 101474307 |
| ENSG00000082458 | ENSMUSG00000000881 | DLG3 | 311 | chrX | 69722124 | 69722224 |
| ENSG00000107341 | ENSMUSG00000036241 | UBE2R2 | 312 | chr9 | 33917308 | 33917408 |
| ENSG00000037637 | ENSMUSG00000028920 | FBXO42 | 313 | chr1 | 16577669 | 16577769 |
| ENSG00000124789 | ENSMUSG00000021374 | NUP153 | 314 | chr6 | 17637651 | 17637751 |
| ENSG00000169641 | ENSMUSG00000001089 | LUZP1 | 315 | chr1 | 23414329 | 23414429 |
| ENSG00000169641 | ENSMUSG00000001089 | LUZP1 | 316 | chr1 | 23415379 | 23415479 |
| ENSG00000169641 | ENSMUSG00000001089 | LUZP1 | 317 | chr1 | 23417824 | 23417924 |
| ENSG00000244462 | ENSMUSG00000089824 | RBM12 | 318 | chr20 | 34241710 | 34241810 |
| ENSG00000244462 | ENSMUSG00000089824 | RBM12 | 319 | chr20 | 34242917 | 34243017 |
| ENSG00000048028 | ENSMUSG00000032267 | USP28 | 320 | chr11 | 113669811 | 113669911 |
| ENSG00000132128 | ENSMUSG00000028703 | LRRC41 | 321 | chr1 | 46751441 | 46751541 |
| ENSG00000108528 | ENSMUSG00000014606 | SLC25A11 | 322 | chr17 | 4840589 | 4840689 |
| ENSG00000015532 | ENSMUSG00000020868 | XYLT2 | 323 | chr17 | 48437504 | 48437604 |
| ENSG00000165934 | ENSMUSG00000041781 | CPSF2 | 324 | chr14 | 92628045 | 92628145 |
| ENSG00000172273 | ENSMUSG00000032119 | HINFP | 325 | chr11 | 119005020 | 119005120 |
| ENSG00000132604 | ENSMUSG00000031921 | TERF2 | 326 | chr16 | 69400773 | 69400873 |
| ENSG00000051382 | ENSMUSG00000032462 | PIK3CB | 327 | chr3 | 138374167 | 138374267 |
| ENSG00000153395 | ENSMUSG00000021608 | LPCAT1 | 328 | chr5 | 1463681 | 1463781 |
| ENSG00000128228 | ENSMUSG00000022769 | SDF2L1 | 329 | chr22 | 21998470 | 21998570 |
| ENSG00000104081 | ENSMUSG00000040093 | BMF | 330 | chr15 | 40383240 | 40383340 |
| ENSG00000100364 | ENSMUSG00000036046 | KIAA0930 | 331 | chr22 | 45592538 | 45592638 |
| ENSG00000166902 | ENSMUSG00000024683 | MRPL16 | 332 | chr11 | 59573855 | 59573957 |
| ENSG00000124151 | ENSMUSG00000027678 | NCOA3 | 333 | chr20 | 46275903 | 46276003 |
| ENSG00000104885 | ENSMUSG00000061589 | DOT1L | 334 | chr19 | 2222387 | 2222487 |
| ENSG00000104885 | ENSMUSG00000061589 | DOT1L | 335 | chr19 | 2226731 | 2226831 |
| ENSG00000177613 | ENSMUSG00000053536 | CSTF2T | 336 | chr10 | 53458231 | 53458331 |
| ENSG00000152137 | ENSMUSG00000041548 | HSPB8 | 337 | chr12 | 119617202 | 119617302 |
| ENSG00000166908 | ENSMUSG00000025417 | PIP4K2C | 338 | chr12 | 57995951 | 57996051 |
| ENSG00000105722 | ENSMUSG00000040857 | ERF | 339 | chr19 | 42752707 | 42752807 |
| ENSG00000105722 | ENSMUSG00000040857 | ERF | 340 | chr19 | 42752961 | 42753061 |
| ENSG00000139651 | ENSMUSG00000046897 | ZNF740 | 341 | chr12 | 53581634 | 53581743 |
| ENSG00000172046 | ENSMUSG00000006676 | USP19 | 342 | chr3 | 49145673 | 49145778 |
| ENSG00000187764 | ENSMUSG00000021451 | SEMA4D | 343 | chr9 | 91993600 | 91993707 |
| ENSG00000185619 | ENSMUSG00000033623 | PCGF3 | 344 | chr4 | 759896 | 759996 |
| ENSG00000169925 | ENSMUSG00000026918 | BRD3 | 345 | chr9 | 136898626 | 136898726 |
| ENSG00000126012 | ENSMUSG00000025332 | KDM5C | 346 | chrX | 53222270 | 53222370 |
| ENSG00000126012 | ENSMUSG00000025332 | KDM5C | 347 | chrX | 53223495 | 53223595 |
| ENSG00000122042 | ENSMUSG00000001687 | UBL3 | 348 | chr13 | 30341200 | 30341300 |
| ENSG00000119139 | ENSMUSG00000024812 | TJP2 | 349 | chr9 | 71869441 | 71869541 |
| ENSG00000108262 | ENSMUSG00000011877 | GIT1 | 350 | chr17 | 27901620 | 27901720 |
| ENSG00000101773 | ENSMUSG00000041238 | RBBP8 | 351 | chr18 | 20573357 | 20573457 |
| ENSG00000137504 | ENSMUSG00000051451 | CREBZF | 352 | chr11 | 85375536 | 85375636 |
| ENSG00000138231 | ENSMUSG00000032469 | DBR1 | 353 | chr3 | 137880791 | 137880891 |
| ENSG00000186834 | ENSMUSG00000048878 | HEXIM1 | 354 | chr17 | 43227588 | 43227688 |
| ENSG00000126947 | ENSMUSG00000033460 | ARMCX1 | 355 | chrX | 100808056 | 100808156 |
| ENSG00000113504 | ENSMUSG00000017756 | SLC12A7 | 356 | chr5 | 1051508 | 1051608 |
| ENSG00000085377 | ENSMUSG00000019849 | PREP | 357 | chr6 | 105725839 | 105725939 |
| ENSG00000121274 | ENSMUSG00000036779 | PAPD5 | 358 | chr16 | 50263276 | 50263376 |
| ENSG00000087157 | ENSMUSG00000017715 | PGS1 | 359 | chr17 | 76399910 | 76400010 |
| ENSG00000082781 | ENSMUSG00000022817 | ITGB5 | 360 | chr3 | 124482375 | 124482475 |
| ENSG00000060237 | ENSMUSG00000045962 | WNK1 | 361 | chr12 | 994900 | 995000 |
| ENSG00000174953 | ENSMUSG00000027770 | DHX36 | 362 | chr3 | 153993949 | 153994049 |
| ENSG00000156381 | ENSMUSG00000037904 | ANKRD9 | 363 | chr14 | 102973218 | 102973318 |
| ENSG00000198408 | ENSMUSG00000025220 | MGEA5 | 364 | chr10 | 103546098 | 103546198 |
| ENSG00000198408 | ENSMUSG00000025220 | MGEA5 | 365 | chr10 | 103558723 | 103558823 |
| ENSG00000198331 | ENSMUSG00000050555 | HYLS1 | 366 | chr11 | 125769870 | 125769970 |
| ENSG00000118523 | ENSMUSG00000019997 | CTGF | 367 | chr6 | 132270307 | 132270407 |
| ENSG00000133275 | ENSMUSG00000003345 | CSNK1G2 | 368 | chr19 | 1969780 | 1969880 |
| ENSG00000063978 | ENSMUSG00000029110 | RNF4 | 369 | chr4 | 2515571 | 2515671 |
| ENSG00000162923 | ENSMUSG00000038733 | WDR26 | 370 | chr1 | 224577350 | 224577450 |
| ENSG00000197122 | ENSMUSG00000027646 | SRC | 371 | chr20 | 36031958 | 36032058 |
| ENSG00000173653 | ENSMUSG00000024889 | RCE1 | 372 | chr11 | 66613552 | 66613652 |
| ENSG00000133895 | ENSMUSG00000024947 | MEN1 | 373 | chr11 | 64571737 | 64571837 |
| ENSG00000133895 | ENSMUSG00000024947 | MEN1 | 374 | chr11 | 64572037 | 64572138 |
| ENSG00000101126 | ENSMUSG00000051149 | ADNP | 375 | chr20 | 49508580 | 49508680 |
| ENSG00000170604 | ENSMUSG00000044030 | IRF2BP1 | 376 | chr19 | 46388328 | 46388428 |
| ENSG00000170606 | ENSMUSG00000020361 | HSPA4 | 377 | chr5 | 132440093 | 132440193 |
| ENSG00000136830 | ENSMUSG00000026796 | FAM129B | 378 | chr9 | 130269093 | 130269193 |
| ENSG00000082641 | ENSMUSG00000038615 | NFE2L1 | 379 | chr17 | 46128178 | 46128278 |
| ENSG00000082641 | ENSMUSG00000038615 | NFE2L1 | 380 | chr17 | 46136056 | 46136159 |
| ENSG00000169692 | ENSMUSG00000026922 | AGPAT2 | 381 | chr9 | 139568071 | 139568171 |
| ENSG00000167258 | ENSMUSG00000003119 | CDK12 | 382 | chr17 | 37618686 | 37618789 |
| ENSG00000123200 | ENSMUSG00000022000 | ZC3H13 | 383 | chr13 | 46541803 | 46541903 |
| ENSG00000119596 | ENSMUSG00000021244 | YLPM1 | 384 | chr14 | 75248186 | 75248286 |
| ENSG00000119596 | ENSMUSG00000021244 | YLPM1 | 385 | chr14 | 75264807 | 75264907 |
| ENSG00000119596 | ENSMUSG00000021244 | YLPM1 | 386 | chr14 | 75266182 | 75266282 |
| ENSG00000148840 | ENSMUSG00000055491 | PPRC1 | 387 | chr10 | 103906865 | 103906965 |
| ENSG00000148843 | ENSMUSG00000025047 | PDCD11 | 388 | chr10 | 105205321 | 105205421 |
| ENSG00000148842 | ENSMUSG00000064105 | CNNM2 | 389 | chr10 | 104836827 | 104836927 |
| ENSG00000008083 | ENSMUSG00000038518 | JARID2 | 390 | chr6 | 15496499 | 15496599 |
| ENSG00000008083 | ENSMUSG00000038518 | JARID2 | 391 | chr6 | 15501212 | 15501312 |
| ENSG00000121236 | ENSMUSG00000072244 | TRIM6 | 392 | chr11 | 5632143 | 5632243 |
| ENSG00000154803 | ENSMUSG00000032633 | FLCN | 393 | chr17 | 17116619 | 17116719 |
| ENSG00000099899 | ENSMUSG00000022721 | TRMT2A | 394 | chr22 | 20103732 | 20103832 |
| ENSG00000165526 | ENSMUSG00000032044 | RPUSD4 | 395 | chr11 | 126072955 | 126073055 |
| ENSG00000101138 | ENSMUSG00000027498 | CSTF1 | 396 | chr20 | 54978760 | 54978860 |
| ENSG00000170633 | ENSMUSG00000029474 | RNF34 | 397 | chr12 | 121855506 | 121855606 |
| ENSG00000174579 | ENSMUSG00000066415 | MSL2 | 398 | chr3 | 135870121 | 135870221 |
| ENSG00000174579 | ENSMUSG00000066415 | MSL2 | 399 | chr3 | 135870946 | 135871046 |
| ENSG00000174579 | ENSMUSG00000066415 | MSL2 | 400 | chr3 | 135914097 | 135914197 |
| ENSG00000206557 | ENSMUSG00000079259 | TRIM71 | 401 | chr3 | 32932375 | 32932476 |
| ENSG00000100084 | ENSMUSG00000022702 | HIRA | 402 | chr22 | 19318425 | 19318525 |
| ENSG00000155287 | ENSMUSG00000040414 | SLC25A28 | 403 | chr10 | 101370706 | 101370806 |
| ENSG00000198646 | ENSMUSG00000038369 | NCOA6 | 404 | chr20 | 33337630 | 33337730 |
| ENSG00000198642 | ENSMUSG00000070923 | KLHL9 | 405 | chr9 | 21333560 | 21333669 |
| ENSG00000100888 | ENSMUSG00000053754 | CHD8 | 406 | chr14 | 21853640 | 21853740 |
| ENSG00000100888 | ENSMUSG00000053754 | CHD8 | 407 | chr14 | 21862023 | 21862123 |
| ENSG00000123473 | ENSMUSG00000028718 | STIL | 408 | chr1 | 47716853 | 47716953 |
| ENSG00000155868 | ENSMUSG00000020397 | MED7 | 409 | chr5 | 156565759 | 156565859 |
| ENSG00000160551 | ENSMUSG00000017291 | TAOK1 | 410 | chr17 | 27869955 | 27870055 |
| ENSG00000156983 | ENSMUSG00000001632 | BRPF1 | 411 | chr3 | 9780801 | 9780901 |
| ENSG00000012232 | ENSMUSG00000021978 | EXTL3 | 412 | chr8 | 28575287 | 28575387 |
| ENSG00000163946 | ENSMUSG00000040651 | FAM208A | 413 | chr3 | 56657659 | 56657760 |
| ENSG00000163946 | ENSMUSG00000040651 | FAM208A | 414 | chr3 | 56675500 | 56675600 |
| ENSG00000185624 | ENSMUSG00000025130 | P4HB | 415 | chr17 | 79801835 | 79801936 |
| ENSG00000077684 | ENSMUSG00000025764 | PHF17 | 416 | chr4 | 129783348 | 129783448 |
| ENSG00000077684 | ENSMUSG00000025764 | PHF17 | 417 | chr4 | 129792890 | 129792990 |
| ENSG00000077684 | ENSMUSG00000025764 | PHF17 | 418 | chr4 | 129793234 | 129793334 |
| ENSG00000005810 | ENSMUSG00000033004 | MYCBP2 | 419 | chr13 | 77619357 | 77619457 |
| ENSG00000153827 | ENSMUSG00000026219 | TRIP12 | 420 | chr2 | 230723562 | 230723662 |
| ENSG00000153827 | ENSMUSG00000026219 | TRIP12 | 421 | chr2 | 230724093 | 230724193 |
| ENSG00000099889 | ENSMUSG00000000325 | ARVCF | 422 | chr22 | 19957471 | 19957571 |
| ENSG00000099889 | ENSMUSG00000000325 | ARVCF | 423 | chr22 | 19957765 | 19957865 |
| ENSG00000196367 | ENSMUSG00000045482 | TRRAP | 424 | chr7 | 98609930 | 98610030 |
| ENSG00000127603 | ENSMUSG00000028649 | MACF1 | 425 | chr1 | 39851434 | 39851534 |
| ENSG00000127603 | ENSMUSG00000028649 | MACF1 | 426 | chr1 | 39853080 | 39853183 |
| ENSG00000132964 | ENSMUSG00000029635 | CDK8 | 427 | chr13 | 26828750 | 26828860 |
| ENSG00000132964 | ENSMUSG00000029635 | CDK8 | 428 | chr13 | 26978256 | 26978356 |
| ENSG00000161547 | ENSMUSG00000034120 | SRSF2 | 429 | chr17 | 74733297 | 74733397 |
| ENSG00000206560 | ENSMUSG00000014496 | ANKRD28 | 430 | chr3 | 15711481 | 15711581 |
| ENSG00000145555 | ENSMUSG00000022272 | MYO10 | 431 | chr5 | 16701316 | 16701416 |
| ENSG00000072364 | ENSMUSG00000049470 | AFF4 | 432 | chr5 | 132216686 | 132216786 |
| ENSG00000072364 | ENSMUSG00000049470 | AFF4 | 433 | chr5 | 132232254 | 132232354 |
| ENSG00000115306 | ENSMUSG00000020315 | SPTBN1 | 434 | chr2 | 54858532 | 54858632 |
| ENSG00000115306 | ENSMUSG00000020315 | SPTBN1 | 435 | chr2 | 54876826 | 54876926 |
| ENSG00000180901 | ENSMUSG00000016940 | KCTD2 | 436 | chr17 | 73059955 | 73060055 |
| ENSG00000134452 | ENSMUSG00000058594 | FBXO18 | 437 | chr10 | 5948372 | 5948472 |
| ENSG00000124486 | ENSMUSG00000031010 | USP9X | 438 | chrX | 41075382 | 41075482 |
| ENSG00000124486 | ENSMUSG00000031010 | USP9X | 439 | chrX | 41075663 | 41075763 |
| ENSG00000111737 | ENSMUSG00000029518 | RAB35 | 440 | chr12 | 120534739 | 120534839 |
| ENSG00000111737 | ENSMUSG00000029518 | RAB35 | 441 | chr12 | 120534962 | 120535062 |
| ENSG00000061938 | ENSMUSG00000022791 | TNK2 | 442 | chr3 | 195590509 | 195590609 |
| ENSG00000061938 | ENSMUSG00000022791 | TNK2 | 443 | chr3 | 195594699 | 195594799 |
| ENSG00000132466 | ENSMUSG00000055204 | ANKRD17 | 444 | chr4 | 73957524 | 73957633 |
| ENSG00000131669 | ENSMUSG00000037966 | NINJ1 | 445 | chr9 | 95884141 | 95884241 |
| ENSG00000143740 | ENSMUSG00000009894 | SNAP47 | 446 | chr1 | 227935784 | 227935892 |
| ENSG00000118193 | ENSMUSG00000041498 | KIF14 | 447 | chr1 | 200522702 | 200522807 |
| ENSG00000115816 | ENSMUSG00000024081 | CEBPZ | 448 | chr2 | 37454836 | 37454936 |
| ENSG00000115816 | ENSMUSG00000024081 | CEBPZ | 449 | chr2 | 37455261 | 37455361 |
| ENSG00000091409 | ENSMUSG00000027111 | ITGA6 | 450 | chr2 | 173369102 | 173369202 |
| ENSG00000090863 | ENSMUSG00000003316 | GLG1 | 451 | chr16 | 74487002 | 74487102 |
| ENSG00000138018 | ENSMUSG00000075703 | EPT1 | 452 | chr2 | 26612047 | 26612156 |
| ENSG00000128731 | ENSMUSG00000030451 | HERC2 | 453 | chr15 | 28356705 | 28356805 |
| ENSG00000141664 | ENSMUSG00000038866 | ZCCHC2 | 454 | chr18 | 60241930 | 60242039 |
| ENSG00000186187 | ENSMUSG00000033545 | ZNRF1 | 455 | chr16 | 75141622 | 75141722 |
| ENSG00000116731 | ENSMUSG00000057637 | PRDM2 | 456 | chr1 | 14113255 | 14113359 |
| ENSG00000088448 | ENSMUSG00000031508 | ANKRD10 | 457 | chr13 | 111532021 | 111532121 |
| ENSG00000175602 | ENSMUSG00000095098 | CCDC85B | 458 | chr11 | 65658550 | 65658650 |
| ENSG00000131016 | ENSMUSG00000038587 | AKAP12 | 459 | chr6 | 151673318 | 151673418 |
| ENSG00000107929 | ENSMUSG00000033499 | LARP4B | 460 | chr10 | 858932 | 859032 |
| ENSG00000174197 | ENSMUSG00000033943 | MGA | 461 | chr15 | 42058995 | 42059095 |
| ENSG00000174197 | ENSMUSG00000033943 | MGA | 462 | chr15 | 42059340 | 42059440 |
| ENSG00000120159 | ENSMUSG00000028578 | C9orf82 | 463 | chr9 | 26842367 | 26842467 |
| ENSG00000170471 | ENSMUSG00000027652 | RALGAPB | 464 | chr20 | 37203566 | 37203666 |
| ENSG00000145911 | ENSMUSG00000001053 | N4BP3 | 465 | chr5 | 177548885 | 177548985 |
| ENSG00000007202 | ENSMUSG00000010277 | KIAA0100 | 466 | chr17 | 26961974 | 26962074 |
| ENSG00000169155 | ENSMUSG00000026788 | ZBTB43 | 467 | chr9 | 129595650 | 129595750 |
| ENSG00000094975 | ENSMUSG00000040297 | C1orf9 | 468 | chr1 | 172558818 | 172558918 |
| ENSG00000106077 | ENSMUSG00000040532 | ABHD11 | 469 | chr7 | 73150418 | 73150518 |
| ENSG00000109670 | ENSMUSG00000028086 | FBXW7 | 470 | chr4 | 153243993 | 153244093 |
| ENSG00000143756 | ENSMUSG00000047539 | FBXO28 | 471 | chr1 | 224345245 | 224345345 |
| ENSG00000143756 | ENSMUSG00000047539 | FBXO28 | 472 | chr1 | 224345537 | 224345637 |
| ENSG00000143751 | ENSMUSG00000038806 | C1orf55 | 473 | chr1 | 226173019 | 226173119 |
| ENSG00000123144 | ENSMUSG00000041203 | C19orf43 | 474 | chr19 | 12841604 | 12841712 |
| ENSG00000180104 | ENSMUSG00000034152 | EXOC3 | 475 | chr5 | 453575 | 453675 |
| ENSG00000180104 | ENSMUSG00000034152 | EXOC3 | 476 | chr5 | 453828 | 453934 |
| ENSG00000168209 | ENSMUSG00000020108 | DDIT4 | 477 | chr10 | 74034991 | 74035091 |
| ENSG00000108100 | ENSMUSG00000024286 | CCNY | 478 | chr10 | 35858297 | 35858397 |
| ENSG00000109323 | ENSMUSG00000028164 | MANBA | 479 | chr4 | 103553098 | 103553198 |
| ENSG00000086102 | ENSMUSG00000028423 | NFX1 | 480 | chr9 | 33294990 | 33295090 |
| ENSG00000158793 | ENSMUSG00000013997 | NIT1 | 481 | chr1 | 161090661 | 161090761 |
| ENSG00000155090 | ENSMUSG00000037465 | KLF10 | 482 | chr8 | 103663820 | 103663920 |
| ENSG00000113282 | ENSMUSG00000006169 | CLINT1 | 483 | chr5 | 157214687 | 157214787 |
| ENSG00000177192 | ENSMUSG00000029507 | PUS1 | 484 | chr12 | 132425930 | 132426030 |
| ENSG00000177192 | ENSMUSG00000029507 | PUS1 | 485 | chr12 | 132426321 | 132426428 |
| ENSG00000161526 | ENSMUSG00000020755 | SAP30BP | 486 | chr17 | 73702657 | 73702757 |
| ENSG00000125447 | ENSMUSG00000020740 | GGA3 | 487 | chr17 | 73234125 | 73234225 |
| ENSG00000140943 | ENSMUSG00000031835 | MBTPS1 | 488 | chr16 | 84135474 | 84135575 |
| ENSG00000123444 | ENSMUSG00000005505 | KBTBD4 | 489 | chr11 | 47594763 | 47594863 |
| ENSG00000177479 | ENSMUSG00000064145 | ARIH2 | 490 | chr3 | 48965027 | 48965127 |
| ENSG00000078687 | ENSMUSG00000025571 | TNRC6C | 491 | chr17 | 76046569 | 76046669 |
| ENSG00000078687 | ENSMUSG00000025571 | TNRC6C | 492 | chr17 | 76046869 | 76046969 |
| ENSG00000123154 | ENSMUSG00000005150 | WDR83 | 493 | chr19 | 12786482 | 12786586 |
| ENSG00000123159 | ENSMUSG00000019433 | GIPC1 | 494 | chr19 | 14589100 | 14589200 |
| ENSG00000141867 | ENSMUSG00000024002 | BRD4 | 495 | chr19 | 15349048 | 15349148 |
| ENSG00000173402 | ENSMUSG00000039952 | DAG1 | 496 | chr3 | 49569904 | 49570005 |
| ENSG00000172977 | ENSMUSG00000024926 | KAT5 | 497 | chr11 | 65486624 | 65486724 |
| ENSG00000162222 | ENSMUSG00000071660 | TTC9C | 498 | chr11 | 62505841 | 62505941 |
| ENSG00000186654 | ENSMUSG00000036106 | PRR5 | 499 | chr22 | 45132995 | 45133101 |
| ENSG00000033800 | ENSMUSG00000032405 | PIAS1 | 500 | chr15 | 68378715 | 68378815 |
| ENSG00000033800 | ENSMUSG00000032405 | PIAS1 | 501 | chr15 | 68480137 | 68480237 |
| ENSG00000125386 | ENSMUSG00000037210 | FAM193A | 502 | chr4 | 2701615 | 2701715 |
| ENSG00000160917 | ENSMUSG00000029625 | CPSF4 | 503 | chr7 | 99054138 | 99054238 |
| ENSG00000128274 | ENSMUSG00000047878 | A4GALT | 504 | chr22 | 43089441 | 43089551 |
| ENSG00000129925 | ENSMUSG00000024180 | TMEM8A | 505 | chr16 | 421883 | 421983 |
| ENSG00000118496 | ENSMUSG00000047648 | FBXO30 | 506 | chr6 | 146126071 | 146126171 |
| ENSG00000118496 | ENSMUSG00000047648 | FBXO30 | 507 | chr6 | 146126811 | 146126911 |
| ENSG00000188157 | ENSMUSG00000041936 | AGRN | 508 | chr1 | 990483 | 990583 |
| ENSG00000259399 | ENSMUSG00000027637 | RP5- | 509 | chr20 | 35240578 | 35240678 |
| | | 977B1.10.1 | | | | |
| ENSG00000074047 | ENSMUSG00000048402 | GLI2 | 510 | chr2 | 121749761 | 121749861 |
| ENSG00000119638 | ENSMUSG00000034290 | NEK9 | 511 | chr14 | 75551173 | 75551273 |
| ENSG00000169398 | ENSMUSG00000022607 | PTK2 | 512 | chr8 | 141669483 | 141669583 |
| ENSG00000122299 | ENSMUSG00000037965 | ZC3H7A | 513 | chr16 | 11844902 | 11845002 |
| ENSG00000116273 | ENSMUSG00000047777 | PHF13 | 514 | chr1 | 6681726 | 6681826 |
| ENSG00000159202 | ENSMUSG00000014349 | UBE2Z | 515 | chr17 | 47004408 | 47004508 |
| ENSG00000164151 | ENSMUSG00000034525 | KIAA0947 | 516 | chr5 | 5464892 | 5464992 |
| ENSG00000117676 | ENSMUSG00000003644 | RPS6KA1 | 517 | chr1 | 26900840 | 26900940 |
| ENSG00000171988 | ENSMUSG00000037876 | JMID1C | 518 | chr10 | 64966495 | 64966595 |
| ENSG00000253729 | ENSMUSG00000022672 | PRKDC | 519 | chr8 | 48686727 | 48686827 |
| ENSG00000008952 | ENSMUSG00000027706 | SEC62 | 520 | chr3 | 169710481 | 169710581 |
| ENSG00000111087 | ENSMUSG00000025407 | GLI1 | 521 | chr12 | 57864678 | 57864778 |
| ENSG00000111087 | ENSMUSG00000025407 | GLI1 | 522 | chr12 | 57865629 | 57865729 |
| ENSG00000176095 | ENSMUSG00000032594 | IP6K1 | 523 | chr3 | 49764420 | 49764520 |
| ENSG00000118482 | ENSMUSG00000048874 | PHF3 | 524 | chr6 | 64423311 | 64423411 |
| ENSG00000074181 | ENSMUSG00000038146 | NOTCH3 | 525 | chr19 | 15272088 | 15272188 |
| ENSG00000187678 | ENSMUSG00000024427 | SPRY4 | 526 | chr5 | 141693475 | 141693575 |
| ENSG00000137055 | ENSMUSG00000028577 | PLAA | 527 | chr9 | 26905765 | 26905865 |
| ENSG00000166145 | ENSMUSG00000027315 | SPINT1 | 528 | chr15 | 41137025 | 41137125 |
| ENSG00000166145 | ENSMUSG00000027315 | SPINT1 | 529 | chr15 | 41149216 | 41149316 |
| ENSG00000164366 | ENSMUSG00000021578 | CCDC127 | 530 | chr5 | 205397 | 205497 |
| ENSG00000164366 | ENSMUSG00000021578 | CCDC127 | 531 | chr5 | 205809 | 205909 |
| ENSG00000172409 | ENSMUSG00000027079 | CLP1 | 532 | chr11 | 57428432 | 57428536 |
| ENSG00000196730 | ENSMUSG00000021559 | DAPK1 | 533 | chr9 | 90321265 | 90321365 |
| ENSG00000198952 | ENSMUSG00000001415 | SMG5 | 534 | chr1 | 156235860 | 156235960 |
| ENSG00000160392 | ENSMUSG00000049643 | C19orf47 | 535 | chr19 | 40827762 | 40827862 |
| ENSG00000146063 | ENSMUSG00000040365 | TRIM41 | 536 | chr5 | 180651400 | 180651500 |
| ENSG00000143393 | ENSMUSG00000038861 | PI4KB | 537 | chr1 | 151265003 | 151265103 |
| ENSG00000179151 | ENSMUSG00000038957 | EDC3 | 538 | chr15 | 74924812 | 74924912 |
| ENSG00000168061 | ENSMUSG00000024790 | SAC3D1 | 539 | chr11 | 64811958 | 64812058 |
| ENSG00000068308 | ENSMUSG00000031154 | OTUD5 | 540 | chrX | 48780066 | 48780166 |
| ENSG00000168246 | ENSMUSG00000044949 | UBTD2 | 541 | chr5 | 171638789 | 171638893 |
| ENSG00000168246 | ENSMUSG00000044949 | UBTD2 | 542 | chr5 | 171639024 | 171639124 |
| ENSG00000166398 | ENSMUSG00000066571 | KIAA0355 | 543 | chr19 | 34832705 | 34832805 |
| ENSG00000166398 | ENSMUSG00000066571 | KIAA0355 | 544 | chr19 | 34833168 | 34833268 |
| ENSG00000177200 | ENSMUSG00000056608 | CHD9 | 545 | chr16 | 53358320 | 53358420 |
| ENSG00000163435 | ENSMUSG00000003051 | ELF3 | 546 | chr1 | 201984436 | 201984543 |
| ENSG00000163435 | ENSMUSG00000003051 | ELF3 | 547 | chr1 | 201984774 | 201984874 |
| ENSG00000173120 | ENSMUSG00000054611 | KDM2A | 548 | chr11 | 67022524 | 67022624 |
| ENSG00000070961 | ENSMUSG00000019943 | ATP2B1 | 549 | chr12 | 90049510 | 90049610 |
| ENSG00000116212 | ENSMUSG00000028617 | LRRC42 | 550 | chr1 | 54417770 | 54417870 |
| ENSG00000144674 | ENSMUSG00000038708 | GOLGA4 | 551 | chr3 | 37365215 | 37365315 |
| ENSG00000103966 | ENSMUSG00000027293 | EHD4 | 552 | chr15 | 42192710 | 42192810 |
| ENSG00000110046 | ENSMUSG00000024773 | ATG2A | 553 | chr11 | 64662114 | 64662214 |
| ENSG00000197299 | ENSMUSG00000030528 | BLM | 554 | chr15 | 91293060 | 91293160 |
| ENSG00000129315 | ENSMUSG00000011960 | CCNT1 | 555 | chr12 | 49086996 | 49087103 |
| ENSG00000131711 | ENSMUSG00000052727 | MAP1B | 556 | chr5 | 71501060 | 71501170 |
| ENSG00000198218 | ENSMUSG00000006673 | QRICH1 | 557 | chr3 | 49094397 | 49094497 |
| ENSG00000124571 | ENSMUSG00000067150 | XPO5 | 558 | chr6 | 43491550 | 43491650 |
| ENSG00000136068 | ENSMUSG00000025278 | FLNB | 559 | chr3 | 58109194 | 58109294 |
| ENSG00000114302 | ENSMUSG00000032601 | PRKAR2A | 560 | chr3 | 48788924 | 48789024 |
| ENSG00000142453 | ENSMUSG00000032185 | CARM1 | 561 | chr19 | 11032510 | 11032610 |
| ENSG00000167965 | ENSMUSG00000024142 | MLST8 | 562 | chr16 | 2258975 | 2259075 |
| ENSG00000180357 | ENSMUSG00000040524 | ZNF609 | 563 | chr15 | 64967232 | 64967332 |
| ENSG00000179950 | ENSMUSG00000002524 | PUF60 | 564 | chr8 | 144898598 | 144898698 |
| ENSG00000116062 | ENSMUSG00000005370 | MSH6 | 565 | chr2 | 48027847 | 48027947 |
| ENSG00000112039 | ENSMUSG00000007570 | FANCE | 566 | chr6 | 35423806 | 35423906 |
| ENSG00000125834 | ENSMUSG00000037885 | STK35 | 567 | chr20 | 2097855 | 2097955 |
| ENSG00000132952 | ENSMUSG00000041264 | USPL1 | 568 | chr13 | 31232291 | 31232391 |
| ENSG00000065183 | ENSMUSG00000033285 | WDR3 | 569 | chr1 | 118502058 | 118502158 |
| ENSG00000136709 | ENSMUSG00000024400 | WDR33 | 570 | chr2 | 128463860 | 128463960 |
| ENSG00000136709 | ENSMUSG00000024400 | WDR33 | 571 | chr2 | 128477586 | 128477688 |
| ENSG00000060749 | ENSMUSG00000074994 | QSER1 | 572 | chr11 | 32975575 | 32975677 |
| ENSG00000110074 | ENSMUSG00000039048 | FOXRED1 | 573 | chr11 | 126147790 | 126147890 |
| ENSG00000197912 | ENSMUSG00000000738 | SPG7 | 574 | chr16 | 89623369 | 89623469 |
| ENSG00000156273 | ENSMUSG00000025612 | BACH1 | 575 | chr21 | 30714781 | 30714881 |
| ENSG00000156273 | ENSMUSG00000025612 | BACH1 | 576 | chr21 | 30715069 | 30715170 |
| ENSG00000140829 | ENSMUSG00000037993 | DHX38 | 577 | chr16 | 72146588 | 72146688 |
| ENSG00000100330 | ENSMUSG00000034354 | MTMR3 | 578 | chr22 | 30421779 | 30421879 |
| ENSG00000111300 | ENSMUSG00000042719 | NAA25 | 579 | chr12 | 112467315 | 112467415 |
| ENSG00000068323 | ENSMUSG00000000134 | TFE3 | 580 | chrX | 48887731 | 48887841 |
| ENSG00000111785 | ENSMUSG00000035620 | RIC8B | 581 | chr12 | 107208767 | 107208867 |
| ENSG00000183155 | ENSMUSG00000042229 | RABIF | 582 | chr1 | 202850072 | 202850172 |
| ENSG00000114315 | ENSMUSG00000022528 | HES1 | 583 | chr3 | 193856056 | 193856156 |
| ENSG00000136280 | ENSMUSG00000000378 | CCM2 | 584 | chr7 | 45115631 | 45115731 |
| ENSG00000133704 | ENSMUSG00000040029 | IPO8 | 585 | chr12 | 30783478 | 30783578 |
| ENSG00000167978 | ENSMUSG00000039218 | SRRM2 | 586 | chr16 | 2817292 | 2817392 |
| ENSG00000130939 | ENSMUSG00000028960 | UBE4B | 587 | chr1 | 10093638 | 10093738 |
| ENSG00000130713 | ENSMUSG00000039356 | EXOSC2 | 588 | chr9 | 133579180 | 133579280 |
| ENSG00000108256 | ENSMUSG00000037857 | NUFIP2 | 589 | chr17 | 27613945 | 27614045 |
| ENSG00000108256 | ENSMUSG00000037857 | NUFIP2 | 590 | chr17 | 27614146 | 27614246 |
| ENSG00000108256 | ENSMUSG00000037857 | NUFIP2 | 591 | chr17 | 27614471 | 27614571 |
| ENSG00000257315 | ENSMUSG00000094410 | ZBED6 | 592 | chr1 | 203768104 | 203768204 |
| ENSG00000075856 | ENSMUSG00000018974 | SART3 | 593 | chr12 | 108920050 | 108920150 |
| ENSG00000159023 | ENSMUSG00000028906 | EPB41 | 594 | chr1 | 29314204 | 29314304 |
| ENSG00000107758 | ENSMUSG00000021816 | PPP3CB | 595 | chr10 | 75197869 | 75197969 |
| ENSG00000156599 | ENSMUSG00000034075 | ZDHHC5 | 596 | chr11 | 57439906 | 57440006 |
| ENSG00000176986 | ENSMUSG00000039367 | SEC24C | 597 | chr10 | 75530820 | 75530920 |
| ENSG00000169018 | ENSMUSG00000032244 | FEM1B | 598 | chr15 | 68582094 | 68582194 |
| ENSG00000169018 | ENSMUSG00000032244 | FEM1B | 599 | chr15 | 68582606 | 68582706 |
| ENSG00000058804 | ENSMUSG00000028614 | TMEM48 | 600 | chr1 | 54233519 | 54233619 |
| ENSG00000167182 | ENSMUSG00000018678 | SP2 | 601 | chr17 | 46005389 | 46005489 |
| ENSG00000162714 | ENSMUSG00000020472 | ZNF496 | 602 | chr1 | 247463981 | 247464081 |
| ENSG00000146576 | ENSMUSG00000039244 | C7orf26 | 603 | chr7 | 6639684 | 6639787 |
| ENSG00000020256 | ENSMUSG00000027551 | ZFP64 | 604 | chr20 | 50768775 | 50768875 |
| ENSG00000165458 | ENSMUSG00000032737 | INPPL1 | 605 | chr11 | 71948631 | 71948731 |
| ENSG00000196510 | ENSMUSG00000029466 | ANAPC7 | 606 | chr12 | 110811954 | 110812063 |
| ENSG00000130764 | ENSMUSG00000029028 | LRRC47 | 607 | chr1 | 3697677 | 3697777 |
| ENSG00000122515 | ENSMUSG00000041164 | ZMIZ2 | 608 | chr7 | 44807321 | 44807421 |
| ENSG00000138182 | ENSMUSG00000024795 | KIF20B | 609 | chr10 | 91497229 | 91497329 |
| ENSG00000142627 | ENSMUSG00000006445 | EPHA2 | 610 | chr1 | 16451467 | 16451567 |
| ENSG00000197329 | ENSMUSG00000020134 | PELI1 | 611 | chr2 | 64321768 | 64321868 |
| ENSG00000197329 | ENSMUSG00000020134 | PELI1 | 612 | chr2 | 64322106 | 64322206 |
| ENSG00000006007 | ENSMUSG00000033917 | GDE1 | 613 | chr16 | 19514682 | 19514782 |
| ENSG00000156925 | ENSMUSG00000067860 | ZIC3 | 614 | chrX | 136649462 | 136649562 |
| ENSG00000016864 | ENSMUSG00000021916 | GLT8D1 | 615 | chr3 | 52728812 | 52728912 |
| ENSG00000160606 | ENSMUSG00000019437 | TLCD1 | 616 | chr17 | 27051425 | 27051525 |
| ENSG00000182866 | ENSMUSG00000000409 | LCK | 617 | chr1 | 32751383 | 32751483 |
| ENSG00000065243 | ENSMUSG00000004591 | PKN2 | 618 | chr1 | 89299082 | 89299182 |
| ENSG00000078902 | ENSMUSG00000025139 | TOLLIP | 619 | chr11 | 1298132 | 1298232 |
| ENSG00000168286 | ENSMUSG00000036442 | THAP11 | 620 | chr16 | 67877695 | 67877795 |
| ENSG00000105663 | ENSMUSG00000006307 | MLL4.1 | 621 | chr19 | 36223396 | 36223496 |
| ENSG00000149782 | ENSMUSG00000024960 | PLCB3 | 622 | chr11 | 64034980 | 64035080 |
| ENSG00000148925 | ENSMUSG00000038187 | BTBD10 | 623 | chr11 | 13410457 | 13410557 |
| ENSG00000176248 | ENSMUSG00000026965 | ANAPC2 | 624 | chr9 | 140082180 | 140082282 |
| ENSG00000162702 | ENSMUSG00000041483 | ZNF281 | 625 | chr1 | 200376434 | 200376534 |
| ENSG00000162702 | ENSMUSG00000041483 | ZNF281 | 626 | chr1 | 200377690 | 200377790 |
| ENSG00000166135 | ENSMUSG00000036450 | HIFIAN | 627 | chr10 | 102307974 | 102308074 |
| ENSG00000166133 | ENSMUSG00000027324 | RPUSD2 | 628 | chr15 | 40866435 | 40866535 |
| ENSG00000115207 | ENSMUSG00000029144 | GTF3C2 | 629 | chr2 | 27549400 | 27549500 |
| ENSG00000115207 | ENSMUSG00000029144 | GTF3C2 | 630 | chr2 | 27549672 | 27549772 |
| ENSG00000119787 | ENSMUSG00000059811 | ATL2 | 631 | chr2 | 38523044 | 38523144 |
| ENSG00000138375 | ENSMUSG00000039354 | SMARCAL1 | 632 | chr2 | 217280043 | 217280143 |
| ENSG00000130772 | ENSMUSG00000066042 | MED18 | 633 | chr1 | 28661407 | 28661507 |
| ENSG00000149503 | ENSMUSG00000024660 | INCENP | 634 | chr11 | 61897738 | 61897838 |
| ENSG00000182871 | ENSMUSG00000001435 | COL18A1 | 635 | chr21 | 46932540 | 46932640 |
| ENSG00000154945 | ENSMUSG00000020864 | ANKRD40 | 636 | chr17 | 48773290 | 48773390 |
| ENSG00000198783 | ENSMUSG00000046010 | ZNF830 | 637 | chr17 | 33289068 | 33289168 |
| ENSG00000198783 | ENSMUSG00000046010 | ZNF830 | 638 | chr17 | 33289610 | 33289710 |
| ENSG00000198780 | ENSMUSG00000041817 | FAM169A | 639 | chr5 | 74077389 | 74077498 |
| ENSG00000143457 | ENSMUSG00000046519 | GOLPH3L | 640 | chr1 | 150620824 | 150620924 |
| ENSG00000181449 | | SOX2 | 641 | | | |
| ENSG00000111704 | | NANOG | 642 | | | |
| ENSG00000175387 | | SMAD2 | 643 | | | |
| ENSG00000166949 | | SMAD3 | 644 | | | |
| ENSG00000136997 | | MYC | 645 | | | |
| ENSG00000137815 | | RTF1 | 646 | | | |
| ENSG00000103479 | | RBL2 | 647 | | | |
| ENSG00000008083 | | JARID2 | 648 | | | |
| ENSG00000131914 | | LIN28 | 649 | | | |
| ENSG00000168036 | | CTNNB1 | 650 | | | |
| ENSG00000125686 | | MED1 | 651 | | | |
| ENSG00000074266 | | EED | 652 | | | |
| ENSG00000245532 | | NEAT1 | 653 | | | |
| ENSG00000258609 | | LINC-ROR | 654 | | | |
| ENSG00000279897 | | MEGAMIND/ | 655 | | | |
| | | TUNA | | | | |
| | | (BIRC6 | | | | |
| | | antisense | | | | |
| | | RNA 2) | | | | |
| ENSG00000163508 | | EOMES | 656 | | | |
| ENSG00000125798 | | FOXA2 | 657 | | | |

TABLE 2
DPMI between undifferentiated (resting)human H1-ESC (T0) and 48 hours after Activin A
induction towards endoderm (mesoendoderm) lineage (T48)
Table 2: List of genes (mRNA transcripts) with Differential Peak Intensities (DPMI) between
undifferentiated (resting)human H1-ESC (T0) and 48 hours after Activin A induction towards
endoderm (mesoendoderm) lineage (T48). Coordinates of m6A peaks in human genome (mm9),
type of transcript, and gene symbols are shown. Each row indicates if DPMI is over 1.5- or 2-fold.
(Related to FIG. 5).

| | | DMPI | DMPI | | | DMPI | DMPI |
|---|---|---|---|---|---|---|---|
| | | (fold | (fold | | | (fold | (fold |
| | | change >2) | change >1.5) | | Gene | change | change |
| Gene | Gene Symbol | (Yes/No) | (Yes/No) | Gene | Symbol | >2) | >1.5) |
| ENSG00000064703 | DDX20 | N | Y | ENSG00000166025 | AMOTL1 | N | Y |
| ENSG00000086015 | MAST2 | N | Y | ENSG00000166025 | AMOTL1 | Y | Y |
| ENSG00000160087 | UBE2J2 | N | Y | ENSG00000175216 | CKAP5 | N | Y |
| ENSG00000160688 | FLAD1 | N | Y | ENSG00000196323 | ZBTB44 | N | Y |
| ENSG00000143476 | DTL | Y | Y | ENSG00000137504 | CREBZF | N | Y |
| ENSG00000142599 | RERE | N | Y | ENSG00000182704 | TSKU | N | Y |
| ENSG00000142599 | RERE | Y | Y | ENSG00000182704 | TSKU | N | Y |
| ENSG00000179403 | VWA1 | N | Y | ENSG00000186635 | ARAP1 | Y | Y |
| ENSG00000203668 | CHML | Y | Y | ENSG00000070047 | PHRF1 | N | Y |
| ENSG00000117523 | PRRC2C | N | Y | ENSG00000173621 | LRFN4 | N | Y |
| ENSG00000162377 | SELRC1 | N | Y | ENSG00000133789 | SWAP70 | N | Y |
| ENSG00000162377 | SELRC1 | Y | Y | ENSG00000166261 | ZNF202 | N | Y |
| ENSG00000204138 | PHACTR4 | N | Y | ENSG00000166261 | ZNF202 | N | Y |
| ENSG00000204138 | PHACTR4 | N | Y | ENSG00000142102 | ATHL1 | N | Y |
| ENSG00000158769 | F11R | N | Y | ENSG00000149428 | HYOU1 | Y | Y |
| ENSG00000143337 | TOR1AIP1 | N | Y | ENSG00000149428 | HYOU1 | N | Y |
| ENSG00000143337 | TOR1AIP1 | Y | Y | ENSG00000162194 | C11orf48 | N | Y |
| ENSG00000143624 | INTS3 | N | Y | ENSG00000134824 | FADS2 | N | Y |
| ENSG00000168159 | RNF187 | N | Y | ENSG00000168040 | FADD | N | Y |
| ENSG00000090273 | NUDC | N | Y | ENSG00000188486 | H2AFX | N | Y |
| ENSG00000090273 | NUDC | N | Y | ENSG00000188486 | H2AFX | N | Y |
| ENSG00000117724 | CENPF | Y | Y | ENSG00000149091 | DGKZ | Y | Y |
| ENSG00000117724 | CENPF | N | Y | ENSG00000175827 | AP001266.1 | N | Y |
| ENSG00000143294 | PRCC | N | Y | ENSG00000110048 | OSBP | N | Y |
| ENSG00000163374 | YY1AP1 | N | Y | ENSG00000121653 | MAPK8IP1 | N | Y |
| ENSG00000158796 | DEDD | N | Y | ENSG00000110060 | PUS3 | Y | Y |
| ENSG00000136636 | KCTD3 | N | Y | ENSG00000165458 | INPPL1 | Y | Y |
| ENSG00000164011 | ZNF691 | N | Y | ENSG00000078902 | TOLLIP | N | Y |
| ENSG00000160710 | ADAR | N | Y | ENSG00000160613 | PCSK7 | Y | Y |
| ENSG00000258465 | RP11- | N | Y | ENSG00000072518 | MARK2 | N | Y |
| | 574F21.3.1 | | | | | | |
| ENSG00000116667 | C1orf21 | N | Y | ENSG00000149016 | TUT1 | N | Y |
| ENSG00000142949 | PTPRF | Y | Y | ENSG00000184281 | TSSC4 | N | Y |
| ENSG00000142949 | PTPRF | N | Y | ENSG00000089597 | GANAB | N | Y |
| ENSG00000142949 | PTPRF | N | Y | ENSG00000198561 | CTNND1 | N | Y |
| ENSG00000083444 | PLOD1 | N | Y | ENSG00000165434 | PGM2L1 | N | Y |
| ENSG00000083444 | PLOD1 | N | Y | ENSG00000196914 | ARHGEF12 | N | Y |
| ENSG00000116863 | ADPRHL2 | N | Y | ENSG00000110711 | AIP | N | Y |
| ENSG00000160803 | UBQLN4 | N | Y | ENSG00000137497 | NUMA1 | N | Y |
| ENSG00000171492 | LRRC8D | N | Y | ENSG00000137497 | NUMA1 | N | Y |
| ENSG00000158195 | WASF2 | N | Y | ENSG00000137497 | NUMA1 | N | Y |
| ENSG00000180198 | RCC1 | N | Y | ENSG00000205213 | LGR4 | N | Y |
| ENSG00000122482 | ZNF644 | N | Y | ENSG00000149532 | CPSF7 | N | Y |
| ENSG00000020129 | NCDN | N | Y | ENSG00000149823 | C11orf2 | Y | Y |
| ENSG00000153187 | HNRNPU | N | Y | ENSG00000137513 | NARS2 | N | Y |
| ENSG00000157184 | CPT2 | Y | Y | ENSG00000166902 | MRPL16 | N | Y |
| ENSG00000157184 | CPT2 | Y | Y | ENSG00000076053 | RBM7 | N | Y |
| ENSG00000107404 | DVL1 | N | Y | ENSG00000174669 | SLC29A2 | N | Y |
| ENSG00000215717 | TMEM167B | Y | Y | ENSG00000168569 | TMEM223 | N | Y |
| ENSG00000171603 | CLSTN1 | N | Y | ENSG00000234857 | RP11- | N | Y |
| | | | | | 831H9.16.1 | | |
| ENSG00000143079 | CTTNBP2NL | Y | Y | ENSG00000120451 | SNX19 | Y | Y |
| ENSG00000135823 | STX6 | N | Y | ENSG00000120451 | SNX19 | N | Y |
| ENSG00000135823 | STX6 | N | Y | ENSG00000135372 | NAT10 | N | Y |
| ENSG00000134690 | CDCA8 | N | Y | ENSG00000162236 | STX5 | N | Y |
| ENSG00000066135 | KDM4A | N | Y | ENSG00000173898 | SPTBN2 | N | Y |
| ENSG00000066135 | KDM4A | N | Y | ENSG00000095139 | ARCN1 | N | Y |
| ENSG00000185630 | PBX1 | N | Y | ENSG00000060749 | QSER1 | N | Y |
| ENSG00000130695 | CEP85 | N | Y | ENSG00000110074 | FOXRED1 | N | Y |
| ENSG00000116754 | SRSF11 | N | Y | ENSG00000167985 | SDHAF2 | Y | Y |
| ENSG00000162783 | IER5 | N | Y | ENSG00000059804 | SLC2A3 | N | Y |
| ENSG00000116128 | BCL9 | Y | Y | ENSG00000057294 | PKP2 | N | Y |
| ENSG00000116128 | BCL9 | N | Y | ENSG00000196498 | NCOR2 | N | Y |
| ENSG00000168264 | IRF2BP2 | Y | Y | ENSG00000189079 | ARID2 | Y | Y |
| ENSG00000188157 | AGRN | N | Y | ENSG00000111602 | TIMELESS | N | Y |
| ENSG00000188157 | AGRN | N | Y | ENSG00000111602 | TIMELESS | N | Y |
| ENSG00000157870 | FAM213B | N | Y | ENSG00000088986 | DYNLL1 | Y | Y |
| ENSG00000213516 | RBMXL1 | N | Y | ENSG00000003056 | M6PR | Y | Y |
| ENSG00000160679 | CHTOP | Y | Y | ENSG00000177084 | POLE | N | Y |
| ENSG00000198492 | YTHDF2 | N | Y | ENSG00000181852 | RNF41 | N | Y |
| ENSG00000198492 | YTHDF2 | N | Y | ENSG00000182500 | ORAI1 | N | Y |
| ENSG00000198492 | YTHDF2 | N | Y | ENSG00000151952 | TMEM132D | N | Y |
| ENSG00000143384 | MCL1 | Y | Y | ENSG00000123094 | RASSF8 | N | Y |
| ENSG00000169641 | LUZP1 | N | Y | ENSG00000171792 | C12orf32 | N | Y |
| ENSG00000169641 | LUZP1 | N | Y | ENSG00000161813 | LARP4 | N | Y |
| ENSG00000116698 | SMG7 | N | Y | ENSG00000161813 | LARP4 | Y | Y |
| ENSG00000116691 | MIIP | Y | Y | ENSG00000139613 | SMARCC2 | N | Y |
| ENSG00000143545 | RAB13 | N | Y | ENSG00000173064 | C12orf51 | N | Y |
| ENSG00000253368 | TRNP1 | N | Y | ENSG00000175215 | CTDSP2 | N | Y |
| ENSG00000143153 | ATP1B1 | N | Y | ENSG00000175215 | CTDSP2 | N | Y |
| ENSG00000197622 | CDC42SE1 | N | Y | ENSG00000183495 | EP400 | Y | Y |
| ENSG00000185483 | ROR1 | Y | Y | ENSG00000126746 | ZNF384 | N | Y |
| ENSG00000054118 | THRAP3 | N | Y | ENSG00000170633 | RNF34 | N | Y |
| ENSG00000082512 | TRAF5 | N | Y | ENSG00000170633 | RNF34 | N | Y |
| ENSG00000143390 | RFX5 | N | Y | ENSG00000139318 | DUSP6 | Y | Y |
| ENSG00000154358 | OBSCN | Y | Y | ENSG00000170855 | TRIAP1 | N | Y |
| ENSG00000130764 | LRRC47 | N | Y | ENSG00000253719 | ATXN7L3B | Y | Y |
| ENSG00000130764 | LRRC47 | N | Y | ENSG00000166225 | FRS2 | Y | Y |
| ENSG00000085552 | IGSF9 | N | Y | ENSG00000139154 | AEBP2 | N | Y |
| ENSG00000162702 | ZNF281 | N | Y | ENSG00000167548 | MLL2 | N | Y |
| ENSG00000162702 | ZNF281 | N | Y | ENSG00000076108 | BAZ2A | N | Y |
| ENSG00000162702 | ZNF281 | Y | Y | ENSG00000134287 | ARF3 | N | Y |
| ENSG00000158710 | TAGLN2 | N | Y | ENSG00000174106 | LEMD3 | N | Y |
| ENSG00000204160 | ZDHHC18 | N | Y | ENSG00000171681 | ATF7IP | Y | Y |
| ENSG00000204160 | ZDHHC18 | N | Y | ENSG00000089094 | KDM2B | N | Y |
| ENSG00000116560 | SFPQ | N | Y | ENSG00000089094 | KDM2B | N | Y |
| ENSG00000023902 | PLEKHO1 | N | Y | ENSG00000247077 | PGAM5 | N | Y |
| ENSG00000134247 | PTGFRN | N | Y | ENSG00000136026 | CKAP4 | N | Y |
| ENSG00000078618 | NRD1 | N | Y | ENSG00000123066 | MED13L | Y | Y |
| ENSG00000116584 | ARHGEF2 | N | Y | ENSG00000166860 | ZBTB39 | N | Y |
| ENSG00000142655 | PEX14 | N | Y | ENSG00000161638 | ITGA5 | N | Y |
| ENSG00000132688 | NES | N | Y | ENSG00000111266 | DUSP16 | N | Y |
| ENSG00000132688 | NES | Y | Y | ENSG00000087448 | KLHDC5 | Y | Y |
| ENSG00000158966 | CACHD1 | N | Y | ENSG00000081760 | AACS | N | Y |
| ENSG00000158966 | CACHD1 | Y | Y | ENSG00000110871 | COQ5 | N | Y |
| ENSG00000058673 | ZC3H11A | N | Y | ENSG00000184047 | DIABLO | N | Y |
| ENSG00000186283 | TOR3A | N | Y | ENSG00000111412 | C12orf49 | N | Y |
| ENSG00000197965 | MPZL1 | Y | Y | ENSG00000133639 | BTG1 | Y | Y |
| ENSG00000053372 | MRTO4 | Y | Y | ENSG00000111752 | PHC1 | Y | Y |
| ENSG00000157933 | SKI | N | Y | ENSG00000150990 | DHX37 | N | Y |
| ENSG00000164008 | C1orf50 | N | Y | ENSG00000166598 | HSP90B1 | N | Y |
| ENSG00000085491 | SLC25A24 | N | Y | ENSG00000185591 | SP1 | N | Y |
| ENSG00000116871 | MAP7D1 | Y | Y | ENSG00000060237 | WNK1 | N | Y |
| ENSG00000198700 | IPO9 | N | Y | ENSG00000120800 | UTP20 | Y | Y |
| ENSG00000162419 | GMEB1 | N | Y | ENSG00000013573 | DDX11 | N | Y |
| ENSG00000160818 | GPATCH4 | N | Y | ENSG00000174718 | C12orf35 | N | Y |
| ENSG00000143486 | EIF2D | Y | Y | ENSG00000082805 | ERC1 | N | Y |
| ENSG00000132716 | DCAF8 | N | Y | ENSG00000136014 | USP44 | Y | Y |
| ENSG00000132716 | DCAF8 | N | Y | ENSG00000136014 | USP44 | N | Y |
| ENSG00000116990 | MYCL1 | Y | Y | ENSG00000167272 | POP5 | Y | Y |
| ENSG00000188976 | NC2L | Y | Y | ENSG00000050405 | LIMA1 | N | Y |
| ENSG00000118200 | CAMSAP2 | Y | Y | ENSG00000089154 | GCN1L1 | N | Y |
| ENSG00000054116 | TRAPPC3 | N | Y | ENSG00000110931 | CAMKK2 | N | Y |
| ENSG00000155380 | SLC16A1 | Y | Y | ENSG00000110931 | CAMKK2 | N | Y |
| ENSG00000143061 | IGSF3 | N | Y | ENSG00000150977 | RILPL2 | N | Y |
| ENSG00000162923 | WDR26 | N | Y | ENSG00000120647 | CCDC77 | N | Y |
| ENSG00000186603 | HPDL | N | Y | ENSG00000178498 | DTX3 | Y | Y |
| ENSG00000065526 | SPEN | N | Y | ENSG00000174437 | ATP2A2 | Y | Y |
| ENSG00000065526 | SPEN | N | Y | ENSG00000175727 | MLXIP | N | Y |
| ENSG00000182827 | ACBD3 | Y | Y | ENSG00000102804 | TSC22D1 | N | Y |
| ENSG00000078808 | SDF4 | N | Y | ENSG00000102804 | TSC22D1 | Y | Y |
| ENSG00000158109 | TPRG1L | N | Y | ENSG00000043355 | ZIC2 | N | Y |
| ENSG00000116473 | RAP1A | N | Y | ENSG00000187498 | COL4A1 | Y | Y |
| ENSG00000160685 | ZBTB7B | N | Y | ENSG00000187498 | COL4A1 | Y | Y |
| ENSG00000224870 | RP4- | N | Y | ENSG00000125249 | RAP2A | N | Y |
| | 758J18.2.1 | | | | | | |
| ENSG00000224870 | RP4- | N | Y | ENSG00000136122 | BORA | Y | Y |
| | 758J18.2.1 | | | | | | |
| ENSG00000162512 | SDC3 | Y | Y | ENSG00000150907 | FOXO1 | N | Y |
| ENSG00000215908 | CROCCP2 | Y | Y | ENSG00000133104 | SPG20 | Y | Y |
| ENSG00000242590 | RP11- | N | Y | ENSG00000234787 | LINC00458 | Y | Y |
| | 54O7.14.1 | | | | | | |
| ENSG00000154305 | MIA3 | Y | Y | ENSG00000134899 | ERCC5 | N | Y |
| ENSG00000127603 | MACF1 | N | Y | ENSG00000122042 | UBL3 | N | Y |
| ENSG00000198837 | DENND4B | N | Y | ENSG00000139514 | SLC7A1 | Y | Y |
| ENSG00000213190 | MLLT11 | N | Y | ENSG00000139514 | SLC7A1 | Y | Y |
| ENSG00000198952 | SMG5 | N | Y | ENSG00000169062 | UPF3A | N | Y |
| ENSG00000143375 | CGN | Y | Y | ENSG00000150510 | FAM124A | N | Y |
| ENSG00000031698 | SARS | N | Y | ENSG00000123200 | ZC3H13 | N | Y |
| ENSG00000060656 | PTPRU | N | Y | ENSG00000123200 | ZC3H13 | Y | Y |
| ENSG00000036549 | ZZZ3 | Y | Y | ENSG00000198894 | KIAA1737 | Y | Y |
| ENSG00000196182 | STK40 | Y | Y | ENSG00000165898 | ISCA2 | N | Y |
| ENSG00000116237 | ICMT | N | Y | ENSG00000100852 | ARHGAP5 | N | Y |
| ENSG00000116237 | ICMT | N | Y | ENSG00000205476 | CCDC85C | N | Y |
| ENSG00000117713 | ARID1A | N | Y | ENSG00000092148 | HECTD1 | N | Y |
| ENSG00000117713 | ARID1A | N | Y | ENSG00000100813 | ACIN1 | N | Y |
| ENSG00000117713 | ARID1A | N | Y | ENSG00000100813 | ACIN1 | N | Y |
| ENSG00000162714 | ZNF496 | N | Y | ENSG00000197102 | DYNC1H1 | N | Y |
| ENSG00000143457 | GOLPH3L | Y | Y | ENSG00000089737 | DDX24 | N | Y |
| ENSG00000180398 | MCFD2 | N | Y | ENSG00000100650 | SRSF5 | Y | Y |
| ENSG00000135916 | ITM2C | N | Y | ENSG00000119596 | YLPM1 | N | Y |
| ENSG00000247626 | MARS2 | N | Y | ENSG00000100461 | RBM23 | N | Y |
| ENSG00000176946 | THAP4 | N | Y | ENSG00000100461 | RBM23 | Y | Y |
| ENSG00000115694 | STK25 | N | Y | ENSG00000006432 | MAP3K9 | Y | Y |
| ENSG00000082258 | CCNT2 | N | Y | ENSG00000100441 | KHNYN | N | Y |
| ENSG00000082258 | CCNT2 | N | Y | ENSG00000015133 | CCDC88C | N | Y |
| ENSG00000163811 | WDR43 | N | Y | ENSG00000100938 | GMPR2 | N | Y |
| ENSG00000198142 | ANKRD57 | N | Y | ENSG00000255242 | C14orf169 | N | Y |
| ENSG00000143970 | ASXL2 | N | Y | ENSG00000255242 | C14orf169 | N | Y |
| ENSG00000143970 | ASXL2 | N | Y | ENSG00000139998 | RAB15 | Y | Y |
| ENSG00000135912 | TTLL4 | N | Y | ENSG00000139998 | RAB15 | Y | Y |
| ENSG00000135912 | TTLL4 | N | Y | ENSG00000119669 | IRF2BPL | N | Y |
| ENSG00000115170 | ACVR1 | Y | Y | ENSG00000100823 | APEX1 | N | Y |
| ENSG00000213160 | KLHL23 | Y | Y | ENSG00000165617 | DACT1 | Y | Y |
| ENSG00000213160 | KLHL23 | Y | Y | ENSG00000072042 | RDH11 | N | Y |
| ENSG00000082898 | XPO1 | N | Y | ENSG00000197119 | SLC25A29 | N | Y |
| ENSG00000114948 | ADAM23 | N | Y | ENSG00000197119 | SLC25A29 | N | Y |
| ENSG00000163251 | FZD5 | N | Y | ENSG00000157227 | MMP14 | N | Y |
| ENSG00000197329 | PELI1 | N | Y | ENSG00000157227 | MMP14 | N | Y |
| ENSG00000152284 | TCF7L1 | N | Y | ENSG00000100796 | SMEK1 | N | Y |
| ENSG00000115464 | USP34 | N | Y | ENSG00000066735 | KIF26A | N | Y |
| ENSG00000136699 | SMPD4 | Y | Y | ENSG00000089916 | C14orf118 | N | Y |
| ENSG00000071051 | NCK2 | N | Y | ENSG00000119707 | RBM25 | N | Y |
| ENSG00000119812 | FAM98A | N | Y | ENSG00000119707 | RBM25 | N | Y |
| ENSG00000134323 | MYCN | N | Y | ENSG00000155463 | OXA1L | N | Y |
| ENSG00000132313 | MRPL35 | Y | Y | ENSG00000100888 | CHD8 | Y | Y |
| ENSG00000115816 | CEBPZ | N | Y | ENSG00000100603 | SNW1 | Y | Y |
| ENSG00000138018 | EPT1 | N | Y | ENSG00000100836 | PABPN1 | Y | Y |
| ENSG00000074054 | CLASP1 | Y | Y | ENSG00000179933 | C14orf119 | Y | Y |
| ENSG00000116062 | MSH6 | Y | Y | ENSG00000165819 | METTL3 | N | Y |
| ENSG00000136720 | HS6ST1 | N | Y | ENSG00000183576 | SETD3 | N | Y |
| ENSG00000136720 | HS6ST1 | Y | Y | ENSG00000126803 | HSPA2 | N | Y |
| ENSG00000170745 | KCNS3 | Y | Y | ENSG00000126803 | HSPA2 | N | Y |
| ENSG00000198522 | GPN1 | Y | Y | ENSG00000100941 | PNN | Y | Y |
| ENSG00000003509 | C2orf56 | N | Y | ENSG00000165588 | OTX2 | Y | Y |
| ENSG00000172845 | SP3 | Y | Y | ENSG00000165588 | OTX2 | Y | Y |
| ENSG00000240857 | RDH14 | Y | Y | ENSG00000133997 | MED6 | Y | Y |
| ENSG00000152518 | ZFP36L2 | Y | Y | ENSG00000250366 | RP11- | N | Y |
| | | | | | 185P18.1.1 | | |
| ENSG00000063660 | GPC1 | Y | Y | ENSG00000140443 | IGF1R | N | Y |
| ENSG00000163166 | IWS1 | N | Y | ENSG00000140443 | IGF1R | N | Y |
| ENSG00000124006 | OBSL1 | N | Y | ENSG00000169375 | SIN3A | Y | Y |
| ENSG00000124006 | OBSL1 | Y | Y | ENSG00000169375 | SIN3A | N | Y |
| ENSG00000144524 | COPS7B | Y | Y | ENSG00000128944 | C15orf23 | N | Y |
| ENSG00000153201 | RANBP2 | Y | Y | ENSG00000182175 | RGMA | N | Y |
| ENSG00000130147 | SH3BP4 | N | Y | ENSG00000140521 | POLG | N | Y |
| ENSG00000115129 | TP53I3 | N | Y | ENSG00000166855 | CLPX | N | Y |
| ENSG00000152291 | TGOLN2 | N | Y | ENSG00000166716 | ZNF592 | N | Y |
| ENSG00000204634 | TBC1D8 | N | Y | ENSG00000185033 | SEMA4B | Y | Y |
| ENSG00000125630 | POLR1B | N | Y | ENSG00000173548 | SNX33 | N | Y |
| ENSG00000152147 | GEMIN6 | N | Y | ENSG00000136383 | ALPK3 | N | Y |
| ENSG00000168758 | SEMA4C | Y | Y | ENSG00000136383 | ALPK3 | Y | Y |
| ENSG00000168758 | SEMA4C | N | Y | ENSG00000179361 | ARID3B | N | Y |
| ENSG00000118242 | MREG | N | Y | ENSG00000104081 | BMF | Y | Y |
| ENSG00000091409 | ITGA6 | N | Y | ENSG00000103994 | ZFP106 | N | Y |
| ENSG00000115825 | PRKD3 | N | Y | ENSG00000140263 | SORD | N | Y |
| ENSG00000163795 | ZNF513 | N | Y | ENSG00000021776 | AQR | N | Y |
| ENSG00000124383 | MPHOSPH10 | N | Y | ENSG00000140464 | PML | N | Y |
| ENSG00000119862 | LGALSL | N | Y | ENSG00000128965 | CHAC1 | N | Y |
| ENSG00000068654 | POLR1A | N | Y | ENSG00000131873 | CHSY1 | N | Y |
| ENSG00000068654 | POLR1A | N | Y | ENSG00000169371 | SNUPN | N | Y |
| ENSG00000115942 | ORC2 | Y | Y | ENSG00000166200 | COPS2 | Y | Y |
| ENSG00000170340 | B3GNT2 | Y | Y | ENSG00000182768 | NGRN | Y | Y |
| ENSG00000170340 | B3GNT2 | Y | Y | ENSG00000033800 | PIAS1 | Y | Y |
| ENSG00000170340 | B3GNT2 | N | Y | ENSG00000225151 | AC103965.1.1 | N | Y |
| ENSG00000115207 | GTF3C2 | N | Y | ENSG00000197299 | BLM | Y | Y |
| ENSG00000144233 | AMMECR1L | N | Y | ENSG00000169018 | FEM1B | Y | Y |
| ENSG00000163812 | ZDHHC3 | N | Y | ENSG00000169018 | FEM1B | Y | Y |
| ENSG00000144746 | ARL6IP5 | Y | Y | ENSG00000169018 | FEM1B | Y | Y |
| ENSG00000178252 | WDR6 | Y | Y | ENSG00000169018 | FEM1B | Y | Y |
| ENSG00000178252 | WDR6 | Y | Y | ENSG00000167196 | FBXO22 | N | Y |
| ENSG00000170266 | GLB1 | Y | Y | ENSG00000104142 | VPS18 | N | Y |
| ENSG00000114019 | AMOTL2 | N | Y | ENSG00000183060 | LYSMD4 | N | Y |
| ENSG00000114019 | AMOTL2 | N | Y | ENSG00000104067 | TJP1 | N | Y |
| ENSG00000168137 | SETD5 | N | Y | ENSG00000104067 | TJP1 | Y | Y |
| ENSG00000175928 | LRRN1 | N | Y | ENSG00000034053 | APBA2 | N | Y |
| ENSG00000181555 | SETD2 | N | Y | ENSG00000159322 | ADPGK | N | Y |
| ENSG00000181555 | SETD2 | N | Y | ENSG00000174498 | IGDCC3 | N | Y |
| ENSG00000173950 | XXYLT1 | N | Y | ENSG00000169926 | KLF13 | N | Y |
| ENSG00000010322 | NISCH | N | Y | ENSG00000140259 | MFAP1 | N | Y |
| ENSG00000154767 | XPC | N | Y | ENSG00000182636 | NDN | N | Y |
| ENSG00000175093 | SPSB4 | N | Y | ENSG00000140474 | ULK3 | N | Y |
| ENSG00000114631 | PODXL2 | N | Y | ENSG00000157483 | MYO1E | N | Y |
| ENSG00000172046 | USP19 | N | Y | ENSG00000166233 | ARIH1 | N | Y |
| ENSG00000164091 | WDR82 | N | Y | ENSG00000180357 | ZNF609 | Y | Y |
| ENSG00000134086 | VHL | N | Y | ENSG00000170776 | AKAP13 | N | Y |
| ENSG00000164045 | CDC25A | N | Y | ENSG00000140320 | BAHD1 | N | Y |
| ENSG00000163684 | RPP14 | N | Y | ENSG00000090238 | YPEL3 | Y | Y |
| ENSG00000163681 | SLMAP | Y | Y | ENSG00000168411 | RFWD3 | Y | Y |
| ENSG00000174579 | MSL2 | N | Y | ENSG00000168411 | RFWD3 | N | Y |
| ENSG00000206557 | TRIM71 | N | Y | ENSG00000066654 | THUMPD1 | Y | Y |
| ENSG00000114867 | EIF4G1 | N | Y | ENSG00000182831 | C16orf72 | N | Y |
| ENSG00000213672 | NCKIPSD | N | Y | ENSG00000140854 | KATNB1 | N | Y |
| ENSG00000170876 | TMEM43 | N | Y | ENSG00000149930 | TAOK2 | Y | Y |
| ENSG00000114120 | SLC25A36 | Y | Y | ENSG00000197562 | RAB40C | N | Y |
| ENSG00000154783 | FGD5 | N | Y | ENSG00000180035 | ZNF48 | N | Y |
| ENSG00000176095 | IP6K1 | N | Y | ENSG00000103356 | EARS2 | N | Y |
| ENSG00000187091 | PLCD1 | Y | Y | ENSG00000103356 | EARS2 | N | Y |
| ENSG00000170837 | GPR27 | N | Y | ENSG00000099381 | SETD1A | Y | Y |
| ENSG00000170837 | GPR27 | N | Y | ENSG00000103549 | RNF40 | N | Y |
| ENSG00000163602 | RYBP | N | Y | ENSG00000141084 | RANBP10 | N | Y |
| ENSG00000163608 | C3orf17 | N | Y | ENSG00000198736 | SEPX1 | N | Y |
| ENSG00000163832 | C3orf75 | Y | Y | ENSG00000131149 | KIAA0182 | N | Y |
| ENSG00000073849 | ST6GAL1 | Y | Y | ENSG00000131149 | KIAA0182 | Y | Y |
| ENSG00000073849 | ST6GAL1 | N | Y | ENSG00000162073 | PAQR4 | N | Y |
| ENSG00000073849 | ST6GAL1 | N | Y | ENSG00000162073 | PAQR4 | Y | Y |
| ENSG00000073849 | ST6GAL1 | N | Y | ENSG00000162073 | PAQR4 | N | Y |
| ENSG00000134077 | THUMPD3 | Y | Y | ENSG00000102921 | N4BP1 | N | Y |
| ENSG00000145041 | VPRBP | N | Y | ENSG00000050820 | BCAR1 | Y | Y |
| ENSG00000163660 | CCNL1 | Y | Y | ENSG00000050820 | BCAR1 | N | Y |
| ENSG00000144749 | LRIG1 | N | Y | ENSG00000131165 | CHMP1A | N | Y |
| ENSG00000144730 | IL17RD | N | Y | ENSG00000118898 | PPL | N | Y |
| ENSG00000082781 | ITGB5 | N | Y | ENSG00000118898 | PPL | N | Y |
| ENSG00000225733 | FGD5-AS1 | N | Y | ENSG00000077238 | IL4R | N | Y |
| ENSG00000225733 | FGD5-AS1 | Y | Y | ENSG00000103335 | PIEZO1 | Y | Y |
| ENSG00000144711 | IQSEC1 | N | Y | ENSG00000083093 | PALB2 | N | Y |
| ENSG00000198585 | NUDT16 | N | Y | ENSG00000179889 | PDXDC1 | N | Y |
| ENSG00000175455 | CCDC14 | Y | Y | ENSG00000157350 | ST3GAL2 | N | Y |
| ENSG00000132155 | RAF1 | N | Y | ENSG00000159579 | RSPRY1 | N | Y |
| ENSG00000051382 | PIK3CB | Y | Y | ENSG00000189091 | SF3B3 | N | Y |
| ENSG00000174738 | NR1D2 | N | Y | ENSG00000166454 | ATMIN | N | Y |
| ENSG00000174953 | DHX36 | N | Y | ENSG00000157106 | SMG1 | N | Y |
| ENSG00000004534 | RBM6 | N | Y | ENSG00000103257 | SLC7A5 | N | Y |
| ENSG00000155893 | ACPL2 | N | Y | ENSG00000103257 | SLC7A5 | N | Y |
| ENSG00000173402 | DAG1 | Y | Y | ENSG00000153815 | CMIP | N | Y |
| ENSG00000073792 | IGF2BP2 | N | Y | ENSG00000140750 | ARHGAP17 | N | Y |
| ENSG00000080819 | CPOX | Y | Y | ENSG00000132603 | NIP7 | N | Y |
| ENSG00000151276 | MAGI1 | N | Y | ENSG00000007392 | LUC7L | N | Y |
| ENSG00000016864 | GLT8D1 | N | Y | ENSG00000168802 | CHTF8 | N | Y |
| ENSG00000136603 | SKIL | N | Y | ENSG00000198211 | TUBB3 | N | Y |
| ENSG00000163872 | YEATS2 | N | Y | ENSG00000167978 | SRRM2 | N | Y |
| ENSG00000162290 | DCP1A | N | Y | ENSG00000104731 | KLHDC4 | Y | Y |
| ENSG00000161217 | PCYT1A | Y | Y | ENSG00000168286 | THAP11 | N | Y |
| ENSG00000169744 | LDB2 | Y | Y | ENSG00000168286 | THAP11 | Y | Y |
| ENSG00000138759 | FRAS1 | N | Y | ENSG00000167191 | GPRC5B | Y | Y |
| ENSG00000109501 | WFS1 | N | Y | ENSG00000103326 | SOLH | N | Y |
| ENSG00000109501 | WFS1 | N | Y | ENSG00000140632 | GLYR1 | N | Y |
| ENSG00000145220 | LYAR | Y | Y | ENSG00000176387 | HSD11B2 | N | Y |
| ENSG00000109265 | KIAA1211 | N | Y | ENSG00000167526 | RPL13 | N | Y |
| ENSG00000109265 | KIAA1211 | N | Y | ENSG00000103429 | BFAR | Y | Y |
| ENSG00000128052 | KDR | Y | Y | ENSG00000103423 | DNAJA3 | N | Y |
| ENSG00000128052 | KDR | Y | Y | ENSG00000140650 | PMM2 | N | Y |
| ENSG00000128052 | KDR | N | Y | ENSG00000103449 | SALL1 | N | Y |
| ENSG00000128052 | KDR | N | Y | ENSG00000169217 | CD2BP2 | N | Y |
| ENSG00000152990 | GPR125 | Y | Y | ENSG00000169217 | CD2BP2 | N | Y |
| ENSG00000152990 | GPR125 | N | Y | ENSG00000166847 | DCTN5 | N | Y |
| ENSG00000168936 | TMEM129 | N | Y | ENSG00000122386 | ZNF205 | N | Y |
| ENSG00000118579 | MED28 | N | Y | ENSG00000090905 | TNRC6A | N | Y |
| ENSG00000083857 | FAT1 | N | Y | ENSG00000102977 | ACD | N | Y |
| ENSG00000083857 | FAT1 | Y | Y | ENSG00000102974 | CTCF | N | Y |
| ENSG00000083857 | FAT1 | Y | Y | ENSG00000182149 | IST1 | N | Y |
| ENSG00000121892 | PDS5A | Y | Y | ENSG00000168488 | ATXN2L | N | Y |
| ENSG00000185619 | PCGF3 | N | Y | ENSG00000122257 | RBBP6 | N | Y |
| ENSG00000168556 | ING2 | Y | Y | ENSG00000162062 | C16orf59 | N | Y |
| ENSG00000077684 | PHF17 | N | Y | ENSG00000103550 | C16orf88 | N | Y |
| ENSG00000077684 | PHF17 | Y | Y | ENSG00000080603 | SRCAP | Y | Y |
| ENSG00000186222 | CN | Y | Y | ENSG00000153406 | NMRAL1 | N | Y |
| ENSG00000152208 | GRID2 | Y | Y | ENSG00000184602 | SNN | Y | Y |
| ENSG00000109814 | UGDH | N | Y | ENSG00000184602 | SNN | N | Y |
| ENSG00000163629 | PTPN13 | N | Y | ENSG00000187555 | USP7 | N | Y |
| ENSG00000168924 | LETM1 | Y | Y | ENSG00000090857 | PDPR | N | Y |
| ENSG00000163694 | RBM47 | N | Y | ENSG00000006327 | TNFRSF12A | N | Y |
| ENSG00000164040 | PGRMC2 | N | Y | ENSG00000103160 | HSDL1 | Y | Y |
| ENSG00000198589 | LRBA | Y | Y | ENSG00000062038 | CDH3 | N | Y |
| ENSG00000157404 | KIT | N | Y | ENSG00000179918 | SEPHS2 | Y | Y |
| ENSG00000218336 | ODZ3 | N | Y | ENSG00000179918 | SEPHS2 | N | Y |
| ENSG00000184160 | ADRA2C | N | Y | ENSG00000129925 | TMEM8A | N | Y |
| ENSG00000118762 | PKD2 | Y | Y | ENSG00000141101 | NB1 | N | Y |
| ENSG00000132466 | ANKRD17 | Y | Y | ENSG00000087258 | GNAO1 | N | Y |
| ENSG00000035928 | RFC1 | N | Y | ENSG00000168872 | DDX19A | Y | Y |
| ENSG00000132405 | TBC1D14 | N | Y | ENSG00000168872 | DDX19A | N | Y |
| ENSG00000179059 | ZFP42 | N | Y | ENSG00000099364 | FBXL19 | Y | Y |
| ENSG00000179010 | MRFAP1 | N | Y | ENSG00000125166 | GOT2 | Y | Y |
| ENSG00000138771 | SHROOM3 | N | Y | ENSG00000197912 | SPG7 | N | Y |
| ENSG00000161021 | MAML1 | N | Y | ENSG00000157349 | DDX19B | Y | Y |
| ENSG00000161021 | MAML1 | N | Y | ENSG00000095906 | NUBP2 | N | Y |
| ENSG00000174136 | RGMB | N | Y | ENSG00000167513 | CDT1 | N | Y |
| ENSG00000113141 | IK | N | Y | ENSG00000167513 | CDT1 | N | Y |
| ENSG00000197226 | TBC1D9B | Y | Y | ENSG00000090565 | RAB11FIP3 | N | Y |
| ENSG00000113161 | HMGCR | Y | Y | ENSG00000167693 | NXN | N | Y |
| ENSG00000120705 | ETF1 | N | Y | ENSG00000167693 | NXN | Y | Y |
| ENSG00000113504 | SLC12A7 | Y | Y | ENSG00000186566 | GPATCH8 | N | Y |
| ENSG00000048140 | TSPAN17 | N | Y | ENSG00000171298 | GAA | N | Y |
| ENSG00000145604 | SKP2 | Y | Y | ENSG00000179409 | GEMIN4 | N | Y |
| ENSG00000153922 | CHD1 | Y | Y | ENSG00000179409 | GEMIN4 | Y | Y |
| ENSG00000164574 | GALNT10 | N | Y | ENSG00000167861 | C17orf28 | N | Y |
| ENSG00000113645 | WWC1 | N | Y | ENSG00000167105 | TMEM92 | Y | Y |
| ENSG00000176788 | BASP1 | Y | Y | ENSG00000109062 | SLC9A3R1 | N | Y |
| ENSG00000122203 | KIAA1191 | Y | Y | ENSG00000141736 | ERBB2 | N | Y |
| ENSG00000153395 | LPCAT1 | N | Y | ENSG00000121057 | AKAP1 | N | Y |
| ENSG00000153395 | LPCAT1 | N | Y | ENSG00000121058 | COIL | Y | Y |
| ENSG00000188725 | C5orf43 | Y | Y | ENSG00000159842 | ABR | N | Y |
| ENSG00000037474 | NSUN2 | Y | Y | ENSG00000029725 | RABEP1 | N | Y |
| ENSG00000037474 | NSUN2 | N | Y | ENSG00000170037 | CNTROB | N | Y |
| ENSG00000169223 | LMAN2 | N | Y | ENSG00000170004 | CHD3 | Y | Y |
| ENSG00000145555 | MYO10 | N | Y | ENSG00000188554 | NBR1 | Y | Y |
| ENSG00000131504 | DIAPH1 | N | Y | ENSG00000173065 | C17orf63 | N | Y |
| ENSG00000164151 | KIAA0947 | Y | Y | ENSG00000132142 | ACACA | N | Y |
| ENSG00000155508 | CNT8 | N | Y | ENSG00000136448 | NMT1 | N | Y |
| ENSG00000250337 | RP11- | Y | Y | ENSG00000136448 | NMT1 | N | Y |
| | 46C20.1.1 | | | | | | |
| ENSG00000135083 | CCNJL | N | Y | ENSG00000136448 | NMT1 | Y | Y |
| ENSG00000164190 | NIPBL | Y | Y | ENSG00000136444 | RSAD1 | N | Y |
| ENSG00000145882 | PCYOX1L | N | Y | ENSG00000213977 | TAX1BP3 | N | Y |
| ENSG00000082516 | GEMIN5 | N | Y | ENSG00000177370 | TIMM22 | Y | Y |
| ENSG00000067248 | DHX29 | Y | Y | ENSG00000108256 | NUFIP2 | N | Y |
| ENSG00000198780 | FAM169A | N | Y | ENSG00000108256 | NUFIP2 | Y | Y |
| ENSG00000150712 | MTMR12 | N | Y | ENSG00000108270 | AATF | N | Y |
| ENSG00000178913 | TAF7 | N | Y | ENSG00000160551 | TAOK1 | Y | Y |
| ENSG00000165671 | NSD1 | N | Y | ENSG00000132475 | H3F3B | N | Y |
| ENSG00000165671 | NSD1 | N | Y | ENSG00000161542 | PRPSAP1 | N | Y |
| ENSG00000165671 | NSD1 | N | Y | ENSG00000108840 | HDAC5 | N | Y |
| ENSG00000165671 | NSD1 | Y | Y | ENSG00000108848 | LUC7L3 | N | Y |
| ENSG00000165671 | NSD1 | N | Y | ENSG00000186185 | KIF18B | N | Y |
| ENSG00000165671 | NSD1 | N | Y | ENSG00000072310 | SREBF1 | N | Y |
| ENSG00000038382 | TRIO | N | Y | ENSG00000197417 | SHPK | Y | Y |
| ENSG00000168246 | UBTD2 | Y | Y | ENSG00000197417 | SHPK | N | Y |
| ENSG00000070814 | TCOF1 | N | Y | ENSG00000175832 | ETV4 | N | Y |
| ENSG00000152684 | PELO | Y | Y | ENSG00000108312 | UBTF | N | Y |
| ENSG00000092421 | SEMA6A | N | Y | ENSG00000185359 | HGS | N | Y |
| ENSG00000092421 | SEMA6A | N | Y | ENSG00000174282 | ZBTB4 | Y | Y |
| ENSG00000112984 | KIF20A | Y | Y | ENSG00000141456 | AC091153.1 | Y | Y |
| ENSG00000113583 | C5orf15 | N | Y | ENSG00000141456 | AC091153.1 | N | Y |
| ENSG00000171604 | CXXC5 | N | Y | ENSG00000072134 | EPN2 | N | Y |
| ENSG00000113657 | DPYSL3 | N | Y | ENSG00000133026 | MYH10 | Y | Y |
| ENSG00000174705 | SH3PXD2B | Y | Y | ENSG00000133026 | MYH10 | N | Y |
| ENSG00000164294 | GPX8 | Y | Y | ENSG00000133026 | MYH10 | N | Y |
| ENSG00000113194 | FAF2 | N | Y | ENSG00000133026 | MYH10 | N | Y |
| ENSG00000113739 | STC2 | N | Y | ENSG00000108424 | KPNB1 | N | Y |
| ENSG00000070614 | NDST1 | N | Y | ENSG00000180340 | FZD2 | N | Y |
| ENSG00000171720 | HDAC3 | Y | Y | ENSG00000178307 | TMEM11 | Y | Y |
| ENSG00000072364 | AFF4 | N | Y | ENSG00000198909 | MAP3K3 | Y | Y |
| ENSG00000072364 | AFF4 | N | Y | ENSG00000125686 | MED1 | Y | Y |
| ENSG00000113758 | DBN1 | N | Y | ENSG00000125686 | MED1 | Y | Y |
| ENSG00000145919 | BOD1 | N | Y | ENSG00000185298 | CCDC137 | Y | Y |
| ENSG00000145911 | N4BP3 | N | Y | ENSG00000167193 | CRK | Y | Y |
| ENSG00000251273 | RP11- | N | Y | ENSG00000067596 | DHX8 | N | Y |
| | 549K20.1.1 | | | | | | |
| ENSG00000187678 | SPRY4 | N | Y | ENSG00000182473 | EXOC7 | N | Y |
| ENSG00000187678 | SPRY4 | N | Y | ENSG00000167699 | GLOD4 | Y | Y |
| ENSG00000131711 | MAP1B | N | Y | ENSG00000109118 | PHF12 | N | Y |
| ENSG00000164615 | CAMLG | N | Y | ENSG00000109111 | SUPT6H | N | Y |
| ENSG00000113048 | MRPS27 | N | Y | ENSG00000185722 | ANKFY1 | N | Y |
| ENSG00000038427 | VCAN | N | Y | ENSG00000131748 | STARD3 | N | Y |
| ENSG00000038427 | VCAN | N | Y | ENSG00000183048 | MRPL12 | N | Y |
| ENSG00000038427 | VCAN | N | Y | ENSG00000091542 | ALKBH5 | Y | Y |
| ENSG00000038427 | VCAN | Y | Y | ENSG00000173821 | RNF213 | Y | Y |
| ENSG00000164244 | PRRC1 | N | Y | ENSG00000173821 | RNF213 | N | Y |
| ENSG00000119900 | OGFRL1 | N | Y | ENSG00000141580 | WDR45L | N | Y |
| ENSG00000119900 | OGFRL1 | N | Y | ENSG00000141720 | PIP4K2B | N | Y |
| ENSG00000247909 | | Y | Y | ENSG00000141720 | PIP4K2B | N | Y |
| ENSG00000153046 | CDYL | N | Y | ENSG00000133028 | SCO1 | N | Y |
| ENSG00000112739 | PRPF4B | N | Y | ENSG00000040633 | PHF23 | Y | Y |
| ENSG00000213079 | SCAF8 | N | Y | ENSG00000091640 | SPAG7 | N | Y |
| ENSG00000137166 | FOXP4 | N | Y | ENSG00000006744 | ELAC2 | N | Y |
| ENSG00000180992 | MRPL14 | N | Y | ENSG00000006744 | ELAC2 | N | Y |
| ENSG00000189241 | TSPYL1 | N | Y | ENSG00000187531 | SIRT7 | N | Y |
| ENSG00000044090 | CUL7 | N | Y | ENSG00000171634 | BPTF | Y | Y |
| ENSG00000151914 | DST | N | Y | ENSG00000179314 | WSCD1 | N | Y |
| ENSG00000112658 | SRF | N | Y | ENSG00000034152 | MAP2K3 | Y | Y |
| ENSG00000236673 | RP11- | N | Y | ENSG00000121067 | SPOP | N | Y |
| | 69I8.2.1 | | | | | | |
| ENSG00000124782 | RREB1 | Y | Y | ENSG00000141564 | RPTOR | N | Y |
| ENSG00000124688 | MAD2L1BP | Y | Y | ENSG00000141569 | TRIM65 | Y | Y |
| ENSG00000181472 | ZBTB2 | N | Y | ENSG00000141568 | FOXK2 | N | Y |
| ENSG00000188112 | C6orf132 | Y | Y | ENSG00000082641 | NFE2L1 | N | Y |
| ENSG00000111817 | DSE | Y | Y | ENSG00000082641 | NFE2L1 | N | Y |
| ENSG00000111817 | DSE | Y | Y | ENSG00000121083 | DYNLL2 | Y | Y |
| ENSG00000196586 | MYO6 | N | Y | ENSG00000108528 | SLC25A11 | N | Y |
| ENSG00000197081 | IGF2R | N | Y | ENSG00000141504 | SAT2 | N | Y |
| ENSG00000118482 | PHF3 | N | Y | ENSG00000172057 | ORMDL3 | N | Y |
| ENSG00000118482 | PHF3 | N | Y | ENSG00000002919 | SNX11 | N | Y |
| ENSG00000085511 | MAP3K4 | Y | Y | ENSG00000108262 | GIT1 | Y | Y |
| ENSG00000112033 | PPARD | N | Y | ENSG00000087152 | ATXN7L3 | N | Y |
| ENSG00000112033 | PPARD | Y | Y | ENSG00000087152 | ATXN7L3 | N | Y |
| ENSG00000152661 | GJA1 | N | Y | ENSG00000188522 | FAM83G | N | Y |
| ENSG00000152661 | GJA1 | N | Y | ENSG00000167258 | CDK12 | N | Y |
| ENSG00000152661 | GJA1 | N | Y | ENSG00000186834 | HEXIM1 | N | Y |
| ENSG00000188428 | MUTED | N | Y | ENSG00000068489 | PRR11 | N | Y |
| ENSG00000146426 | TIAM2 | N | Y | ENSG00000007202 | KIAA0100 | N | Y |
| ENSG00000049618 | ARID1B | N | Y | ENSG00000177469 | PTRF | N | Y |
| ENSG00000146072 | TNFRSF21 | Y | Y | ENSG00000177469 | PTRF | Y | Y |
| ENSG00000156639 | ZFAND3 | Y | Y | ENSG00000141295 | SCRN2 | N | Y |
| ENSG00000130396 | MLLT4 | N | Y | ENSG00000125445 | MRPS7 | N | Y |
| ENSG00000130396 | MLLT4 | N | Y | ENSG00000141378 | PTRH2 | Y | Y |
| ENSG00000164442 | CITED2 | N | Y | ENSG00000173894 | CBX2 | N | Y |
| ENSG00000085377 | PREP | Y | Y | ENSG00000173894 | CBX2 | N | Y |
| ENSG00000196821 | C6orf106 | N | Y | ENSG00000108819 | PPP1R9B | N | Y |
| ENSG00000196821 | C6orf106 | Y | Y | ENSG00000176658 | MYO1D | N | Y |
| ENSG00000008083 | JARID2 | N | Y | ENSG00000141219 | C17orf80 | Y | Y |
| ENSG00000111961 | SASH1 | N | Y | ENSG00000004142 | POLDIP2 | N | Y |
| ENSG00000096070 | BRPF3 | N | Y | ENSG00000133030 | MPRIP | N | Y |
| ENSG00000096696 | DSP | Y | Y | ENSG00000120063 | GNA13 | N | Y |
| ENSG00000135316 | SYNCRIP | Y | Y | ENSG00000169727 | GPS1 | N | Y |
| ENSG00000057663 | ATG5 | N | Y | ENSG00000060069 | CTDP1 | N | Y |
| ENSG00000146457 | WTAP | Y | Y | ENSG00000154845 | PPP4R1 | N | Y |
| ENSG00000146457 | WTAP | Y | Y | ENSG00000170677 | SOCS6 | N | Y |
| ENSG00000146457 | WTAP | Y | Y | ENSG00000170677 | SOCS6 | N | Y |
| ENSG00000112029 | FBXO5 | N | Y | ENSG00000081913 | PHLPP1 | N | Y |
| ENSG00000112249 | ASCC3 | N | Y | ENSG00000256463 | SALL3 | N | Y |
| ENSG00000182952 | HMGN4 | N | Y | ENSG00000176014 | TUBB6 | Y | Y |
| ENSG00000106443 | PHF14 | N | Y | ENSG00000168461 | RAB31 | N | Y |
| ENSG00000136231 | IGF2BP3 | N | Y | ENSG00000141644 | MBD1 | N | Y |
| ENSG00000106636 | YKT6 | N | Y | ENSG00000141424 | SLC39A6 | N | Y |
| ENSG00000065883 | CDK13 | Y | Y | ENSG00000101544 | ADNP2 | N | Y |
| ENSG00000106263 | EIF3B | N | Y | ENSG00000171703 | TCEA2 | N | Y |
| ENSG00000166526 | ZNF3 | N | Y | ENSG00000124193 | SRSF6 | N | Y |
| ENSG00000164535 | DAGLB | N | Y | ENSG00000240849 | TMEM189 | Y | Y |
| ENSG00000006453 | BAIAP2L1.1 | Y | Y | ENSG00000101407 | TTI1 | Y | Y |
| ENSG00000160963 | EMID2 | N | Y | ENSG00000101407 | TTI1 | N | Y |
| ENSG00000160963 | EMID2 | N | Y | ENSG00000101407 | TTI1 | N | Y |
| ENSG00000243335 | KCTD7 | N | Y | ENSG00000101447 | FAM83D | Y | Y |
| ENSG00000158321 | AUTS2 | N | Y | ENSG00000171552 | BCL2L1 | N | Y |
| ENSG00000158321 | AUTS2 | N | Y | ENSG00000171940 | ZNF217 | N | Y |
| ENSG00000158321 | AUTS2 | N | Y | ENSG00000171940 | ZNF217 | Y | Y |
| ENSG00000129103 | SUMF2 | N | Y | ENSG00000101337 | TM9SF4 | N | Y |
| ENSG00000185274 | WBSCR17 | N | Y | ENSG00000101337 | TM9SF4 | N | Y |
| ENSG00000185274 | WBSCR17 | N | Y | ENSG00000126003 | PLAGL2 | Y | Y |
| ENSG00000188191 | PRKAR1B | Y | Y | ENSG00000132823 | C20orf111 | N | Y |
| ENSG00000154978 | VOPP1 | N | Y | ENSG00000149658 | YTHDF1 | N | Y |
| ENSG00000154978 | VOPP1 | N | Y | ENSG00000197122 | SRC | N | Y |
| ENSG00000154978 | VOPP1 | N | Y | ENSG00000053438 | NNAT | N | Y |
| ENSG00000154978 | VOPP1 | Y | Y | ENSG00000101189 | C20orf20 | N | Y |
| ENSG00000075624 | ACTB | Y | Y | ENSG00000132640 | BTBD3 | N | Y |
| ENSG00000002822 | MAD1L1 | Y | Y | ENSG00000132640 | BTBD3 | N | Y |
| ENSG00000146776 | ATXN7L1 | Y | Y | ENSG00000125844 | RRBP1 | Y | Y |
| ENSG00000106624 | AEBP1 | N | Y | ENSG00000101040 | ZMYND8 | Y | Y |
| ENSG00000128567 | PODXL | N | Y | ENSG00000124222 | STX16 | N | Y |
| ENSG00000128567 | PODXL | N | Y | ENSG00000088325 | TPX2 | N | Y |
| ENSG00000106459 | NRF1 | N | Y | ENSG00000177732 | SOX12 | Y | Y |
| ENSG00000075213 | SEMA3A | N | Y | ENSG00000196227 | C20orf177 | Y | Y |
| ENSG00000198742 | SMURF1 | N | Y | ENSG00000101158 | TH1L | N | Y |
| ENSG00000128602 | SMO | N | Y | ENSG00000101150 | TPD52L2 | N | Y |
| ENSG00000106665 | CLIP2 | N | Y | ENSG00000101150 | TPD52L2 | N | Y |
| ENSG00000106665 | CLIP2 | Y | Y | ENSG00000158470 | B4GALT5 | N | Y |
| ENSG00000106665 | CLIP2 | N | Y | ENSG00000124181 | PLCG1 | Y | Y |
| ENSG00000158457 | TSPAN33 | Y | Y | ENSG00000132819 | RBM38 | N | Y |
| ENSG00000164880 | INTS1 | N | Y | ENSG00000124164 | VAPB | N | Y |
| ENSG00000146830 | GIGYF1 | N | Y | ENSG00000244462 | RBM12 | N | Y |
| ENSG00000146830 | GIGYF1 | N | Y | ENSG00000244462 | RBM12 | Y | Y |
| ENSG00000146834 | MEPCE | N | Y | ENSG00000244462 | RBM12 | Y | Y |
| ENSG00000157224 | CLDN12 | N | Y | ENSG00000025293 | PHF20 | N | Y |
| ENSG00000091732 | ZC3HC1 | N | Y | ENSG00000101115 | SALL4 | N | Y |
| ENSG00000180233 | ZNRF2 | N | Y | ENSG00000124145 | SDC4 | N | Y |
| ENSG00000165215 | CLDN3 | N | Y | ENSG00000092758 | COL9A3 | Y | Y |
| ENSG00000164889 | SLC4A2 | N | Y | ENSG00000092758 | COL9A3 | Y | Y |
| ENSG00000146535 | GNA12 | N | Y | ENSG00000118707 | TGIF2 | N | Y |
| ENSG00000242265 | PEG10 | Y | Y | ENSG00000149600 | COMMD7 | N | Y |
| ENSG00000242265 | PEG10 | N | Y | ENSG00000101246 | ARFRP1 | N | Y |
| ENSG00000174469 | CNTNAP2 | N | Y | ENSG00000101412 | E2F1 | N | Y |
| ENSG00000128595 | CALU | Y | Y | ENSG00000101193 | C20orf11 | N | Y |
| ENSG00000147155 | EBP | Y | Y | ENSG00000196700 | ZNF512B | N | Y |
| ENSG00000186462 | NAP1L2 | Y | Y | ENSG00000101019 | UQCC | Y | Y |
| ENSG00000147050 | KDM6A | N | Y | ENSG00000089195 | TRMT6 | N | Y |
| ENSG00000147050 | KDM6A | Y | Y | ENSG00000165246 | NLGN4Y | Y | Y |
| ENSG00000169084 | DHRSX | N | Y | ENSG00000114374 | USP9Y | N | Y |
| ENSG00000188021 | UBQLN2 | N | Y | ENSG00000105127 | AKAP8 | N | Y |
| ENSG00000203950 | FAM127B | N | Y | ENSG00000142449 | FBN3 | N | Y |
| ENSG00000123562 | MORF4L2 | N | Y | ENSG00000005007 | UPF1 | Y | Y |
| ENSG00000102081 | FMR1 | N | Y | ENSG00000160888 | IER2 | N | Y |
| ENSG00000147274 | RBMX | Y | Y | ENSG00000142252 | GEMIN7 | N | Y |
| ENSG00000172534 | HCFC1 | N | Y | ENSG00000167470 | MIDN | N | Y |
| ENSG00000172534 | HCFC1 | Y | Y | ENSG00000108107 | RPL28 | N | Y |
| ENSG00000067445 | TRO | N | Y | ENSG00000119559 | C19orf25 | N | Y |
| ENSG00000067445 | TRO | Y | Y | ENSG00000105429 | MEGF8 | N | Y |
| ENSG00000196368 | NUDT11 | N | Y | ENSG00000105186 | ANKRD27 | N | Y |
| ENSG00000182195 | LDOC1 | N | Y | ENSG00000105401 | CDC37 | N | Y |
| ENSG00000184481 | FOXO4 | N | Y | ENSG00000117877 | CD3EAP | Y | Y |
| ENSG00000125352 | RNF113A | N | Y | ENSG00000187867 | PALM3 | N | Y |
| ENSG00000196998 | WDR45 | N | Y | ENSG00000213753 | AC016629.2.1 | N | Y |
| ENSG00000197021 | CXorf4OB | N | Y | ENSG00000167600 | CYP2S1 | N | Y |
| ENSG00000147162 | OGT | Y | Y | ENSG00000167600 | CYP2S1 | N | Y |
| ENSG00000187601 | MAGEH1 | N | Y | ENSG00000011243 | AKAP8L | N | Y |
| ENSG00000131263 | RLIM | N | Y | ENSG00000072071 | LPHN1 | N | Y |
| ENSG00000126012 | KDM5C | N | Y | ENSG00000127527 | EPS15L1 | N | Y |
| ENSG00000071859 | FAM50A | N | Y | ENSG00000130382 | MLLT1 | N | Y |
| ENSG00000169093 | ASMTL | N | Y | ENSG00000064607 | SUGP2 | N | Y |
| ENSG00000182378 | PLCXD1 | Y | Y | ENSG00000064607 | SUGP2 | N | Y |
| ENSG00000101849 | TBL1X | N | Y | ENSG00000104880 | ARHGEF18 | Y | Y |
| ENSG00000071889 | FAM3A | N | Y | ENSG00000104885 | DOT1L | N | Y |
| ENSG00000214717 | ZBED1 | Y | Y | ENSG00000105270 | CLIP3 | Y | Y |
| ENSG00000146938 | NLGN4X | Y | Y | ENSG00000153879 | CEBPG | N | Y |
| ENSG00000124486 | USP9X | Y | Y | ENSG00000133275 | CSNK1G2 | N | Y |
| ENSG00000186871 | ERCC6L | Y | Y | ENSG00000133275 | CSNK1G2 | N | Y |
| ENSG00000183943 | PRKX | N | Y | ENSG00000105732 | ZNF574 | N | Y |
| ENSG00000169188 | APEX2 | N | Y | ENSG00000075702 | WDR62 | N | Y |
| ENSG00000134590 | FAM127A | N | Y | ENSG00000254858 | MPV17L2 | Y | Y |
| ENSG00000180964 | TCEAL8 | N | Y | ENSG00000181896 | ZNF101 | N | Y |
| ENSG00000011201 | KAL1 | N | Y | ENSG00000184635 | ZNF93 | Y | Y |
| ENSG00000056998 | GYG2 | Y | Y | ENSG00000105085 | MED26 | N | Y |
| ENSG00000155959 | VBP1 | N | Y | ENSG00000129951 | LPPR3.1 | Y | Y |
| ENSG00000173273 | TNKS | Y | Y | ENSG00000141867 | BRD4 | N | Y |
| ENSG00000158669 | AGPAT6 | N | Y | ENSG00000129932 | DOHH | N | Y |
| ENSG00000168575 | SLC20A2 | N | Y | ENSG00000105323 | HNRNPUL1 | N | Y |
| ENSG00000183808 | RBM12B | Y | Y | ENSG00000105323 | HNRNPUL1 | Y | Y |
| ENSG00000179041 | RRS1 | N | Y | ENSG00000105325 | FZR1 | N | Y |
| ENSG00000153317 | ASAP1 | Y | Y | ENSG00000071564 | TCF3 | Y | Y |
| ENSG00000171316 | CHD7 | Y | Y | ENSG00000127663 | KDM4B | Y | Y |
| ENSG00000171316 | CHD7 | N | Y | ENSG00000007047 | MARK4 | N | Y |
| ENSG00000136986 | DERL1 | N | Y | ENSG00000141994 | DUS3L | N | Y |
| ENSG00000185728 | YTHDF3 | Y | Y | ENSG00000131116 | ZNF428 | N | Y |
| ENSG00000185728 | YTHDF3 | N | Y | ENSG00000213024 | NUP62 | N | Y |
| ENSG00000205268 | PDE7A | Y | Y | ENSG00000213024 | NUP62 | N | Y |
| ENSG00000173281 | PPP1R3B | N | Y | ENSG00000105281 | SLC1A5 | N | Y |
| ENSG00000170619 | COMMD5 | N | Y | ENSG00000105131 | EPHX3 | N | Y |
| ENSG00000104331 | IMPAD1 | Y | Y | ENSG00000246181 | | Y | Y |
| ENSG00000104312 | RIPK2 | N | Y | ENSG00000125505 | MBOAT7 | N | Y |
| ENSG00000182319 | PRAGMIN.1 | N | Y | ENSG00000167658 | EEF2 | N | Y |
| ENSG00000178764 | ZHX2 | N | Y | ENSG00000105173 | CCNE1 | N | Y |
| ENSG00000133874 | RNF122 | N | Y | ENSG00000115255 | REEP6 | N | Y |
| ENSG00000147596 | PRDM14 | Y | Y | ENSG00000167460 | TPM4 | Y | Y |
| ENSG00000160957 | RECQL4 | N | Y | ENSG00000130312 | MRPL34 | N | Y |
| ENSG00000180900 | SCRIB | Y | Y | ENSG00000167674 | AC011498.1 | Y | Y |
| ENSG00000180900 | SCRIB | N | Y | ENSG00000130311 | DDA1 | N | Y |
| ENSG00000157110 | RBPMS | N | Y | ENSG00000160570 | DEDD2 | N | Y |
| ENSG00000012232 | EXTL3 | N | Y | ENSG00000105197 | TIMM50 | N | Y |
| ENSG00000180921 | FAM83H | N | Y | ENSG00000187266 | EPOR | Y | Y |
| ENSG00000182372 | CLN8 | Y | Y | ENSG00000182087 | C19orf6 | N | Y |
| ENSG00000147457 | CHMP7 | Y | Y | ENSG00000130669 | PAK4 | N | Y |
| ENSG00000147454 | SLC25A37 | N | Y | ENSG00000125755 | SYMPK | Y | Y |
| ENSG00000183309 | ZNF623 | N | Y | ENSG00000167635 | ZNF146 | N | Y |
| ENSG00000120885 | CLU | N | Y | ENSG00000125912 | NCLN | N | Y |
| ENSG00000136997 | MYC | N | Y | ENSG00000031823 | RANBP3 | N | Y |
| ENSG00000181090 | EHMT1 | Y | Y | ENSG00000227500 | SCAMP4 | N | Y |
| ENSG00000130560 | UBAC1 | N | Y | ENSG00000198683 | AC012615.1 | N | Y |
| ENSG00000165661 | QSOX2 | N | Y | ENSG00000105245 | NUMBL | N | Y |
| ENSG00000159884 | CCDC107 | N | Y | ENSG00000105245 | NUMBL | N | Y |
| ENSG00000148143 | ZNF462 | N | Y | ENSG00000198093 | ZNF649 | Y | Y |
| ENSG00000107130 | NCS1 | N | Y | ENSG00000198093 | ZNF649 | Y | Y |
| ENSG00000137124 | ALDH1B1 | N | Y | ENSG00000079999 | KEAP1 | N | Y |
| ENSG00000147869 | CER1 | Y | Y | ENSG00000179115 | FARSA | N | Y |
| ENSG00000238227 | C9orf69 | N | Y | ENSG00000125651 | GTF2F1 | Y | Y |
| ENSG00000238227 | C9orf69 | N | Y | ENSG00000125651 | GTF2F1 | Y | Y |
| ENSG00000078725 | DBC1 | Y | Y | ENSG00000160007 | ARHGAP35 | N | Y |
| ENSG00000127191 | TRAF2 | N | Y | ENSG00000142549 | IGLON5 | N | Y |
| ENSG00000107341 | UBE2R2 | N | Y | ENSG00000085872 | CHERP | N | Y |
| ENSG00000107341 | UBE2R2 | Y | Y | ENSG00000129347 | KRI1 | N | Y |
| ENSG00000169925 | BRD3 | N | Y | ENSG00000129347 | KRI1 | Y | Y |
| ENSG00000148300 | REXO4 | N | Y | ENSG00000134815 | DHX34 | N | Y |
| ENSG00000233137 | RP11- | Y | Y | ENSG00000074181 | NTCH3 | Y | Y |
| | 220I1.1.1 | | | | | | |
| ENSG00000119335 | SET | N | Y | ENSG00000131941 | RHPN2 | N | Y |
| ENSG00000155827 | RNF20 | N | Y | ENSG00000218891 | ZNF579 | N | Y |
| ENSG00000137055 | PLAA | N | Y | ENSG00000065000 | AP3D1 | N | Y |
| ENSG00000196730 | DAPK1 | N | Y | ENSG00000065000 | AP3D1 | N | Y |
| ENSG00000130723 | PRRC2B | Y | Y | ENSG00000132024 | CC2D1A | N | Y |
| ENSG00000148296 | SURF6 | N | Y | ENSG00000130881 | LRP3 | N | Y |
| ENSG00000148297 | MED22 | Y | Y | ENSG00000099942 | CRKL | N | Y |
| ENSG00000221829 | FANCG | Y | Y | ENSG00000099942 | CRKL | N | Y |
| ENSG00000137038 | C9orf123 | Y | Y | ENSG00000099942 | CRKL | N | Y |
| ENSG00000136908 | DPM2 | N | Y | ENSG00000183864 | TOB2 | N | Y |
| ENSG00000197579 | TOPORS | N | Y | ENSG00000100116 | GCAT | N | Y |
| ENSG00000197579 | TOPORS | N | Y | ENSG00000040608 | RTN4R | N | Y |
| ENSG00000097007 | ABL1 | N | Y | ENSG00000183579 | ZNRF3 | Y | Y |
| ENSG00000097007 | ABL1 | N | Y | ENSG00000182541 | LIMK2 | Y | Y |
| ENSG00000168795 | ZBTB5 | N | Y | ENSG00000182541 | LIMK2 | N | Y |
| ENSG00000044574 | HSPA5 | N | Y | ENSG00000185651 | UBE2L3 | N | Y |
| ENSG00000197724 | PHF2 | N | Y | ENSG00000185651 | UBE2L3 | N | Y |
| ENSG00000107362 | FAM108B1 | N | Y | ENSG00000100379 | KCTD17 | N | Y |
| ENSG00000136943 | CTSL2 | N | Y | ENSG00000100393 | EP300 | N | Y |
| ENSG00000107104 | KANK1 | N | Y | ENSG00000100401 | RANGAP1 | N | Y |
| ENSG00000167106 | FAM102A | N | Y | ENSG00000100403 | ZC3H7B | N | Y |
| ENSG00000099810 | MTAP | N | Y | ENSG00000100403 | ZC3H7B | N | Y |
| ENSG00000176248 | ANAPC2 | N | Y | ENSG00000170638 | TRABD | Y | Y |
| ENSG00000147874 | HAUS6 | N | Y | ENSG00000196588 | MKL1 | N | Y |
| ENSG00000198722 | UNC13B | N | Y | ENSG00000100139 | MICALL1 | N | Y |
| ENSG00000148358 | GPR107 | Y | Y | ENSG00000138867 | C22orf13 | N | Y |
| ENSG00000107290 | SETX | N | Y | ENSG00000100058 | CRYBB2P1 | N | Y |
| ENSG00000138835 | RGS3 | Y | Y | ENSG00000100014 | SPECC1L | N | Y |
| ENSG00000167110 | GOLGA2 | N | Y | ENSG00000185721 | DRG1 | Y | Y |
| ENSG00000198642 | KLHL9 | N | Y | ENSG00000100226 | GTPBP1 | N | Y |
| ENSG00000187713 | TMEM203 | N | Y | ENSG00000099954 | CECR2 | Y | Y |
| ENSG00000186193 | C9orf140 | N | Y | ENSG00000099954 | CECR2 | N | Y |
| ENSG00000155876 | RRAGA | N | Y | ENSG00000099954 | CECR2 | N | Y |
| ENSG00000125484 | GTF3C4 | N | Y | ENSG00000099991 | CABIN1 | N | Y |
| ENSG00000125484 | GTF3C4 | N | Y | ENSG00000128294 | TPST2 | N | Y |
| ENSG00000066697 | C9orf30 | Y | Y | ENSG00000100325 | ASCC2 | N | Y |
| ENSG00000157657 | ZNF618 | N | Y | ENSG00000159873 | CCDC117 | N | Y |
| ENSG00000241978 | AKAP2 | N | Y | ENSG00000100345 | MYH9 | N | Y |
| ENSG00000241978 | AKAP2 | N | Y | ENSG00000100345 | MYH9 | N | Y |
| ENSG00000241978 | AKAP2 | Y | Y | ENSG00000100345 | MYH9 | Y | Y |
| ENSG00000165138 | ANKS6 | N | Y | ENSG00000133424 | LARGE | N | Y |
| ENSG00000148248 | SURF4 | N | Y | ENSG00000133424 | LARGE | N | Y |
| ENSG00000188986 | COBRA1 | N | Y | ENSG00000093000 | NUP50 | Y | Y |
| ENSG00000198917 | C9orf114 | N | Y | ENSG00000093000 | NUP50 | Y | Y |
| ENSG00000130558 | OLFM1 | N | Y | ENSG00000093000 | NUP50 | N | Y |
| ENSG00000130558 | OLFM1 | Y | Y | ENSG00000100297 | MCM5 | N | Y |
| ENSG00000130559 | CAMSAP1 | N | Y | ENSG00000100105 | PATZ1 | N | Y |
| ENSG00000148468 | FAM171A1 | N | Y | ENSG00000100105 | PATZ1 | N | Y |
| ENSG00000107719 | KIAA1274 | N | Y | ENSG00000100105 | PATZ1 | N | Y |
| ENSG00000156374 | PCGF6 | N | Y | ENSG00000128245 | YWHAH | N | Y |
| ENSG00000107816 | LZTS2 | N | Y | ENSG00000253352 | TUG1 | N | Y |
| ENSG00000107815 | C10orf2 | N | Y | ENSG00000099904 | ZDHHC8 | N | Y |
| ENSG00000095637 | SORBS1 | N | Y | ENSG00000099968 | BCL2L13 | Y | Y |
| ENSG00000148600 | CDHR1 | N | Y | ENSG00000099968 | BCL2L13 | Y | Y |
| ENSG00000156521 | TYSND1 | N | Y | ENSG00000099968 | BCL2L13 | N | Y |
| ENSG00000151893 | C10orf46 | N | Y | ENSG00000099968 | BCL2L13 | Y | Y |
| ENSG00000107651 | SEC23IP | N | Y | ENSG00000159140 | SON | N | Y |
| ENSG00000065809 | FAM107B | N | Y | ENSG00000159140 | SON | N | Y |
| ENSG00000099204 | ABLIM1 | N | Y | ENSG00000159140 | SON | N | Y |
| ENSG00000148680 | HTR7 | N | Y | ENSG00000159128 | IFNGR2 | N | Y |
| ENSG00000107949 | BCCIP | N | Y | ENSG00000184787 | UBE2G2 | N | Y |
| ENSG00000107949 | BCCIP | N | Y | ENSG00000233393 | AP000688.29.1 | N | Y |
| ENSG00000148840 | PPRC1 | N | Y | ENSG00000183255 | PTTG1IP | Y | Y |
| ENSG00000155256 | ZFYVE27 | N | Y | ENSG00000160298 | C21orf58 | N | Y |
| ENSG00000138166 | DUSP5 | N | Y | ENSG00000160299 | PCNT | Y | Y |
| ENSG00000168209 | DDIT4 | N | Y | ENSG00000160299 | PCNT | N | Y |
| ENSG00000035403 | VCL | N | Y | ENSG00000182871 | COL18A1 | Y | Y |
| ENSG00000151208 | DLG5 | Y | Y | ENSG00000107872 | FBXL15 | N | Y |
| ENSG00000197444 | OGDHL | N | Y | ENSG00000095739 | BAMBI | N | Y |
| ENSG00000198954 | KIAA1279 | N | Y | ENSG00000176986 | SEC24C | N | Y |
| ENSG00000095787 | WAC | Y | Y | ENSG00000077147 | TM9SF3 | N | Y |
| ENSG00000148429 | USP6NL | Y | Y | ENSG00000107779 | BMPR1A | N | Y |
| ENSG00000148429 | USP6NL | Y | Y | ENSG00000110514 | MADD | N | Y |
| ENSG00000175029 | CTBP2 | Y | Y | ENSG00000166833 | NAV2 | N | Y |
| ENSG00000165886 | UBTD1 | N | Y | ENSG00000014216 | CAPN1 | N | Y |
| ENSG00000052749 | RRP12 | Y | Y | ENSG00000162337 | LRP5 | N | Y |
| ENSG00000171206 | TRIM8 | N | Y | ENSG00000048649 | RSF1 | N | Y |
| ENSG00000107957 | SH3PXD2A | N | Y | ENSG00000256591 | RP11- | N | Y |
| | | | | | 286N22.8.1 | | |
| ENSG00000134463 | ECHDC3 | Y | Y | ENSG00000171067 | C11orf24 | N | Y |
| ENSG00000107937 | GTPBP4 | Y | Y | ENSG00000171067 | C11orf24 | N | Y |
| ENSG00000122378 | FAM213A | N | Y | ENSG00000149260 | CAPN5 | N | Y |
| ENSG00000182180 | MRPS16 | N | Y | ENSG00000175575 | PAAF1 | Y | Y |
| ENSG00000148773 | MKI67 | N | Y | ENSG00000132749 | MTL5 | N | Y |
| ENSG00000062650 | WAPAL | Y | Y | ENSG00000149503 | INCENP | N | Y |
| ENSG00000062650 | WAPAL | Y | Y | ENSG00000149503 | INCENP | N | Y |
| ENSG00000062650 | WAPAL | Y | Y | ENSG00000118058 | MLL | N | Y |
| ENSG00000171307 | ZDHHC16 | Y | Y | ENSG00000137710 | RDX | N | Y |

In some embodiments, the assays, arrays and kits for assessing m⁶A levels in the RNA obtained from a population of stem cells, e.g., human stem cell can comprises measuring the m⁶A levels 10 or more mRNA transcripts selected from any of those listed in Tables S1, S2, S3, S4, S5 and S6, disclosed in Batista et al., Cell Stem Cell, 2014, 15(6), 707-719, entitled “m6A RNA Modification Controls Cell Fate Transition in Mammalian Embryonic Stem Cells”, (available online at the world-wide web address: “//dx.doi.org/10.1016/j.stem.2014.09.019”), which is incorporated herein in its entirety by reference.

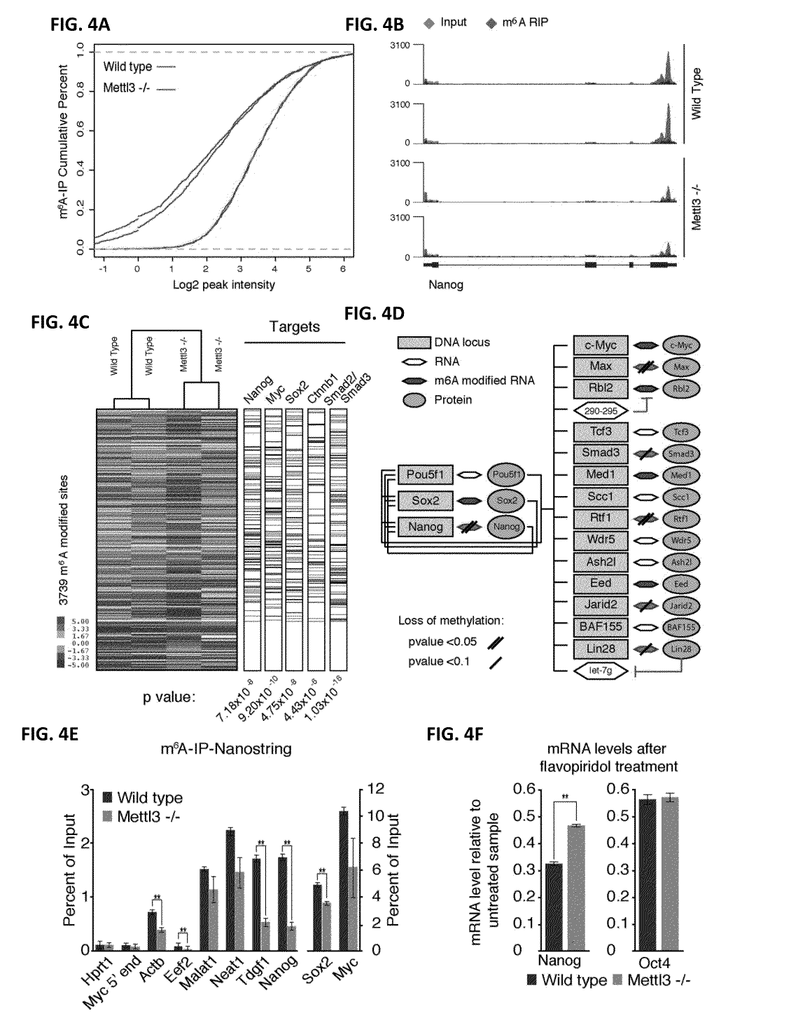

More specifically, Table S1 in Batista et al., discloses all Mouse High-Confidence Peaks (and relates to FIG. 1 and FIG. 4 herein) and shows the coordinates of m6A peaks in mouse genome (mm9), position of the m6A peak in the transcript, type of transcript, and gene symbol are displayed. For the Difference in Mettl3, the ratio between the IP and the Input is represented. Table S2 in Batista et al., discloses nanostring Counts after m6A-IP, and is related to FIG. 1 disclosed herein. Gene symbols with counts for Input, m6A IP, and IgG are shown. The ratios of the Input and Fold enrichment over the gene body of Actb are represented. Table S3 in Batista et al., discloses all Human High-Confidence Peaks and is related to FIG. 5 herein. Coordinates of m6A peaks in human genome (mm9), type of transcript, and gene symbols are shown. Table S4 in Batista et al., is reproduced as Table 2 herein and shows DPMI between T0 (undifferentiated) and T48 (endoderm differentiated) human stem cell populations. Table 2 is related to FIG. 5 herein and shows coordinates of m6A peaks in human genome (mm9), type of transcript, and gene symbols are shown. Each row indicates if DPMI is over 1.5- or 2-fold. Table S5 in Batista et al., discloses human and Mouse Methylated Gene Comparison, and is related to FIG. 6 herein and lists the Gene ID in human and mouse and type of homology are shown. Table S6 in Batista et al., is reproduced as Table 1 herein, and lists 632 gene transcripts that have common peaks between hESC and mESCs, and lists the Gene ID in human and mouse and chromosome coordinates of common peaks.

In some embodiments, the array comprises 10 or more oligonucleotides that hybridize to at least 10 mRNA transcripts, or to at least 10 3′UTR or other untranslated regions of at least 10 genes selected from any of those listed in Table 1 or Table 2, or any from Tables S1-S3 or S5, and (ii) contacting the array with at least one reagent which binds to m6A in the RNA, such as an anti-m⁶A antibody, or fragment thereof, such as an anti-m⁶A antibody which is fluorescently labeled or otherwise has a detectable label, therefore allowing the measurements of the levels of m6A in the at least selected 10 mRNA transcripts, or to at least 10 3′UTR or other untranslated regions of at least 10 genes selected from any of those listed in Table 1 or Table 2 or any from Tables S1-S3 or S5.

A further aspect of the technology described herein relates to methods, compositions, assays, arrays and kits for use in a method for determining the cell state of a stem cell population comprising performing the assay of claim 10, and comparing the levels of m6A (i.e., peak intensities) of at least 10 genes selected from any of Table 1 or Table 2, or any from Tables S1-S3 or S5, in the RNA from the stem cell population with the levels of m6A (i.e., peak intensities) in a reference stem cell population, and based on this comparison, determining the cell state of the stem cell population.

Another aspect of the present invention relates to a kit comprising: (i) an array composition for characterizing the cell state of a population of stem cells, comprising at least 10 oligonucleotides that hybridize to the RNA (i.e., mRNA transcripts, 3′UTR or other untranslated RNAs) of at least 10 genes selected from any of those in Table 1 or Table 2 or any from Tables S1-S3 or S5, as disclosed herein; and (ii) at least one regent to detect the m6A in RNA, such as, for example, an anti-m⁶A antibody, or fragment thereof, for example an anti-m6A antibody or fragment thereof which is detectably labeled (e.g., with a florescent label, colorimetric marker etc.).

A. Methods of m6A Analysis

B. Arrays

Methods of measure m6A are known by one of ordinary skill in the art. For example, as disclosed herein, one can use anti-m6A antibodies. Commercial m6A RNA methylation quantification kits are commercially available and encompassed for use in the methods, kits and assays as disclosed herein, e.g., such as those from AbCam (Cat No: ab185912) or Epigentek (Cat No:P-9005-96).

Accordingly, an array as disclosed herein encompasses an array of oligonucleotides which hybridize to the target RNA species (e.g., 10 or more genes selected from any listed in Table 1, Table 2, Table S1-S3 or Table S5), and contacting the array with RNA obtained from the stem cell population (e.g, human stem cell population) and allowing the RNA to hybridize to the oligonucleotides, washing the array to remove any unbound (non-hybridized) RNA, then adding an anti-m6A antibody. After removal of the unbound anti-m6A antibody, the bound anti-m6A antibody can be detected by methods commonly known in the art, e.g., where the anti-m6A antibody is fluorescently labeled, using flursecent detection, or using a different colormetic method known in the art.

In some embodiments, the oligonucleotides on the array are at least 90% identical to, or specifically hybridize to the RNA or mRNA of the genes selected from any listed in Table 1, Table 2, Table S1-S3 or Table S5). In some embodiments, the array comprises oligonucleotides (e.g., probes or primers) which specifically hybridize to the mRNA expressed by the genes selected from any listed in Table 1, Table 2, Table S1-S3 or Table S5).

In some embodiments, the array comprises at least 10, or at least about 20, or at least about 30, or 30-60, or 60-90 or more than 90 nucleic acid sequences (e.g. oligonucleotides), or at least 10, or at least about 20, or at least about 30, or 30-60, or 60-90 or more than 90 pairs of nucleic acid sequences (e.g., primers), that can be used to measure m6A levels of a combination of 10 or more genes selected from any listed in Table 1, Table 2, Table S1-S3 or Table S5).

In some embodiments, any of the genes listed in Table 1, Table 2, Table S1-S3 or Table S5 can be substituted for alternative genes. For example, in some embodiments, in addition to comprising probes (e.g., oligonucleotides and/or primers) which specifically hybridize to the mRNA of at least 10, or at least 20 genes selected from any listed in Table 1, Table 2, Table S1-S3 or Table S5), the array can comprise additional reagents (e.g., probes, e.g., oligonucleotides and/or primers) which specifically hybridize to the mRNA of other genes for measuring the m6A levels of genes not listed in Table 1, Table 2, Table S1-S3 or Table S5). Such genes are known by persons of ordinary skill in the art and are envisioned for use in the assays, kits, methods, systems as disclosed herein.

In some embodiments, the array further comprises nucleic acid sequences (e.g., oligonucleotides and/or primers) which specifically hybridize to the mRNA of at least 1, or at least 2, or at least 3, or at least 4 or least 5 control genes. Control genes include those listed in Table 3, but are not limited to ACTB, JARID2, CTCF, SMAD1, β-actin, GAPDH and the like. In some embodiments, nucleic acid sequences that amplify a control gene can be present at multiple locations in the same array.

In some embodiments, the array comprises nucleic acid sequences, e.g., oligonucleotides or primers, that amplify the mRNA of at least sequences corresponding to 1-10 control genes, such as, but not limited to the control genes selected from the group consisting of: ACTB, JARID2, CTCF, SMAD1, GAPDH, β-actin, EIF2B, RPL37A, CDKN1B, ABL1, ELF1, POP4, PSMC4, RPL30, CASC3, PES1, RPS17, RPSL17L, CDKN1A, MRPL19, MT-ATP6, GADD45A, PUM1, YWHAZ, UBC, TFRC, TBP, RPLP0, PPIA, POLR2A, PGK1, IPO8, HMBS, GUSB, B2M, HPRT1 or 18S.

In some embodiments, the array comprises no more than 100, or no more than 90, or no more than 50 nucleic acid sequences, e.g., oligonucleotides or primers. In some embodiments, the nucleic acid sequences present on the array are sets of primers. In some embodiments, the nucleic acid sequences, e.g., oligonucleotides or primers are immobilized on, or within a solid support. Nucleic acid sequences can be immobilized on the solid support by the 5′ end of said oligonucleotides. In some embodiments, the solid support is selected from a group of materials comprising silicon, metal, and glass. In some embodiments, the solid support comprises oligonucleotides at assigned positions defined by x and y coordinates.

Accordingly, the present invention contemplates a method of generating an array, comprising providing a solid support comprising a plurality of positions for oligonucleotides, the positions defined by x and y coordinates; a plurality of different oligonucleotides (or primer pairs), each comprising a sequence which is complementary to at least a portion of the sequence of an gene being measured, where each oligonucleotide (or primer pair) is placed in a known position on the solid support to create an ordered array.

In one embodiment of the present invention, oligonucleotides that are immobilized by the 5′ end on a solid surface by a chemical linkage are contemplated. In some embodiments, the oligonucleotides are primers, and can be approximately 17 bases in length, although other lengths are also contemplated.

In another embodiment of the present invention, a method of hybridizing target nucleic acid fragments is contemplated which comprises providing an ordered array of immobilized oligonucleotides representing sequences in selected from any listed in Table 1, Table 2, Table S1-S3 or Table S5 and providing a plurality of fragments of a target nucleic acid; and bringing the fragments of the target nucleic acid into contact with the array under conditions such that at least one of the fragments hybridizes to one of the immobilized oligonucleotides on the array.

In some embodiments, when RNA from the stem cell population hybridizes to an oligonucleotide attached on the surface of the array, it is detected with an antibody, e.g., anti-m6A antibody that is detectably labeled or has a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review see Jones et al. (1999) Trends Biotechnol. 17(12):477-81). Thus, antibodies can be genetically modified to provide a fluorescent dye as part of their structure. Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques.

Hybridization to arrays may be performed, where the arrays can be produced according to any suitable methods known in the art. For example, methods of producing large arrays of oligonucleotides are described in U.S. Pat. No. 5,134,854, and U.S. Pat. No. 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505. Methods for collection of data from hybridization of samples with an array are also well known in the art. For example, the polynucleotides of the cell samples can be generated using a detectable fluorescent label, and hybridization of the polynucleotides in the samples detected by scanning the microarrays for the presence of the detectable label. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample is compared to the fluorescent signal from another sample, and the relative signal intensity determined. Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e. data deviating from a predetermined statistical distribution, and calculating the relative binding affinity of the targets from the remaining data. The resulting data can be displayed as an image with the intensity in each region varying according to the binding affinity between targets and probes. Pattern matching can be performed manually, or can be performed using a computer program. Methods for preparation of substrate matrices (e.g., arrays), design of oligonucleotides for use with such matrices, labeling of probes, hybridization conditions, scanning of hybridized matrices, and analysis of patterns generated, including comparison analysis, are described in, for example, U.S. Pat. No. 5,800,992. General methods in molecular and cellular biochemistry can also be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

In some embodiments, the detection agent, e.g., anti-m6A antibody is further labeled with a detectable marker, for example a fluorescent marker. Such detectable labels include, but are not limited to, for example but not limited to metallic beads and streptavidin.

RNA can be isolated from eukaryotic cells by procedures that involve lysis of the cells and denaturation of the proteins contained therein. Stem cells of interest include pluripotent stem cells, including but not limited to ES cells, adult stem cells and iPSC cells, from mammals including human species. Additional steps can be employed to remove DNA. Cell lysis can be accomplished with a nonionic detergent, followed by microcentrifugation to remove the nuclei and hence the bulk of the cellular DNA. In one embodiment, RNA is extracted from cells of the various types of interest using guanidinium thiocyanate lysis followed by CsCl centrifugation to separate the RNA from DNA (Chirgwin et al., Biochemistry 18:5294-5299 (1979)). Poly(A)+ RNA is isolated by selection with oligo-dT cellulose (see Sambrook et al, MOLECULAR CLONING—A LABORATORY MANUAL (2ND ED.), Vols. 1-3, Cold Spring Harbor Laboratory, Cold Spring Harbor, N.Y. (1989). Alternatively, separation of RNA from DNA can be accomplished by organic extraction, for example, with hot phenol or phenol/chloroform/isoamyl alcohol. If desired, RNase inhibitors can be added to the lysis buffer. Likewise, for certain cell types, it can be desirable to add a protein denaturation/digestion step to the protocol.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

For many applications, it is desirable to preferentially enrich mRNA with respect to other cellular RNAs, such as transfer RNA (tRNA) and ribosomal RNA (rRNA). Most mRNAs contain a poly(A) tail at their 3′ end. This allows them to be enriched by affinity chromatography, for example, using oligo(dT) or poly(U) coupled to a solid support, such as cellulose or Sephadex. (see Ausubel et al., CURRENT PROTOCOLS IN MOLECULAR BIOLOGY, vol. 2, Current Protocols Publishing, New York (1994). Once bound, poly(A)+mRNA is eluted from the affinity column using 2 mM EDTA/0.1% SDS.

The sample of RNA can comprise a plurality of different mRNA molecules, each different mRNA molecule having a different nucleotide sequence. In a specific embodiment, the mRNA molecules in the RNA sample comprise at least 100 different nucleotide sequences. In another specific embodiment, the RNA sample is a mammalian RNA sample.

In a specific embodiment, total RNA or mRNA from the pluripotent stem cell population is used in the assays and methods as disclosed herein. The source of the RNA can be pluripotent cells or stem cells of an animal, human, mammal, primate, non-human animal, dog, cat, mouse, rat, bird, etc. In specific embodiments, the methods of the invention are used with a sample containing mRNA or total RNA from 1×10⁶ cells or less. In another embodiment, proteins can be isolated from the foregoing sources, by methods known in the art, for use in expression analysis at the protein level.

Probes to the homologs of the target gene sequences disclosed herein in Tables 1, 2 or S1-S3 or S5 can be employed preferably wherein non-human nucleic acid is being assayed.

### Assays to Determine the Differentiation Potential of Pluripotent Stem Cells

In some embodiments, the present invention provides a method for selecting a stem cell line, e.g., a pluripotent stem cell line, comprising measuring the m6A RNA modification (or m6A peak intensities) of target genes (e.g., selected from any listed in Table 1, Table 2, Table S1-S3 or Table S5) in a stem cell line; and comparing the m6A peak intensity with a reference level of the same genes.

In some embodiments, a stem cell line, e.g., a pluripotent stem cell line is a mammalian pluripotent stem cell line, such as a human pluripotent stem cell line.

In some embodiments, the assay is a high-throughput assay for assaying a plurality of different stem cell lines, for example, but not limited to permitting one to assess a plurality of different induced pluripotent stem cells derived from reprogramming a somatic cell obtained from the same or a different subject, e.g., a mammalian subject or a human subject. In some embodiments, the assay is a 96-well format, and in some embodiments, the assay is in a 384-well format, permitting multiple pluripotent stem cell lines to be assayed at the same time. In some embodiments, the assay is an automated format, enabling high-throughput analysis of 96- and/or 384-well plates.

In additional aspects, the stem cell line, e.g., pluripotent stem cells are cultured under different conditions and in different culture media and analyzed for m6A peak intensities in target genes, e.g. genes selected from any listed in Table 1, Table 2, Table S1-S3 or Table S5. This allows for differences in analysis of stem cells in different maintenance culture conditions, such as the cultivation to high density which can influence stem cells transitioning from an undifferentiated to differentiated phenotype.

In some embodiments, the differentiation assay can be configured to be automated e.g., to be run by a robot. In some embodiments, a robot can also perform RNA extraction of an entire multiwell plate, and pipettes the RNA from each well into separate assay plates (e.g., when using 96-well qPCR plates) or into ¼ of a plate (e.g., when using 384-well qPCR plates). For example, where one stem cell line is to be analyzed, the RNA from the stem cell line can be pipetted into each well of a 96-well plate, and each well of the 96-well plate used to measure the m6A levels of different genes and/or control. In some embodiments, were multiple stem cell lines are to be analyzed, the RNA from each stem cell line can be plated into ¼ of the individual wells of a 384-well plate, where a 384-well plate can be used for the analysis of 4 stem cell lines at the same time.

Another aspect of the present invention relates to the use of a stem cell line, e.g., a pluripotent stem cell line, which has been validated and characterized using the methods and arrays and assays disclosed herein, for treatment of a subject by administering to a subject a stem cell population, for example a treatment of a mammalian subject, e.g., a mouse or rodent animal model or a human subject, such as for regenerative medicine and cell replacement/enhancement therapy. In some embodiments, a subject suffers from or is diagnosed with a disease or condition selected from the group consisting of cancer, diabetes, cardiac failure, muscle damage, Celiac Disease, neurological disorder, neurodegenerative disorder, lysosomal storage disease, and any combinations thereof. In some embodiments, the pluripotent stem cell is administered locally, or alternatively, administration is transplantation of the pluripotent stem cell into the subject.

In some embodiments, the stem cell populations for use in the methods, assays, arrays and kits as disclosed herein can be a pluripotent human stem cell population, e.g., a stem cell population that has the ability to differentiate along a lineage selected from the group consisting of mesoderm, endoderm, ectoderm, neuronal, hematopoietic lineages, and any combinations thereof, or differentiated into an insulin producing cell (pancreatic cell, beta-cell, etc.), neuronal cell, muscle cell, skin cell, cardiac muscle cell, hepatocyte, blood cell, adaptive immunity cell, innate immunity cell and the like.

In some embodiments, the methods, assays, arrays and systems as disclosed herein can be performed by a service provider, for example, where an investigator can have one or more samples (e.g., an array of samples) each sample comprising a stem cell line, or a different population of stem cells, for assessment using the methods, differentiation assays, kits and systems as disclosed herein in a diagnostic laboratory operated by the service provider. In such an embodiment, after performing the assays of the invention as disclosed, the service provider performs the analysis and provide the investigator a report, e.g., levels of m6A of the target genes, or list of m6A peak intensities of each stem cell line analyzed. In alternative embodiments, the service provider can provide the investigator with the raw data of the assays and leave the analysis to be performed by the investigator. In some embodiments, the report is communicated or sent to the investigator via electronic means, e.g., uploaded on a secure web-site, or sent via e-mail or other electronic communication means. In some embodiments, the investigator can send the samples to the service provider via any means, e.g., via mail, express mail, etc., or alternatively, the service provider can provide a service to collect the samples from the investigator and transport them to the diagnostic laboratories of the service provider. In some embodiments, the investigator can deposit the samples to be analyzed at the location of the service provider diagnostic laboratories. In alternative embodiments, the service provider provides a stop-by service, where the service provider send personnel to the laboratories of the investigator and also provides the kits, apparatus, and reagents for performing the assays on the investigators stem cell lines in the investigators laboratories, and analyze the results and provides a report to the investigator of the characteristics of each stem cell line analyzed, or plurality of stem cell lines analyzed.

### Kits

Another aspect of the present invention relates to kits for characterizing the cell state of a population of stem cells, e.g., human stem cells, comprising an array as disclosed herein. In some embodiments, a kit comprises an array as disclosed herein and reagents for measuring the levels of m6A RNA modification, including m6A peak intensities of a set of genes selected from any listed in Table 1 or Table 2, or any listed in Tables S1-S3 or S5 in Batista et al., which is incorporated herein in its entirety by reference. The kit can further comprise instructions for use.

In some embodiments, the kit for carrying out the methods as disclosed herein comprises probes (e.g., oligonucleotides and/or primers) which specifically hybridize to the mRNA of at least about 20, or at least about 30, or at least about 40, or at least about 50, or at least about 60, or at least about 70, or at least about 80, or at least about 90 or more than 90 genes selected from any of those listed in Table 1 or Table 2, or any from Tables S1-S3 or S5. In some embodiments, the kit comprises probes (e.g., oligonucleotides and/or primers) which specifically hybridize to the mRNA of at least about 3 or more genes selected from Table 1 or Table 2.

Another aspect of the present invention relates to a kit for carrying out a methods and assays as disclosed herein, where the kit comprises: reagents for measuring the m6A levels of a set of genes selected from any of at least 20 or at least 30 from the genes listed in Table 1 or Table 2, or any from Tables S1-S3 or S5. In some embodiments, the reagents are antibodies to m6A RNA, or antibody fragments or epitope binding portions thereof. In some embodiments, the reagents, e.g., antibodies or fragments thereof are detectably labeled. In some embodiments, the probes, e.g., oligonucleotides can be immobilized on a solid support. In some embodiments, in addition to comprising oligonucleotides that hybridize to at least 20 genes selected from Table 1 or Table 2, or any from Tables S1-S3 or S5., the kit can comprise additional reagents for measuring the m6A levels of different genes not listed in Table 1. In some embodiments, the kit comprises an array which also comprises oligos for at least 1, or at least 2, or at least 3, or at least 4 or least 5 control genes. Control genes include, but are not limited to any of combination of: ACTB, JARID2, CTCF, SMAD1, β-actin, GAPDH, EIF2B, RPL37A, CDKN1B, ABL1, ELF1, POP4, PSMC4, RPL30, CASC3, PES1, RPS17, RPSL17L, CDKN1A, MRPL19, MT-ATP6, GADD45A, PUM1, YWHAZ, UBC, TFRC, TBP, RPLP0, PPIA, POLR2A, PGK1, IPO8, HMBS, GUSB, B2M, HPRT1 or 18S and the like. In some embodiments, a probe for a control gene can be present multiple times in the same assay or kit.

In some embodiments, the kit further comprises instructions for use. In some embodiments, the kit comprises a computer readable medium comprising instructions encoded thereupon for running a software program on a computer to compare the levels of m6A modification on the RNA of a set of gene targets in a test stem cell population with reference m6A levels of the same genes. In some embodiments, the kit comprises instructions to access a software program available online (e.g., on a cloud) to compare the measured m6A levels of the genes from the test stem cell population (e.g., human stem cell population) with reference m6A levels from a control stem cell population.

In some embodiments, the array include probes e.g., hybridization probes that specifically hybridize to a set of target genes selected from a subset of at least 20 genes from any listed in Table 1 or Table 2, or any from Tables S1-S3 or S5. In some embodiments, the probes, e.g., oligos can be immobilized on a solid support. In some embodiments, the kit and/or assay as disclosed herein comprises probes (e.g., oligos) for at least about 10, or at least about 20, or at least about 30, or more than 30 genes listed in Table 1 or 2.

In some embodiments, the kit is in a 96-well or 384-well format and comprises probes to hybridize with a set of target genes selected from any of those listed in Table 1 or Table 2, or any from Tables S1-S3 or S5. In some embodiments, the kit can be configured to be automated e.g., to be run by a robot. For example, samples can be added to the array of the kit using a robot etc., and the robot can perform the hybridization method, wash the array to remove non-hybridized RNA, add the detection reagent (e.g., an anti-m6A antibody, such as a detectably labeled anti-m6A antibody), wash the array to remove non-bound detection agent, and detection of m6A levels using an anti-m6A antibody (e.g., a detectably labeled anti-m6A antibody) and readout of the levels of m6A levels of the measured target genes. In some embodiments, the robot can perform computer or comparative analysis of the detected m6A levels to provide peak intensities of the m6A levels for each target gene assessed.

In some embodiments, a kit as disclosed herein also comprises at least one reagent for selecting a desired stem cell line, e.g., a stem cell line among many cell lines, e.g., reagents to select one or more appropriate stem cell lines for the intended use of the stem cell line. Such agents are well known in the art, and include without limitation, labeled antibodies to select for cell-specific lineage markers and the like. In some embodiments, the labeled antibodies are fluorescently labeled, or labeled with magnetic beads and the like. In some embodiments, a kit as disclosed herein can further comprise at least one or more reagents for profiling and annotating an existing ES cell and/or iPS cell bank in high throughput, according to the methods as disclosed herein.

In one aspect the invention provides a kit comprising one or more control stem cell populations, e.g., a control undifferentiated human stem cell population, and/or a control differentiated human cell cell population, which can be used for comparative analysis with a test human stem cell population being assessed using the methods, arrays and assays as disclosed herein. In addition to the above mentioned component(s), the kit can also include informational material. The informational material can be descriptive, instructional, marketing or other material that relates to the methods described herein and/or the use of the components for the assays, methods and systems described herein. For example, the informational material can describe methods for selecting a stem cell population, for measuring m6A levels, etc.

### Uses

In some embodiments, the methods, arrays, assays and kits as disclosed herein can be used in a variety of ways clinically and in research applications. For instance, methods, arrays, assays and kits as disclosed herein are useful for identifying the cell state of a stem cell population (e.g., a human stem cell population), e.g., if it is in an undifferentiated (i.e., resting) pluripotent state, or if it has started or undergone lineage differentiation. In some embodiments, the fingerprinting of m6A levels or peak intensities as disclosed herein is useful for assessing the phenotype or differentiation of a stem cell population in response to a drug, and therefore can be used for drug screening purposes. Additionally, the methods, arrays and assays as disclosed herein are useful to ensure stem cell populations used in a drug screening assay are consistant and are in the same cell state, and do not differ from each other, thus enabling the drug screening to identify potential hits/drugs are the effect of the drug rather than due to variations in the different stem cell lines.

In some embodiments, the methods, arrays, assays and kits as disclosed herein are useful for identifying and selecting a stem cell line, e.g., a pluripotent stem cell line which would be suitable for therapeutic use, e.g., stem cell therapy or other regenerative medicine. In some embodiments, the methods, arrays, assays and kits as disclosed herein can be used in clinics to determine clinical safety and utility of a particular pluripotent stem cell line.

In some embodiments, the methods, arrays, assays and kits as disclosed herein can be used as a quality control to monitor the characteristics of a stem cell population, e.g., a human stem cell line, over multiple passages and/or before and after cryopreservation procedures, for example, to ensure that the cell remains in an undifferentiated (e.g., resting) state and no significant epigenetic or functional genomic changes have occurred over time (e.g., over passages and after cryopreservation). For example, the methods, arrays, assays and kits as disclosed herein can be used to characterize stem cell populations before, and during storage, e.g., in a stem cell bank, to catalogue each stem cell line (e.g., human stem cell line) which is placed in the bank, and to ensure that the stem cells have the same properties after thawing as they did prior to cryopreservation. In some embodiments, a stem cell population can be contacted with a METTL3 and/or METTL4 inhibitor as disclosed herein, before, after or during crypopreservation, e.g., a METTL3 and/or METTL4 inhibitor can be present in a cryopreservation media.

In some embodiments, the raw data of m6A levels and/or m6A peak intensities for target genes for each stem cell line can be stored in a centralized database, where the data can be used to select a pluripotent stem cell line for a particular use or utility, e.g., for selection of a stem cell line in a stem cell bank.

In some embodiments, the methods, arrays, assays and kits as disclosed herein can be used in research to monitor functional genomic changes as a stem cell line, e.g., a pluripotent stem cell line, differentiates along different lineages. In some embodiments, aspects as disclosed herein can be used to monitor and determine the characteristics of stem cell lines from subjects with particular diseases, e.g., one can monitor stem cell lines, e.g., a stem cell line from subjects with genetic defects or particular genetic polymorphisms, and/or having a particular disease. For example, one can monitor and determine the m6A levels between an iPSC cell derived from a subject with a neurodegenerative disease, such as ALS, as compared to a normal iPSC cell from a healthy subject (or a non-ALS subject), such as a healthy sibling. Similarly, one can determine if iPS cells has comparable m6A levels (or peak intensities) of selected target genes as compared to human ES cells or other pluripotent stem cells. Additionally, the aspects as disclosed herein can fully characterize the cell state of a stem cell population, e.g. human stem cell population without the need for teratoma assays and/or generation of chimera mice, therefore significantly increasing the high-throughput ability of characterizing pluripotent stem cell lines.

In some embodiments, the methods, arrays, assays and kits as disclosed herein can be used in creating a database, where such a database would be useful in organizing and cataloging a human stem cell repository, e.g., a central repository (e.g., a tissue and/or cell bank) containing a large number of quality-controlled and utility-predicted pluripotent cell lines, such that one can use a database comprising the m6A levels (or m6A peak intensities) of specific target genes for each stem cell line in the bank to specifically select a particular pluripotent stem cell line for the investigators' intended use. In some embodiments, the use of such a database can be easily extended such that a user can upload the data from the array or assays as disclosed herein (e.g., m6A levels, and/or m6A peak intensities for selected target genes) for a particular stem cell population of interest. In a simple analogy, the database could function similar to Google's “search for similar sites”, whereby the database could be used as an efficient way to select useful cell lines for novel and/or mixed tissue types, or to identify stem cell lines in a cell bank that can have are in the undifferentiated (i.e. resting) cell state or are differentiated along a specific lineage.

In some embodiments, the methods, arrays, assays and kits as disclosed herein can be used for identification and selection of a desired stem cell line, e.g., a pluripotent stem cell line for mass production. For example, methods to inhibit MEETTL3 and/or METTL4 can be used to maintain the cells in an undifferentiated state of culturing and expanding a stem cell population efficiently in large quantities, e.g., large batch cultures or in bioreactors, and the fingerprinting methods, and uses of the assays and arrays as disclosed herein can be used as a quality control to ensure the expanded stem cell population remained in an undifferentiated cell state during expansion in a bulk culture.

In another embodiment, the methods, arrays, assays and kits as disclosed herein can be used for assessing drug responsiveness of a stem cell population, for example, a stem cell line can be assessed using the methods, arrays, assays and kits as disclosed herein prior to, during, and after contacting with a drug or other agent or stimulus (e.g., electric stimuli for cardiac pluripotent progenitors) to generate m6A signature of the stem cell line in the presence or absence of the drug.

In another embodiment, the methods, arrays, assays and kits as disclosed herein can be used for selection of a stem cell line, e.g., a pluripotent stem cell line, based on its safety profile. For example, a stem cell population can be selected that has a m6A signature indicating it is in an undifferentiated state etc.

In another embodiment, the methods, arrays, assays and kits as disclosed herein can be used for selection and/or quality control, and/or validation of a stem cell population in different or new states of pluripotency or multipotency, for example to provide information regarding which stem cell lines are in an undifferentiated state (i.e., pluripotent state) but do not fall under the usual definition of human ES cell lines (e.g., human ground-state ES cell and partially reprogrammed cell lines, e.g., partially induced pluripotent stem (piPS) cells, which are capable of being reprogrammed further to a pluripotent stem cell).

It has been shown that continued in vitro culture and passaging improves the quality of iPS cell lines (see Polo et al., Nat Biotechnol. 2010 August; 28(8):848-55, and Nat Rev Mol Cell Biol. 2010 September; 11(9):601, and Nat Rev Genet. 2010 September; 11(9): 593). On the other hand, continued passaging is expensive. Accordingly, in some embodiments, the methods, arrays, assays and kits as disclosed herein can be used for measuring how much passaging is sufficient for improving the quality of the stem cell line, e.g., the pluripotent stem cell line.

In further embodiments, the methods, arrays, assays and kits as disclosed herein can be used in a variety of different research and clinical uses to characterize, monitor and assess if a stem cell line is in an undifferentiated state. For example, typical application includes in areas such as, but not limited to, (i) labs and/or companies interested in disease mechanisms (e.g., using the kits or services as disclosed herein to reduce the complexity of generating iPS cell lines, as well as differentiated cells for disease modeling and small-scale drug screening, (ii) labs and/or companies trying to identify small molecules and/or biologicals for a given disease target (e.g., using the kits and/or services as disclose herein to enable the production of large numbers of highly standardized cells for drug screening), (iii) clinical and pre-clinical research groups for quality control and validating stem cell lines where they are interested in producing cells for implantation into humans or animals (e.g., using a kit and/or service as disclosed herein to permits quality control at a level of accuracy that will be sufficient for regulatory approval, e.g., FDA approval), (iv) tissue banks that desire to give their customers information, including advice and data about the undifferentiated state of the stem cell population, and quality and utility of the stem cell lines, e.g., pluripotent stem cell lines on offer (e.g., using a kit and/or service as disclosed herein to provide unbiased assessment of the quality and/or utility of a large number of pluripotent cell lines, in an inexpensive high throughput manner, —it is contemplated that the assays can ultimately be performed on 1,000-100,000s of pluripotent stem cell lines to cover the whole population of cell lines stored in the cell bank), (v) private consumers who desire to generate, and optionally, bank at least one or more stem cell lines, e.g., pluripotent stem cell lines, e.g., iPS cell lines (or piPS cell lines) generated from their somatic differentiated cells, either for themselves and/or their children or other offspring, for example, as a type of health insurance policy for future regenerative medicine purposes.

### Stem Cell Populations for Analysis of m6A Levels (or m6A Peak Intensities)

As disclosed herein, m6A levels (e.g., m6A peak intensities) of target genes can be used to assess if the cell state of any stem cell line or population, from any species, e.g. a mammalian species, such as a human. In some embodiments, the present invention specifically contemplates using the methods, arrays, assays and kits as disclosed herein to determine if a stem cell is pluripotent. Any type of stem cell can be assessed. For simplicity, when referring to analysis of a pluripotent stem cell herein, this encompasses analysis of both pluripotent and non-pluripotent stem cells.

In some embodiments, the stem cell is a pluripotent stem cell. Generally, a pluripotent stem cell to be analyzed according to the methods described herein can be obtained or derived from any available source. Accordingly, a pluripotent cell can be obtained or derived from a vertebrate or invertebrate. In some embodiments, the pluripotent stem cell is mammalian pluripotent stem cell. In all aspects as disclosed herein, pluripotent stem cells for use in the methods, arrays, assays and kits as disclosed herein can be any pluripotent stem cell.

In some embodiments, the pluripotent stem cell is a primate or rodent pluripotent stem cell. In some embodiments, the pluripotent stem cell is selected from the group consisting of chimpanzee, cynomologous monkey, spider monkey, macaques (e.g. Rhesus monkey), mouse, rat, woodchuck, ferret, rabbit, hamster, cow, horse, pig, deer, bison, buffalo, feline (e.g., domestic cat), canine (e.g. dog, fox and wolf), avian (e.g. chicken, emu, and ostrich), and fish (e.g., trout, catfish and salmon) pluripotent stem cell.

In some embodiments, the pluripotent stem cell is a human pluripotent stem cell. In some embodiments, the pluripotent stem cell is a human stem cell line known in the art. In some embodiments, the pluripotent stem cell is an induced pluripotent stem (iPS) cell, or a stably reprogrammed cell which is an intermediate pluripotent stem cell and can be further reprogrammed into an iPS cell, e.g., partial induced pluripotent stem cells (also referred to as “piPS cells”). In some embodiments, the pluripotent stem cell, iPSC or piPSC is a genetically modified pluripotent stem cell.

In some embodiments, the pluripotent state of a pluripotent stem cell used in the present invention can be confirmed by various methods. For example, the pluripotent stem cells can be tested for the presence or absence of characteristic ES cell markers. In the case of human ES cells, examples of such markers include SSEA-4, SSEA-3, TRA-1-60, TRA-1-81 and OCT 4, and are known in the art.

While the methods of the present invention allow the pluripotency (or lack thereof) to be assessed by measuring m6A levels (or peak intensities) of a subset of genes listed in Table 1 and/or 2, the pluripotency of a stem cell line can also be confirmed by injecting the cells into a suitable animal, e.g., a SCID mouse, and observing the production of differentiated cells and tissues. Still another method of confirming pluripotency is using the subject pluripotent cells to generate chimeric animals and observing the contribution of the introduced cells to different cell types. Methods for producing chimeric animals are well known in the art and are described in U.S. Pat. No. 6,642,433, which is incorporated by reference herein.

Yet another method of confirming pluripotency is to observe ES cell differentiation into embryoid bodies and other differentiated cell types when cultured under conditions that favor differentiation (e.g., removal of fibroblast feeder layers). This method has been utilized and it has been confirmed that the subject pluripotent cells give rise to embryoid bodies and different differentiated cell types in tissue culture.

In this regard, it is known that some mouse embryonic stem (ES) cells have a propensity of differentiating into some cell types at a greater efficiency as compared to other cell types. Similarly, human pluripotent (ES) cells can possess selective differentiation capacity. Accordingly, the present invention can be used to identify and select a pluripotent stem cell with desired characteristics and differentiation propensity for the desired use of the pluripotent stem cell. For example, where the pluripotent cell line has been screened according to the methods of the invention, a pluripotent stem cell can be selected due to its increased efficiency of differentiating along a particular cell line, and can be induced to differentiate to obtain the desired cell types according to known methods. For example, a human pluripotent stem cell, e.g., a ES cell or iPS cell can be induced to differentiate into hematopoietic stem cells, muscle cells, cardiac muscle cells, liver cells, islet cells, retinal cells, cartilage cells, epithelial cells, urinary tract cells, etc., by culturing such cells in differentiation medium and under conditions which provide for cell differentiation, according to methods known to persons of ordinary skill in the art. Medium and methods which result in the differentiation of ES cells are known in the art as are suitable culturing conditions.

In some embodiments, the stem cell population is a iPS cell, e.g., a hiPSC. One can use any method for reprogramming a somatic cell to an iPS cell or an piPS cell, for example, as disclosed in International patent applications; WO2007/069666; WO2008/118820; WO2008/124133; WO2008/151058; WO2009/006997; and U.S. Patent Applications US2010/0062533; US2009/0227032; US2009/0068742; US2009/0047263; US2010/0015705; US2009/0081784; US2008/0233610; U.S. Pat. No. 7,615,374; U.S. patent application Ser. No. 12/595,041, EP2145000, CA2683056, AU8236629, Ser. No. 12/602,184, EP2164951, CA2688539, US2010/0105100; US2009/0324559, US2009/0304646, US2009/0299763, US2009/0191159, the contents of which are incorporated herein in their entirety by reference. In some embodiments, an iPS cell for use in the methods as described herein can be produced by any method known in the art for reprogramming a cell, for example virally-induced or chemically induced generation of reprogrammed cells, as disclosed in EP1970446, US2009/0047263, US2009/0068742, and 2009/0227032, which are incorporated herein in their entirety by reference. In some embodiments, iPS cells can be reprogrammed using modified RNA (mod-RNA) as disclosed in US2012/0046346, which is incorporated herein in its entirety by reference.

In some embodiments, an iPS cell for use in the methods, arrays, assays and kits as disclosed herein can be produced from the incomplete reprogramming of a somatic cell by chemical reprogramming, such as by the methods as disclosed in WO2010/033906, the content of which is incorporated herein in its entirety by reference. In alternative embodiments, the stable reprogrammed cells disclosed herein can be produced from the incomplete reprogramming of a somatic cell by non-viral means, such as by the methods as disclosed in WO2010/048567 the contents of which is incorporated herein in its entirety by reference.

Other stem cells for use in the methods as disclosed herein can be any stem cell known to persons of ordinary skill in the art. Exemplary stem cells include embryonic stem cells, adult stem cells, pluripotent stem cells, neural stem cells, liver stem cells, muscle stem cells, muscle precursor stem cells, endothelial progenitor cells, bone marrow stem cells, chondrogenic stem cells, lymphoid stem cells, mesenchymal stem cells, hematopoietic stem cells, central nervous system stem cells, peripheral nervous system stem cells, and the like. Descriptions of stem cells, including methods for isolating and culturing them, can be found in, among other places, Embryonic Stem Cells, Methods and Protocols, Turksen, ed., Humana Press, 2002; Weisman et al., Annu. Rev. Cell. Dev. Biol. 17:387 403; Pittinger et al., Science, 284:143 47, 1999; Animal Cell Culture, Masters, ed., Oxford University Press, 2000; Jackson et al., PNAS 96(25):14482 86, 1999; Zuk et al., Tissue Engineering, 7:211 228, 2001 (“Zuk et al.”); particularly Chapters 33 41; and U.S. Pat. Nos. 5,559,022, 5,672,346 and 5,827,735. Descriptions of stromal cells, including methods for isolating them, can be found in, among other places, Prockop, Science, 276:71 74, 1997; Theise et al., Hepatology, 31:235 40, 2000; Current Protocols in Cell Biology, Bonifacino et al., eds., John Wiley & Sons, 2000 (including updates through March, 2002); and U.S. Pat. No. 4,963,489.

Additional pluripotent stem cells for use in the methods, arrays, assays and kits as disclosed herein can be any cells derived from any kind of tissue (for example embryonic tissue such as fetal or pre-fetal tissue, or adult tissue), which stem cells have the characteristic of being capable under appropriate conditions of producing progeny of different cell types that are derivatives of all of the 3 germinal layers (endoderm, mesoderm, and ectoderm). These cell types can be provided in the form of an established cell line, or they can be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). In some embodiments, an embryo has not been destroyed in obtaining a pluripotent stem cell for use in the methods, assays, systems as disclosed herein.

In another embodiment, the stem cells, e.g., adult or embryonic stem cells can be isolated from tissue including solid tissues (the exception to solid tissue is whole blood, including blood, plasma and bone marrow) which were previously unidentified in the literature as sources of stem cells. In some embodiments, the tissue is heart or cardiac tissue. In other embodiments, the tissue is for example but not limited to, umbilical cord blood, placenta, bone marrow, or chondral villi.

Stem cells of interest for use in the methods, arrays, assays and kits as disclosed herein also include embryonic cells of various types, exemplified by human embryonic stem (hES) cells, described by Thomson et al. (1998) Science 282:1145; embryonic stem cells from other primates, such as Rhesus stem cells (Thomson et al. (1995) Proc. Natl. Acad. Sci USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also of interest are lineage committed stem cells, such as mesodermal stem cells and other early cardiogenic cells (see Reyes et al. (2001) Blood 98:2615-2625; Eisenberg & Bader (1996) Circ Res. 78(2):205-16; etc.).

### Drug Screening and Other Uses

Existing assays for drug screening/testing and toxicology studies have several shortcomings because they can include pluripotent stem cells which are poorly characterized and/or pluripotent stem cell lines which are abnormal or deviate from a typical pluripotent stem cell line in terms of its differentiation capacity and potential. Accordingly, by measuring m6A levels of a set of target genes as disclosed herein, one can identify and choose a stem cell line which is in an undifferentiated state which suitable for use in drug screening assay. Such identified stem cells then can be chosen for use in screening assays to screen a test compound and or in disease modeling assays.

Furthermore, the methods, arrays, assays and kits as disclosed herein are useful to determine the cell state of specific cell types from all developmental stages and even from blastocysts etc.

### Uses to Optimize Stem Cell Maintenance Media

In some embodiments, the methods, arrays, assays and kits as disclosed herein can be used to optimize culture media for maintaince and/or passage of stem cell populations in an undifferentiated state. For example, one can measure m6A levels (or peak intensities) of selected target genes selected from any listed in Table 1 and/or Table 2 in a stem cell population in the presence of different culture media and/or culture conditions, and using the m6A levels measured to assist in selecting the culture media and/or culture conditions which maintains the stem cell population in an undifferentiated state.

Accordingly, aspects of the present invention relate to culture media, e.g., culture media comprising a METTL3 and/or METTL4 inhibitor as disclosed herein for maintaining a stem cell population in an undifferentiated state. In some embodiments, the culture media is a cryopreservation culture media. By way of an example only, in some embodiments, the methods, arrays, assays and kits as disclosed herein can be used to confirm that a stem cell media, e.g., a pluripotent stem cell media maintains a stem cell in a pluripotent state and does not result in m6A modification which indicates that the stem cell lines is in an undifferentiated state.

Another aspect of the present invention relates to a container comprising a stem cell population, e.g. a human stem cell population in the presence of culture media comprising a METTL3 and/or METTL4 inhibitor as disclosed herein.

### EXAMPLES

Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

The developmental potential of human pluripotent stem cells suggests that they can produce disease-relevant cell types for biomedical research as well as cells for transplantation to address a disease. However, substantial variation has been reported among pluripotent cell lines, which could affect their utility and clinical safety. Disclosed herein are methods to maintain a stem cell line, e.g., human stem cell population in an undifferentiated state, and assays and arrays to assess the cell state of a stem cell population, e.g., if it is an undifferentiated state, and/or progressed along a lineage differentiation pathway.

In summary, the inventors have developed methods for maintaining human stem cell in an undifferentiated state, and assays and arrays to assess the cell state of a stem cell population in a rapid, cost effective, high-throughput method that is independent of gene expression levels.

### Methods and Materials

Mouse Cell Culture and Differentiation

J-1 murine embryonic stem cells were grown under typical feeder free ES cell culture conditions. For cardiomyocyte formation, mESCs were differentiated in cardiomyocyte differentiation media and scored on day 12. For neuron formation, mESCs were differentiated in MEF and ITSFn medium and scored after 10 days in ITSFn medium. For the cell proliferation assay 5000 cells where cultured in 24 well plates and the assay performed according to the manufacturer's protocol (MTT assay, Roche). For the single colony assays and Nanog staining, 1000 cells where cultured per well, on a six well plate. For alkaline phosphatase staining, cells were stained according to the manufacturer's protocol (Vector Blue Alkaline Phosphatase Substrate Kit).

mESC Cell Culture and Differentiation

J-1 murine embryonic stem cells were grown under typical feeder free ES cell culture conditions. Cells were grown in gelatinized (0.2% Gelatin) tissue culture plates in mESC media (KnockOut DMEM (Gibco, Life Technologies; 10829-018) supplemented with 1000 U/ml leukemia inhibitory factor (Millipore; ESG1107), lx non-essential amino acids (Gibco, Life Technologies; 11140-050), lx Glutamax (Gibco, Life Technologies; 35050-061), 10% Pen Strep (Gibco, Life Technologies; 151140-122) and 15% Fetal Bovine Serum (HyClone, SH30071.03)).

For cardiomyocite differentiation, mESCs were plated at a density of 2×10⁵ cells/mL in ultra-low attachment plates in cardiomyocyte differentiation media (CMD) (DMEM [GIBCO], 15% FBS [Hyclone], 1% penicillin/streptomycin, 1% GlutaMax and 1 mM Ascorbic Acid [Sigma]) to induce EB formation. Media was changed on day 3 and on day 6, EBs were re-suspended in fresh CMD media and replated on 0.2% gelatin coated dishes. Media was changed on day 9 and on day 12 the number of contracting patches of cells was quantified in triplicate for each cell line.

For Neuron differentiation, Mouse embryonic stem cells were grown in mESC medium (DMEM (Invitrogen), 12% knockout replacement serum (Invitrogen), 3% cosmic calf serum (Thermo Scientific) supplemented with non-essential amino acids (Invitrogen), penicillin-streptomycin (Invitrogen), sodium pyruvate (Invitrogen), 2-mercaptoethanol (Invitrogen) and LIF). Cells were dissociated in 2.5% trypsin for 5 minutes, pelleted, and resuspended on a gelatinized plate in MEF medium (DMEM, 10% cosmic calf serum, non-essential amino acids, penicillin-streptomycin, sodium pyruvate, 2-mercaptoethanol) for 30 minutes to remove feeders. 5×10̂6 mESCs were then replated onto 10 cm bacterial plates in MEF medium and cultured for 4 days. On day 4, cells were replated under adherent culture conditions. Medium was replaced with ITSFn medium (DMEM:F12 (Invitrogen), insulin [5 ug/ml], apotransferrin [50 ug/ml], sodium selenate [30 nM], fibronectin [250 ng/ml]) the following day and replaced every other day. Cells were cultured for 10 days in ITSFn before fixation.

For the cell proliferation assay (MTT) 5 thousand cells where cultured in 24 well dish and the assay performed according to the manufacturer's protocol (Roche; 11465007001). For the single colony assays and Nanog staining, 1 thousands cells where cultured, per well, on a six well dish.

For Alkaline Phosphatase Staining, at day 6 cells were fixed (50% Methanol, 50% Acetone) and stained for Alkaline Phosphatase with Vector Blue Alkaline Phosphatase Substracte Kit (Vector; 5300), according to manufacturer's protocol.

For Nanog and Oct4 staining cells where fixed with 4% paraformaldehyde (PFA) (Thermo Scientific, 28909). Cardiomyocites were cultured in chamber slides and fixed on day 12 with 4% PFA and N cells where fixed for 20 minutes in 4% PFA. Cells where washed 3 times with PBS and blocked in PBS with 0.1% Triton and 5% FBS (for N cells, CCS was used instead of FBS) for 20 minutes. Cells where then incubated with primary antibody [Rabbit anti-Nanog Antibody, Bethyl; mouse anti-Oct-3/4, Santa cruz, mMF20, Developmental studies Hybridoma bank; anti-Tuj1, Covance (1:1000), rabbit anti-Nanog, ReproCell (1:200)] for 30 minutes in blocking medium. After 3 PBS washes, cells where incubated with secondary antibody (Alexa 488 Goat anti-mouse, Alexa Goat anti-Rabbit, donkey Alexa-555 anti-mouse, donkey Alexa-488 anti-Rabbit (1:1000; Invitrogen)) in blocking medium. Cells where washed 3 times and Nuclei were counterstained with DAPI. Images where collected on a Zeiss Observer.Z1 using AxioVision software.

hESCs Cell Culture, Transfection and Differentiation

H1 (WA01) cells were cultured in feeder-free conditions as described (Sigova et al., 2013). Stable hESC lines were created that expressed shMETTL3 RNA or scrambled shRNA by transfecting hESCs with plasmids encoding shMETTL3 or scrambled shRNA and a puromycin resistance gene. Cells were treated with puromycin for six days beginning two days after transfection. For each shRNA, two independent puromycin-resistant colonies were picked and expanded. Endodermal differentiation was then induced by Activin A, as described (Sigova et al., 2013). Day 2 and Day 4 of differentiation were measured from the time that Activin was added. Puromycin was removed from the media one day prior to endodermal differentiation. Neuronal induction was induced through treated with potent and specific inhibitors of SMAD signaling.

H1 (WA01) cells were cultured in feeder-free condition using mTESR1 media (Stem Cell Technologies Cat.#05850) on 6-well plates coated with matrigel (BD Biosciences, Cat.#354603), as described (Sigova et al., 2013). Transfection of shMETTL3 RNA (DF/HCC DNA Resource Core Cat.#HsSH00253093) and scrambled shRNA (DF/HCC DNA Resource Core, pLKO-scramble, Cat.#EvNO00438085) was performed using Lipofectamine LTX (Life Technologies Cat.#25338100). Two days after transfection, cells were treated with 0.5 microgram per milliliter of puromycin (Life Technologies Cat.# A113802) for 6 days. For each shRNA, two independent puromycin-resistant colonies were picked from independent wells and expanded and Maintained under puromycin for analysis. Before Endodermal differentiation puromycin was withdrawn. Endodermal differentiation was then induced by resting cells in RPMI (Life Technologies Cat.#11875-093) with B27 supplement (Life Technologies Cat.#17504-044) for 24 hours followed by addition of Activin (R&D Systems), as described (Sigova et al., 2013). Day 2 and Day 4 of differentiation were measured from the time that Activin was added.

RNA Extraction, DNASE I Treatment and Poly a Selection

mESC total RNA was isolated from cells according to manufacturer's instructions using TRIzol reagent (Ambion). The RNA was re-suspended in ultrapure H₂O, treated with DNAse I (Ambion) for 30 min at 37° C. and subjected to RNA clean up reaction with RNeasy Midi Kit (Qiagen), according to manufacture's protocol. RNA was eluted in ultrapure H₂O. PolyA RNA selection was performed using MicroPoly(A) Purist (Life Technologies) according to the manufacturer's protocol. The second polyA RNA selection was performed using the eluate of the first polyA RNA selection as starting material according to the manufacture's instruction.

hESC total RNA was isolated from cells according to manufacturer's instructions using TRIzol LS reagent (Ambion). Total RNA was treated using DNAse I (Promega) for 20 minutes at 37° C. The treated RNA was then acid phenol/chloroform extracted and chloroform extracted. The RNA was precipitated using 300 mM final concentration of NaCl₂ spiked with 1 μl of 50 mg/ml of Ultra Pure Glycogen (Promega) and 2.5 volume of 100% ethanol at −20° C. either for 2 hours or overnight. The precipitated RNA was then centrifuged using a refrigerated table-top at maximum speed (>13,000 g) at 4° C. for 20 minutes. The precipitated RNA was then washed with 70° C. ethanol and centrifuged at maximum speed for an additional 10 minutes. The final pellet was then re-suspended in ultra pure H₂O. PolyA RNA selection was performed twice using Dynabeads mRNA Purification Kit (Invitrogen Cat. #610.06) according to the manufacturer's protocol. The second polyA RNA selection was performed using the eluate of the first polyaA RNA selection as starting material according to the manufacture's instruction. For all RNA samples, the concentration, purity and integrity of the RNA were verified using a NanoDrop and Bioanalyzer.

Immunofluorescence Staining

Cells were fixed with 4% paraformaldehyde (Thermo Scientific). Washes were performed with PBS. After blocking, cells were incubated with primary antibody in blocking medium. Cells were washed and incubated with secondary antibody in blocking medium. Nuclei were counterstained with DAPI.

RNA m⁶A IP

The detailed anti-m⁶A RIP and library preparation protocols are described in detail in the Extended Experimental Procedures. RNA was extracted with TRIzol (Ambion) according to manufacturer's protocol. After polyA RNA selection, RNA was fragmented in fragmentation buffer (10 nM ZnCl2, 10 mM Tris HCl, pH7.0). Fragmented RNA was incubated with anti-m⁶A polyclonal antibody (Synaptic Systems) and after extensive washing, bound RNA eluted. Input and anti-m⁶A polyclonal antibody enriched RNA were used to construct RNA libraries.

Mouse ESC Protocol 1—

PolyA+ RNA was purified with one round of selection with MicroPoly(A)Purist Kit (Ambion; AM1919). The PolyA+ RNA was fragmented to ˜100 nucleotide fragments by incubation with Zinc Chloride buffer (10 mM ZnCl2, 10 mM Tris-HCl, pH 7.0). After the RNA was incubated at 94° C. for 30 seconds, Zinc Chloride buffer, previously warmed to 94° C., was added and incubated for 2 minutes. The reaction was stopped with 0.2M EDTA, and the RNA precipitated with standard ethanol precipitation. 15 μg of anti-m6A polyclonal antibody (Synaptic Systems) were pretreated with agarose beads coated with ssDNA to reduced background (PMID:21472695). Antibody was conjugated to Dynabeads Protein G (Life Technologies; 10003D) overnight at 4° C. 200 μg of fragmented RNA were incubated with the antibody in 1×DamIP buffer (10 mM sodium phosphate buffer, pH 7.0, 0.3 M NaCl, 0.05% (w/v) Triton X-100) supplemented with 1% SuperRNAse Inhibitor (Ambion), for 3 hours at 4° C. After incubation, the antibody was washed 5 times with DamIP buffer and the RNA eluted with 0.5 mg ml-1 N6-methyladenosine (Sigma-Aldrich) in DamIP buffer (Xiao and Moore, 2011). 1 volume of Ethanol was added to the eluted RNA, and the RNA recovered an RNeasy mini column.

Library Construction:

The imunoprecipitated RNA, and an equivalent amount of input RNA where used for library generation with the dUTP protocol, as described (Levin et al., 2010) except libraries were size selected by gel purification after ligation and after PCR amplification. Libraries where sequenced using an Illumina HiSeq at the Stanford Center for Genomics and Personalized Medicine.

Mouse ESC Protocol 2—

Second set of libraries was generated as described in (Schwartz et al., 2013). Total RNA was subjected to two rounds of selection with MicroPoly(A)Purist Kit (Ambion; AM1919). 5 ug of RNA were fragmented as described above. After fragmentation RNA was incubated with 30 units of Polynucleotide Kinase in 50 mM Tris-HCl pH 7.6, 8 mM EDTA and 2 mM DTT. RNA was purified on a quiagen RNeasy column, and 10% was saved to be used as input. RNA was denatured and incubated with 25 ul of protein G beads (previously bound to 3 ug of anti-m6A polyclonal antibody (Synaptic Systems) in 1×IPP buffer (150 mM NaCl, 10 mM TRIS-HCL and 0.1% NP-40). After 3 hours, beads where washed 2 times with IPP buffer, 2 times with low salt buffer, 2 times with high salt buffer and 1 time with IPP buffer. RNA was eluted from the beads with 30 ul of RLT buffer, for 5 minutes. The RNA eluate was added to 20 ul of myone Silane beads re-suspended in 30 ul of RLT. 60 ul of Ethanol where added to the beads and incubated for 2 minutes. The beads where then washed 2 times with 70% Ethanol and the RNA eluted in 160 ul of IPP buffer. The eluted RNA was added to 25 ul of Protein A beads previously bound to 3 ug of anti-m6A polyclonal antibody (Synaptic Systems). After 3 hour incubation beads where washed and RNA eluted as described above. RNA was eluted in 100 ul of RNAse free water.

Library Construction:

After isolating fragmented m6A enriched RNA we constructed deep sequencing libraries as Rouskin et al. with the following modifications. RNA was first ligated to 25 pmol of pre-adenylated L3 (IDT) adaptor overnight at 16° C. The ligated samples were subjected to 8% PAGE separation, stained and imaged with SybrGold (Life Technologies) and ligated material was excised. The resulting gel slices were crushed and the RNA was eluted in 400 uL of Crush Soak Buffer (500 mM NaCl and 1 mM EDTA) and 5 uL of SUPERaseIn (Life Technologies) overnight at 4° C. Eluted RNA was purified with SpinX columns (Corning), precipitated, and reverse transcribed (RT) with RT oligos modified from the iCLIP method ((Konig et al., 2010), sequences below). cDNAs size selected on a 6% PAGE and eluted in 400 uL of Crush Soak Buffer at 50° C. overnight. Eluted cDNA was purified with SpinX columns, precipitated, and circularized using CircLigasell (Epicentre) for 2 hours at 60° C. in a 20 uL reaction. Circular cDNAs were purified with MiniElute columns and Buffer PNI (Qiagen) and eluted in 20 uL of EB Buffer. PCR amplification was performed in 50 uL reactions with 25 uL 2× Phusion High Fidelity Master Mix, 2.5 uL of 10 uM P3/P5 PCR primers (Ule, NSMB 2009/2010), and 22.5 uL of circularized cDNA. Samples required between 15-25 cycles of PCR. PCR reactions were purified using AMPure XP beads (Beckman) and final library DNA was eluted in 20 uL of water. Quantification was performed by BioAnalyzer analysis of the DNA, which was then sent for deep sequencing on an Illumina HiSeq2500 machine (Elim Biopharm, Hayward, Calif.).

Oligo and Adapater Sequences:

preA_L3/SrApp/AGA TCG GAA GAG CGG TTC AG (SEQ ID NO: 661) /3ddC/; P5 AAT GAT ACG GCG ACC ACC GAG ATC TAC ACT CTT TCC CTA CAC GAC GCT CTT CCG ATC T (SEQ ID NO: 662); P3 CAA GCA GAA GAC GGC ATA CGA GAT CGG TCT CGG CAT TCC TGC TGA ACC GCT CTT CCG ATC T (SEQ ID NO: 663); RToligol (Barcode) /5phos/NNN NNA ACC NNN NAG ATC GGA AGA GCG TCG TGA (SEQ ID NO: 664) T/iSp18/GGATCC/iSp18/TACTGAACCGC (SEQ ID NO: 665).

Human ESC Protocol:

Of note for each biological replicate for m⁶A-seq, we started with 400 □g of total RNA yielding approximately 10 μg of double polyA selected RNA which was re-suspended in a final volume of 50 μl using UltraPure H₂O (Life Technologies). 250 μl of digestion/fragmentation buffer (10 nM ZnCl₂, 10 mM Tris HCl, pH7.0) was added to the 50 μl of 2× polyA RNA. The 300 μl of PolyA RNA/fragmentation buffer was heated at 94° C. for exactly 5 minutes. 50 μl of 0.5M EDTA was added to stop the fragmentation reaction and immediately put on ice.

The 2× polyA fragmented RNA was then heated at 65° C. for 5 minutes and immediately put on ice. 50 μl of m⁶A-DynaBeads (The m⁶A antibody-Synaptic Systems was coupled to Dynabeads using the Life Technologies coupling kit cat#14311D) were equilibrated by washing twice for 5 minutes in 500 μl of m⁶A-Binding Buffer (50 mM Tris-HCl, 150 mM NaCl2, 1% NP-40, 0.05% EDTA). The RNA was then added to the equilibrated m⁶A-DynaBeads. The RNA was allowed to bind to the m⁶A-Dynabeads (in 500 μl volume of m⁶A-Dynabeads/m⁶A-Binding Buffer at room temperature while rotating (tail-over-head) at 7 rotations per minutes for 1 hour. The tubes containing the samples were placed on a magnet allowing the beads complexes to cluster for one minute or until the solution become clear. The liquid phase was carefully collected and placed on ice as this 500 μl fraction represents the “Supernatant” of the m⁶A IP. Following the collection of the supernatant fraction, series of washes were performed using various buffers (see as follow). For all wash steps to the exception of the elution step, the beads were washed 3 minutes then place on a magnet and the wash buffers were discarded. Following the supernatant collection. Wash step 1: The reminding fractions bound to the beads were washed twice in 500 μl of m⁶A-Binding Buffer (Tris-HCl 50 mM, NaCl₂ 150 mM, NP-40 1%, EDTA 0.05%). Wash Step 2: The RNA/beads complexes were washed once in 500 μl of Low Salt Buffer (SSPE 0.25×, EDTA 0.001M, Tween-20 0.05%, NaCl 37.5 mM). Wash Step 3: The RNA/beads complexes were washed once in 500 μl of High Salt Buffer (SSPE 0.25×, EDTA, 0.001M, Tween-20 0.05%, NaCl 137.5 mM). Wash Step 4: The RNA/beads complexes were washed twice in 500 μl of in TET (T.E.+0.05% Tween-20). Elution Step: The m⁶A-RNA was eluted from the beads by repeating four times the following: 125 μl of Elution Buffer (DTT 0.02M, NaCl 0.150M, Tris-HCl pH7.5 0.05M, EDTA 0.001M, SDS 0.10%) was added to the beads and incubated at 42° C. for 5 minutes. At the end of the 5 minutes the beads were gently vortexed and placed on the magnet. The liquid phase was collected and transferred to a fresh tube as this will represent the eluate fraction containing the m⁶A “enriched RNA”. An additional 125 μl of elution buffer was then added to the beads and the processed was repeated. The liquid phase obtained at each step was added to the “fresh tube” containing the 125 μl of eluate from the previous step so the total final eluate volume was 500 μl.

All RNA fractions were extracted as follow. 500 μl of acid phenol-chloroform (acid-phenol:chloroform, pH 4.5 (with IAA, 125:24:1) Ambion) were added to the 500 μl sample. The sample was centrifuged at 4° C. at 10,000 g for 7.5 minutes. The upper phase was carefully collected making sure not to touch the inter-phase and transfer to a clean 1.5 ml tube. 500 ml of chloroform was added to the fresh tube vortexed briefy and centrifuged at 4° C. at 10,000 g for 7.5 minutes. The upper phase was transferred to a fresh 1.5 ml tube and NaCl₂ ethanol precipitated overnight at −20° C. in presence 1 μl of (20 mg/ml) Ultra Pure Glycogen. The following day the sample was centrifuged at 4° C. for 20 minutes at 16,000 g. The pellet was then washed in 70% ethanol centrifuged and additional 10 minutes at 4° C. at 16,000 g. The pellet was then let to dry at room temperature for 10 minutes prior to be re-suspended in the desired volume of Ultra-Pure H₂O (Invitrogen Cat#10977-015).

Library Construction:

100 ng (100 ng of input and 100 ng of post m⁶A-IP positive fraction) were used for library construction and RNAseq using TrueSeq Stranded mRNA Sample Preparation Guide, entering the protocol by adding the Fragment, Prime, Finish Mix, skipping the elution step and proceeding immediately to the synthesis of the First Strand cDNA. From that point on, the exact steps of the Illumina TruSeq Stranded mRNA sample Preparation Guide were followed to the end. RNA Sequencing. Each individual library fragment size was verified on Agilent Bioanalyzer 2100 with High Sensitivity chip. Final quantification was done by qPCR on Perkin Elmer 2500Fast with Kapa library quantification kit (#KK4824). Libraries were pooled at equimolar concentrations according to the manufacturer guidelines (TruSeq Stranded mRNA Sample Preparation Guide—September 2012). After clustering on Illumina cBot, samples were run on Illumina HiSeq 2000.

For m6AIP-RT-qPCR, and m6AIP-Nanostring, experiment were performed as described above (protocol 1), except 2 ug of fragmented RNA, and 1 ug of antibody were used. Rabbit IgG was used as a non-specific antibody control for immunoprecipitation in parallel to the anti-m6A polyclonal antibody (Synaptic Systems).

Real Time PCR

For the mouse experiments, RNA was analyzed on a LightCycler 480 by RT-qPCR with One-Step RT-PCR Master Mix SYBR Green (Stratagene). For gene expression experiments, each PCR reaction was performed in 12 μl with 45 ng of total RNA, 0.8 μl of RT block/enzyme mixture, 1.2 μl primers at 1.25 μM each and 6 μl of MasterMix (final volume 12 μl). The PCR was carried on using a standard protocol with melting curve. The amount of target were calculated using the formula: Amount of target=2^{-ΔΔC(T)} (Livak and Schmittgen, 2001). Two tailed T test for unequal, unpaired data sets with heteroscedastic variation was used to compare samples. Primer sequences available upon request.

For human experiments, a first mixed made of 10 pg to 5 μg of RNA in 5 μl volume, 411 of random hexamers (Roche), 1 μl of dNTPmix (10 mM each) and 5 μl of ultrapure H₂O was first generated, heated at 65° C. for 5 minutes and immediately put on ice. 4 μl of 5× First Strand Buffer was added along with 1 μl of 0.1M DTT, 1 μl RNAse inhibitor and 1 μl of Superscript III reverse transcriptase (Invitrogen). The 20 μl reverse transcription reaction was then incubated 5 minutes at room temperature, then 60 minutes at 50° C. then 15 minutes at 70° C. The freshly synthesized cDNA was treated with 1 μl of RNAse H at 37° C. for 20 minutes. For Sybergreen quantitative real time PCR assays, each PCR reaction was done in a 20 μl volume made of 10 μl of master mix (SYBR GreenER qPCR SuperMix for iCycler-Invitrogen), 5 μl of primer mix at 1.2 μM (each) and 5 μl of cDNA template at 20 ng/μl. The PCR was carried on using a standard protocol with melting curve. The amount of target were calculated using the formula: Amount of target=2^{-ΔΔC(T)} (Livak and Schmittgen, 2001). The qPCR using Taqman reagents was done in a 10 μl volume made of 5 μl of Universal PCR Master Mix (Applied Bosystems Cat.#4304437), 0.5 μl of TaqMan probe mix (each), 2 μl of cDNA template at 50 ng/μl and 2.5 μl of H₂O. The PCR was carried on using a standard protocol with melting curve. The amount of target were calculated as above. The TaqMan probes were purchased from Applied Biosystems; 18s (AB Hs99999901_s1), FOXA2 (AB Hs00232764_m1), SOX17 (AB Hs 00751752_s1), NANOG (AB Hs 02387400_g1), and SOX2 (AB 010533049_s1).

RNA Stability Assay

Wild type and Mettl3 KO cells were treated with 0.8 μM Flavopiridol for 3 hours. RNA extraction and qRT_PCR as described above.

shRNAs Targeting shRNAs

Short Hairpin RNAs targeting the mouse Mettl3 sequences GCACACTGATGAATCTTTA (SEQ ID NO: 658) and GCACTTCCTTACAAAGCT (SEQ ID NO: 659) were generated in the pSicoR plasmid backbone (Addgene 12084, (Ventura et al., 2004)). The plasmid pSicoR shluc (Addgene 14782, (Konig et al., 2010)) was used as a negative control. The plasmids were co-transfected into 293T cells with pMd2G and psPAX2 with Fugene HD (Promega, E2311) according to manufacturer's instructions. Virus where collected after 48 hours. The collected media was filtered through a 0.45 μm membrane and the virus concentrated with Lenti-X concentrator (Clontech; 631231). J-1 mESC cells were infected in the presence of 2 μg per ml polybrene. After 24 hours, cells where selected with puromycin. After selection, cells where replated at low density and single clones where collected. Real time PCR was used to choose determine efficiency of the Knock Down.

The shRNA hairpins targeting human Mettl3 were purchased from DF/HCC DNA Resource Core. Multiple sh clones were purchased against METTL3 (HsSH00253093, HsSH00253439, HsSH00253446, HsSH00253487, HsSH00253494). After testing of their individual knockdown efficiency both by qRT-PCR and anti-METTL3 western blot in 293T, we identified number HsSH00253093 (insert Sequence: CCG GGC TGC ACT TCA GAC GAA TTA TCT CGA GAT AAT TCG TCT GAA GTG CAG CTT TTT (SEQ ID NO: 660); Target Sequence: GCTGCACTTCAGACGAATTAT; SEQ ID NO: 3) as giving optimal knockdown and this was used to generate H1-ESCs knockdown cell lines. The scrambled shRNA control pLKO-Scramble (Cat# Ev000438085) was also obtained from the DF/HCC DNA Resource Core.

CRISPR-Mediated Mettl3 Knockout

gRNA sequences where chosen and designed a CRISPR design tool (Hsu et al., 2013). Plasmids for guide RNA were co-nucleofected (Lonza; VPH-1001), with a human codon optimized Cas9 expression plasmid and a plasmid with a puromycine resistance cassette. Cells were plated at low density for single colony isolation and selected single colonies tested by western blot for loss of protein. More specifically, RNA sequences where chosen and designed from CRISPR design tool (Hsu et al., 2013). DNA blocks containing all of the components necessary for gRNA expression (Mali et al., 2013) were synthesized by IDT and cloned in Topo-Blunt plasmid (Invitrogen). Plasmids for guide RNA were co-nucleofected (Lonza; VPH-1001), according to manufacturer's instructions, with a human codon optimized Cas9 expression plasmid and a plasmid with a puromicine resistance cassette. Cells were plated at low density for single colony isolation. The remaining cells were cultured for surveyor assay. After 24 hours, cells were selected with puromicine for 48 hours. DNA extraction and surveyor assay as described in (Cong et al., 2013). Single colonies where selected and tested by western blot for loss of Protein. DNA sequencing of the targeted locus was used to confirm presence of mutations that abrogate protein production.

Annexin V Analysis

Cells were labeled with Live/Dead Fixable Aqua (Life Technologies) and fluorochrome conjugated Annexin V. Samples were analyzed on a special order FACS Aria II (BD Biosciences). More specifically, one million cells were collected and washed twice with PBS. The cells were incubated with 1 μl of Live/Dead Fixable Aqua (Life Technologies) for 30 minutes, protected from light. The cells were then washed twice with FACS buffer and re-suspended in 1× Binding buffer followed by an incubation with 5 μl of fluorochrome conjugated Annexin V for 15 min. The cells were washed once with FACS buffer and resuspended in 500 μl of Binding buffer. Samples were analyzed on a special order FACS Aria II (BD Biosciences).

Western Blot

Cell extracts where resolved on a NuPAGE 4-12% Bis-Tris Mini Gel and transferred to Immobilon-FL membrane. Images were collected on a Licor Odyssey imaging system. More specifically, cells were collected and lysed in RIPA buffer (400 mM NaCl, 1% Igepal, 0.5% Sodium Deoxycholate, 0.1% SDS and 10 mM Tris-Cl pH 8.0) for 30 min on ice. The lysate was centrifuged for 10 minute and the supernatant collected. Protein was quantified with BCA Protein Assay Kit (Pierce). Proteins where resolved on a NuPAGE 4-12% Bis-Tris Midi Gel and transferred to Immobilon-FL membrane. Primary antibodies used are: (Rabbit anti-METTL3/MT-A70, Bethyl A301-568; Mouse anti-beta actin, mAbcam 8224 and Rabbit anti-PARP, Cell Signaling, 9542). Secondary antibodies used: IRDye 680RD Goat anti-Mouse IgG (H+L) (Licor) and IRDye 800CW Goat anti-Rabbit IgG (H+L) (Licor). Images where collected on a Licor Odyssey imaging system.

Determination of m⁶A Levels

2D-TLC was performed as described by (Jia et al., 2011). For dot-blots, the indicated amounts of RNA were applied to the membrane and cross-linked by UV. The m⁶A primary antibody was then added to the blocked membrane at a concentration of 1:500. The membrane was incubated with the secondary antibody and exposed to an auto-radiographic film. m⁶A RNA mass-spectrometry was performed as described in the Extended Experimental Procedures. More specifically, 2D-TLC was performed as described by (Jia et al., 2011). 100 to 200 ng of polyA+ RNA, selected for two rounds, was digested with 2000 units of RNAse T1 (Ambion) in a final volume of 25 μl, with 1×PNK buffer and incubated at 37° C. for 1 hour. The RNA was labeled with 10 units of PNK (NEB) and 1 μl [Γ-32P]ATP (6000 Ci/mmol; Perkin-Elmer). The reaction was cleaned with a G25 column and precipitated with Standard Ethanol precipitation. The RNA was re-suspended in 10 μl of 50 mM sodium acetate (pH 5.5) and digested with 1 Unit of nuclease P1 (USBiological; N7000). 1 μl was loaded on a Cellulose TLC glass plate (EMD chemicals; 5716-7). The first dimension was resolved in isobutyric acid:0.5 M NH4OH (5:3, v/v) and the second dimension resolved in isopropanol:HCl:water. The plates were exposed on a phosphor screen and scanned on a GE typhoon TRIO at the Stanford Functional Genomics Facility.

m⁶A Level Dot-Blots

Amersham Hybond-XL (Cat.# RPN303s) membrane was rehydrated in H₂O for 3 minutes. The membrane was then “sandwiched” in Bio-Dot Microfiltration Apparatus (BioRad, cat. #170-6545). Each well was then filled with H₂O and flushed by gentle suction vacuum until it appeared dry. 5 μl of H₂O alone was then applied to the membrane in each well followed by addition of indicated amount of RNA and this was allowed to bind to the membrane by gravity. The apparatus was disassembled and the membrane was cross-linked in a UV STRATALINKER 1800 using the automatic function and then the membrane was placed back into the apparatus. The membrane was then blocked 10 minutes using sterile RNAse DNase free TBST+5% milk. The m⁶A primary antibody (Anti-m⁶A, Synaptic Systems, Cat. #202 003) was then added at a concentration of 1:500 at room temperature for 1 hour in TBST+5% milk. The membrane was then washed four times in PBST. The membrane was then incubated with the secondary anti rabbit antibody (1:5000 dilution) for 30 minutes in TBST+5% milk. The membrane was washed 4 times 5 minutes in TBST and expose on an auto radiographic film using Pierce ECL Western Blotting Substrate.

Mass Spectrometric Quantification of m6A

Enzymatic hydrolysis of RNA to ribonucleosides was carried out as described previously, (Taghizadeh et al., 2008) with modifications. Following addition of 100 nM [¹⁵N]-ethenocytidine and 10 μM [¹⁵N]-guanosine as internal standards for m⁶A and adenosine respectively (due to similar masses and retention times), RNA (200 ng) was digested with 2 U nuclease P1 (Sigma Aldrich, St. Louis, Mo.) at 37° C. for 3 h in 55 μl in buffer containing 16 mM sodium acetate (pH 6.8), 1.8 mM zinc chloride, 9 μg/mL coformycin, 45 μg/mL tetrahydrouridine, 2.3 mM desferroxamine, 0.45 mM butylated hydroxytoluene, followed by addition of 45 μl of 27 mM of sodium acetate (pH 7.8), 17 U calf thymus alkaline phosphatase (New England Biolabs, Ipswich, Mass.) and 0.1 U snake venom phosphodiesterase (Sigma Aldrich) with incubation overnight at 37° C. The digestion mixture was later deproteinized by centrifugal filtration (Nanosep 10K; Pall Corporation, Port Washington, N.Y.), and 10 μl of the mixture was analyzed by a liquid chromatography-coupled triple quadrupole mass spectrometry (LC-QQQ). HPLC was performed on an Agilent series 1200 instrument (Agilent Technologies, Santa Clara, Calif.) consisting of a binary pump, a solvent degasser, a thermostatted column compartment and an autosampler. The nucleosides were resolved on a Dionex Acclaim PolarAdvantage C16 column (3 μm particles, 120 Å pores, 2.1×150 mm; 30° C.) at 300 μL/min using a solvent system consisting of 0.1% acetic acid in H₂O (A) and 0.1% acetic acid in acetonitrile (B), with the elution performed isocratically at 0% B for 29 min, followed by a column washing at 70% B and column equilibration. Mass spectrometry detection was achieved using an Agilent 6410 QQQ mass spectrometer in positive electrospray ionization mode with the following parameters: ESI capillary voltage, 3000 V; gas temperature, 340° C.; drying gas flow, 10 L/min; nebulizer pressure, 20 psi; fragmentor voltage, 150 V. The nucleosides were quantified using the nucleoside→base ion mass transitions of 282.1→150.1 (m⁶A), and 268.1→136.1 (A). Absolute quantities of m⁶A and A were determined from calibration curves prepared daily.

Microarray Data Acquisition and Data Analysis.

RNA was extracted as described above and submitted for Hybridization on GeneChip Mouse Exon 1.0 ST Array at the Protein and Nucleic Acid Facility of the Stanford School of Medicine. For gene expression analysis, arrays were RMA normalized using justRMA package in R. After normalization, probes with average expression of all arrays less than 100 were filtered out as not expressed probes. For each expressed probe, its expressions were log 2ed, and the gene expression was defined as the average expression of all the expressed probes that attached to this gene. Student T-test comparing wide-type versus knockout signals in the arrays were used to calculate the significance of the expression changes, and false discovery rate (FDR) was estimated using p.adjust package in R. Differential expression was defined using the following filters: significance analysis of microarrays 3.0 (Tusher et al., 2001) with a false discovery rate less than 5%, an average fold change≧2 in any group, and an average raw expression intensity≧100 in any group.

m⁶A Methylation IP RNA-Sequencing Analysis

Libraries generated with iCLIP adaptors where separated by barcode, and perfectly matching reads were collapsed. Sequencing reads were mapped using TopHat (Trapnell et al., 2009). A non-redundant mm9 transcriptome was assembled from UCSC RefSeq genes, UCSC genes, and predictions from (Ulitsky et al., 2011) and (Guttman et al., 2011). For human datasets, the Ensembl genes (release 64) was used. Search for enriched peaks was performed by scanning each gene using 100-nucleotide sliding windows, and calculating an enrichment score for each sliding window (Dominissini et al., 2012). HOMER software package (Heinz et al., 2010) was used for de novo discovery of the methylation motif. More specifically, libraries generated with iCLIP adaptors (mouse, protocol 2) where separated by barcode, and perfectly matching reads were collapsed and barcodes removed. For all libraries, single-end RNA-Seq reads were mapped to the mouse (mm9 assembly) of human genome (hg19 assembly) using TopHat (version 1.1.3) (Trapnell et al., 2009). Only uniquely mapped reads were subjected to downstream analyses.

The mouse RNA-seq reads, recorded in BAM/SAM format were transformed to bedGraph format, indicating the number of reads on each genomic position. A non-redundant mm9 transcriptome was assembled from UCSC RefSeq genes, UCSC genes, and predictions from (Ulitsky et al., 2011) and (Guttman et al., 2011). Gene expression in the form of RPKM was calculated using a self-developed script.

For human RNA-seq reads, FPKMs of Ensembl genes (release 64) were calculated using Cufflinks (version 2.0.2) (Trapnell et al., 2010) and differentially expressed genes between input RNAs of T0 and T48 were determined by Cuffdiff (version v2.0.2) (Trapnell et al., 2013).

To make UCSC read coverage tracks, the read coverage at each single nucleotide was normalized to library size for input and eluate (m⁶A RIP) respectively. For human samples, we normalized the read densities by adjusting the library sizes (total uniquely mapped reads) to be the same (average total uniquely mapped reads of initial sequencing runs of 4 samples) for input and eluate (m⁶A RIP) respectively. The average normalized read densities of replicates A and B were shown in the Figures.

m6A Peak Calling and Intensity Calling and Analysis

Search for enriched peaks was performed by scanning each gene using 100-nucleotide sliding windows, and calculate an enrichment score for each sliding window (Dominissini et al., 2012). Windows with RPKM≧5 in the eluate, enrichment score≧2 in genes with RPKM in the input sample≧1 were defined as enriched in m6A pull down. Enriched windows with score greater than neighboring windows where selected as m6A peaks. To determine “high-confidence”, we first intersected the peaks in biological replicates, requiring at least 0.5 overlap using the BedTools package (Quinlan and Hall, 2010). Peaks that did not intersect where merged, and peaks that merged end to end where also kept for downstream analysis. The peaks where re-defined as 100 nt windows centered at the middle of the intersected/merged peaks. For Human m6A peak detection, eluate window RPKM≧10 instead of 5 were used. Common peaks were determined in the same way as described in mouse. For each time point, the common peaks of the two replicates were referred to as “high-confidence” peaks.

To study the peak distributions on transcripts, the inventors assigned each “high-confidence” peak (using middle point) to the collapsed transcript (mouse) or to the longest isoform of each Ensembl gene. 100 bins of equal length were made for 5′UTR, CDS and 3′UTR respectively and the average number of peaks for each bin was calculated. The peak intensity was calculated as the ratio of window RPKM between eluate and input for each peak. To compare the peak intensities between two samples, we used sample specific peaks as well as common peaks and required input window RPKM≧20 to obtain reliable peak intensity values.

More specifically, the inventors searched for m6A peaks by scanning each gene using 100-nucleotide sliding windows, and calculate an enrichment score for each sliding window (Dominissini et al., 2012). Windows with RPKM≧5 and RPKM≧10 for mouse and human respectively were used. A enrichment score≧2 in genes with RPKM in the input sample≧1 were defined as enriched in m6A pull down. Enriched windows with score greater than neighboring windows where selected as m6A peaks. To determine “high confidence”, we first intersected the peaks in biological replicates, requiring at least 0.5 overlap using the BedTools package (Quinlan and Hall, 2010). Peaks that did not intersect where merged, and peaks that merged end to end where also kept for downstream analysis. The peaks where re-defined as 100 nt windows centered at the middle of the intersected/merged peaks. For each time point, the common peaks of the two replicates were referred to as “high-confidence” peaks. The peak intensity was calculated as the ratio of window RPKM between eluate and input for each peak. To compare the peak intensities between two samples, the inventors used sample specific peaks as well as common peaks and required input window RPKM≧20 to obtain reliable peak intensity values.

Comparing Mouse and Human Peaks.

The inventors common peaks of 3 mESC samples and common peaks of 2 hESC samples for mouse and human ESC m6A comparison. To compare the methylated genes between mESC and hESC at gene level, only Ensembl genes with the annotated one to one ortholog between human and mouse were considered in the comparison, and the genes must have gene expression value (RPKM or FPKM) greater than 1 in all samples of both hESC and mESC. To compare the m6A peak intensities between human and mouse ESCs, the inventors aligned all the mESC peaks to human genome based on the UCSC pairwise genome alignment (http://hgdownload.soe.ucsc.edu/), the orthologous mouse-human regions of merged peaks (at least 1 bp overlap) and species specific peaks were used for the comparison. For merged peaks, the inventors took the center 100 bp regions and only used those had window.

A gene's enrichment score was defined as the maximum enriched window in this gene. HOMER software package (Heinz et al., 2010) was used for de novo discovery of the methylation motif, using the high confidence peaks. Random windows for control where obtained using the BedTools package (Quinlan and Hall, 2010).

GO (Gene Ontology) analyses for methylated genes were conducted using DAVID (Huang da et al., 2009) with genes with RPKM≧1 (mouse) or FPKM≧1 (human) as background.

Fingerprinting m6A During Endoderm Differentiation (Similar Strategy for any Comparison in Same Organism would Apply)

To determine the amount of dynamic regulation or extent of differential m6A peaks during differentiation in hESC, the m6A peaks of undifferentiated ESCs (T0) and after 48 hours of differentiation (T48) that that meet the following criteria between T0 and T48 were identified: 1) Input gene FPKM≧1 in all 4 samples; 2) Input window RPKM≧10 in all 4 samples; 3) At least 1.5 fold (or 2 fold) change of peak intensities in both replicates in the same direction; 4) The maximum peak intensity of all samples≧2; 5) In each replicate, the sample with higher peak intensity must be called as having peak. To determine the union of m6A peaks of T0 and T48, the inventors pooled all the peaks of the samples and merged the same peaks and peaks with 50 bp overlapped, the unmerged peaks were then merged if they were end-to-end peaks spanning 200 bp. The inventors took the center 100 bp of merged peaks as union peaks if they meet the following criteria in either T0 or T48: 1) both replicates had the peaks; 2) The center 100 bp had window score≧2 in both replicates. Subsequently a heatmap and clustering analysis was performed. The heatmaps of all samples were made based on Z score scaled log 2 values for peak intensities. For peak intensity analysis, the peaks and samples were clustered using 1-Pearson correlation coefficient of log 2(peak intensity) as the distance metric.

Dataset Comparison

Mouse Pol II occupancy data, mRNA half life and Protein translation efficiency were obtained from (Ingolia et al., 2011; Rahl et al., 2010; Sharova et al., 2009) Plotting and statistical tests were performed in R. Multi-dimensional gene set enrichment analysis over DAVID Gene Ontology terms and stem cell gene sets (Wong et al., 2008) were performed using Genomica (Segal et al., 2005; Segal et al., 2004; Segal et al., 2003). A P-value of <0.01 from a hyper geometric test between a gene group and gene set was defined as significant.

More specifically, Pol II occupancy, obtained from (Rahl et al., 2010), at transcriptional start sites was determined using an in-house developed script based on annotations downloaded from the UCSC table browser. Mouse mRNA half life and Protein translation efficiency was extracted from (Ingolia et al., 2011; Sharova et al., 2009) for genes with RPKM>=1 in the input. Plotting and statistical test performed in R. For genes with multiple Half life values reported, the average value was used. We obtained human mRNA half-life of induced pluripotent stem (IPS) cells from published thesis (Neff et al., 2012). The m6A enrichment score was calculated as the maximum window scores of all windows of each gene including unmethylated genes, the windows with input window RPKM<1 were removed from the calculation.

Gene Set Enrichment Analysis

Genes were ranked by their enrichment score, and equally divided into 10 groups. For each group, a multi-dimensional gene set enrichment analysis over DAVID Gene Ontology terms and stem cell gene sets

(Wong et al., 2008) was performed using Genomica (Segal et al., 2005; Segal et al., 2004; Segal et al., 2003). A P-value of <0.01 from hyper geometric test between a gene group and gene set was defined as significant.

Determination of Differentially Methylated Peaks

To determine effects of Mettl3 loss of function on m6A peaks, we calculated the peak intensity for the high confidence peaks identified in wild type cells. Peaks with significant changes in peak intensity (p.value<0.05) where considered for further analysis. To determine the effect of differentiation in hESC, the union of m⁶A peaks of T0 and T48 (initial sequencing run, with comparable sequencing depth for both time points) were analyzed to determine the differentially methylated peaks between T0 and T48 that meet the following criteria: 1) Input gene FPKM≧1 in all 4 samples; 2) Input window RPKM≧10 in all 4 samples; 3) At least 1.5 fold (or 2 fold) change of peak intensities in both replicates in the same direction; 4) The maximum peak intensity of all samples≧2; 5) In each replicate, the sample with higher peak intensity must be called as having peak. To determine the union of m6A peaks of T0 and T48, we pooled all the peaks of 4 samples and merged the same peaks and peaks with 50 bp overlapped, the unmerged peaks were then merged if they were end-to-end peaks spanning 200 bp. We took the center 100 bp of merged peaks as union peaks if they meet the following criteria in either T0 or T48: 1) both replicates had the peaks; 2) The center 100 bp had window score≧2 in both replicates.

Heatmap and Clustering Analysis

Heatmaps of all 4 samples were made based on Z score scaled log 2 values for peak intensities or gene expression levels (FPKMs) respectively. For analysis of the differentially expressed genes, the genes and samples were clustered by average linkage hierarchical clustering using 1-Pearson correlation coefficient of log 2(FPKM) as the distance metric. For peak intensity analysis, the peaks and samples were clustered in the same way using 1-Pearson correlation coefficient of log 2(peak intensity) as the distance metric.

Analysis of m6A Sites in Non-Coding RNAs

The longest isoforms of Ensembl genes were used to study the distribution of m6A peaks on coding and noncoding transcripts. Noncoding transcripts overlapping with any isoforms of coding genes were removed, and transcripts with less than 3 exons were also removed. The analysis used the peaks found wild type mESC cells or the union of H1 T0 (all data), H1 T48, 293T, HepG2 (including stimulated samples) and human brain (Dominissini et al., 2012; Meyer et al., 2012). To study the m6A peak distributions on transcripts, in each transcript we made 10 bins of equal length for the first exon, internal exons and the last exon respectively, and the percentage of peaks in each bin was calculated for coding and noncoding transcripts. Additionally, the peak coverage around the last exon-exon splice junction was also analyzed for coding and noncoding transcripts. The peaks used in this analysis included the wild type mESC or H1 T0 (all data), H1 T48, 293T, HepG2 (including stimulated samples) and human brain (Dominissini et al., 2012; Meyer et al., 2012). The peak coverage (number of peaks covering the site) normalized by the total number of overlapped peaks was calculated for the 750 bp regions flanking the last splice junction. Therefore, the transcripts with less than 750 bp on either side were also removed from the analysis.

Exon Length Analysis

Middle points of all high-confidence peaks in the two time points were assigned to exons of the longest isoforms of Ensembl coding genes. Only internal exons were used in the subsequent analysis. Exon length and number of m6A motifs were used to normalize the number of peaks in each exon. Error bar indicates variations estimated via 1000 times of bootstrapping for each bin of exon length.

Single Exon Gene Analysis

Ensembl genes without any multi-exon isoforms were considered as single exon genes. The peak distribution of the longest isoform of single exon protein-coding genes was analyzed in the same way as for multi-exon protein-coding genes, except that 10 bins were made for each 5′UTR, CDS and 3′UTR.

Comparison of m6A Peaks Between Mouse and Human ESCs

We used common peaks of 3 mESC and common peaks of 2 hESC for mouse and human ESC m6A comparison. To compare the methylated genes between mESC and hESC at gene level, only Ensembl genes with the annotated one to one ortholog between human and mouse were considered in the comparison, and the genes must have gene expression value (RPKM or FPKM) greater than 1 in all samples of both hESC and mESC. To compare the m6A peak intensities between human and mouse ESCs, we aligned all the mESC peaks to human genome based on the UCSC pairwise genome alignment (http://hgdownload.soe.ucsc.edu/), the orthologous mouse-human regions of merged peaks (at least 1 bp overlap) and species specific peaks were used for the comparison. For merged peaks, we took the center 100 bp regions and only used those had window scores≧2 in all samples of both species. Only Ensembl genes with the annotated one to one orthologs between human and mouse were considered. To obtain reliable peak intensity values, we required gene RPKM or FPKM≧1 and input window RPKM≧5 in all samples of both species.

GRO-Seq Analyses and RNA Polymerase II Traveling Ratio Calculation

GRO-seq data for hESCs (replicate 1-3) and GRO-seq data for 48 hours of endodermal differentiation (replicate 1) (Sigova et al., 2013) (GSE 41009) were analyzed. FASTQ files were mapped to hg19 using Bowtie2 with the parameters −k2−L24−N1—local. Calculation of the traveling ratio was adapted from (Rahl et al., 2010). Briefly, each gene was divided into the proximal promoter and gene body. The proximal promoter was defined as the region from 30 bp upstream to 300 bp downstream of the transcription start site. The gene body was defined as 300 bp downstream of the TSS to the end of the annotated gene. The number of GRO-seq reads that mapped to the promoter proximal region and gene body was determined for each gene in each experimental condition. The total number of reads mapped to each region was divided by the length of the region to determine the read density. The RNA polymerase II traveling ratio (TR) was calculated for each gene by dividing the density of the promoter proximal region by the density of the gene body region.

Analysis of the Relationship Between m⁶A and RNA Polymerase II Travelling Ratio

To compare the m⁶A peak intensity and RNA polymerase II travelling ratio, the m6A enrichment score was calculated as the maximum window scores of all windows of each gene including unmethylated genes, the windows with input window RPKM<1 were removed from the calculation.

Teratoma Generation and Histopathology

Mettl3 wild type and mutant cells (2.5×10̂6) were subcutaneously injected into 8-week-old female SCID/Beige mice (Charles River). In the fourth week after injection, the mice were euthanized and the tumors were harvested, weighed, measured and processed for histological analysis. All animal studies were approved by Stanford University IACUC guidelines. For histological analysis, slides were stained with hematoxylin and eosin (H&E); or stained by immunohistochemistry (IHC) with VECTASTAIN ABC Kit (PK-4000, Vector laboratories) and DAB Peroxidase Substrate Kit (SK-4100, Vector laboratories) following the manufacturer's instructions. Analyses were performed by a boarded veterinarypathologist (DMB).

Mettl3 wild type and mutant cells were trypsinized and 2.5×10̂6 cells were subcutaneously injected into 8-week-old female SCID/Beige mice (Charles River). Teratoma progression was monitored by volume measurement every other day after a visible tumor mass formed. In the fourth week after injection, the mice were euthanized and the tumors were harvested, weighed, measured and then were processed for histological analysis. All the animal studies were approved by Stanford University IACUC guidelines.

For histological analysis, teratomas were fixed with 4% paraformaldehyde, processed for routine histopathology, embedded in paraffin and 4 micron sections were stained with hematoxylin and eosin (H&E); or stained by immunohistochemistry (IHC) with VECTASTAIN ABC Kit (PK-4000, Vector laboratories) and DAB Peroxidase Substrate Kit (SK-4100, Vector laboratories) following the manufacturer's instructions. Antibodies used for IHC were: anti-Nanog (1:500; A300-397A, Bethyl) and anti-Ki67 (1:100; RM-9106, Thermo). Tumors were evaluated and images where captured using a Zeiss Axioskop 2 microscope with a DS-Ri1 camera and NIS-Elements D image software.

Antibodies Used in this Study.

Rabbit polyclonal anti-m⁶A (Synaptic Systems, 202 003); Rabbit polyclonal anti-METTL3 (Proteintech, 15073-1-AP); Rabbit polyclonal anti-METTL3 (Bethyl, A301-568); Rabbit pre-immune serum (Sigma, R9133); Mouse monoclonal anti-beta actin (mAbcam, 8224); Rabbit polyclonal anti-PARP (Cell Signaling, 9542); Rabbit polyclonal anti-Nanog (Bethyl, A300-397A); Rabbit polyclonal anti-Nanog (ReproCell); Mouse monoclonal anti-Oct-3/4 (Santa cruz, sc-5279); Mouse monoclonal anti-Tuj1 (MMS-435P); mMF20 (Developmental studies Hybridoma bank); Rabbit monoclonal anti-Ki67 (Thermo, RM-9106); Donkey anti-Rabbit antibody (Amersham, NA934); Goat anti-Mouse IgG (H+L) IRDye 680RD (Licor); Goat anti-Rabbit IgG (H+L) IRDye 800CW (Licor); Goat anti-mouse Alexa-488; Goat anti-Rabbit Alexa-555; Donkey anti-mouse Alexa-555; Donkey anti-rabbit Alexa-488.

m⁶A Antibody Titration

We generated an m⁶A antibody titration curve to identify the point of saturation of the anti-m⁶A antibody in the context of performing m⁶A RIPs (FIG. S1). To do so, we utilized an in vitro generated transcript from a plasmid containing full length GAPDH transcript. The plasmid was first linearized by restriction digest using SalI just downstream of the GAPDH cDNA cloning site. The linearized plasmid was gel purified and in vitro T7 mediated transcription was performed using the Ambion MEGAscript Kit (AM1334) as described in the user manual. The incorporation of m⁶A to the m⁶A transcripts was done by adding TriLink N⁶-Methyladenosine-5′-Triphosphate (cat# N1013) at the indicated concentration to unmodified ATP of the kit (ex a 2% m⁶A transcript was made by mixing 98% ATP with 2% m⁶A nucleotide) according to the manufacturer instructions. The anti-m⁶A RIP was performed as described in the m⁶A-seq section, with the exception that intact full length GAPDH transcript was utilized as input for the RIP step.

### Example 1

N6-methyl-adenosine (m6A) is the most abundant covalent modification on messenger RNAs in somatic cells and is linked to human diseases, but its functions in mammalian development are poorly understood. Here, the inventors demonstrate an evolutionary conservation and function of m6A by mapping the m6A methylome in mouse and human embryonic stem cells (ESCs). Thousands of messenger and long noncoding RNAs show conserved m6A modification, including transcripts encoding core pluripotency transcription factors Nanog and Sox2. m6A was discovered to be enriched over 3′ untranslated regions at defined sequence motifs, and marks unstable transcripts, including transcripts that need to be turned over upon differentiation. Genetic inactivation or depletion of mouse and human Mettl3, one of the known m6A methylases, led to m6A erasure on select target genes, prolonged Nanog expression upon differentiation, and impaired ESC's exit from self-renewal towards differentiation into several lineages in vitro and in vivo. Thus, the inventors have discovered that m6A is a mark of transcriptome flexibility required for stem cells to differentiate to specific lineages.

Thousands of mESC Transcripts Bear m⁶A

To understand the role of the m⁶A RNA modification in early development, the inventors mapped the locations of m⁶A modification across the transcriptome of mouse (mESC) and human (hESC) embryonic stem cells. Polyadenylated RNA was subjected to fragmentation, and m⁶A-bearing fragments were enriched by immunoprecipitation with an m⁶A-specific antibody, followed by high throughput sequencing (Methods). For each experiment, libraries were built for multiple biological replicates and concordant peaks for each experiment were used for subsequent bioinformatic analyses.

In mESCs, m⁶A-seq revealed a total of 9754 peaks in 5578 transcripts (˜2 peaks per transcript) with RPKM>1. The majority of m⁶A peaks are found in protein coding genes, with 9588 m⁶A peaks found in 5461 protein coding transcripts (out of 9923 protein coding transcripts). Considering the lower expression levels of lncRNA as a class, it is likely that the fraction of modified noncoding transcripts is underestimated. 166 m⁶A peaks are found in 117 noncoding transcripts (out of 485 long noncoding RNA transcripts) (Table S1, as disclosed in Batista et al., Cell Stem Cell, 2014, 15(6), 707-719). Thus, thousands of mESC transcripts, including mRNAs and lncRNAs, are m⁶A-modified (Dominissini et al., 2012; Meyer et al., 2012).

m⁶A in mRNAs of mESC Core Pluripotency Factors

The inventors herein discovered that mRNAs encoding the core pluripotency regulators in mESCs are modified with m⁶A. Nanog, Klf4, and Myc mRNAs all showed regions of m⁶A enrichment, whereas Pou5f1 (also known as Oct4) lacked m⁶A modification (FIG. 1A). Furthermore, the m⁶A-seq results were confirmed with independent m⁶A IP-qRT-PCR. (FIG. 9A). A medium throughput validation assay was deployed using m⁶A-IP followed by Nanostring nCounter analysis (m⁶A-string), which again validated m⁶A enrichment of Nanog, Sox2, Myc mRNAs and select mESC lncRNAs over the gene body of beta-actin (Table S2, as disclosed in Batista et al., Cell Stem Cell, 2014, 15(6), 707-719). These validation results suggest that the m⁶A-seq data are accurate and robust. Extending downstream of the ESC master regulators, it was discovered that m⁶A marks the mRNAs of 9 of 14 second-tier regulators important for ESC self-renewal and repression of lineage-specific transcription (Young, 2011), including Myc, Lin28, Med1, Jarid2, and Eed (FIG. 1B). The mRNAs of eight out of twelve key regulatory proteins recently reported to account for a majority of ESC cell fate decisions are m6A modified (Dunn et al., 2014). Dividing the modified genes into five groups based on the degree of modification revealed that the top group (corresponding to the top 20% modified genes) was enriched for several functional groups, including: chordate embryonic development, embryonic development, gastrulation and cell cycle (FIG. 1C). Thus, m⁶A extensively marks mRNAs encoding the ESC core pluripotency network, many of which are dynamically controlled at the level of transcription during differentiation.

m⁶A Location and Motif in mESCs Suggest a Common Mechanism Shared with Somatic Cells

De novo motif analysis of mESC m⁶A sites revealed a motif that recapitulates the previously described m⁶A sequence motif (FIG. 1D) (Canaani et al., 1979; Csepany et al., 1990; Dominissini et al., 2012; Harper et al., 1990; Horowitz et al., 1984; Meyer et al., 2012; Rana and Tuck, 1990; Rottman et al., 1994; Wei and Moss, 1977). The frequency of motif occurrence peaks near the center of experimentally mapped m⁶A sites. Control motif analysis on a random group of windows of the same size, extracted from genes with comparable level of expression, failed to identify the methylation motif, demonstrating specificity (FIG. 9B). m⁶A sites in mESC are significantly enriched near the stop codon and beginning of the 3′ UTR of protein coding genes (FIGS. 1E and 1F), as previously described for somatic cells. Although the largest fraction of m⁶A sites was within the coding sequence (CDS, 35%), the stop codon neighborhood showed the strongest enrichment, as a 400 nt window around stop codons contained 33% of m⁶A sites in the mESC transcriptome but represented just 12% of the motif occurrence. In genes with only one modification site, the bias for modification at the neighborhood of the STOP codon is even more pronounced (FIG. 1F). Comparison of transcript read coverage between input and wild type revealed no bias for read accumulation around the STOP codon in the input sample (FIG. 9C).

Next, the relationship between exon length of the coding sequence (CDS) and m⁶A modification of mRNAs was analysed, purposefully excluding the last exon, frequently the longest exon in a coding gene, and often including part of the CDS along with the stop codon and 3′-UTR. The inventors discovered that methylated internal exons were significantly longer than non-methylated control internal exons (median exon length of 737 bp vs 124 bp; P<2.2×10⁻¹⁶; two-sided Wilcoxon test). The strong bias for m⁶A modification occurring in long internal exons remained even when the number of peaks per exon was normalized by exon length (FIGS. 9D and 9E). Alternatively, this enrichment in long internal exons of mRNAs could be the result of higher probability of finding RRACU motif in longer sequence space. Analysis of number of peaks per exon after normalizing by the number of motifs in such exons revealed a strong enrichment of m⁶A modification(s) in long exons, independent of the number of potential motifs (FIG. 9F). These results demonstrate the possibility that processing of long exons is coupled mechanistically to m⁶A targeting through as yet unclear systems and/or that m⁶A modification itself may play a role in controlling long exon processing. The topological enrichment of m⁶A peaks surrounding stop codons in mRNAs is a poorly understood aspect of the m⁶A methylation system. Therefore, to understand if there was a topological enrichment or constraint on m⁶A modification in non-coding RNAs (ncRNAs), which by definition have no stop codons, the inventors parsed both ncRNAs and protein coding RNAs with three or more exons into three normalized bins including: the 1st exon, all internal exons and last exon. The inventors determined that there was an enrichment of m⁶A near the last exon-exon splice junction for both coding and ncRNAs (FIG. 1G), demonstrating that the enrichment of m⁶A peaks around the STOP codon is independent of the Stop codon itself. Furthermore, the inventors also discovered m⁶A enrichment in mRNAs and non-coding RNAs as the last splice junction is crossed (FIG. 9G). Interestingly, the inventors also identified increasing frequency of m⁶A approaching the 3′ end of single-exon genes (FIG. 9H), consistent with high m⁶A at the 3′end/last codon-3′UTR of multi-exonic genes.

Together, the location and sequence features identified in mESCs demonstrate a mechanism for m⁶A deposition that is similar if not identical in somatic cells. Thus, the inventors have discovered that that the m6A methylome is hardwired into transcripts based on their primary sequence, and is present in pluripotent cells that are a model of early embryonic life.

### Example 2

### m⁶A is a Mark for RNA Turnover

Next, the inventors assessed if transcript levels are correlated with the presence of m⁶A modification. Comparison of m⁶A enrichment level versus the absolute abundance of RNAs revealed no correlation between level of enrichment and gene expression (FIG. 1H). A separate, quartile based analysis found a higher percentage of m⁶A-modified transcripts in the middle quartiles of transcript abundance (FIG. S1I). Thus, the methylome analysis demonstrates that m⁶A modification is not simply a random modification that occurs on abundant cellular transcripts; rather, m⁶A preferentially marks transcripts expressed at a medium level.

To further define potential mechanisms of m⁶A function, the inventors assessed whether m⁶A-marked transcripts differ from unmodified transcripts at the level of transcription, RNA decay, or translation by leveraging published genome-wide datasets in mESCs (Methods). RNA polymerase II occupancy at the promoter region of both unmodified and m⁶A-marked RNAs is similar (FIG. 9J). In contrast, m⁶A-marked transcripts had significantly shorter RNA half-life—2.5 hours shorter on average (p=<2.2⁻¹⁶, FIG. 14 and increased rate of mRNA decay (average decay rate of 9 min vs 5.4 min for m⁶A vs. unmodified, p=<2.2⁻¹⁶). m⁶A modified transcripts have slightly lower translational efficiency than unmodified transcripts (1.32 vs. 1.51, respectively) (Ingolia et al., 2011) (FIG. 9K). These results demonstrated that m⁶A is a chemical mark associated with transcript turnover.

Mettl3 Knockout Decreases m⁶A and Promotes ESC Self-Renewal

To understand the role of m⁶A methylation in ESC biology, the inventors inactivated Mettl3, which is one of the components of the m⁶A methylase complex. No genetic study of Mettl3 has been performed in human stem cell populations to rigorously define its requirement for m⁶A modification, as all previously reported studies have relied on knock down. Herein, the inventors targeted Mettl3 by CRISPR-mediated gene editing (see Methods section), and generated several homozygous Mettl3 KO ESC lines. DNA sequencing confirmed homozygous stop codons that terminate translation within the first 75 amino acids, and immunoblot analysis confirmed the seabsence of Mettl3 protein (FIG. 2A, FIG. 10A). Two dimensional thin layer chromatography (2D-TLC) of single nucleotides digested from purified poly(A) RNA showed a significant (˜60%) but incomplete reduction of m⁶A in Mettl3 KO ESC (FIG. 2B and FIG. 10B). Interestingly and contrary to a recent publication (Wang et al., 2014b), the inventors suprizingly discovered that Mettl3 KO reduced but did not prevent the stable accumulation of Mettl14 (FIG. 10C). Thus, these experiments demonstrated that Mettl3 is a major, but not the sole, m⁶A methylase in mESC.

Furthermore, in contrast to prior reports, the inventors demonstrated herein that Mettl3 KO ESCs are viable and surprisingly demonstrated improved self-renewal. In fact, Mettl3 KO in mESCs were unexpectedly viable and could be maintained indefinitely over months, and Mettl3 KO ESCs exhibited low levels of apoptosis, similar to wild type mESCs, as judged by PARP cleavage and Annexin V flow cytometry (FIG. 2A, FIG. 10D). The inventors next assessed whether Mettl3 KO affected the ability of stem cells to remain pluripotent. Mettl3 KO ESC colonies were consistently larger than WT ESCs, and still retained the round and compact ESC colony morphology with intense alkaline phosphatase staining comparable to wild type colonies as well as uniform expression of Nanog and Oct4 (FIG. 2C, 2D, 2E, FIG. 10E and data not shown). Quantitative cell proliferation assay confirmed the increased proliferation rate of KO over WT ESCs (FIG. 2F). These observations demonstrate that Mettl3 KO enables enhanced ESC self-renewal. To rule out potential off-target effects from CRISPR-mediated gene targeting, an orthogonal approach to knockdown Mettl3 in ESCs was used. In particular, the inventors used two independent short hairpin RNAs (shRNAs) knocked down Mettl3 to ˜20% (FIG. 10F). 2D-TLC showed a ˜40% loss of m⁶A in poly(A) RNAs (FIG. 10G), and apoptosis assays confirmed lack of cell death induction. Importantly, Mettl3 depletion also increased ESC proliferation compared to control shRNA for one hairpin (FIG. 10H). Thus, two independent approaches confirm that Mettl3 inactivation enhanced self-renewal of ESCs.

Mettl3 KO Blocks Directed Differentiation In Vitro and Teratoma Differentiation In Vivo

These findings, coupled with the discovery that modified genes tend to have a shorter half-life, demonstrate that Mettl3, and by extension m⁶A, is needed to fine-tune and limit the level of many ESC genes, including pluripotency regulators. Since Mettl3 KO cells are capable of self-renewal, their capacity for directed differentiation in vitro toward two lineages: cardiomyocytes (CM) or the neural lineage was assessed. While the wild type control cells were able to generate beating CM (˜50% of colonies), only ˜3% of Mettl3 KO colonies of two independent clones produced beating CMs. Furthermore, differentiated colonies of Mettl3 KO cells retained high levels of Nanog expression but lacked expression of the CM structural protein Myh6, reflecting a larger number of cells that failed to exit the mESC program in the mutant cells. (FIG. 3A and data not shown). Similarly, upon directed differentiation to the neural lineage, a marked difference between the ability of the two cells types to differentiate was detected. To assay for neural differentiation, the cells were stained for Tuj1, a beta-3 tubulin which is expressed in mature and immature neurons. While ˜53% of wild type colonies had Tuj1+ projections, less than 6% of Mettl3 KO colonies had Tuj1+ projections in both knock-out clones (FIG. 3B). Additionally, differentiated Mettl3 KO cells showed an impaired ability to repress Nanog and activate Tuj1 mRNA (FIG. 3B). To confirm the role of Mettl3 in ESC differentiation in vivo, Mettl3 KO or wild type cells were injected subcutaneously into the right or left flank respectively, of SCID/Beige mice (n=5). Both wild type and Mettl3KO cells formed tumors consistent in morphology with teratomas. Mutant tumors tended to be larger, in accordance with mutant cell growth curves observed in vitro (FIG. 3C). Histological analysis of H&E stained tumor sections revealed consistent differences between the two populations: While both groups of cells formed teratomas that contained differentiation to some degree, into all three germ layers, the teratomas derived from KO cells were predominantly composed of poorly differentiated cells with very high mitotic indices and numerous apoptotic bodies, whereas wild type cells differentiated predominantly into neuroectoderm (FIG. 3D). Analysis of adjacent sections revealed that the mutant teratomas have markedly higher staining of proliferation marker Ki67 and ESC protein Nanog, which highlight the poorly differentiated cells (FIG. 3D and FIG. 11A). RNA analysis confirmed that Mettl3 KO tumors had higher levels of Nanog, Oct4 and Ki67 and lower levels of Tuj1, Myh6 and Sox17 (FIG. 11B). Thus, the inventors discovered that inhibition of Mettl3 leads to insufficient m⁶A, which in turn leads to a block in ESC differentiation and persistence of a stem-like, highly proliferative state (i.e., mettl3 inhibition leads to self-renewal and proliferation of ESCs).

### Example 3

### Mettl3 Target Genes in mESCs

The incomplete loss of bulk m⁶A in Mettl3 KO may result either because Mettl3 is soley responsible for the methylation of a subset of genes or sites and/or Mettl3 functions in a redundant fashion with another methylase on all m⁶A-modified genes. To distinguish these possibilities, the m⁶A methylome was mapped in Mettl3 KO cells. Comparison of the methylomes of wild type vs. Mett3 KO ESCs revealed a global loss of methylation across m6A sites identified in wild type (FIG. 4A). The inventors detected changes in 3739 sites (in 3122 genes), including modification sites in Nanog mRNA. Thus, this unbiased analysis suggested a set of targets that rely more exclusively on Mettl3, including Nanog and other pluripotency mRNAs (FIGS. 4B and 4C) (Table S1, as disclosed in Batista et al., Cell Stem Cell, 2014, 15(6), 707-719). Gene Set Enrichment Analysis confirmed that Mettl3-target genes significantly overlap functional gene sets important for pluripotency, including targets of Ctnnb1 (8.8×10⁻¹⁰), targets of Smad2 or Smad3 (1.6×10⁻²³), targets of Myc (2.7×10⁻¹²), targets of Sox2 (6.5×10⁻¹⁴), and targets of Nanog (8.5×10¹⁴) (FIG. 4C). Five of eleven core ESC regulators lost m6A modification in Mettl3 KO, including Nanog, Rlf1, Jarid2, and Lin28 (FIG. 4D). Independent validation by m⁶A RIP followed by Nanostring detection confirmed loss of m⁶A in Nanog, and other mRNAs in KO vs. wild type ESCs (FIG. 4E). Following transcription arrest by flavopiridol treatment, Nanog mRNA showed delayed turnover in Mettl3 KO cells compared to wild type, consistent with a requirement for m⁶A in Nanog mRNA turnover (FIG. 4F). However, RNA-seq analysis of Mettl3 KO cells revealed modest perturbations in mRNA steady state levels with only ˜300 genes demonstrating significant changes over 1.5 fold. Collectively, these results suggest that Mettl3 plays a selective role in regulating the dynamics of ESC gene expression.

Wide Spread m⁶A Modification of Human ESCs

The identification of thousands of m⁶A sites raises the challenge of defining the functional importance of each and every one of the sites. To this end, the inventors mapped m⁶A sites in hESCs and during endoderm differentiation to elucidate the patterns and potential conservation of m⁶A methylome (FIG. 5A). In basal (undifferentiated or resting) state hESCs (T=0), m⁶A-seq identified 16,943 peaks in 7,871 genes representing 7530 coding and 341 non-coding RNAs. Upon differentiation towards endoderm (T=48, “endoderm differentiation” thereafter), m⁶A-seq identified 15,613 m⁶A peaks in 7,195 genes representing 6909 coding and 286 non-coding RNAs (Table S3, as disclosed in Batista et al., Cell Stem Cell, 2014, 15(6), 707-719). As shown in FIG. 5B, 11322 peaks (6004 genes) were common between the undifferentiated (T=0) and differentiated hESCs (T=48), while 5348 (3979 genes) vs 4087 peaks (3024 genes) were unique respectively.

Many Master Regulators of hESC Maintenance and Differentiation are Modified with m6A

Interestingly, similar to mESC, transcripts encoding many hESC master regulators, including human NANOG, SOX2, and NR5A2, were m⁶A modified. Like mESC, the transcripts for OCT4 (POUF51) in hESC did not harbor an m⁶A modification (FIG. 5D). These results show a high level of specificity and conservation of m⁶A targets among core-pluripotency/maintenance factors in mouse and human ESCs. The inventors also identified human specific lncRNAs with known roles in hESC maintenance such as LINC-ROR and MEGAMIND/TUNA to contain m⁶A modification(s) (FIG. 5D; FIG. 13A) (Lin et al., 2014; Loewer et al., 2010). Upon induction of differentiation, the inventors identified transcripts encoded by several key regulators of endodermal differentiation also to have m⁶A modifications including EOMES and FOXA2 (FIG. 5D). Gene ontology (GO) analyses of methylated genes in undifferentiated hESC (T=0) were significantly enriched in biological functions such as regulation of transcription (FDR=1.2×10⁻¹⁴), chordate embryonic development (FDR=1.1×10⁻⁴), and regulation of cell morphogenesis (FDR=0.01). The same analysis after endodermal differentiation retained enrichment in the similar GO terms. Upon differentiation toward endoderm, 1356 peaks in 1137 genes showed quantitative differences of at least 1.5 fold in m6A intensity, after normalization for input transcript abundance (FIGS. 5E and 5F, Table 2, as disclosed as Table S6 in Batista et al., Cell Stem Cell, 2014, 15(6), 707-719). The majority of these differential m⁶A sites represented quantitative differences at existing sites (i.e. 59.1% of the peaks were called in both time points), rather then state-specific de novo appearance or erasure of modification (FIG. 5G) (see methods). This is consistent with the discovery that 74.9% of sites overlapped observed in 293T data (Meyer et al., 2012) and the little change seen in m6A sites in a recent survey of cell types (Schwartz et al., 2014), demonstrating that transcripts exhibit dynamic differential peak m6A methylation intensity largely at “hard wired sites” during differentiation under the conditions examined and when compared to other tissue types.

Conserved Features of m⁶A Modifications Spanning Different Species

The inventors determined that three salient features of the m⁶A methylome are conserved in hESCs. First, m⁶A sites in hESCs are also dominated by the identical RRACU motif seen in mESC and somatic cells (Dominissini et al., 2012; Meyer et al., 2012) (FIG. 5C). There was also a strong preference of targeting long-internal exons at the RRACU motif even after normalizing for exon length and number of m⁶A motifs (FIG. 5H). Second, there was a significant enrichment in m⁶A peaks at 3′ end of transcripts, near the stop codons of coding genes or the last exon in non-coding RNAs (FIG. 5I, FIG. 13B, 13C). Furthermore, the topology of m⁶A modification is preserved upon endodermal differentiation (FIG. 5I). As in mESCs, moderate to lowly expressed genes have higher probability of becoming methylated (FIG. 13E). Lastly, hESC m⁶A is not correlated with transcription rate as judged by GRO-seq (Sigova et al., 2013), but is strongly anti-correlated with measured mRNA half-life in human pluripotent cells (Neff et al., 2012), strongly suggesting that m⁶A modification also marks RNA turnover in hESCs, as observed for mESCs (FIG. 5J, FIGS. 13F and 13G).

Evolutionary Conservation and Divergence of the m⁶A Epi-Transcriptomes of Human and Mouse ESCs

Previous studies report conservation of m⁶A modified genes between mouse and human in somatic cell types (˜51%-45%), but the comparisons are limited by non-matched tissue types and transformed vs. untransformed cell types (Dominissini et al., 2012; Meyer et al., 2012). Herein, the inventors assessed the evolutionary conservation of human and mouse ESC m⁶A methylomes. At the gene level, 69.4% (3609 of 5204) of hESC genes are also m⁶A modified in the orthologus mouse gene (p-value=8.3×10⁻¹⁷⁹; Fisher exact test) (FIG. 6A; Table S5, as disclosed in Batista et al., Cell Stem Cell, 2014, 15(6), 707-719). Furthermore, the inventors identified 632 conserved m⁶A peak sites (46.1%) between hESCs and mESCs (Table 1, which is a modified version of Table S6 disclosed in Batista et al., Cell Stem Cell, 2014, 15(6), 707-719). Notably, conserved sites tended to have higher m⁶A peak intensities compared to m⁶A peak sites that are not conserved (FIGS. 6B and 6C, p-values=1.3×10⁻¹⁵ and 8.7×10⁻²³ for hESC or mESC, respectively; Wilcoxon test). The species specificity of gene methylation in mouse and human showed multiple patterns as shown through the indicated examples, starting with genes found exclusively methylated in one species or another (FIGS. 6D and 6E, Table 2, also disclosed as Table S4 in Batista et al., Cell Stem Cell, 2014, 15(6), 707-719). In terms of commonly methylated genes, regulators of ESC pluripotency demonstrate m⁶A modification sites at nearly equivalent locations such as SOX2 (FIG. 6F), but not identical sites based on our analyses. While other genes, such as GLI1 had methylation at identical site(s). Yet, other genes such as CHD6 were found to have a conserved m⁶A site, along with a mouse or human-specific m⁶A peaks at different exons (FIG. 6F). Thus, while the inventors data reveals a substantial overlap at the gene level, demonstrating broad functional significance of m⁶A modification in ESCs in both species, the inventors also discovered numerous species-specific m⁶A patterns that may contribute to specific aspects of human ESC biology (Schnerch et al., 2010).

### Example 4

### METTL3 is Required for hESC Differentiation

To address the function of m⁶A in hESCs, hESC colonies were generated with stable knockdown of METTL3, shRNA control, or wild-type cells (FIG. 7A). Knockdown of METTL3 in hESCs resulted in reduction in METTL3 mRNA levels and reduction in m⁶A level based on serial dilution analysis of polyA+RNA (FIGS. 7B and 7C and FIGS. 13B and 13C). METTL3-depleted hESCs could be stably maintained, demonstrating the dispensability of METTL3 for hESC self-renewal. Furthermore there was no difference in viability between control and knockdown hESCs (data not shown). Strikingly, differentiation of METTL3-depleted hESCs into neural stem cells (NSCs) by dual inhibition of SMAD signaling, using Dorsomorphin and SB-431542 revealed a block in neuronal differentiation (Methods). While 44% (±3.5% s.d.) of the control cells were Sox1 positive, only 10% (±3.1% s.d) of the METTL3-depleted were Sox1 positive (FIG. 13A).

Similarly, knockdown of METTL3, in three independently generated ES colony clones selected for METTL3 knockdown, led to a profound block in endodermal differentiation at day 2 and day 4 based on failure to express the endoderm markers EOMES and FOXA2 compared to either two shRNA control colony clones (FIG. 7D) or wildtype hESCs (FIG. 13D). Consistently, METTL3-depleted ESCs retain high levels of expression of the master regulators NANOG and SOX2 throughout the differentiation time course in contrast to their diminishing expression in wild type cells (FIG. 7E and FIG. 13E). These results indicate that METTL3 and m⁶A control differentiation of hESCs.

### Example 5

In previous reports of m⁶A sites in transformed HepG2 cells under a variety of conditions showed the majority of m⁶A sites were invariant, a subset of dynamically regulated m⁶A sites was also reported (Dominissini et al., 2012). However, the Dominissini and colleagues study lacked sufficient replicates of stimulated samples to allow for accurate assessment of m6A sites. Chen et al., (Chen, Cell Stem Cell Mar. 5 2015; FIG. 1D) also report that among 3,880 commonly expressed transcripts in four different mouse cell/tissue types, 89% of 3,880 genes had variable or dynamically regulated m⁶A peaks in at least two cell types, however, as there were was insufficient replicates, the results cannot be accurately assessed, in addition, Chen and colleges fail to specify the criteria for identifying differential peaks. Herein, the inventors rely upon replicates is critical for the concordance of peak calling. In contrast, previous published studies hover at ˜70-80%, making it a challenge to call differential m⁶A peaks in single replicates, due to the inherent noise in m⁶A-seq. In addition, it was unclear from the previous reports whether differential peaks truly represent novel and unique sites vs “latent” sites that can be found in other cells/conditions or tissue/cell types. Lastly, before the present invention, it was not clear how human m6A peak intensity compared to mouse tissues.

In contrast to the previous reports, herein, the inventors analyzed the degree of dynamic modulation of m⁶A peaks across at least two replicates during human ESC endoderm differentiation. Only genes that showed an FPKM of >=1 in their input at both time points were analysed and used to calculate the intensity of m⁶A peaks identified by Pirhana. Peaks were then identified as exhibiting differential m⁶A peaks intensities (DMPIs) between t=0 and t=48. The inventors detected 5.3% (n=194/3674; 156 genes) and 18.8% (n=691/3674; 481 genes) of m⁶A sites exhibited DMPIs over a threshold of 2 fold or 1.5 fold, respectively (Table S3, as disclosed in Batista et al., Cell Stem Cell, 2014, 15(6), 707-719).

Of these 691 DMPIs using 1.5 fold threshold, 77.1% occurred in genes that showed no differential gene expression (FIG. 4A). Furthermore, 44.4% of these DMPIs represent m⁶A peaks called in both time points (T=0 vs T=48). Examples of genes showing DMPI during differentiation include LRRC47 and C-MYC, which show an increase in m⁶A peak intensities following differentiation. By contrast, genes such as RBMX show a decrease in m⁶A peak intensities following differentiation. In addition, genes such as RANGAP1, which have two methylation sites, only exhibit dynamic regulation of one site (FIG. 4B). A gene ontology (GO) analyses did not yield a significant recognizable pattern. As shown in FIG. 4C supervised hierarchical clustering of the DMPI set was able to distinguish the hESC samples. Accordingly, the present technology demonstrates the utility and the power of using m⁶A methylation status to distinguish hESC in their basal (undifferentiated or resting) state (t=0) from the differentiated cells (t=48). To perform an unbiased assessment, the inventors carried out unsupervised clustering of the log(2) peak intensities for high confidence peaks in genes with FPKM>1 and large coefficient of variation in peak intensities across all samples. Importantly, this unsupervised clustering analysis was able to distinguish differentiated from undifferentiated cells (FIG. 12). Importantly, the inventors demonstrate herein the potential of m⁶A site peak intensity as novel cellular classifiers. Biologically, this analysis elucidates a restricted but dynamic m⁶A modification program triggered by hESC endoderm differentiation.

### Example 6

### m⁶A Methylome in ES Cells

The inventors demonstrate herein that the ESC m⁶A methylome in mouse and human cells reveals extensive m⁶A modification of ESC genes, including most key regulators of ESC pluripotency and lineage control. The pattern and sequence motif associated with ESC m⁶A are similar to those previously reported in somatic cells, indicating a single mechanism that deposits m⁶A modification in early embryonic life. This conserved mechanism for m⁶A contrasts with the complexity of 5-methyl-cytosine in DNA and histone lysine methylations that undergo extensive reprogramming with distinct rules in pluripotent vs. somatic cells.

Importantly, the inventors discovered a general and conserved topological enrichment of m⁶A sites at the 3′ end of genes among single-exon and multi-exon mRNAs as well as ncRNAs. Thus, neither the stop codon nor the last exon-exon splice junction can alone explain the observed m⁶A topology in RNA. However, all species examined to date including Saccharomyces cerevisae and Arabidopsis thalania exhibit a strong 3′ bias in m⁶A localization, suggest an evolutionary constraint that may target the m⁶A modification to the 3′ ends of genes regardless of gene structure or coding potential (Bodi et al., 2012; Schwartz et al., 2013). This bias may be achieved by preferential m⁶A methylases recruitment to 3′ sites or preferential action of demethylases in upstream regions of the transcript. Although the role of de-methylases cannot be excluded in the patterning of the m⁶A methylome, the observation of 3′ end m⁶A bias in S. cerevisiae, which lacks known m⁶A demethylases argues against the latter mechanism (Jia et al., 2011; Schwartz et al., 2013; Zheng et al., 2013). The functional importance of m⁶A location vs. its specific molecular outcome need to be addressed in future studies.

Mettl3 Selectively Targets mRNAs Including Pluripotency Regulators

While previous reports had approached Mettl3 function by RNAi knock down (Dominissini et al., 2012; Fustin et al., 2013; Liu et al., 2014; Wang et al., 2014b), herein the inventors used genetic ablation of Mettl3 KO (using CRISPR) to examine the true loss-of-function phenotypes. The importance of using definitive genetic models is highlighted by recent studies in the DNA methylation field where shRNA experiments led to mis-assigned functions of Tet proteins that were later recognized in genetic knockouts (Dawlaty et al., 2013; Dawlaty et al., 2011). We found that both Mettl3 KO and depletion led to incomplete reduction of the global levels m⁶A in both mESCs and hESCs, demonstrating redundancy in m⁶A methylases. However, m⁶A profiling in Mettl3 KO cells revealed a subset of targets, approximately 33% of m⁶A peaks, that are preferentially dependent on Mettl3, and these included Nanog, Sox2, and additional pluripotency genes. A second m⁶A methylase, Mettl14, could also regulate m⁶A on some of the identified target genes.

RNAi knockdown of Mettl3 in somatic cancer cells led to apoptosis (Dominissini et al., 2012), and Wang and colleagues reported ectopic differentiation of mESC with Mettl3 depletion (Wang et al., 2014b). In contrast, herein the inventors suprizingly discovered that Mettl3 KO does not affect ESC cell viability or self-renewal, and in fact mESC renewed at an improved rate.

Conservation of m6A Methylome in Mammalian ESCs

The conserved methylation patterns of many ESC master regulators and the shared phenotype observed upon inactivation of METTL3 suggest that METTL3 operates to control stem cell differentiation. It is known that human and mouse ESCs are not equivalent (Schnerch et al., 2010), and are cultured in different conditions. By focusing in on orthologous genes, the inventors were able to catalog both shared and species-specific methylation sites. The observation that certain methylation sites are modified whenever a target transcript is expressed in both species, despite cell state or culture differences, demonstrates that these modification events have been preserved under strong purifying selection during evolution. Herein, the inventors genomic analyses also pave the way to further understand potential biological differences between mouse and human ESCs at the level of m6A epitranscriptome, given the unique patterns of some methylation sites between the species.

RNA “Anti-Epigenetics”: m⁶A as a Mark of Transcriptome Flexibility

Stem cell gene expression programs need to balance fidelity and flexibility. On one hand, stem cell genes need sufficient stability to maintain self-renewal and pluripotency over multiple cell generations, but on the other hand, gene expression needs to change dynamically and rapidly in response to differentiation cues. It has been proposed that ESC gene expression programs are in constant flux between competing fates, and pluripotency is a statistical average (Loh and Lim, 2011; Montserrat et al., 2013; Shu et al., 2013). Herein, the inventors have demonstrated that mRNAs with m⁶A tend to have a shorter half-life, and Nanog and Sox2 mRNAs could not be properly down-regulated on differentiation in Mettl3-deficient mESC and hESC. However, Mettl3 deficiency has only modest effects on steady state gene expression, which could arise from the non-stoichiometric nature of the m⁶A modification. The application of methods and assays disclosed herein are useful to determine level of modification of each RNA species are useful for determining the state of the stem cell population (Harcourt et al., 2013; Liu et al., 2013). Herein and in contrast to prior reports, the inventors demonstrate that Mettl3 KO ESCs suprizingly results in enhanced self-renewal but hindered differentiation, concomitant with decreased ability to down regulate ESC mRNAs. WTAP, a conserved Mettl3 interacting partner from yeast to human cells (Horiuchi et al., 2013; Schwartz et al., 2014), is also required for endodermal and mesodermal differentiation (Fukusumi et al., 2008). The observed phenotypes in ESC and teratomas are all the more notable because we have significantly reduced but not eliminated m⁶A.

Accordingly, the inventors have demonstrated a model where m⁶A serves as the necessary flexibility factor to counter balance epigenetic fidelity—a RNA “anti-epigenetics” (FIG. 7F). m⁶A marks ESC fate determinants to limit their level of expression, and also ensures their continual degradation so that ESC can rapidly exit the pluripotent state upon differentiation. The inability of stem cell populations, e.g., human stem cells to exit the stem cell state (i.e., undifferentiated state) and continue proliferation upon insufficient m⁶A correlates with the association of FTO with human cancers (Loos and Yeo, 2013). METTL3 depletion also leads to elongation of the circadian clock (Fustin et al., 2013), also suggesting a role for m⁶A in resetting the transcriptome. In yeast, m⁶A is active during meiosis (Clancy et al., 2002; Schwartz et al., 2013), where diploid gene expression programs are reset to generate haploid offspring.

Herein, the inventors have demonstrated that m⁶A is important for the transition between cell states, by facilitating a reset mechanism between stages in both mouse and human cells. In contrast to epigenetic mechanisms that provide cellular memory of gene expression states, m⁶A enforces the transience of genetic formation—helping cells to forget the past and thereby embrace the future.

### REFERENCES

The references are incorporated herein in their entirety by reference.
Agarwala, S. D., Blitzblau, H. G., Hochwagen, A., and Fink, G. R. (2012). RNA methylation by the MIS complex regulates a cell fate decision in yeast. PLoS Genet 8, e1002732.
Bodi, Z., Zhong, S., Mehra, S., Song, J., Graham, N., Li, H., May, S., and Fray, R. G. (2012). Adenosine Methylation in Arabidopsis mRNA is Associated with the 3′ End and Reduced Levels Cause Developmental Defects. Front Plant Sci 3, 48.
Bokar, J. A., Shambaugh, M. E., Polayes, D., Matera, A. G., and Rottman, F. M. (1997). Purification and cDNA cloning of the AdoMet-binding subunit of the human mRNA (N6-adenosine)-methyltransferase. Rna 3, 1233-1247.
Canaani, D., Kahana, C., Lavi, S., and Groner, Y. (1979). Identification and mapping of N6-methyladenosine containing sequences in simian virus 40 RNA. Nucleic Acids Res 6, 2879-2899.
Clancy, M. J., Shambaugh, M. E., Timpte, C. S., and Bokar, J. A. (2002). Induction of sporulation in Saccharomyces cerevisiae leads to the formation of N6-methyladenosine in mRNA: a potential mechanism for the activity of the IME4 gene. Nucleic Acids Res 30, 4509-4518.
Csepany, T., Lin, A., Baldick, C. J., Jr., and Beemon, K. (1990). Sequence specificity of mRNA N6-adenosine methyltransferase. J Biol Chem 265, 20117-20122.
Dawlaty, M. M., Breiling, A., Le, T., Raddatz, G., Barrasa, M. I., Cheng, A. W., Gao, Q., Powell, B. E., Li, Z., Xu, M., et al. (2013). Combined deficiency of Tea and Tet2 causes epigenetic abnormalities but is compatible with postnatal development. Dev Cell 24, 310-323.
Dawlaty, M. M., Ganz, K., Powell, B. E., Hu, Y. C., Markoulaki, S., Cheng, A. W., Gao, Q., Kim, J., Choi, S. W., Page, D. C., et al. (2011). Tea is dispensable for maintaining pluripotency and its loss is compatible with embryonic and postnatal development. Cell Stem Cell 9, 166-175.
Dominissini, D., Moshitch-Moshkovitz, S., Schwartz, S., Salmon-Divon, M., Ungar, L., Osenberg, S., Cesarkas, K., Jacob-Hirsch, J., Amariglio, N., Kupiec, M., et al. (2012). Topology of the human and mouse m6A RNA methylomes revealed by m6A-seq. Nature 485, 201-206.
Dunn, S. J., Martello, G., Yordanov, B., Emmott, S., and Smith, A. G. (2014). Defining an essential transcription factor program for naive pluripotency. Science 344, 1156-1160.
Fu, Y., and He, C. (2012). Nucleic acid modifications with epigenetic significance. Curr Opin Chem Biol 16, 516-524.
Fukusumi, Y., Naruse, C., and Asano, M. (2008). Wtap is required for differentiation of endoderm and mesoderm in the mouse embryo. Dev Dyn 237, 618-629.
Fustin, J. M., Doi, M., Yamaguchi, Y., Hida, H., Nishimura, S., Yoshida, M., Isagawa, T., Morioka, M. S., Kakeya, H., Manabe, I., et al. (2013). RNA-Methylation-Dependent RNA Processing Controls the Speed of the Circadian Clock. Cell 155, 793-806.
Gulati, P., Cheung, M. K., Antrobus, R., Church, C. D., Harding, H. P., Tung, Y. C., Rimmington, D., Ma, M., Ron, D., Lehner, P. J., et al. (2013). Role for the obesity-related FTO gene in the cellular sensing of amino acids. Proc Natl Acad Sci USA 110, 2557-2562.
Guttman, M., Donaghey, J., Carey, B. W., Garber, M., Grenier, J. K., Munson, G., Young, G., Lucas, A. B., Ach, R., Bruhn, L., et al. (2011). lincRNAs act in the circuitry controlling pluripotency and differentiation. Nature 477, 295-300.
Harcourt, E. M., Ehrenschwender, T., Batista, P. J., Chang, H. Y., and Kool, E. T. (2013). Identification of a selective polymerase enables detection of N(6)-methyladenosine in RNA. J Am Chem Soc 135, 19079-19082.
Harper, J. E., Miceli, S. M., Roberts, R. J., and Manley, J. L. (1990). Sequence specificity of the human mRNA N6-adenosine methylase in vitro. Nucleic Acids Res 18, 5735-5741.
Heinz, S., Benner, C., Spann, N., Bertolino, E., Lin, Y. C., Laslo, P., Cheng, J. X., Murre, C., Singh, H., and Glass, C. K. (2010). Simple combinations of lineage-determining transcription factors prime cis-regulatory elements required for macrophage and B cell identities. Mol Cell 38, 576-589.
Hess, M. E., Hess, S., Meyer, K. D., Verhagen, L. A., Koch, L., Bronneke, H. S., Dietrich, M. O., Jordan, S. D., Saletore, Y., Elemento, O., et al. (2013). The fat mass and obesity associated gene (Fto) regulates activity of the dopaminergic midbrain circuitry. Nat Neurosci 16, 1042-1048.
Hongay, C. F., and Orr-Weaver, T. L. (2011). Drosophila Inducer of MEiosis 4 (IME4) is required for Notch signaling during oogenesis. Proc Natl Acad Sci USA 108, 14855-14860.
Horiuchi, K., Kawamura, T., Iwanari, H., Ohashi, R., Naito, M., Kodama, T., and Hamakubo, T. (2013). Identification of Wilms' tumor 1-associating protein complex and its role in alternative splicing and the cell cycle. J Biol Chem.
Horowitz, S., Horowitz, A., Nilsen, T. W., Munns, T. W., and Rottman, F. M. (1984). Mapping of N6-methyladenosine residues in bovine prolactin mRNA. Proc Natl Acad Sci USA 81, 5667-5671.
Hsu, P. D., Scott, D. A., Weinstein, J. A., Ran, F. A., Konermann, S., Agarwala, V., Li, Y., Fine, E. J., Wu, X., Shalem, O., et al. (2013). DNA targeting specificity of RNA-guided Cas9 nucleases. Nat Biotechnol 31, 827-832.
Ingolia, N. T., Lareau, L. F., and Weissman, J. S. (2011). Ribosome profiling of mouse embryonic stem cells reveals the complexity and dynamics of mammalian proteomes. Cell 147, 789-802.
Jia, G., Fu, Y., Zhao, X., Dai, Q., Zheng, G., Yang, Y., Yi, C., Lindahl, T., Pan, T., Yang, Y. G., et al. (2011). N6-methyladenosine in nuclear RNA is a major substrate of the obesity-associated FTO. Nat Chem Biol 7, 885-887.
Kang, H. J., Jeong, S. J., Kim, K. N., Baek, I. J., Chang, M., Kang, C. M., Park, Y. S., and Yun, C. W. (2014). A novel protein, Pho92, has a conserved YTH domain and regulates phosphate metabolism by decreasing the mRNA stability of PHO4 in Saccharomyces cerevisiae. Biochem J 457, 391-400.
Lin, N., Chang, K. Y., Li, Z., Gates, K., Rana, Z. A., Dang, J., Zhang, D., Han, T., Yang, C. S., Cunningham, T. J., et al. (2014). An Evolutionarily Conserved Long Noncoding RNA TUNA Controls Pluripotency and Neural Lineage Commitment. Mol Cell 53, 1005-1019.
Liu, J., Yue, Y., Han, D., Wang, X., Fu, Y., Zhang, L., Jia, G., Yu, M., Lu, Z., Deng, X., et al. (2014). A METTL3-METTL14 complex mediates mammalian nuclear RNA N6-adenosine methylation. Nat Chem Biol 10, 93-95.
Liu, N., Parisien, M., Dai, Q., Zheng, G., He, C., and Pan, T. (2013). Probing N6-methyladenosine RNA modification status at single nucleotide resolution in mRNA and long noncoding RNA. Rna.
Loewer, S., Cabili, M. N., Guttman, M., Loh, Y. H., Thomas, K., Park, I. H., Garber, M., Curran, M., Onder, T., Agarwal, S., et al. (2010). Large intergenic non-coding RNA-RoR modulates reprogramming of human induced pluripotent stem cells. Nat Genet 42, 1113-1117.
Loh, K. M., and Lim, B. (2011). A precarious balance: pluripotency factors as lineage specifiers. Cell Stem Cell 8, 363-369.
Loos, R. J., and Yeo, G. S. (2013). The bigger picture of FTO—the first GWAS-identified obesity gene. Nat Rev Endocrinol.
Meyer, K. D., Saletore, Y., Zumbo, P., Elemento, O., Mason, C. E., and Jaffrey, S. R. (2012). Comprehensive analysis of mRNA methylation reveals enrichment in 3′ UTRs and near stop codons. Cell 149, 1635-1646.
Montserrat, N., Nivet, E., Sancho-Martinez, I., Hishida, T., Kumar, S., Miguel, L., Cortina, C., Hishida, Y., Xia, Y., Esteban, C. R., et al. (2013). Reprogramming of human fibroblasts to pluripotency with lineage specifiers. Cell Stem Cell 13, 341-350.
Neff, A. T., Lee, J. Y., Wilusz, J., Tian, B., and Wilusz, C. J. (2012). Global analysis reveals multiple pathways for unique regulation of mRNA decay in induced pluripotent stem cells. Genome Res 22, 1457-1467.
Niu, Y., Zhao, X., Wu, Y. S., Li, M. M., Wang, X. J., and Yang, Y. G. (2013). N6-methyl-adenosine (m6A) in RNA: an old modification with a novel epigenetic function. Genomics Proteomics Bioinformatics 11, 8-17.
Rahl, P. B., Lin, C. Y., Seila, A. C., Flynn, R. A., McCuine, S., Burge, C. B., Sharp, P. A., and Young, R. A. (2010). c-Myc regulates transcriptional pause release. Cell 141, 432-445.
Rana, A. P., and Tuck, M. T. (1990). Analysis and in vitro localization of internal methylated adenine residues in dihydrofolate reductase mRNA. Nucleic Acids Res 18, 4803-4808.
Rottman, F. M., Bokar, J. A., Narayan, P., Shambaugh, M. E., and Ludwiczak, R. (1994). N6-adenosine methylation in mRNA: substrate specificity and enzyme complexity. Biochimie 76, 1109-1114.
Schnerch, A., Cerdan, C., and Bhatia, M. (2010). Distinguishing between mouse and human pluripotent stem cell regulation: the best laid plans of mice and men. Stem Cells 28, 419-430.
Schwartz, S., Agarwala, S. D., Mumbach, M. R., Jovanovic, M., Mertins, P., Shishkin, A., Tabach, Y., Mikkelsen, T. S., Satija, R., Ruvkun, G., et al. (2013). High-resolution mapping reveals a conserved, widespread, dynamic mRNA methylation program in yeast meiosis. Cell 155, 1409-1421.
Schwartz, S., Mumbach, M. R., Jovanovic, M., Wang, T., Maciag, K., Bushkin, G. G., Mertins, P., Ter-Ovanesyan, D., Habib, N., Cacchiarelli, D., et al. (2014). Perturbation of m6A Writers Reveals Two Distinct Classes of mRNA Methylation at Internal and 5′ Sites. Cell Rep 8, 284-296.
Segal, E., Friedman, N., Kaminski, N., Regev, A., and Koller, D. (2005). From signatures to models: understanding cancer using microarrays. Nat Genet 37 Suppl, S38-45.
Segal, E., Friedman, N., Koller, D., and Regev, A. (2004). A module map showing conditional activity of expression modules in cancer. Nat Genet 36, 1090-1098.
Segal, E., Shapira, M., Regev, A., Pe'er, D., Botstein, D., Koller, D., and Friedman, N. (2003). Module networks: identifying regulatory modules and their condition-specific regulators from gene expression data. Nat Genet 34, 166-176.
Shah, J. C., and Clancy, M. J. (1992). IME4, a gene that mediates MAT and nutritional control of meiosis in Saccharomyces cerevisiae. Mol Cell Biol 12, 1078-1086.
Sharova, L. V., Sharov, A. A., Nedorezov, T., Piao, Y., Shaik, N., and Ko, M. S. (2009). Database for mRNA half-life of 19 977 genes obtained by DNA microarray analysis of pluripotent and differentiating mouse embryonic stem cells. DNA Res 16, 45-58.
Shu, J., Wu, C., Wu, Y., Li, Z., Shao, S., Zhao, W., Tang, X., Yang, H., Shen, L., Zuo, X., et al. (2013). Induction of pluripotency in mouse somatic cells with lineage specifiers. Cell 153, 963-975.
Sibbritt, T., Patel, H. R., and Preiss, T. (2013). Mapping and significance of the mRNA methylome. Wiley Interdiscip Rev RNA 4, 397-422.
Trapnell, C., Pachter, L., and Salzberg, S. L. (2009). TopHat: discovering splice junctions with RNA-Seq. Bioinformatics 25, 1105-1111.
Ulitsky, I., Shkumatava, A., Jan, C. H., Sive, H., and Bartel, D. P. (2011). Conserved function of lincRNAs in vertebrate embryonic development despite rapid sequence evolution. Cell 147, 1537-1550.
Wang, X., Lu, Z., Gomez, A., Hon, G. C., Yue, Y., Han, D., Fu, Y., Parisien, M., Dai, Q., Jia, G., et al. (2014a). N6-methyladenosine-dependent regulation of messenger RNA stability. Nature 505, 117-120.
Wang, Y., Li, Y., Toth, J. I., Petroski, M. D., Zhang, Z., and Zhao, J. C. (2014b). N6-methyladenosine modification destabilizes developmental regulators in embryonic stem cells. Nat Cell Biol 16, 191-198.
Wei, C. M., and Moss, B. (1977). Nucleotide sequences at the N6-methyladenosine sites of HeLa cell messenger ribonucleic acid. Biochemistry 16, 1672-1676.
Wong, D. J., Liu, H., Ridky, T. W., Cassarino, D., Segal, E., and Chang, H. Y. (2008). Module map of stem cell genes guides creation of epithelial cancer stem cells. Cell Stem Cell 2, 333-344.
Young, R. A. (2011). Control of the embryonic stem cell state. Cell 144, 940-954.
Zheng, G., Dahl, J. A., Niu, Y., Fedorcsak, P., Huang, C. M., Li, C. J., Vagbo, C. B., Shi, Y., Wang, W. L., Song, S. H., et al. (2013). ALKBH5 is a mammalian RNA demethylase that impacts RNA metabolism and mouse fertility. Mol Cell 49, 18-29.
Zhong, S., Li, H., Bodi, Z., Button, J., Vespa, L., Herzog, M., and Fray, R. G. (2008). MTA is an Arabidopsis messenger RNA adenosine methylase and interacts with a homolog of a sex-specific splicing factor. Plant Cell 20, 1278-1288.
Cong, L., Ran, F. A., Cox, D., Lin, S., Barretto, R., Habib, N., Hsu, P. D., Wu, X., Jiang, W., Marraffini, L. A., et al. (2013). Multiplex genome engineering using CRISPR/Cas systems. Science 339, 819-823
Dominissini, D., Moshitch-Moshkovitz, S., Schwartz, S., Salmon-Divon, M., Ungar, L., Osenberg, S., Cesarkas, K., Jacob-Hirsch, J., Amariglio, N., Kupiec, M., et al. (2012). Topology of the human and mouse m6A RNA methylomes revealed by m6A-seq. Nature 485, 201-206.
Guttman, M., Donaghey, J., Carey, B. W., Garber, M., Grenier, J. K., Munson, G., Young, G., Lucas, A. B., Ach, R., Bruhn, L., et al. (2011). lincRNAs act in the circuitry controlling pluripotency and differentiation. Nature 477, 295-300.
Heinz, S., Benner, C., Spann, N., Bertolino, E., Lin, Y. C., Laslo, P., Cheng, J. X., Murre, C., Singh, H., and Glass, C. K. (2010). Simple combinations of lineage-determining transcription factors prime cis-regulatory elements required for macrophage and B cell identities. Mol Cell 38, 576-589.
Hsu, P. D., Scott, D. A., Weinstein, J. A., Ran, F. A., Konermann, S., Agarwala, V., Li, Y., Fine, E. J., Wu, X., Shalem, O., et al. (2013). DNA targeting specificity of RNA-guided Cas9 nucleases. Nat Biotechnol 31, 827-832.
Huang da, W., Sherman, B. T., Zheng, X., Yang, J., Imamichi, T., Stephens, R., and Lempicki, R. A. (2009). Extracting biological meaning from large gene lists with DAVID. Curr Protoc Bioinformatics Chapter 13, Unit 13 11.
Ingolia, N. T., Lareau, L. F., and Weissman, J. S. (2011). Ribosome profiling of mouse embryonic stem cells reveals the complexity and dynamics of mammalian proteomes. Cell 147, 789-802.
Jia, G., Fu, Y., Zhao, X., Dai, Q., Zheng, G., Yang, Y., Yi, C., Lindahl, T., Pan, T., Yang, Y. G., et al. (2011). N6-methyladenosine in nuclear RNA is a major substrate of the obesity-associated FTO. Nat Chem Biol 7, 885-887.
Konig, J., Zarnack, K., Rot, G., Curk, T., Kayikci, M., Zupan, B., Turner, D. J., Luscombe, N. M., and Ule, J. (2010). iCLIP reveals the function of hnRNP particles in splicing at individual nucleotide resolution. Nat Struct Mol Biol 17, 909-915.
Levin, J. Z., Yassour, M., Adiconis, X., Nusbaum, C., Thompson, D. A., Friedman, N., Gnirke, A., and Regev, A. (2010). Comprehensive comparative analysis of strand-specific RNA sequencing methods. Nat Methods 7, 709-715.
Livak, K. J., and Schmittgen, T. D. (2001). Analysis of relative gene expression data using real-time quantitative PCR and the 2(-Delta Delta C(T)) Method. Methods 25, 402-408.
Mali, P., Yang, L., Esvelt, K. M., Aach, J., Guell, M., DiCarlo, J. E., Norville, J. E., and Church, G. M. (2013). RNA-guided human genome engineering via Cas9. Science 339, 823-826.
Neff, A. T., Lee, J. Y., Wilusz, J., Tian, B., and Wilusz, C. J. (2012). Global analysis reveals multiple pathways for unique regulation of mRNA decay in induced pluripotent stem cells. Genome research 22, 1457-1467.
Quinlan, A. R., and Hall, I. M. (2010). BEDTools: a flexible suite of utilities for comparing genomic features. Bioinformatics 26, 841-842.
Rahl, P. B., Lin, C. Y., Seila, A. C., Flynn, R. A., McCuine, S., Burge, C. B., Sharp, P. A., and Young, R. A. (2010). c-Myc regulates transcriptional pause release. Cell 141, 432-445.
Schwartz, S., Agarwala, S. D., Mumbach, M. R., Jovanovic, M., Mertins, P., Shishkin, A., Tabach, Y., Mikkelsen, T. S., Satija, R., Ruvkun, G., et al. (2013). High-resolution mapping reveals a conserved, widespread, dynamic mRNA methylation program in yeast meiosis. Cell 155, 1409-1421.
Segal, E., Friedman, N., Kaminski, N., Regev, A., and Koller, D. (2005). From signatures to models: understanding cancer using microarrays. Nat Genet 37 Suppl, S38-45.
Segal, E., Friedman, N., Koller, D., and Regev, A. (2004). A module map showing conditional activity of expression modules in cancer. Nat Genet 36, 1090-1098.
Segal, E., Shapira, M., Regev, A., Pe'er, D., Botstein, D., Koller, D., and Friedman, N. (2003). Module networks: identifying regulatory modules and their condition-specific regulators from gene expression data. Nat Genet 34, 166-176.
Sharova, L. V., Sharov, A. A., Nedorezov, T., Piao, Y., Shaik, N., and Ko, M. S. (2009). Database for mRNA half-life of 19 977 genes obtained by DNA microarray analysis of pluripotent and differentiating mouse embryonic stem cells. DNA Res 16, 45-58.
Sigova, A. A., Mullen, A. C., Molinie, B., Gupta, S., Orlando, D. A., Guenther, M. G., Almada, A. E., Lin, C., Sharp, P. A., Giallourakis, C. C., et al. (2013). Divergent transcription of long noncoding RNA/mRNA gene pairs in embryonic stem cells. Proc Natl Acad Sci USA 110, 2876-2881.
Taghizadeh, K., McFaline, J. L., Pang, B., Sullivan, M., Dong, M., Plummer, E., and Dedon, P. C. (2008). Quantification of DNA damage products resulting from deamination, oxidation and reaction with products of lipid peroxidation by liquid chromatography isotope dilution tandem mass spectrometry. Nat Protoc 3, 1287-1298.
Trapnell, C., Hendrickson, D. G., Sauvageau, M., Goff, L., Rinn, J. L., and Pachter, L. (2013). Differential analysis of gene regulation at transcript resolution with RNA-seq. Nat Biotechnol 31, 46-53.
Trapnell, C., Pachter, L., and Salzberg, S. L. (2009). TopHat: discovering splice junctions with RNA-Seq. Bioinformatics 25, 1105-1111.
Trapnell, C., Williams, B. A., Pertea, G., Mortazavi, A., Kwan, G., van Baren, M. J., Salzberg, S. L., Wold, B. J., and Pachter, L. (2010). Transcript assembly and quantification by RNA-Seq reveals unannotated transcripts and isoform switching during cell differentiation. Nat Biotechnol 28, 511-515.
Tusher, V. G., Tibshirani, R., and Chu, G. (2001). Significance analysis of microarrays applied to the ionizing radiation response. Proc Natl Acad Sci USA 98, 5116-5121.
Ulitsky, I., Shkumatava, A., Jan, C. H., Sive, H., and Bartel, D. P. (2011). Conserved function of lincRNAs in vertebrate embryonic development despite rapid sequence evolution. Cell 147, 1537-1550.
Ventura, A., Meissner, A., Dillon, C. P., McManus, M., Sharp, P. A., Van Parijs, L., Jaenisch, R., and Jacks, T. (2004). Cre-lox-regulated conditional RNA interference from transgenes. Proc Natl Acad Sci USA 101, 10380-10385.
Wong, D. J., Liu, H., Ridky, T. W., Cassarino, D., Segal, E., and Chang, H. Y. (2008). Module map of stem cell genes guides creation of epithelial cancer stem cells. Cell Stem Cell 2, 333-344.
Xiao, R., and Moore, D. D. (2011). DamIP: using mutant DNA adenine methyltransferase to study DNA-protein interactions in vivo. Curr Protoc Mol Biol Chapter 21, Unit 21 21.

1. A method for maintaining a stem cell population in an undifferentiated state, comprising contacting the stem cell population with an inhibitor of METTL3 or METTL4.

2. The method of claim 1, wherein the stem cell population is a human stem cell population.

3. The method of claim 1, wherein the human stem cell population is a population of hESCs.

4. The method of claim 1, wherein the stem cell population is prevented from differentiating along an endoderm lineage.

5. The method of claim 1, wherein the inhibitor of METTL3 or METTL4 is a RNAi inhibitor or miRNA.

6. A method of promoting a stem cell population to differentiate along an endoderm lineage comprising contacting the stem cell population with an agent which increases m6A of mRNA in the stem cell population.

7. The method of claim 6, wherein the agent is a m6A methyltransferase.

8. The method of claim 7, wherein the m6A methyltransferase is METTL3 or METTL4. The method of claim 6, wherein the stem cell population is a human stem cell population.

9. A method to characterize a stem cell population, comprising performing m6A sequencing on the population of stem cells, and assessing the intensity of the m6A levels of the mRNA of at least 10 genes selected from any of those in Table 1 or Table 2.

10. An assay for assessing m6A levels in the RNA of at least 10 genes selected from any of those listed in Table 1, comprising contacting an array comprising at oligonucleotides that hybridize to at least 10 genes selected from any of Table 1 or Table 2 with RNA isolated from a cell population, and contacting the array with at least one reagent which binds to m6A in the RNA.

11. The assay of claim 10, wherein the reagent which binds to m6A is an anti-m6A antibody, or fragment thereof.

12. The assay of claim 11, wherein the anti-m6A antibody or fragment thereof is detectably labeled.

13. A method for determining the cell state of a stem cell population comprising performing the assay of claim 10, and comparing the levels of m6A of at least 10 genes selected from any of Table 1 in the RNA from the stem cell population with the levels of m6A in a reference stem cell population, and based on this comparison, determining the cell state of the stem cell population.

14. The method of claim 13, wherein the levels of m6A are peak intensity levels.

15. A kit comprising:
a. an array composition for characterizing the cell state of a population of stem cells, comprising at least 10 oligonucleotides that hybridize to the RNA of at least 10 genes selected from any of those in Table 1; and
b. at least one regent to detect the m6A in RNA.

16. The kit of claim 15, wherein the regent is an anti-m6A antibody, or fragment thereof.

17. The kit of claim 16, wherein the anti-m6A antibody or fragment thereof is detectably labeled.

18. A culture media comprising an inhibitor of METTL3 or METTL4.

19. The culture media of claim 18, wherein the culture media is a cryopreservation media.

20. The culture media of claim 18, further comprising a population of human stem cells.
